(19) United States
(12) Patent Application Publication
(10) Pub. No.: US 2006/0053500 A1
(43) Pub. Date: Mar. 9, 2006

(54) Modification of sugar metabolic processes in transgenic cells, tissues and animals

(75) Inventor: Chihiro Koike, Pittsburgh, PA (US)

Correspondence Address: KING & SPALDING LLP, 191 PEACHTREE STREET, N.E., 45TH FLOOR, ATLANTA, GA, 30303-1763, US

(73) Assignee: Univ. of Pittsburgh of the Commonwealth System of Higher Education, Office of Technology Management, Pittsburgh, PA (US)

(21) Appl. No.: 11/141,611

(22) Filed: May 31, 2005

Related U.S. Application Data
(60) Provisional application No. 60/575,539, filed on May 28, 2004.

(51) Int. Cl. A01K 67/027; C12P 21/06; C12N 9/10

(52) U.S. Cl. 800/8; 435/69.1; 435/193; 435/320.1; 435/325; 800/17

(57) ABSTRACT

The present invention provides natural or transgenic galactose deficient cells, tissues, organs and animals that have been genetically modified to compensate for the abnormalities in galactose metabolic pathways. The present invention modifies sugar metabolic pathways to to prevent the deleterious accumulation of sugar metabolites in animals, tissues, organs, cells and cell lines that possess natural or transgenic abnormalities in the sugar metabolic pathways. Such cells, tissues, organs and animals can be used in research and medical therapy, including xenotransplantation.

This application claims priority to U.S. Provisional Application No. 60/575,539, filed on May 28, 2004, which is herein incorporated by reference in its entirety.

### FIELD OF THE INVENTION

The present invention provides natural or transgenic galactose deficient cells, tissues, organs and animals that have been genetically modified to compensate for the abnormalities in galactose metabolic pathways. The present invention modifies sugar metabolic pathways to to prevent the deleterious accumulation of sugar metabolites in animals, tissues, organs, cells and cell lines that possess natural or transgenic abnormalities in the sugar metabolic pathways. Such cells, tissues, organs and animals can be used in research and medical therapy, including xenotransplantation.

### BACKGROUND OF THE INVENTION

Metabolism can be defined as the sum of all enzyme-catalyzed reactions occurring in a cell. Metabolism is highly coordinated, and individual metabolic pathways are linked into complex networks through common, shared substrates. A series of nested and cascade feedback loops are employed to allow flexibility and adaptation to changing environmental conditions and demands. Negative feedback prevents the over-accumulation of intermediate metabolites and contributes to the maintenance of homeostasis in the cell.

Understanding the mechanisms involved in metabolic regulation has important implications in both biotechnology and medicine. For example, it is estimated that one third of all serious health problems such as coronary heart disease, diabetes, and stroke are caused by metabolic disorders. Due to the highly coordinated nature of metabolism, it is often difficult to predict how changing the activity of a single enzyme will affect the entire reaction pathway.

Metabolism has two essential functions. First, it provides the energy required to maintain the internal composition of the cell and support its functions. Second, it provides the metabolites the cell requires to synthesize its constituents and products.

Carbohydrates play a major role in metabolism. Carbohydrates, also known as saccharides, are essential components of all living organisms and they are the most abundant class of biological molecules. Carbohydrates serve as energy sources and cell wall components. The metabolic pathways of monosaccharides such as glucose have been extensively studied and characterized.

Research focusing on sugar chains residing on the surface of cells began with the discovery of the ABO-blood type by Karl Landsteiner in 1900. Since then, large numbers of sugar chains have been identified. Such knowledge led to the development of modern medical practices, including transfusion and transplantation. Carbohydrates also serve as molecules that allow environmental recognition, including cell-cell and cell-antibody recognitions (Medical Biochemistry 4^{th} Ed. Bhagavan, N. V. Harcourt Brace & Co., New York; Lippincott's Illustrated Reviews: Biochemistry 2^{nd} Ed. Champs, P. C., Harvey, R. A. Lippincott Williams & Wilkins. Philadelphia, Pa. (1994)). This type of recognition between cells in part allows for the idenitification of “self” versus “non-self”, and can contribute to complex medical issues, such as those involved with xenotransplantation. These fundamental discoveries, coupled with modern molecular biology and animal cloning technology, have resulted in new possibilities that may render xenotransplantation feasible (Phelps, C. et al. Science 299, 411414 (2003)).

The basic units of carbohydrates are known as monosaccharides. The metabolic breakdown of monosaccharides provides most of the energy used to power biological processes. Monosaccharides, or simple sugars, are aldehyde or ketone derivatives of straight-chain polyhydroxyl alcohols containing at least three carbon atoms. The most common monosaccharides include glucose, galactose, and fructose, which can be linked to form more complex sugars, including disaccharides such as lactose and maltose, as well as polysaccharides such as glycogen and cellulose.

The internal equilibrium of the body, known as homeostasis, involves the maintenance of a constant rate of concentration in the blood and cellular environment of certain molecules and ions that are essential to cellular function and maintenance. Homeostasis is largely maintained through metabolic processes. Sugars, and particularly monosaccharides, play an important role in this cellular homeostasis through their roles in a large number of cellular pathways and reactions of the metabolic process. Claude Bernard first proposed the concept of “homeostasis” in 1865, which was extended by Lewis B. Cannon in 1932.

Sugar metabolism is highly regulated, with multiple feedback mechanisms and controls. Sugar chains serve as a reservoir for un-utilized galactose and its metabolites. This mechanism helps maintain blood galactose concentrations at certain physiological levels. Even after sporadic ingestion of lactose or intravenous administration of galactose, the blood galactose level is relatively constant compared to glucose (Medical Biochemistry 4^{th} Ed. Bhagavan, N. V. Harcourt Brace & Co., New York). Abnormalities in the mechanisms of sugar metabolism can lead to phenotypic manifestations ranging from mild irritations to life threatening conditions, due largely to the toxic accumulation of sugar metabolites. Illustrative of this are the phenotypic manifestations associated with galactose sugar metabolism disruptions, which indicate the importance this particular monosaccharide plays in the maintenance of cellular homeostasis.

Galactose

galactose is a hexose sugar found in the disaccharide lactose, and a major component of many cellular reactions. Lactose (β-galactosyl-(1→4)-glucose) can be synthesized in the mammary gland by lactose synthase. The donor sugar is UDP-galactose and the acceptor sugar is glucose. Upon digestion, the disaccharide lactose is cleaved by the enzyme lactase into glucose and galactose in the small intestine.

Organisms lacking the ability to digest lactose suffer from a number of phenotypic manifestations. Since the 1930s it has been known that cataracts can be experimentally generated in many animals by either inducing diabetes in the animal or feeding the animals a diet high in lactose (Albert, D. M., Jakobiec, F. A. Ed. Principles and Practice of Ophthalmology. Chapter 9. pp. 152. W.B.Saunders Co., Philadelphia (1994); Segal, S., Berry, G. Disorder of galactose Metabolism. Chapter 25. p. 967-1000). It was further demonstrated in 1954 that galactose supplementation could accelerate the rate and severity of diabetic cataract formation (Albert, D. M., Jakobiec, F. A. Ed. Principles and Practice of Ophthalmology. Chapter 9. pp. 152. W.B.Saunders Co., Philadelphia (1994); Segal, S., Berry, G. Disorder of galactose Metabolism. Chapter 25. p. 967-1000). These dietary manipulations, however, do not lead to cataract formation in mice, which has led to the hypothesis that the mouse may be a highly galactose tolerant species.

Lactate deficient humans suffer from gastrointestinal problems, such as diarrhea, and metabolic acidosis can result in these people after ingestion of lactose (Medical Biochemistry 4^{th} Ed. Bhagavan, N. V. Harcourt Brace & Co., New York; Albert, D. M., Jakobiec, F. A. Ed. Principles and Practice of Ophthalmology. Chapter 9. pp. 152. W.B. Saunders Co., Philadelphia (1994); Segal, S., Berry, G. Disorder of galactose Metabolism. Chapter 25. p. 967-1000). Additional manifestations of congenital lactate intolerance in humans includes vomiting, failure to thrive, dehydration, disacchariduria including lactosuria, renal tubular acidosis, aminoaciduria, and liver damage (Hirashima, Y. et al. Europ. J Pediat. 130: 41-45 (1979); Hoskova, A. et al. Arch. Dis. Child. 55: 304-316, (1980); Russo, G. et al. Acta Paediat. Scand. 63: 457-460 (1974)).

Galactose in Sugar Catabolism (FIGS. 1A, 2, 3)

Once in the cell, galactose can enter the glycolysis pathway via its conversion to glucose, and thus serves as a major energy source in sugar catabolism. Galactose, like glucose, has six carbons. Galactose differs from glucose only in the stereochemistry of the C4 carbon. Despite this high degree of similarity, the highly specific enzymes of carbohydrate metabolism require the conversion of galactose to glucose before it can enter glycolysis. The metabolic pathway for the galactose conversion to glucose includes: 1) galactose being phosphorylated at C1 by ATP in a reaction catalyzed by galactokinase (GALK) to produce galactose-1-phosphate (Gal-1-P); 2) galactose-1-phosphate uridyl transferase (GALT) transfers the uridyl group of UDP-glucose to galactose-1-phosphate to yield glucose-1-phosphate (G-1-P) and UDP-galactose by the reversible cleavage of UDP-glucose's pyrophosphoryl bond; 3) UDP-galactose-4-epimerase (GALE) converts UDP-galactose back to UDP-glucose through the sequential oxidation and reduction of the hexose C4 atom; 4) glucose-1-phosphate (G-1-P) is converted to the glycolytic intermediate glucose-6-phosphate (G-6-P) by phosphoglucomutse; and 5) glucose-6-phosphate enters the glycolytic/hexosamine pathway (See FIG. 3).

GALE activity is highly regulated in the cell. In 1946, Stenstam reported that galactose metabolism by GALE was inhibited by ethanol administration (Chylack, L. T. Jr, Friend, Exo. Eye Res. 50, 575-582 (1990)). In 1961, Isselbacher and Krane noted that intracellular pH is an important factor in the GALE reaction (Isselbacher, K. J., Krane, S. M. J. Biol. Chem. 236, 2394-2398 (1961)). In 1965 Robinson et al confirmed that NADH and a higher hydrogen concentration (i.e., intracellular acidosis) inhibited GALE reactions (Robinson, E. A. et al. Biol. Chem. 241, 2737-2745 (1966)).

Deficiencies in each one of the enzymes involved in sugar catabolism can result in disease conditions that are collectively known as galactosemias. Animal models of galactosemia have been generated to study these diseases. Early onset cataracts is one common indicator used to diagnose galactosemia in animal models. GALK knockout mice have been created, however, these mice do not form cataracts even when fed a high galactose diet. If GALK knockout mice are crossbred with transgenic mice that express a human aldose reductase gene (Ai, Y. et al. Hum. Mol. Genet. 9, 1821-1827 (2000)), then early onset cataracts develop. GALT-KO mice also do not develop early onset cataracts (Ning, C. et al. Mol. Genet. Metab. 72, 306-315 (2001)). Another interesting animal model is the neonatal kangaroo. Stephens et al. reported cataract formation accompanied with diarrhea in orphan kangaroos fed cow's milk during lactation due to enzyme deficiencies in galactokinase (GALK) and galactose 1-phosphate uridyl transferase (GALT) (Stephens, T. et al. Nature 248, 524-525 (1974)).

Mutations in galactose-1-phosphate uridyl transferase (GALT) in humans also result in the clinical manifestation known as classical galactosemia. It is characterized by a failure to thrive, cataracts, hepatomegaly, progressive liver dysfunction, ovarian failure due to hypergonadotropic, hypogonadism, elevated blood galactose urine reducing substances (galactosuria), hyperchloremic metabolic acidosis, aminoaciduria, elevated liver enzymes, and albuminuria (see #230400 galactosemia in the Online Mendalian Inheritance in Man (OMIM) database, available at: http://www.ncbi.nlm.nih.gov/htbin-post/Omim). Deficiencies in the galactose 4-epimerase (GALE) enzyme lead to similar clinical manifestations as those seen in galactosemia (see, for example, OMIM # 230350-galactose Epimerase Deficiency). The most common disorder associated with deficiencies in the galactokinase (GALK) enzyme is the development of cataracts (Bosch, A. M. et al. J. Inherit. Metab. Dis. 25: 629-634 (2002)).

Galactose in the Hexosamine Pathway (FIG. 4)

Galactose also plays a role in the hexosamine pathway. In the hexosamine pathway, discovered by LeLoir (Albert, D. M., Jakobiec, F. A. Ed. Principles and Practice of Ophthalmology. Chapter 9. pp. 152. W.B.Saunders Co., Philadelphia (1994); Segal, S., Berry, G. Disorder of Galactose Metabolism. Chapter 25. p. 967-1000), N-acetylated sugars are produced in the coupling reaction with glutamine and the rate-limiting enzyme glutamine:fructose-6-phosphate amidotransferase (GFAT) (EC1.6.1.16). The amide nitrogen of glutamine is transferred to F-6-P, producing glucosamine 6-P (Figure) and glutamate by the rate-limiting enzyme GFAT (glutamine:fructose-6-phosphate amidotransferase, EC 1.6.1.16). This is followed by the production of CMP-N-acetylneuraminic acids (CMP-NANA) and hexosamine such as UDP-GlcNAc and UDP-GalNAc (Medical Biochemistry 4^{th} Ed. Bhagavan, N. V. Harcourt Brace & Co., New York, Lippincott's Illustrated Reviews: Biochemistry 2^{nd} Ed. Champe, P. C., Harvey, R. A. Lippincott Williams & Wilkins. Philadelphia, Pa. (1994)). In the reaction, after galactose has been converted to glucose 6-phosphate (G-6-P), glucose 6-phosphate is converted to fructose-6-phosphate by the enzyme phosphoglucoisomerase. Fructose-6-phosphate (F-6-P) is then converted to glucosamine 6-phosphate with the concomitant conversion of glutamine to glutamate by glucosamine:fructose-6-phosphate amindotransferase (GFAT). Glucosamine 6-phosphate is then rapidly converted through a series of steps to produce UDP-GlcNac, UDP-GalNAc, and sialic acid (See FIG. 4).

GFAT controls the flux of glucose into the hexosamine pathway, and thus formation of hexosamine products, and is most likely involved in regulating the availability of precursors for N- and O-linked glycosylation of proteins. It is an insulin-regulated enzyme that plays a key role in the induction of insulin resistance in cultured cells. Increased flux of sugars through the hexosamine synthesis pathway has been implicated in the development of insulin resistance (Marshall et al. J. Biol. Chem. 266 (1991) 47064712). In addition, it was recently reported that a single nucleotide polymorphism (SNP) in the GFAT2 is associated with type 2 diabetes mellitus (Wakabayashi, S. et al. Physiol. Res. 77, 51-74 (1994)).

Sialic acids, generated through the hexosamine pathway (see FIG. 4), are ubiquitous and confer negative charges on cell surfaces (Medical Biochemistry 4^{th} Ed. Bhagavan, N. V. Harcourt Brace & Co., New York, Lippincott's Illustrated Reviews: Biochemistry 2^{nd} Ed. Champe, P. C., Harvey, R. A. Lippincott Williams & Wilkins. Philadelphia, Pa. (1994)). Sialic acids are distributed in all vertebrates (mammalian, Aves, reptilian, Amphibian, and Pisces) and ubiquitous in essentially all tissues (Ogiso, M et al Exp. Eye Res. 59, 653-663 (1994); T. Hennet, CMLS 59; 1081-1095: 2002). More than 20 sialyltransferases with different substrate specificity are known, comprising the sialyltransferase super family (Paulson, J. C., Colley, K. J. J. Biol. Chem. 264, 17615-17618 (1989)). The mammalian central nervous system has the highest sialic acid concentration. Total sialic acid concentration in the human brain is almost 2- to 4-fold that of eight other mammalian species, whose rank order is as follows: human, rat, mouse, rabbit, sheep, cow, and pig (Ogiso, M et al Exp. Eye Res. 59, 653-663 (1994); T. Hennet, CMLS 59; 1081-1095: 2002).

Importantly, the hexosamine synthesis process inevitably results in the production of hydrogen ions, as well as NH₃ (ammonia) (See FIG. 1A, 2, 4). The nitrogen cannot be stored, and amino acids in excess of the biosynthetic needs of the cell are immediately degraded (Medical Biochemistry 4^{th} Ed. Bhagavan, N. V. Harcourt Brace & Co., New York, Lippincott's Illustrated Reviews: Biochemistry 2^{nd} Ed. Champe, P. C., Harvey, R. A. Lippincott Williams & Wilkins. Philadelphia, Pa. (1994)) by the reactions of aminotransferase and glutamate dehydrogenase, forming ammonia and the corresponding α-ketoacids. These reactions are tightly regulated since even slight elevations concentration of ammonia can be toxic, particularly to brain cells. Thus, the hexosamine pathway is particularly important from the viewpoint of ammonia metabolism since the synthesis of nucleotide sugars such as sialic acids precludes the accumulation of and reduces the production of intracellular ammonia (FIGS. 1A, 2, 4).

The hexosamine pathway inevitably results in the production of hydrogen ions, which are generally excreted from the cell by the NHE (sodium-hydrogen exchanger) (Zhang, H. et al. J. Clin. Endo.& Metabol. 89, 748-755 (2004)) (See, for example, FIGS. 23 and 24). The NHE helps to maintain the intra- and extra-cellular pH within a narrow range (7.20±0.04, in general, and 7.40±0.04, respectively). Schultheis et al. generated mice lacking NHE function (Schultheis, P. J. et al. Nature Genet. 19: 282-285 (1998)). Homozygous mutant mice survived but suffered from diarrhea, and blood analysis revealed that they were mildly acidotic. NHE serves as a major Na(+)/H(+) exchanger in kidney and intestine. Loss of NHE function impairs acid-base balance and Na(+)-fluid volume homeostasis. Modifications in ammonia homeostasis can plays a role in the manifestation of certain diseases (see, for example, Seiler Neurochem Res. 1993 March; 18(3):235-45).

Galactose in Sugar Chain Synthesis (FIGS. 1B, 2, 5)

Galactose is also a prominent monosaccharide involved in sugar chain synthesis. Galactose is present in several classes of glycoconjugates, including N-glycans, O-linked GalNAc glycans, O-linked fucose glycans; glycosaminoglycans, galactosylceramide, and glycolipids. Galactose is transferred via several linkages to acceptor structures by a subset of glycotransferase enzymes (See FIG. 1) known as galactosyltransferases. In mammals, 19 distinct galactosyltransferases have been characterized to date (T. Hennet, CMLS 59; 1081-1095: 2002). Galactosyltransferases (GT) catalyze the addition of galactose in two anomeric configurations through α1-2, α 1-3, α 14, β1-6, β 1-3, or β 14 linkages in the following standard reaction: UDP-galactose+acceptor→Galacatose-acceptor+UDP. Through this linkage ability, galactosyltransferases serve as a shunt to transport galactose out of the cell via glycoconjugate linkages. The variety of galactosylation reactions significantly contributes to the tremendous diversity of oligosaccharide structures expressed by living organisms (T. Hennet, CMLS 59; 1081-1095: 2002). Evolutionary issues in relating oligosaccharide diversity to biological function have been the topic of much consideration (see, for example, Gagneux & Varki Glycobiology. 1999 August; 9(8):747-55).

The vast diversity of galactosylated structures in higher eukaryotes is paralleled by several GT gene duplication events that give rise to several groups of enzymes with different acceptor specificities and distinct patterns of tissue expression. The activity and biological functions of galactosyltransferases have been most thoroughly characterized in mammals. In mammals, galactose can occur β1-4, β1-3, α1-3 and α1-4 linked to accepting templates in various types of glycoconjugates. It was initially believed that a specific enzyme catalyzed each glycosidic linkage. However, the discovery of multiple isozymes for several glycosyltransferase activities has changed this ‘one linkage, one enzyme’ rule to become ‘one linkage, many enzymes’ (T. Hennet, CMLS 59; 1081-1095: 2002).

β-1,3-Galactosyltransferase (β-1,3-GT)

In the early eighties, Sheares et al. (Sheares et al. 1982 J. Biol. Chem. 257: 599-602; Sheares et al. 1983 J. Biol. Chem. 258: 9893-9898) identified a β-1,3-GT activity derived from pig trachea. They found that this β-1,3-GT activity was directed toward N-acetylgalactosaminyltransferase (GlcNAc)-based acceptors and was not inhibited by α-lactalbumin or by elevated GlcNAc concentrations. About ten years later, the first β-1,3-GT genes were cloned and characterized as recombinant proteins. At least seven β-1,3-GT genes have now been described. There is no significant homology between β-1,3-GT and β-1,3-GT proteins, suggesting a separate evolutionary lineage. In fact, β-1,3-GT share some similarities with bacterial galactosyltransferases such as LgtB and LgtE (Gotschlich 1994 J Exp Med 180:2181-2190). β-1,3-GT proteins are structurally related to β-1,3 GlcNAc-transferases (Zhou et al 1999 PNAS 97: 11673-11675; Shiraishi et al 2000 J Biol Chem 276: 3498-3507; Togayachi et al 2001 J Biol Chem 276: 22032-22040; Henion et al 2001 J Biol Chem 276: 30261-30269) indicating that the maintenance of a β1-3 linkage, rather than of the donor substrate, has dictated the conservation of domains within these proteins. The β-1,3-GT gene family encodes type II membrane-bound glycoproteins with diverse enzymatic functions.

β-1,4-Galactosyltransferase (β-1,4-GT)

At least seven β-1,4-GT enzymes have been described. These proteins share an extensive homology and encode type II membrane-bound glycoproteins that have specificity for the donor substrate UDP-galactose. Recent searches of mammalian genome databases using known β-1,4-GT sequences as queries has failed to reveal additional related genes. However, these searches do not exclude the existence of other β-1,4-GT genes that may present little structural similarity to the known enzymes. In most cases, the identity of β-1,4-GT proteins has been confirmed by heterologous expression of recombinant proteins. This approach establishes the enzymatic activity, but a comparison of the β-1,4-GT isozymes is difficult to address because the expression systems as well as the type of recombinant β-1,4-GT proteins often differ in the first reports. For example, the acceptor substrate specificity attributed to single β-1,4-GT may have to be revised or extended to the light of new experiments. A recent study investigating the specificity of six β-1,4-GT expressed under identical conditions showed that all the enzymes can transfer galactose to N-glycan acceptors (Guo et al. (2001) Glycobiology 11: 813-820).

β-1,4-GT knockout mice have been created. These mice exhibit growth retardation, semi-lethality, skin lesions, decreased fertility, an absence of lactose in milk (Asano et al. The EMBO Journal Vol. 16 No. 8 pp. 1850-1857, 1997), abnormalities of the intestine, and a lack of lactase in suckling mice. The lack of lactase (i.e., similar to lactose intolerance) may be a result of a negative feedback mechanism in response to the overload of UDP-galactose.

α-1,4-Galactosyltransferase (α-1,4-GT)

In mammals, the occurrence of α-1-4-linked galactose is restricted to glycolipids. α-1,4-GT activities have been related to the formation of Gb3 [Gal(α1-4)Gal(β1-4)Glc(β1-)ceramide], also known as the B cell differentiation marker CD77 (Mageney et al. (1991) Eur. J. Immunol. 21: 1131-1140), and to the formation of the P₁ glycolipid [Gal(α1-4)Gal(β1-4) GlcNAc(β1-3)Gal(β1-4)Glc(β1-)ceramide]. Differential presentation of the glycolipids P [GalNAc(β1-3)Gal(α1-4)Gal(β1-4)Glc(β1-)ceramide] and P₁ constitutes the basis of the P histo-blood group system (Carton (1996) Transfus. Clin. Biol. 3:181-210).

α-1,3-Galactosyltransferase (α1,3GT)

The α-1,3-GT gene and cognate α-1,3-galactose epitope have attracted special attention because of the immunological reciprocal relationship, similar to the ABO-histo blood type system (Medical Biochemistry 4^{th} Ed. Bhagavan, N. V. Harcourt Brace & Co., New York; Lippincott's Illustrated Reviews: Biochemistry 2^{nd} Ed. Champe, P. C., Harvey, R. A. Lippincott Williams & Wilkins. Philadelphia, Pa. (1994)). Except for Old World monkeys, apes and humans, most mammals carry glycoproteins on their cell surfaces that contain the α-1,3-galactose epitope (Galili et al., J. Biol. Chem. 263: 17755-17762, 1988). Humans, apes and Old World monkeys have a naturally occurring anti-alpha galactose antibody that is produced in high quantities (Cooper et al., Lancet 342:682-683, 1993). It binds specifically to glycoproteins and glycolipids bearing the α-1,3-galactose epitope.

The ramifications of this divergent α-1,3-galactose epitope expression has been apparent in recent attempts at xenotransplantation. A direct outcome of the divergent expression is the potential rejection of xenografts from an α-1,3-galactose epitope containing species to non-α-1,3-galactose epitope containing species, such as a porcine organ transplanted into a human, due to hyper acute rejection of the α-1,3-galactose epitope containing organ. A variety of strategies have been implemented to eliminate or modulate the anti-galactose humoral response caused by xenotransplantation, including enzymatic removal of the epitope with alpha-galactosidases (Stone et al., Transplantation 63: 640-645, 1997), specific anti-galactose antibody removal (Ye et al., Transplantation 58: 330-337, 1994), and the introduction of complement inhibitory proteins (Dalmasso et al., Clin. Exp. Immunol. 86: 31-35, 1991, Dalmasso et al. Transplantation 52:530-533 (1991)).

Another strategy that has received a lot of attention has been the capping of the α-1,3-galactose epitope with other carbohydrate moieties which failed to eliminate alpha-1,3-GT expression (Tanemura et al., J. Biol. Chem. 27321: 16421-16425, 1998 and Koike et al., Xenotransplantation 4: 147-153, 1997). Costa et al. (FASEB J 13, 1762 (1999)) reported that competitive inhibition of α-1,3-GT in H-transferase transgenic pigs results in only partial reduction in epitope numbers. Miyagawa et al. (J. Biol. Chem 276, 39310 (2001)) reported that attempts to block expression of galactose epitopes in N-acetylglucosaminyltransferase III transgenic pigs also resulted in only partial reduction of galactose epitopes numbers and failed to significantly extend graft survival in primate recipients.

Ramsoondar et al. (Biol of Reproduc 69, 437-445 (2003) reported the generation of heterozygous alpha-1,3-GT knockout pigs that also express human alpha-1,2-fucosyltransferase (HT), which expressed both the HT and alpha-1,3-GT epitopes.

U.S. Pat. No. 6,331,658 to Integris Baptist Medical Center, Inc. & Oklahoma Medical Research Foundation claims methods of making transgenic animals that express a sialyltransferase or a fucosyltransferase that results in a reduction of α1,3GT epitopes on the surface of at least some of the cells.

WO 02/074948 and U.S. 2003/0068818 to Geron Corporation describes methods for generating animal tissues with carbohydrate antigens that are compatible for xenotransplantation by inactivating both alleles of the α-1,3-GT allele and inserting an α-1,2-fucosyltransferase.

WO 95/34202 to Alexion Pharmaceuticals and the Austin Research Institute describes methods to produce xenogenic organs that express a protein having fucosyltransferase activity, which causes a substantial reduction in the binding of natural preformed human or Old World monkey antibodies.

WO 98/07837 and U.S. Pat. No. 6,399,758 to the Austin Research Institute describes nucleic acid contructs that encode a glycosyltransferase that is able to compete with a second glysosyltransferase for a subtrate. U.S. Pat. No. 6,399,758 claims a method of producing an isolated cell having reduced levels of Galα-1,3-Gal epitope on the cell surface wherein the carbohydrate epitope is recognized as non-self by a human, by transforming or transfecting said cell with a particular nucleic acid under conditions such that a specific porcine secretor glycosyltransferase is produced.

A more recent approach to reduce the immunogenicity of the α-1,3-galactose epitope has been to knock out the α-1,3-GT enzyme responsible for its addition. Single allele knockouts of the alpha-1,3-GT locus in porcine cells and live animals have been reported. Denning et al. (Nature Biotechnology 19: 559-562, 2001) reported the targeted gene deletion of one allele of the alpha-1,3-GT gene in sheep. Harrison et al. (Transgenics Research 11: 143-150, 2002) reported the production of heterozygous alpha-1,3-GT knock out somatic porcine fetal fibroblasts cells. In 2002, Lai et al. (Science 295: 1089-1092, 2002) and Dai et al. (Nature Biotechnology 20: 251-255, 2002) reported the production of pigs, in which one allele of the alpha-1,3-GT gene was successfully rendered inactive. Sharma et al. (Transplantation 75:430436 (2003) published a report demonstrating a successful production of fetal pig fibroblast cells homozygous for the knockout of the α-1,3-GT gene.

WO 01/30992 to the University of Pittsburgh describes the genomic sequence of the porcine α-1,3-GT gene and promoter as well as targeting cassettes to inactivate the porcine α-1,3-GT gene.

WO 01/23541 to Alexion Pharmaceuticals describes genomic sequence of the porcine α-1,3-GT gene as well as “promoter trap” gene targeting constructs to inactivate the α-1,3-GT gene.

An α-1,3-GT gene knockout mouse has been created (Shinkel, T. A. et al. Transplant. 64, 197-204 (1997); Tearle, R. G. et al. The α-1,3-glactosyltransferase knockout mouse. Transplantation. 61, 13-19 (1996); Thall, A. et al J. Biol. Chem. 270, 21437-21440 (1995)) as a research model for xenotransplanation (Cooper, D. K. et al Transplant. Immunol. 1, 198-205 (1993).). Studies on these animals have indicated that non-naturally occurring anti-α-1,3-Gal antibodies are produced in these mice and that there is an increase in the production of sialic acid moieties on the cell surface (Shinkel, T. A et al. Transplant. 64, 197-204 (1997).). In addition, α-1,3-GT knockout mice develop early onset bilateral cataracts (EOC, or opacity) (Tearle, R. G. et al. Transplantation. 61, 13-19 (1996)).

Phelps et al. recently reported the successful production of the first live pigs lacking any functional expression of alpha 1,3 galactosyltransferase (homozygous knockout animals) (Science 299:411-414 (2003); WO 04/028243).

IsoGloboside 3 (iGb3) Synthase

α-1,3-GT is not the only enzyme that synthesizes the Galα(1,3)Gal motif. IsoGloboside 3 (iGb3) synthase is also capable of synthesizing Galα-1,3-Gal motifs (Taylor S G, et al Glycobiology 13(5): 327-337 (2003)). Taylor et al. found that two independent genes encode distinct glycosyltransferases, α-1,3-GT and iGb3 synthase, and that both are capable of synthesizing the Galα-1,3-Gal motif (Taylor et al. (2003) Glycobiology 13(5):327-337). These separate and distinct glycosyltransferases act through two different glycosylation pathways. Transfection studies have shown that CL-1,3-GT synthesizes Galα-1,3-Gal on glycoproteins, whereas the synthesis of the Galα-1,3-Gal motif on the glycolipid is facilitated by iGB3 synthase. In addition, it has been shown that α-1,3-GT is incapable of synthesizing the Galα-1,3-Gal on glycolipids (Taylor et al. (2003) Glycobiology 13(5):327-337). These findings have refuted the previously held belief that α-1,3-GT was the sole Gal α(1,3)Gal motif synthesizing enzyme.

In contrast to α(1,3)GT, iGb3 synthase preferentially modifies glycolipids over glycoprotein substrates (Keusch et al. (2000) J. Bio. Chem. 275:25308-25314). iGb3 synthase acts on lactosylceramide (LacCer (Galβ1,4Glcβ1Cer)) to form the glycolipid isogloboid structure iGb3 (Galα1,3Galβ1,4Glcβ1Cer), initiating the synthesis of the isoglobo-series of glycoshingolipids.

The presence of the iGb3 synthase gene, and its contribution to the biosynthesis of the highly immunogenic Galα(1,3)Gal epitope, potentially presents an additional hurdle to overcome in the quest for the production of immuno-tolerable xenotransplants.

Keusch J J et al have previously reported the cloning of the rat iGb3 synthase gene (J. Biol. Chem 2000). The gene is reported as GenBank sequence NM 138524.

PCT Publication No. WO 02/081688 to The Austin Research Institute discloses a partial cDNA sequence encoding a portion of exon 5 (480 base pairs) of the porcine iGb3 synthase gene. This application also discloses a cell in which the iGb3 synthase gene has been disrupted and an α-1,2-fucosyltransferase gene has been inserted. This application further purports to cover the use of this DNA sequence to disrupt this gene in cells, tissues and organs for xenotransplantation.

PCT publication No. WO 05/04769 by the University of Pittsburgh provides porcine isolgloboside 3 synthase protein, cDNA, genomic organization and regulatory regions. In addition WO 05/04769 also describes porcine animals, tissue and organs as well as cells and cell lines derived from such animals, tissue and organs, which lack expression of functional porcine iGb3 synthase, for use in in research and in medical therapy, including xenotransplantation.

Depletion of the glycoconjugates that contain the α1,3 galactose epitope by eliminating the enzyme(s) responsible for its addition is an advantageous approach for the production of animals for xenotransplantation. The ramifications of knocking out the α1,3GT continue to be evaluated.

Forssman Synthetase

Glycolipids that contain the Forssman (FSM) antigen (pentaglycosylceramide) (GalNAcα(1,3)GalNAcβ(1,3)Galα(1,4)Galβ(1,4)Glcβ(1,1)Cer) are found on the cells of many mammals, including pigs (Copper et al. (1993) Transplant Immunol 1:198-205). This antigen is chemically related to the human A, B, and O blood antigens. However, the glycolipids of Old World monkeys, apes, and humans do not normally contain FSM antigens, although certain malignancies in humans have been shown to express this particular antigen (Hansson G C et al. (1984) FEBS Lett. 170:15-18; -Stromberg N et al. (1988) FEBS Lett. 232:193-198). Although humans do express the FSM antigen precursor globotriaosylceramide (Xu H et. al. (1999) 274(41):29390-29398), it is not converted to the FSM antigen. In other mammals, the modification of this FSM antigen precursor with the addition of an N-acetylgalactosamine via the FSM synthetase enzyme creates the Forssman antigen.

Because humans lack the FSM antigen, exposure to discordant cells, tissues or organs containing the antigen can lead to the development of anti-FSM antigen antibodies. This antibody development can ultimately play a role in the rejection of FSM antigen containing xenografts. Because pig cells express FSM antigen (see, for example, Cooper et al. (1993) Transplant Immunol 1:198-205), the use of pig organs in a xenotransplant strategy could potentially be compromised due to the potential of organ rejection induced by the FSM antigen.

Haslam D B et al. (Biochemistry 93:10697-10702 (1996) describes a cDNA sequence that encodes for canine Forssman synthetase isolated from a canine kidney cDNA library.

Xu H et al. (J. Bio. Chem. 274(41):29390-29398 (1999) describe a cDNA sequence that encodes for human Forssman synthetase isolated from human brain and kidney cDNA libraries.

U.S. Pat. No. 6,607,723 to the Alberta Research Council and Integris Baptist Medical Center describes removing preformed antibodies to various identified carbohydrate xenoantigens, including the FSM antigen, from a recipient's circulation prior to transplantation. The method provides for the extracorporeal perfusion of the recipient's blood over a biocompatible solid support to which the xenoantigens are bound and/or parenterally administering a xenoantibody-inhibiting amount of an identified xenoantigen to the recipient shortly before graft revascularization.

U.S. Pat. App. No. 2003/0153044 to Liljedahl et al. discloses a partial cDNA sequence, including portions of exons 4, 5, 6, and 7, of the porcine Forssman synthetase gene.

PCT Publication No. WO 04/108904 to Univerity of Pittsburgh provides the full length cDNA sequence, peptide sequence, and genomic organization of the porcine CMP-Neu5Ac hydroxylase gene. In addition, this publication provides porcine animals, tissues, and organs, as well as cells and cell lines derived from such animals, tissue, and organs, which lack expression of functional CMP-Neu5Ac hydroxylase, which can be used in research and medical therapy, including xenotransplantation.

N-acetylgalactosaminyltransferases (GalNAcT)

N-acetylgalactosaminyltransferases can catalyze the addition of N-acetylgalactosamine in anomeric configurations through specific linkages, such as α 1-4 (α-1,4-N-acetylgalactosaminyltransferase) and β 1-4 (β-1,4-N-acetylgalactosaminyltransferase), in the following standard reaction: UDP-N-acetylgalactosamine+acceptor→N-acetylgalactosamine-acceptor+UDP. GALNACTs initiate mucin-type O-linked glycosylation in the Golgi apparatus by catalyzing the transfer of GalNAC.

N-acetylglucosaminyltransferases

Glucose N-acetylglucosaminyltransferases can catalyze the addition of N-acetylglucosamine in anomeric configurations through specific linkages, such as β 1-3 (β-1,3-N-acetylglucosaminyltransferases; Sasaki et al. (1997) PNAS 94: 14294-14299) and β 1-6 (β-1,6-N-acetylglucosaminyltransferases), in the following standard reaction: UDP-N-acetylglucosamine+acceptor→N-acetylglucosamine-acceptor+UDP.

β-1,6-N-acetylglucosaminyltransferase is a branching enzyme. The human i and I antigens are characterized as linear and branched repeats of N-acetyllactosamine, respectively. Expression of i and I antigens has a reciprocal relationship and is developmentally regulated, the i antigen is expressed on fetal and neonatal red blood cells, whereas the I antigen is predominantly expressed on adult red blood cells. After birth, the quantity of i antigen gradually decreases, while the quantity of I antigen increases. The tandem repeats of NA-Lac dramatically changes from the linear type (i.e., “i-antigens”) to the branched type (i.e., “I-antigen”) beginning with the addition of GlcNAc molecules through the activity of β-1,6-N-acetylglucosaminyltransferase during lactation periods (24,25). The normal Ii status of red blood cells is reached after about 18 months of age. Conversion of the i to the I structure requires I-branching beta-1,6-N-acetylglucosaminyltransferase activity. It has been noted that the null phenotype of I, the adult i phenotype, is associated with congenital cataracts (Yu et al. Blood. 2003 Mar. 15; 101(6):2081-8).

The complex regulation of galactose plays a central role in cellular homeostasis given its pivotal role in the catabolism of sugars and sugar chain synthesis. Disruption of the galactose pathway can lead to the accumulation of toxic metabolites, which can lead to the disruption of cellular homeostasis.

It is an object of the present invention to provide methods for modifying sugar metabolic pathways in cells, tissues, organs, and animals to compensate for abnormalities in the sugar metabolic pathways.

It is another object of the present invention to provide cells, tissues, organs, and animals that have been modified to compensate for abnormalities in the sugar metabolic pathways.

It is a futher object to provide natural or transgenic galactose deficient cells, tissues, organs and animals that have been genetically modified to compensate for the abnormalities in galactose metabolic pathways.

### SUMMARY OF THE INVENTION

The present invention provides natural or transgenic galactose deficient cells, tissues, organs and animals that have been genetically modified to compensate for the abnormalities in galactose metabolic pathways. In particular, the present invention provides cells, tissues, organs and animals that have been genetically modified to compensate for abnormalities in galactose metabolic pathways to prevent the toxic accumulations of galactose metabolites. Such abnormalities can be either endogenously present, such as an in-born genetic defect, or genetically engineered, in the galactose deficient cell, tissue, organ or animal. The present invention provides methods to compensate for these abnormalities by genetically modifying the galactose deficient cells, tissues, organs and/or animals to express at least one additional protein of the galactose metabolic pathway. The cells, organs, tissues and animals of the present invention are useful as medical therapeutics, particularly in xenotransplanatation.

Proteins involved in galactose metabolism include proteins associated with sugar catabolism, the hexosamine pathway and sugar chain synthesis. Proteins involved in sugar catabolism include, but are not limited to, galactokinase (GALK), galactose-1-phosphate uridyl transferase (GALT) and UDP-galactose-4-epimerase (GALE). Proteins associated with the hexosamine pathway include, but are not limited to, glutamine: fructose-6-phosphate amidotransferase (GFAT), the sodium-calcium exchanger (NCX) and the sodium-hydrogen exchanger (NHE). Proteins associated with sugar chain synthesis include, but are not limited to, β-1,3-galactosyltransferase (β-1,3-GT), β1,4-galactosyltransferase (β-1,4-GT), α-1,4-galactosyltransferase (α-1,4-GT), α-1,3-galactosyltransferase (α-1,3-GT), IsoGlobide 3 synthase (iGb3), Forssman synthase (FSM), N-acetylgalactosaminyltransferases (GalNAcT), and N-acetylglucosaminyltransferases (GlcNAc-T), such as β-1,6 GlcNac-T.

In particular embodiments of the present invention, the protein of the galactose metabolic pathway that is used to compensate for the galactose deficiency is a non-xenogenic protein (i.e., does not cause rejection when transplanted into another species). In one embodment, the non-xenogenic protein is present in both the donor species, for example, but not limited to, pig, and the recipient speicies, for example, but not limited to human. In a particular embodiment, the non-xenogenic protein is any protein in the galactose metabolic pathway, such as those described above, except the following: alpha-1,3-galactosyltransferase, the Forssman synthetase and/or isoGloboside 3 (iGb3) synthase.

In one aspect of the invention, transgenic cells, tissues, organs and animals are provided in which at least one allele of the alpha-1,3-galactosyltransferase gene, the Forssman synthetase gene and/or the isoGloboside 3 (iGb3) synthase gene has been inactivated, which have been genetically modified to express at least one additional protein associated with sugar catabolism, the hexosamine pathway, or sugar chain synthesis. Alternatively, animals, tissues, organs and cells are provided in which both alleles (homozygous knock-outs) of the alpha-1,3-galactosyltransferase (α-1,3-GT) gene, the Forssman synthetase gene and/or the isoGloboside 3 (iGb3) synthase gene have been rendered inactive, which have been genetically modified to express at least one additional protein associated with galactose transport. Proteins involved in galactose transport can include, but are not limited to proteins involved in sugar catabolism, the hexosamine pathway, or sugar chain synthesis. These genetic modifications decrease the accumulation of toxic metabolites, such as UDP-galactose (UDP-Gal) or UDP-N-acetyl-D-galactosamine (UDP-GalNAc), which result from the inactivation of the alpha-1,3-galactosyltransferase gene, the Forssman synthetase gene and/or the isoGloboside 3 (iGb3) synthase gene.

In one embodiment, cells, tissues, organs and animals are provided that lack functional expression of the alpha-1,3-galactosyltransferase (α-1,3-GT) gene, which have at least one additional protein associated with galactose transport, such as sugar catabolism associated proteins, such as GALE, hexosamine pathway associated proteins, such as GFAT and/or NHE, or sugar chain synthesis associated proteins, such as β-1,3-GT, β-1,4-GT, α-1, 4-GT, α-1,4-GalNAcT, β-1,4-GalNAcT, β-1,3-GlcNAcT and/or β-1,6-GlcNAcT inserted into their genome. These sugar-related proteins from any known prokaryote or eukaryote, such as humans or porcine, can be inserted into the genome via random or targeted insertion, or expressed transiently. These proteins can be under the control of the endogenous α-1,3-GT promoter or a constitutively active promoter, such as a housekeeping gene promoter or viral promoter.

In an alternative embodiment, animals, tissues, organs and cells are provided that lack functional expression of the isoGloboside 3 (iGb3) synthase gene, which have at least one additional protein associated with galactose transport, such as sugar catabolism associated proteins, such as GALE, hexosamine pathway associated proteins, such as GFAT and/or NHE, or sugar chain synthesis associated proteins, such as β-1,3-GT, β-1,4-GT, α-1,4-GT, α-1,4-GalNAcT, β-1,4-GalNAcT, β-1,3-GlcNAcT and/or β-1,6-GlcNAcT inserted into their genome. These sugar-related proteins from any known prokaryote or eukaryote, such as humans or porcine, can be inserted into the genome via random or targeted insertion, or expressed transiently. These proteins can be under the control of the endogenous iGb3 synthase promoter or a constitutively active promoter, such as a housekeeping gene promoter or viral promoter.

In another embodiment, animals, tissues, organs and cells are provided that lack functional expression of the Forssman (FSM) synthetase gene, which have at least one additional protein associated with galactose transport, such as sugar catabolism associated proteins, such as GALE, hexosamine pathway associated proteins, such as GFAT and/or NHE, or sugar chain synthesis associated proteins, such as β-1,3-GT, β1,4-GT, α-1,4-GT, α-1,4-GalNAcT, β-1,4-GalNAcT, β1,3-GlcNAcT and/or β-1,6-GlcNAcT inserted into their genome. These sugar-related proteins from any known prokaryote or eukaryote, such as humans or porcine, can be inserted into the genome via random or targeted insertion, or expressed transiently. These proteins can be under the control of the endogenous Forssman synthetase promoter or a constitutively active promoter, such as a housekeeping gene promoter or a viral promoter.

Another aspect of the present invention provides nucleic acid constructs that contain cDNA encoding galactose transport-related proteins, such as those associated with sugar catabolism, such as GALE, the hexosamine pathway, such as GFAT and/or NHE, or sugar chain synthesis, such as β-1,3-GT, β-1,4-GT, α-1,4-GT, α-1,4-GalNAcT, β1,4-GalNAcT, β-1,3-GlcNAcT and/or β-1,6-GlcNAcT. These cDNA sequences can be derived from any prokaryotic or eukaryotic nucleic acid sequence that encodes for a galactose transport-related protein. The construct can contain a single cassette encoding a single galactose transport-related protein (see, for example, FIG. 9), double cassettes (see, for example, FIG. 10) encoding two galactose transport-related proteins, or multiple cassettes encoding more than two galactose transport-related proteins. Constructs can further contain one, or more than one, internal ribosome entry site (IRES). The construct can also contain a promoter operably linked to the nucleic acid sequence encoding galactose transport-related proteins, or, alternatively, the construct can be promoterless. The nucleic acid constructs can further contain nucleic acid sequences that permit random or targeted insertion into a host genome.

In one embodiment, the nucleic acid construct contains a single cassette encoding a galactose transport-related protein, such as GALE, GFAT, NHE, NCX, β1,3-GT, β1,4-GT, α-1,4-GT, α-1,4-GalNAcT, β1,4-GalNAcT, β-1,3-GlcNAcT and β1,6-GlcNAcT (see, for example, FIG. 9). In another embodiment, the nucleic acid construct contains more than one cassette encoding the same galactose transport-related protein. In still another embodiment, the nucleic acid construct contains more than one cassette encoding more than one galactose transport-related protein in combination. Such combination include, but are not limited to, β-1,6-GlcNAcT and β-1,4-GT, β-1,3-GlcNAcT and β-1,4-GT, β-1,3-GlcNAcT and NHE, β-1,3-GT and α-1,4-GT, and NHE and NCX (see, for example, FIG. 10).

Nucleic acid constructs useful for targeted insertion of the galactose transport-related cDNA can include 5′ and 3′ recombination arms for homologous recombination. In one embodiment, targeting vectors are provided wherein homologous recombination in somatic cells can be rapidly detected. These targeting vectors can be transformed into mammalian cells to target a gene via homologous recombination. In one embodiment, the targeting vectors can target a gene associated with galactose transport. In another embodiment, the targeting construct can target a house keeping gene. In a further embodiment, the targeting construct can target a galactose transport-related gene that has been rendered inactive. In another embodiment, the targeting construct can target a galactose transport-related gene or a housekeeping gene so as to be in reading frame with the upstream sequence, which can allow it to be expressed under the control of the endogenous promoter of the galactose transport-related or housekeeping gene. In an alternate embodiment, the targeting construct can be constructed to render the galactose transport-related gene inactive, i.e., it can be used to knock-out the gene. In another embodiment, the targeting construct also contains a selectable marker gene. Cells can be transformed with the constructs using the methods of the invention and are selected by means of the selectable marker and then screened for the presence of recombinants.

In another embodiment, the targeting vectors can contain a 3′ recombination arm and a 5′ recombination arm that is homologous to the genomic sequence of a galactose-related gene, such as, but not limited to the α-1,3-GT, iGb3 or the FSM gene (see, for example, FIGS. 14A-E, 15-17). The homologous DNA sequence can include at least 10 bp, 15 bp, 20 bp, 25 bp, 50 bp, 100 bp, 500 bp, 1 kbp, 2 kbp, 4 kbp, 5 kbp, 10 kbp, 15 kbp, 20 kbp, or 50 kbp of sequence homologous to the galactose transport-related gene. In another embodiment, the homologous DNA sequence can include intron and exon sequence. In a specific embodiment, the DNA sequence can be homologous to Intron 2, Exon 2 and/or Intron 3 of the α-1,3-GT gene (see, for example, FIGS. 14A, 14B, 14C, 15). In another specific embodiment, the DNA sequence can be homologous to Intron 2 and/or Exon 2 of the iGb3 synthase gene (see, for example, FIGS. 14A, B, D, 15). In a further specific embodiment, the DNA sequence can be homologous to Intron 2, Exon 2, Exon 6 and/or Intron 7 of the FSM synthase gene (see, for example, FIGS. 14A, 14B, 14E, 15).

Another aspect of the present invention provides methods to produce a cell which has at least one additional protein (referred to herein as “sugar-related proteins”) associated with sugar catabolism, such as GALE, the hexosamine pathway, such as GFAT and/or NHE, or sugar chain synthesis, such as β-1,3-GT, β-1,4-GT, α-1,4-GT, α-1,4-GalNAcT, β-1,4-GalNAcT, β-1,3-GlcNAcT and/or β-1,6-GlcNAcT transfected into a cell that already lacks functional expression of α1,3-galactosyltransferase, iGb3 synthase, Forssman synthetase, or another gene associated with xenotransplant rejection. In one embodiment, the nucleic acid construct can be transiently transfected into the cell. In another embodiment, the nucleic acid construct can be inserted into the genome of the cell via random or targeted insertion. In a further embodiment, the contruct can be inserted via homologous recombination into a targeted genomic sequence within the cell such that it can be under the control of an endogenous promoter. In a specific embodiment, the nucleic acid construct can be inserted into the α1,3-galactosyltransferase genomic sequence, iGb3 synthase genomic sequence, Forssman synthetase genomic sequence, or a xenotransplant rejection-associated genomic sequence via homologous recombination such that the galactose transport-related cDNA can be under the control of the α-1,3-GT, iGb3 synthase or FSM promoter (see, for example, FIGS. 20, 21, 22).

In one embodiment of the present invention, the cells provided herein can be used as xenografts in cell transplantation therapy. Accordingly, there is provided in a further aspect of the invention a method of therapy comprising the administration of genetically modified transgenic cells which have at least one sugar-related protein associated with sugar catabolism transfected into a cell that already lacks functional expression of α1,3-galactosyltransferase, iGb3 synthase, Forssman synthetase, or a gene associated with xenotransplant rejection to a patient. In one embodiment, an animal can be prepared by a method in accordance with any aspect of the present invention. The genetically modified animals can be used as a source of cells, tissues and/or organs for human transplantation therapy. In one embodiment, an animal embryo prepared in this manner or a cell line developed therefrom can also be used in cell-transplantation therapy. In one embodiment, the animal utilized is a pig. This aspect of the invention can include the use of such cells in medicine, e.g. cell-transplantation therapy, and also the use of cells derived from such embryos in the preparation of a cell or tissue graft for transplantation. The cells can be organized into tissues or organs, for example, heart, lung, liver, kidney, pancreas, corneas, nervous (e.g. brain, central nervous system, spinal cord), skin, or the cells can be islet cells, blood cells (e.g. haemocytes, i.e. red blood cells, leucocytes) or haematopoietic stem cells or other stem cells (e.g. bone marrow).

Another aspect of the present invention includes methods for modifying sugar metabolic processes within a cell by inserting a nucleic acid construct encoding at least one sugar-related protein associated with sugar catabolism, such as GALE, the hexosamine pathway, such as GFAT and/or NHE, or sugar chain synthesis, such as β-1,3-GT, β-1,4-GT, α-1,4-GT, α-1,4-GalNAcT, β-1,4-GalNAcT, β-1,3-GlcNAcT and/or β-1,6-GlcNAcT. In one embodiment, the nucleic acid construct is inserted into a cell that lacks functional expression of a sugar-related protein. In a more particular embodiment, the inserted construct encodes for a sugar-related protein that is different from the sugar-related protein that is lacking functional expression.

In an alternative aspect of the present invention, methods for modifying sugar metabolism in animals, tissues, organs, or cells lacking functional expression of a particular sugar-related protein can be provided wherein sugar intake is restricted, such as low galactose or lactose. In a more particular embodiment, animals lacking functional expression of α1,3-galactosyltransferase can be fed a diet lacking galactose and lactose.

In broad embodiments, the present invention is based on the discovery that in the instance of sugar metabolic pathway disruptions there is a limited endogenous ability of sugar metabolic pathways to reduce the accumulation of toxic sugar metabolites. Thus, the prevention of galactose transport out of the cell can lead to the toxic accumulation of galactose metabolites within the cell. Therefore, the present invention provides animals, tissues, organs and cells that have deficiencies in sugar metabolism, such as galactose metabolism, which have been genetically modified to compensate for the metabolic deficiency. This modification serves to decrease the accumulation of toxic metabolites, such as UDP-galactose, in the cell caused by the metabolic deficiency. Such animals, tissues, organs and cells can be used in research and in medical therapy, including in xenotransplantation. In addition, methods are provided to produce such animals, organs, tissues, and cells. Furthermore, methods are provided for reducing toxic metabolite accumulation in animals, tissues, organs, and cells, which have metabolic deficiencies.

### BRIEF DESCRIPTION OF THE DRAWINGS

FIG. 1A is a schematic depicting the integrated galactose metabolic pathways. FIG. 1B is a schematic depicting the role galactose plays in sugar chain synthesis.

FIG. 2 provides an overview of sugar chain pathways, including sugar catabolism, the hexosamine pathway and sugar chain synthesis pathways.

FIG. 3 provides an overview of a sugar catabolism pathway.

FIG. 4 illustrates a hexosamine pathway.

FIG. 5 depicts sugar chain synthesis pathways.

FIG. 6 provides a schematic of the genomic organization of the porcine alpha-1,3-galactosyltransferase gene. [symbol] denote the location of the start and stop codons, respectively. “P” represents the promoter sequence and exon numbers are shown at the top. Distance between exons does not represent exact length.

FIG. 7 provides a schematic of the genomic organization of the porcine iGb3 synthase gene. [symbol] denote the location of the start and stop codons, respectively. “P” represents the promoter sequence and exon numbers are shown at the top. The length of the intronic sequences is also provided.

FIG. 8 provides a schematic of the genomic organization of the Forssman Synthetase (FSM) gene. [symbol] denote the location of the start and stop codons, respectively. “P” represents the promoter sequence and exon numbers are shown at the top. The length of the intronic sequences is also provided.

FIG. 9 illustrates a schematic representing single cassette DNA constructs for homologous recombination. Left and right arms represent nucleic acid sequence homologous to a target genomic sequence. FIG. 10 illustrates a schematic representing double cassette DNA constructs for homologous recombination. Left and right arms represent nucleic acid sequence homologous to a target genomic sequence. The IRES represents the location of the internal ribosome entry site.

FIG. 11 depicts a schematic illustrating: 1. primers used to clone β-1,6-GlcNAcT cDNA; and 2. restriction enzymes used to insert β-1,6-GlcNAcT cDNA into a vector.

FIG. 12 depicts a schematic illustrating: 1. primers used to clone β-1,4-GT cDNA; and 2. restriction enzymes used to insert β-1,4-GT cDNA into a vector.

FIG. 13 illustrates the insertion of a double cassette containing cDNA encoding β-1,6-GlcNAcT and β-1,4-GT into a vector containing an internal ribosome entry site (IRES).

FIG. 14A is an illustration of primers (a-1, a-2, f-1, f-2, b-1, b-2) that can be used to clone nucleic acid sequences, which can be used as a 5′ arm for homologous recombination. FIG. 14B illustrates primers (a-3, a-4, f-3, f-4, b-3, b-4) that can be used to clone nucleic acid sequence that can be used as a 3′ arm for homologous recombination. FIG. 14C provides example primer sequences a-1, a-2, a-3, and a-4 that can be used to for produce 5′ and 3′-recombination arms that are homologous to the porcine alpha-1,3-GT gene. FIG. 14D provides example primer sequences f-1, f-2, f-3, and f-4 that can be used to for produce 5′ and 3′-recombination arms that are homologous to the porcine FSM synthase gene. FIG. 14E provides example primer sequences a-1, a-2, a-3, and a-4 that can be used to for produce 5′ and 3′-recombination arms that are homologous to the porcine iGb3 synthase gene.

FIG. 15 illustrates the location that primers a-1, a-2, a-3 and a-4 target on the alpha-1,3-GT gene.

FIG. 16 illustrates the location that primers b-1, b-2, b-3 and b-4 target on the iGb3 synthase gene.

FIG. 17 illustrates the location that primers f-1, f-2, f-3 and f-4 target on the FSM synthase gene.

FIG. 18 provides a schematic illustrating the construction of a targeting vector that contains a 5′-recombination arm, β-1,6-GlcNAcT cDNA, an internal ribosome entry site (IRES), β-1,4-GalT cDNA and a 3′-recombination arm.

FIG. 19 depicts a targeting vector that contains a 5′-recombination arm, β-1,6-GlcNAcT cDNA, an internal ribosome entry site (IRES), β-1,4-GalT cDNA and a 3′-recombination arm.

FIG. 20 illustrates homologous recombination between a double cDNA cassette and genomic DNA.

FIG. 21 provides a schematic that represents the resultant genomic DNA organization after homologous recombination has occurred between a single cassette DNA construct and genomic DNA.

FIG. 22 provides a schematic that represents the resultant genomic DNA organization after homologous recombination has occurred between a double cassette DNA construct and genomic DNA.

FIG. 23 depicts a conventional schematic representation of ammonia pathways. Specifically, galactose (Gal) as well as glucose (Glc) ingested can enter hepatocytes through GLUT (glucose transporter) system via the portal vein. galactose is converted by a sequential reaction of GALK (galactose kinase), GALT (galactose-1-phosphate uridyltransferase) and GALE (UDP-galactose-4′-epimerase) to UDP-Glucose and Glucose-1-Phopsphate (G-1-P). Accumulation of galactose can be converted to galactitiol by AR (aldose reductase). G-1-P can be converted by PGM (phosphoglucomutase) to G-6-P as energy source or to UDP-Glc by UGP (UDP-glucose pyrophosphorylase). G-6-P can be converted from Glc by GK (glucokinase). In addition, the schematic depicts the entry of amino acids (AA) into hepatocytes through SLCs (soluble carriers). AA are used to produce peptides. AA that are not used can be transported to other cells via SLCs, converted to a-KA (a-keto acids) or a-KG (a-ketoglutarate as energy in the TCA cycle (not shown) by AT (aminotransferase) or GDH (glutamate dehydrogenase), or degraded to NH₃ (ammonia). NH₃ produced via GDH or GA (glutaminase) enters the urea cycle that is present in the liver to form urea, or is converted to Gln (glutamine) in the coupled reaction with Glu (glutamate) by GS (glutamine synthetase). Urea is ultimately secreted in urine from the kidney.

FIG. 24 illustrates a conventional schematic representation of brain energy metabolism. Specifically the figure illustrates how amino acids (AA) and glucose (Glc) in the blood enter astrocytes, and then transported to neurons. Glutamate (Glu) and glutamine (Gln) can be shuttled via a “Gln-Glu shuttle”. Gln is converted to Glu in neuron by GA. Note that NH₃ is produced in this reaction.

FIG. 25 provides a schematic representing amino sugar pathways. Specifically, excess amino acids are converted to glutamine (Gln), which is further converted to fructose-6-phosphate (F-6-P) by GFAT (glutamate:fructose-6-phosphate transferase) to produce GlcN-6-P (glucosamine-6-phosphate). GlcN-6-P is acetylated by GAAT (glucosamine-6-P acetyl transferase) to produce GlcNAc-6-P (glucNAc-6-P), which is ultimately converted to UDP-GlcNAc, UDP-GalNAc, or CMP-NANA. These nucleotide sugars are transported to Golgi apparatus and used to produce sugar chains. Note that H+ (hydrogen) is produced in the reaction of GFAT. Also, mono- or di-phosphates are produced in these processes.

FIG. 26 illustrates the phenotype of wild type and alpha-1,3-GT knockout (KO) mice. A and B show the eye of a WT mouse before and after exposure of carbon dioxide (30 seconds), respectively. No changes were observed. C and D show the eye of an alpha-1,3-GT-KO mouse before and after exposure of carbon dioxide (30 seconds), respectively. The pinhead size cataracts in the alpha-1,3GT-KO mouse enlarged (arrow) promptly upon exposure of carbon dioxide: E shows the eye of an alpha-1,3GT-KO mouse after exposure of carbon dioxide (15 seconds) followed by spontaneous respiration in room air. Note that the size with opacity decreased with spontaneous respiration (reversible).

FIG. 27 provides a graphical representation of survival ratio versus age of the animal. Horizontal and vertical bars indicate age and survival rate compared to the pups number born from wild type mothers fed normal diet. Group A, B, or C was fed normal, 20%, or 40% galactose-rich diet, respectively. (+) or (−) denotes wild type (+/+) or alpha-1,3-GT-KO (−/−).

FIG. 28 depicts the organization of a portion of the alpha-1,3-GT promoter.

FIG. 29 illustrates a schematic representation of a promoter trap construct that can be used to inactivate the alpha-1,3-GT gene.

FIG. 30 depicts 7 α1,3Gal-positive and 5 α1,3Gal-negative mammals with non-synonymous mutations (i.e. a change in amino acid) and synonymous mutations (no amino acid change) in portions of aligned exons 7, 8, and 9 of the α1,3GT gene variants. Marmoset amino acids and their positions (top line) were used for reference. Similar data were obtained for the entire coding region (exons 4-9), except for a mutation-rich portion of exon 7 (see FIG. 2). The era of evolution during which each individual mutation occurred (bottom line) could then be estimated as summarized in FIG. 32.

FIGS. 31A and 31B identify triplet deletions [- - -] in the first half of exon 7 of the rodent, porcine, bovine, and lemur gene when alignment was with the marmoset (61G to 81K) and catarrhine counterparts. Despite the multiple mutations that corresponded to the stem region, the gene remained active throughout in the lower mammalian species. Exon 7 bp in the different species: ( ).

FIG. 32 shows four proto α1,3GT genes thought to have been expressed between 56-23 million years ago (MYA). Note that the 16 key amino acids are identical in α1,3Gal-positive mammals.

FIG. 33 illustrates the evolutionary tree of primates based on studies of the α1,3GT gene. The following is the figure legend: L: lemur. M: marmoset. R: rhesus. O: orangutan. H: human. ACT: active gene (bold lines). UPG: unprocessed pseudogene (dotted line). PPG: processed pseudogene (dotted one). ( ): number non-synonymous mutations. [ ]: total mutations.

FIG. 34 represents a table summarizing the occurrence of ACT, UPG and PPG in various species.

### DETAILED DESCRIPTION OF THE INVENTION

The present invention provides natural or transgenic galactose deficient cells, tissues, organs and animals that have been genetically modified to compensate for the abnormalities in galactose metabolic pathways. In particular, the present invention provides cells, tissues, organs and animals that have been genetically modified to compensate for abnormalities in galactose metabolic pathways to prevent the toxic accumulations of galactose metabolites. Such abnormalities can be either endogenously present, such as an in-born genetic defect, or genetically engineered, in the galactose deficient cell, tissue, organ or animal. The present invention provides methods to compensate for these abnormalities by genetically modifying the galactose deficient cells, tissues, organs and/or animals to express at least one additional protein of the galactose metabolic pathway.

Proteins involved in galactose metabolism include proteins associated with sugar catabolism, the hexosamine pathway and sugar chain synthesis. Proteins involved in sugar catabolism include, but are not limited to, galactokinase (GALK), galactose-1-phosphate uridyl transferase (GALT) and UDP-galactose-4-epimerase (GALE). Proteins associated with the hexosamine pathway include, but are not limited to, glutamine: fructose-6-phosphate amidotransferase (GFAT), the sodium-calcium exchanger (NCX) and the sodium-hydrogen exchanger (NHE). Proteins associated with sugar chain synthesis include, but are not limited to, β-1,3-galactosyltransferase (1-1,3-GT), β1,4-galactosyltransferase (1-1,4-GT), α-1,4-galactosyltransferase (α-1,4-GT), α-1,3-galactosyltransferase (α-1,3-GT), IsoGlobide 3 synthase (iGb3), Forssman synthase (FSM), N-acetylgalactosaminyltransferases (GalNAcT), and N-acetylglucosaminyltransferases (GlcNAc-T), such as β-1,6 GlcNac-T.

In another aspect of the invention, animals, tissues, organs and cells are provided in which at least one allele of the alpha-1,3-galactosyltransferase gene, the Forssman synthetase gene and/or the isoGloboside 3 (iGb3) synthase gene has been inactivated, which have been genetically modified to express at least one additional protein associated with sugar catabolism, the hexosamine pathway, or sugar chain synthesis. Alternatively, animals, tissues, organs and cells are provided in which both alleles (homozygous knock-outs) of the alpha-1,3-galactosyltransferase (α-1,3-GT) gene, the Forssman synthetase gene and/or the isoGloboside 3 (iGb3) synthase gene have been rendered inactive, which have been genetically modified to express at least one additional protein associated with galactose transport. Proteins involved in galactose transport can include, but are not limited to proteins involved in sugar catabolism, the hexosamine pathway, or sugar chain synthesis. These genetic modifications decrease the accumulation of toxic metabolites, such as UDP-Gal or UDP-GalNAc, which result from the inactivation of the alpha-1,3-galactosyltransferase gene, the Forssman synthetase gene and/or the isoGloboside 3 (iGb3) synthase gene.

Definitions

A “target DNA sequence” is a DNA sequence to be modified by homologous recombination. The target DNA can be in any organelle of the animal cell including the nucleus and mitochondria and can be an intact gene, an exon or intron, a regulatory sequence or any region between genes.

A “homologous DNA sequence or homologous DNA” is a DNA sequence that is at least about 85%, 90%, 95%, 98% or 99% identical with a reference DNA sequence. A homologous sequence hybridizes under stringent conditions to the target sequence, stringent hybridization conditions include those that will allow hybridization occur if there is at least 85% and preferably at least 95% or 98% identity between the sequences.

An “isogenic or substantially isogenic DNA sequence” is a DNA sequence that is identical to or nearly identical to a reference DNA sequence. The term “substantially isogenic” refers to DNA that is at least about 97-99% identical with the reference DNA sequence, and preferably at least about 99.5-99.9% identical with the reference DNA sequence, and in certain uses 100% identical with the reference DNA sequence.

“Homologous recombination” refers to the process of DNA recombination based on sequence homology.

“Gene targeting” refers to homologous recombination between two DNA sequences, one of which is located on a chromosome and the other of which is not.

“Non-homologous or random integration” refers to any process by which DNA is integrated into the genome that does not involve homologous recombination.

A “selectable marker gene” is a gene, the expression of which allows cells containing the gene to be identified. A selectable marker can be one that allows a cell to proliferate on a medium that prevents or slows the growth of cells without the gene. Examples include antibiotic resistance genes and genes which allow an organism to grow on a selected metabolite. Alternatively, the gene can facilitate visual screening of transformants by conferring on cells a phenotype that is easily identified. Such an identifiable phenotype can be, for example, the production of luminescence or the production of a colored compound, or the production of a detectable change in the medium surrounding the cell.

The term “mammal” is meant to include any human or non-human mammal, including but not limited to porcine, ovine, bovine, canine, equine, feline, rodents, ungulates, pigs, swine, sheep, lambs, goats, cattle, deer, mules, horses, monkeys, apes, dogs, cats, rats, and mice.

The term “porcine” refers to any pig species, including pig species such as Large White, Landrace, Meishan, Minipig.

The term “oocyte” describes the mature animal ovum which is the final product of oogenesis and also the precursor forms being the oogonium, the primary oocyte and the secondary oocyte respectively.

DNA (deoxyribonucleic acid) sequences provided herein are represented by the bases adenine (A), thymine (T), cytosine (C), and guanine (G).

The term “cDNA” refers to a chain of nucleotides, an isolated polynucleotide, nucleotide, nucleic acid molecule, or any fragment or complement thereof. It may have originated recombinantly or synthetically and be double-stranded or single-stranded, coding and/or noncoding, an exon or an intron of a genomic DNA molecule, or combined with carbohydrate, lipids, protein or inorganic elements or substances.

Amino acid sequences provided herein are represented by the following abbreviations:

| | |
|---|---|
| A | alanine |
| P | proline |
| B | aspartate or asparagine |
| Q | glutamine |
| C | cysteine |
| R | arginine |
| D | aspartate |
| S | serine |
| E | glutamate |
| T | threonine |
| F | phenylalanine |
| G | glycine |
| V | valine |
| H | histidine |
| W | tryptophan |
| I | isoleucine |
| Y | tyrosine |
| Z | glutamate or glutamine |
| K | lysine |
| L | leucine |
| M | methionine |
| N | asparagine |

“Transfection” refers to the introduction of DNA into a host cell. Cells do not naturally take up DNA. Thus, a variety of technical “tricks” are utilized to facilitate gene transfer. Numerous methods of transfection are known to the ordinarily skilled artisan, for example, CaPO₄ and electroporation. (J. Sambrook, E. Fritsch, T. Maniatis, Molecular Cloning: A Laboratory Manual, Cold Spring Laboratory Press, 1989). Transformation of the host cell is the indicia of successful transfection.

A “knock-in” approach refers to the procedure of inserting the gene or the portion of a gene into the genome of a host. This can include, for instance, localizing the polynucleotide encoding a mutant polypeptide or protein to the locus encoding such polypeptide or protein or replacing an entire gene or coding region with a polynucleotide sufficient to encode a mutant polypeptide or protein. Accordingly, a “knock-in mammal” refers to a transgenic mammal produced using a “knock-in approach”.

The term “galactose deficient” as used herein refer to a reduction in galactose levels over that normally observed as a result of a natural or induced abnormality in galactose metabolism. Galactose deficient cells, tissues, organs and/or animal can be, for example, galactose deficient due to an endogenously present error in metabolism, such as an inborn genetic defect, or genetically engineered in such a way that galactose metabolism is affected.

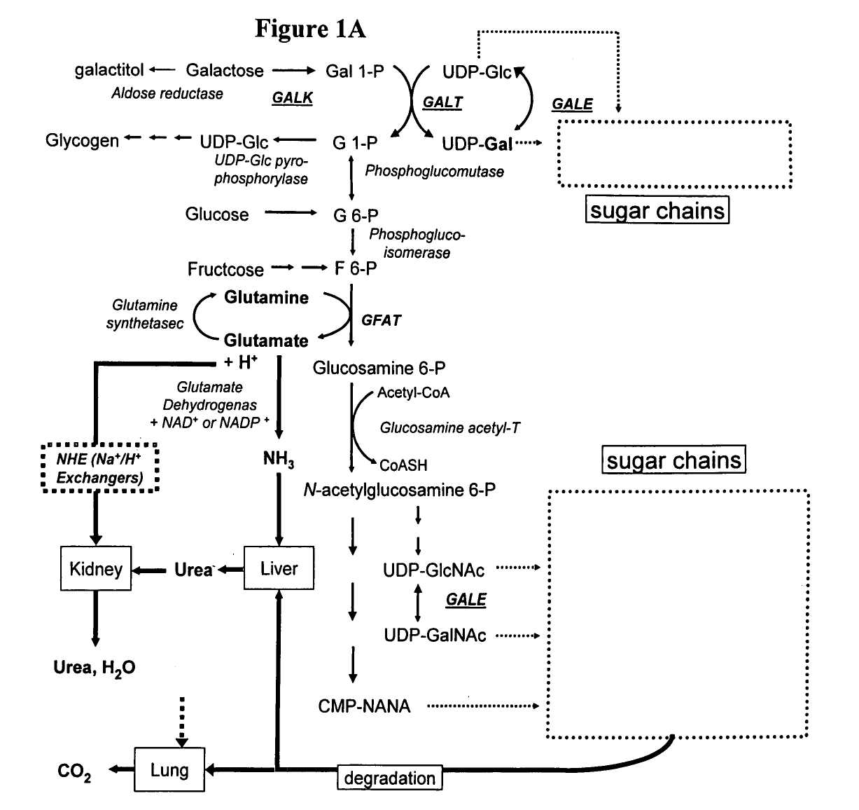

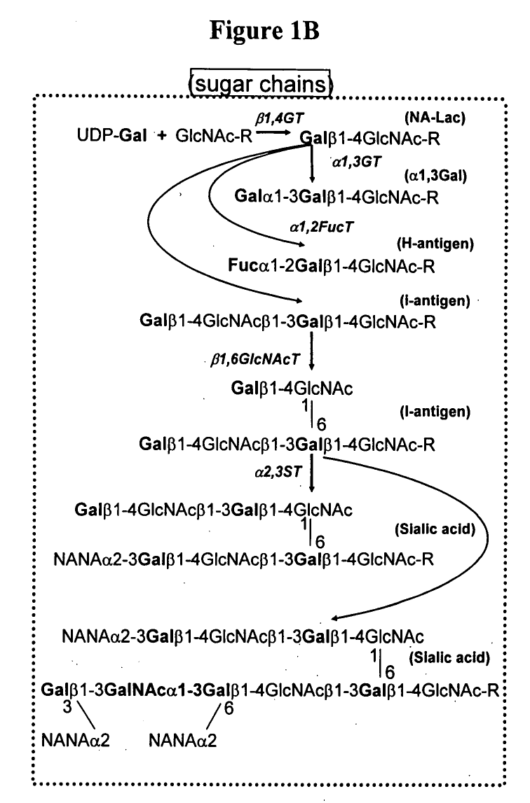

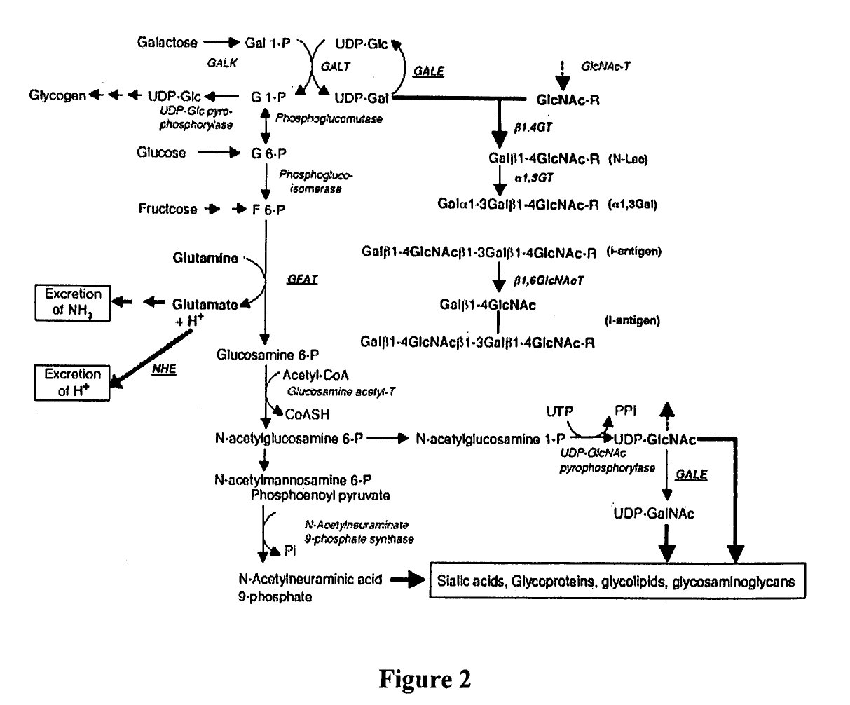

I. Sugar Metabolic Pathways (See, for Example, FIGS. 1A, 2)

In one aspect of the invention, cells, tissues, organs and animals are provided in which at least one allele of a gene involved in galactose transport has been inactivated, which have been genetically modified to express at least one additional protein that can transport galactose out of the cell to compensate for this deficiency. Proteins involved in galactose transport include: proteins involved in: sugar catabolism, such as, but not limited to, galactokinase (GALK), galactose-1-phosphate uridyl transferase (GALT) and UDP-galactose-4-epimerase (GALE); the hexosamine pathway, such as, but not limited to, glutamine: fructose-6-phosphate amidotransferase (GFAT), the sodium-calcium exchanger (NCX) and the sodium-hydrogen exchanger (NHE); sugar chain synthesis, such as, but not limited to, β-1,3-galactosyltransferase (β-1,3-GT), β-1,4-galactosyltransferase (β-1,4-GT), α-1,4-galactosyltransferase (α-1,4-GT), α-1,3-galactosyltransferase (α-1,3-GT), IsoGlobide 3 synthase (iGb3), Forssman synthase (FSM), N-acetylgalactosaminyltransferases (GalNAcT), and N-acetylglucosaminyltransferases (GlcNAc-T), such as β-1,6 GlcNac-T.

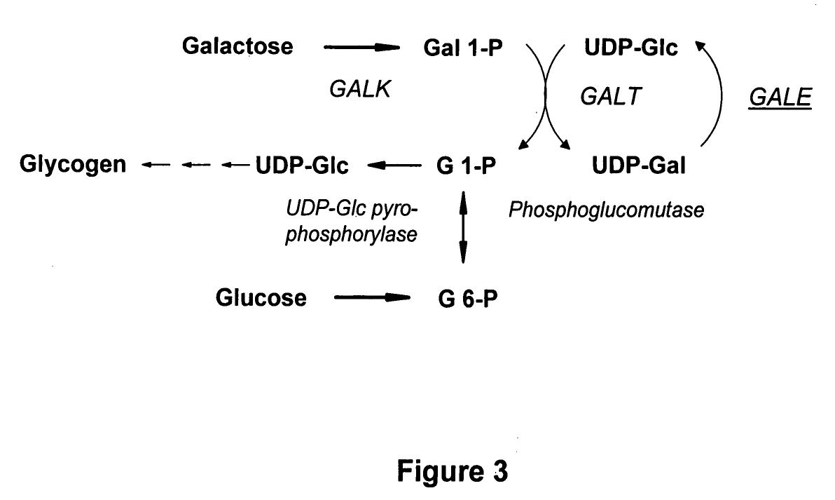

a. Sugar Catabolic Pathways (See, for Example, FIG. 3)

The sugar catabolic pathways are essential in the derivation of energy for the cell, and a diverse group of saccharides can be utilized as fuel sources. Proteins involved in sugar catabolism include, but are not limited to, galactokinase (GALK), galactose-1-phosphate uridyl transferase (GALT) and UDP-galactose-4-epimerase (GALE).

The invention provides modification of the expression of proteins associated with the catabolic pathways of monosaccharides having the general formula (CH₂O)ₙ, wherein n can be 3, 4, 5, 6, 7, or 8 and have two or more hydroxyl groups, such as, for example, trioses, including glyceraldehyde and dihydroxyacetone, tetroses, including erythrose, pentoses, including ribose, hexoses, including glucose, galactose, mannose, and fructose, heptoses, including sedoheptulose, and nonoses, including neuraminic acid.

Proteins associated with monosaccharide catabolism that can be utilized for compensation in the present invention include, but are not limited to, hexokinase, phosphoglucose isomerase (PGI), phosphofructokinase (PFK), adolase A, adolase B, triose phosphate isomerase (TIM), glyceraldehyde-3-phosphate dehydrogenase (GAPDH), phosphoglycerate kinase (PGK), phosphoglycerate mutase (PGM), alcohol deydrogenase, glycerol kinase, enolase, pyruvate kinase, fructokinase, fructose 1-phosphate adolase, alcohol dehydrogenase, glycerol kinase, glycerol phosphate dehydrogenase, glyceraldehyde kinase, galactokinase, galactose-phosphate uridylyl transferase, UDP-galactose-4-epimerase, phosphoglucomutase, fructose 1,6-biphosphatase, phosphomannose isomerase, aldose reductase, sorbitol dehydrogenase, glucose 6-phosphate dehydrogenase, gluconolactonase, 6-phosphogluconate dehydrogenase, ribulose 5-phosphate epimerase, ribulose 5-phosphate 3 epimerase, transketolase, transaldolase, glutathione peroxidase, glyceraldehydes 3 phosphate dehydrogenase, bisphosphoglycerate mutase, phosphoglycerate kinase, 2,3-bisphosphoglycerate phosphatase, 3 Dehydroquinate synthase, 3-Dehydroquinate dehydratase, Shikimate dehydrogenase, Shikimate kinase, 3-phosphoshikimate-1-carboxyvinyl transferase (EPSP synthase), Chorismate synthase, and related homologs and isoforms.

The invention also includes modifying the expression of proteins associated with the catabolic pathways of disaccharides. Disaccharides consist of two polymerized monosaccharide molecules of one type or two alternating types, such as, for example, lactose, maltose, and sucrose. An enzyme generally hydrolyzes the glycosidic bond between the two monosaccharides, and the monosaccharides are then catabolized. Proteins associated with disaccharide catabolism that can be utilized for compensation in the present invention include, but are not limited to, α-amylase, lactase, sucrase, maltase, invertase, xylanase, isomaltase, and related homologs and isoforms.

The invention further includes the modification of proteins associated with the catabolic pathways of oligosaccharides containing 3 or more monosaccharide units bound by glycosidic linkages, such as, for example, fructo-oligosaccharides, glucose-oligosaccharides, and insulin. Alternatively, the invention includes compensation with proteins associated with polysaccharide metabolism containing 12 or more monosaccharide units, including homopolysaccharides containing only a single monosaccharide species such as, for example, glycogen, cellulose, and starch, and heteropolysaccharides containing a number of different monosaccharide species, such as glycosaminoglycans including heparin, keratin sulfate, hyaluronic acid, heparan sulfate, dermatan sulfate, and chondroitin sulfate. Additional proteins associated with polysaccharides catabolism that can be utilized for compensation in the present invention include, but are not limited to, glycogen phosphorylase, glucosyl transferase, amylo-α-(1,6)-glucosidase, endoglycosidases, iduronate sulfatase, α-L-iduronidase, heparin sulfamidase, N-acetyltransferase, N-acetylglucosaminidase, β-glucuronidase, N-acetylglucosamine 6 sulfatase, diastase, glucoamylase, and associated homologs and isoforms.

TABLE 1
cDNA encoding GALE

| | | | |
|---|---|---|---|
| Protein | | Correspond- | |
| Associated | | ing | |
| with Sugar | | Assession | Sequence |
| Metabolism | cDNA Sequence | Number | Identifier |

| | | | | |
|---|---|---|---|---|
| galatose4- | gactctccag tcctcagtca ccttggacaa | | NM_000403 | Seq ID No. 1 |
| epimerase | agaagtgtgg atcctcagat tccatctttt | 61 | | |
| (GALE) | ccaactccaa ggtgccatgg cagagaaggt | | | |
| | gctggtaaca ggtggggctg gctacattgg | 121 | | |
| | cagccacacg gtgctggagc tgctggaggc | | | |
| | tggctacttg cctgtggtca tcgataactt | 181 | | |
| | ccataatgcc ttccgtggag ggggctccct | | | |
| | gcctgagagc ctgcggcggg tccaggagct | 241 | | |
| | gacaggccgc tctgtggagt ttgaggagat | | | |
| | ggacattttg gaccagggag ccctacagcg | 301 | | |
| | tctcttcaaa aagtacagct ttatggcggt | | | |
| | catccacttt gcggggctca aggccgtggg | 361 | | |
| | cgagtcggtg cagaagcctc tggattatta | | | |
| | cagagttaac ctgaccggga ccatccagct | 421 | | |
| | tctggagatc atgaaggccc acggggtgaa | | | |
| | gaacctggtg ttcagcagct cagccactgt | 481 | | |
| | gtacgggaac ccccagtacc tgccccttga | | | |
| | tgaggcccac cccacgggtg gttgtaccaa | 541 | | |
| | cccttacggc aagtccaagt tcttcatcga | | | |
| | ggaaatgatc cgggacctgt gccaggcaga | 601 | | |
| | caagacttgg aacgtagtgc tgctgcgcta | | | |
| | tttcaacccc acaggtgccc atgcctctgg | 661 | | |
| | ctgcattggt gaggatcccc agggcatacc | | | |
| | caacaacctc atgccttatg tctcccaggt | 721 | | |
| | ggcgatcggg cgacgggagg ccctgaatgt | | | |
| | ctttggcaat gactatgaca cagaggatgg | 781 | | |
| | cacaggtgtc cgggattaca tccatgtcgt | | | |
| | ggatctggcc aagggccaca ttgcagcctt | 841 | | |
| | aaggaagctg aaagaacagt gtggctgccg | | | |
| | gatctacaac ctgggcacgg gcacaggcta | 901 | | |
| | ttcagtgctg cagatggtcc aggctatgga | | | |
| | gaaggcctct gggaagaaga tcccgtacaa | 961 | | |
| | ggtggtggca cggcgggaag gtgatgtggc | | | |
| | agcctgttac gccaacccca gcctggccca | 1021 | | |
| | agaggagctg gggtggacag cagccttagg | | | |
| | gctggacagg atgtgtgagg atctctggcg | 1081 | | |
| | ctggcagaag cagaatcctt caggctttgg | | | |
| | cacgcaagcc tgaggaccct cccctaccaa | 1141 | | |
| | ggaccaggaa aagcagcagc tgcctgctct | | | |
| | ccagcctctg gaggaactca gggccctgga | 1201 | | |
| | gctgctgggg ccaagccaag ggcctcccct | | | |
| | acctcaaacc ccagctgggc ccgcttagcc | 1261 | | |
| | caccaggcat gaggccaagg ctccactgac | | | |
| | caggaggccg aggtctctaa ctcttatctt | 1321 | | |
| | ccacagggtc caagagttca tcaggacccc | | | |
| | caagagtgag tgagggggca aggctctggc | 1381 | | |
| | acaaaacctc ctcctcccag gcactcattt | | | |
| | atattgctct gaaagagctt tccaaagtat | 1441 | | |
| | ttaaaaataa aaacaagttt tcttacactg g | | | |

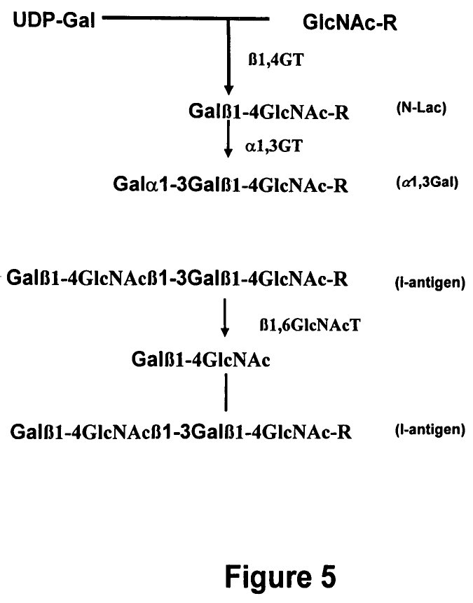

b. Sugar Chain Synthesis Pathways (See, for Example, FIGS. 1B, 5)

The sugar chain synthesis pathways play an important role the production of glycoconjugates. The major types of glycoconjugates are glycoproteins, glycopeptides, peptidoglycans, proteoglycans, glycolipids and lipopolysaccharides. Proteins associated with sugar chain synthesis include, but are not limited to, β-1,3-galactosyltransferase (β-1,3-GT), β-1,4-galactosyltransferase (β-1,4-GT), α-1,4-galactosyltransferase (α-1,4-GT), α-1,3-galactosyltransferase (α-1,3-GT), IsoGlobide 3 synthase (iGb3), Forssman synthase (FSM), N-acetylgalactosaminyltransferases (GalNAcT), and N-acetylglucosaminyltransferases (GlcNAc-T), such as β-1,6 GlcNac-T.

Glycoproteins are proteins to which oligosaccharides are covalently attached in relatively short chains (usually two to ten sugar residues in length, although they can be longer) (Lippincott's Illustrated Reviews: Biochemistry 2^{nd} Ed. Champe, P. C., Harvey, R. A. Lippincott Williams & Wilkins. Philadelphia, Pa. (1994)). Membrane bound glycoproteins participate in a broad range of cellular phenomena, including cell surface recognition, cell surface antigenicity, and as components of the extracellular matrix and of the mucins of the gastrointestinal and urogenital tract (Medical Biochemistry 4^{th} Ed. Bhagavan, N. V. Harcourt Brace & Co., New York; Lippincott's Illustrated Reviews: Biochemistry 2^{nd} Ed. Champe, P. C., Harvey, R. A. Lippincott Williams & Wilkins. Philadelphia, Pa. (1994)).

Glycolipids are compounds containing one or more monosaccharide residues bound by a glycosidic linkage to a hydrophobic moiety such as an acylglycerol, a sphingoid, a ceramide (N-acylsphingoid) or a prenyl phosphate. Glycoglycerolipids are glycolipids containing one or more glycerol residues. Glycosphingolipids are lipids containing at least one monosaccharide residue and either a sphingoid or a ceramide.

Glycophosphatidylinositols are glycolipids which contain saccharides glycosidically linked to the inositol moiety of phosphatidylinositols Glycoconjugates serve as major exporters of saccharides out of the intracellular environment. The components utilized in the formation of glycoconjugates are sugar nucleotides, include, but are not limited to, UDP-glucose, UDP-galactose, UDP-N-acetylglucosamine, UDP-galactosamine, GDP-mannose, GDP-L-fucose, and CMP-N-acetylneuraminic acid.

Proteins associated with sugar chain synthesis that can be utilized for compensation in the present invention include, but are not limited to, β-1,3-galactosyltransferases, β-1,4-galactosyltransferases, α-1,3 galactosyltransferase, isogloboside 3 synthase (iGb3 synthase), Forssman synthase (FSM synthase), α-1,4 galactosyltransferases, or galactosylceramides, β1,3-N-acetylgalactoseaminyltransferases, β1,4-N-acetylgalactosaminyltransferases, α-1,4-N-acetylgalactosaminyltransferases, and β-1,6-N-acetylgalactoaminyltransferases, β1,6-acetylglucoseaminyltransferases, β1,4-Acetylglucoseaminyltransferases, β-1,2-acetylglucoseaminyltransferases α-2,3-sialyltransferase, α-2,6-sialyltransferase, α-2,8-sialyltransferase, and related homologs and isoforms.

TABLE 2
Mammalian Galactosyltransferases

| | | | | GenBank accession # | |
|---|---|---|---|---|---|
| | | | | (refers to the human genes, | |
| | | | | except for the two α1-3 GalT | |
| | | | | Ggta 1 and iGb3 synthase, where | |
| | | Human | Expression | the numbers point to the mouse | |
| Enzyme | Gene | chromosome | (UniGene) | and rat cDNA, respectively) | Reference(s) |
| β1-4 GalT | B4GALT1 | 9p13 | ubiquitous | NM_001497 | Shaper et al. (1986) Proc. Natl. |
| | | | | | Acad. Sci. USA 83: 1573-1577. |
| β1-4 GalT | B4GALT2 | 1p34-p33 | ubiquitous | NM_030587 | Almeida R. et al (1997) J. |
| | | | | | Biol. Chem. 272: 31979-31991 |
| β1-4 GalT | B4GALT3 | 1q21-q23 | ubiquitous | NM_003779 | Almeida R. et al (1997) J. |
| | | | | | Biol. Chem. 272: 31979-31991 |
| β1-4 GalT | B4GALT4 | 3q13 | ubiquitous | NM_003778 | Schwientek T. et al. (1998) J. |
| | | | | | Biol. Chem. 273: 29331-29340 |
| β1-4 GalT | B4GALT5 | 20q13 | ubiquitous | NM_004776 | Sato et al. (1998) Proc. Natl. |
| | | | | | Acad. Sci. USA 95: 472-477 |
| β1-4 GalT | B4GALT6 | 18q11 | Bone marrow, | NM_004775 | Nomura T. et al. (1998) J. Biol. |
| | | | brain, breast, | | Chem. 273: 13570-13577 |
| | | | lung, pancreas, | | |
| | | | skin, whole embryo | | |
| β1-4 GalT | B4GALT7 | 5q35 | ubiquitous | NM_007255 | Almeida R (1999) J. Biol. |
| | | | | | Chem. 274: 26165-26171 |
| β1-3 GalT | B3GALT1 | 2p14 | Germ cells, brain | NM_020981 | Hennet T (1998) J. Biol. Chem. |
| | | | | | 273: 58-65 |
| β1-3 GalT | B3GALT2 | 1q31 | blood, bone, brain, | NM_003783 | Hennet T (1998) J. Biol. Chem. |
| | | | colon, heart, pancreas, | | 273: 58-65; |
| | | | skin, whole embryo, | | Kolbinger F et al. (1998) J. Biol. |
| | | | lung, nervous | | Chem. 273: 433-440; |
| | | | system, prostate | | Amado M. (1998) J. Biol. Chem. |
| | | | | | 273: 12770-12778 |
| β1-3 GalT | B3GALT3 | 3q25 | bladder, bone, brain, | NM_003781 | Hennet T (1998) J. Biol. Chem. |
| | | | breast, colon, | | 273: 58-65; |
| | | | foreskin, germ cell, | | Kolbinger F et al. (1998) |
| | | | heart, kidney, lung, | | J. Biol. Chem. 273: 433-440; |
| | | | ovary, prostate, | | Amado M. (1998) J. Biol. Chem. |
| | | | testis, uterus, | | 273: 12770-12778 |
| | | | whole embryo | | |
| β1-3 GalT | B3GALT4 | 6p21 | Brain, colon, | NM_003782 | Miyazaki H (1997) J. Biol. |
| | | | lung, ovary, | | Chem. 272: 24794-24799 |
| | | | pancreas, lung, | | |
| | | | testis, kidney, | | |
| | | | stomach, prostate | | |
| β1-3 GalT | B3GALT5 | 21q22 | breast, colon, | NM_006057 | Isshiki S. et al. (1999) J. Biol. |
| | | | pancreas, testis, | | Chem. 274: 12499-12507. |
| | | | nervous system | | Zhou D. et al. (1999) Eur. J. |
| | | | | | Biochem. 263: 571-576 |
| | | | | | Zhou D et al. (2000) J. Biol. |
| | | | | | Chem. 275: 22631-22634 |
| β1-3 GalT | B3GALT6 | 1 | ubiquitous | AY050570 | Bai X (2001) J. Biol. Chem. |
| | | | | | 276: 48189-48195 |
| β1-3 GalT | B3GALT7, | 7 | bone marrow, brain, | NM_020156 | Ju T (2002) J. Biol. |
| | C1GALT1 | | colon, germ cell, | | Chem. 277: 178-186 |
| | | | kidney, pancreas, | | |
| | | | placenta, small | | |
| | | | intestine, | | |
| | | | stomach, uterus | | |
| α1-3 GalT | ABO | 9q34 | Colon, blood | NM_020469 | Yamamoto F (1990) |
| | | | | | Nature 345: 229-233 |
| α1-3 GalT | Ggtal | — | embryo, heart, lung, | NM_010283 | Joziasse D. H (1989) J. Biol. |
| | | | mammary gland, | | Chem. 264: 14290-14297 |
| | | | pancreas, | | |
| | | | salivary gland, | | |
| | | | skin, spleen, uterus | | |
| α1-3 GalT | (iGb3s) | — | lung, uterus, | AF248543 | Keusch J. J (2000) J. Biol. |
| | | | pituitary, thymus, | | Chem. 275: 25308-25314 |
| | | | skeletal muscle, | | |
| | | | brain, spleen, kidney | | |
| α1-4 GalT | A4GALT1 | 22q13 | ubiquitous | NM_017436 | Keusch J. J. (2000) J.Biol. |
| | | | | | Chem. 275: 25315-25321 |
| | | | | | Steffensen R (2000) J. Biol. |
| | | | | | Chem. 275: 16723-16729 |
| Cer GalT | CGT | 4q26 | Brain, kidney | NM_003360 | Steffensen R (2000) J. Biol. |
| | | | | | Chem. 275: 16723-16729 |

TABLE 3
cDNA Sequences encoding Proteins Involved in Sugar Chain Sythesis

| Protein | | Correspond- | |
|---|---|---|---|
| Associated | | ing | |
| with Sugar | | Assession | Sequence |
| Metabolism | cDNA Sequence | Number | Identifier |
| β-1,3 | ggctacgcagcttgctcctggcacgggcaccttgaatctc | NM_020981 | Seq ID No. 2 |
| galactosyl- | ctcctcacacagatggagaccatgcttgatttcctgaact | | |
| transferase | tgtagtaagaagaaggaaaacacagcacgctggagccaac | | |
| | agagttaagaggaagatttatgagtcatggaaccctccat | | |
| | cagatttggaagaaagtagaatgagcgcagaggtgacaga | | |
| | cagccactgaggcccatggacaatctccacctcacgcttc | | |
| | tctatcaaacttgaagatttattagtaatatgctgccttt | | |
| | ggaagatgaaaacaaactagtgccaaggaggcgtattctt | | |
| | caatatttggaatagacgtgttctcaagacaatggcttca | | |
| | aaggtctcctgtttgtatgttttgacagttgtgtgctggg | | |
| | ccagcgctctctggtacttgagtataactcgccctacttc | | |
| | ttcttacactggctccaaaccattcagccacctaacagtt | | |
| | gccaggaaaaacttcacctttggcaacataagaactcgac | | |
| | ctatcaacccacattcttttgaatttcttatcaacgagcc | | |
| | caataaatgtgagaaaaacattccttttcttgttatcctc | | |
| | atcagcaccactcacaaggaatttgatgcccgtcaggcaa | | |
| | tcagagagacgtggggggatgagaacaactttaaggggat | | |
| | caagatagccaccctgttcctcctgggcaagaatgctgat | | |
| | cctgttctcaatcagatggtggagcaagagagccaaatct | | |
| | tccatgatatcatcgtggaggactttattgactcctacca | | |
| | taaccttaccctcaaaacat | | |
| | taatggggatgagatgggtggccacttttt gttcaaaagc | | |
| | caagtatgtc atgaaaacag acagcgacat | | |
| | ttttgtaaac 901 atggacaatc ttatttataa | | |
| | attactgaaa ccctccacca agccacgaag | | |
| | aaggtatttt 961 actggctatg tcattaatgg | | |
| | aggaccgatt cgggatgtcc gcagtaaatg | | |
| | gtatatgccc 1021 agggatttgt acccagacag | | |
| | taactaccca cctttctgtt cggggactgg | | |
| | ctacatcttt 1081 tcagccgatg tagctgaact | | |
| | catttacaag acctcactcc acacaaggct | | |
| | gcttcacctt 1141 gaagacgtat atgtgggact | | |
| | gtgtcttcga aagctgggca tacatccttt | | |
| | ccagaacagt 1201 ggcttcaatc actggaaaat | | |
| | ggcctacagt ttgtgtaggt atcgccgagt | | |
| | tatcactgtg 1261 catcagatct ctccagaaga | | |
| | aatgcacaga atctggaatg acatgtcaag | | |
| | caagaaacat 1321 ctcagatgtt aggattttta | | |
| | ccaatgtaaa tatgtttctt ttcttttttt | | |
| | aagaaatggg 1381 acctaaggtg ttggtatttt | | |
| | ccaggtgtcg ggggaaatga actggtgaag | | |
| | gggttttgta 1441 aagtttttgc ttcctgctat | | |
| | aagttctttt cttggattac caatttatga | | |
| | atgttagact 1501 ctggtcatag aaacaataaa | | |
| | tgagttagaa gggccagatt tcattctcag | | |
| | tcccagagca 1561 ttgctattta tctcaaaaag | | |
| | tgacttccaa acaactctta ggattgacgt | | |
| | accgtgcatc 1621 tgagataaaa atttggttct | | |
| | gggaaactga aactcacagt aatgtgtcat | | |
| | atcatccctg 1681 caaaaattaa tacacaaata | | |
| | gaaaccattt tcaaaagcaa ttcagaaagg | | |
| | atgcacagtc 1741 aggaagacac actggatgtg | | |
| | attattaata tcgtgtgtgt tgttacatta | | |
| | tatttttaca 1801 tatattccca tgtaatgtgt | | |
| | acagtctttg cagttccacc aagaaatgaa | | |
| | cttggtacct 1861 gcagagtggc tgcagttaaa | | |
| | tagatgggag tttaaatttg agaatcaaac | | |
| | attctatgtg 1921 tttggaagac aactctgctt | | |
| | gctcatccaa ggattaaatc tggtcagcag | | |
| | gtggaatgtg 1981 tataaaatgc tacttaacaa | | |
| | agtaaacaaa agattttttt tttctttttt | | |
| | tttctttctt 2041 ttttgttttg ctctttcaga | | |
| | acaaacatta aatggtgcct ccaaggaaac | | |
| | tttgccaaat 2101 ataatctcac ctgcttcctt | | |
| | ccagacagtg tcgctaagtg catttcacag | | |
| | tttttggatc 2161 tggcaggc | | |
| β-1,4 | gcgcctgcgg cgccgcgggc gggtcgcctc | NM_001497 | Seq ID No.3 |
| galactosyl | ccctcctgta gcccacaccc ttcttaaagc 61 | | |
| transferase | ggcggcggga agatgaggct tcgggagccg | | |
| | ctcctgagcg gcagcgccgc gatgccaggc 121 | | |
| | gcgtccctac agcgggcctg ccgcctgctc | | |
| | gtggccgtct gcgctctgca ccttggcgtc 181 | | |
| | accctcgttt actacctggc tggccgcgac | | |
| | ctgagccgcc tgccccaact ggtcggagtc 241 | | |
| | tccacaccgc tgcagggcgg ctcgaacagt | | |
| | gccgccgcca tcgggcagtc ctccggggag 301 | | |
| | ctccggaccg gaggggcccg gccgccgcct | | |
| | cctctaggcg cctcctccca gccgcgcccg 361 | | |
| | ggtggcgact ccagcccagt cgtggattct | | |
| | ggccctggcc ccgctagcaa cttgacctcg 421 | | |
| | gtcccagtgc cccacaccac cgcactgtcg | | |
| | ctgcccgcct gccctgagga gtccccgctg 481 | | |
| | cttgtgggcc ccatgctgat tgagtttaac | | |
| | atgcctgtgg acctggagct cgtggcaaag 541 | | |
| | cagaacccaa atgtgaagat gggcggccgc | | |
| | tatgccccca gggactgcgt ctctcctcac 601 | | |
| | aaggtggcca tcatcattcc attccgcaac | | |
| | cggcaggagc acctcaagta ctggctatat 661 | | |
| | tatttgcacc cagtcctgca gcgccagcag | | |
| | ctggactatg gcatctatgt tatcaaccag 721 | | |
| | gcgggagaca ctatattcaa tcgtgctaag | | |
| | ctcctcaatg ttggctttca agaagccttg 781 | | |
| | aaggactatg actacacctg ctttgtgttt | | |
| | agtgacgtgg acctcattcc aatgaatgac 841 | | |
| | cataatgcgt acaggtgttt ttcacagcca | | |
| | cggcacattt ccgttgcaat ggataagttt 901 | | |
| | ggattcagcc taccttatgt tcagtatttt | | |
| | ggaggtgtct ctgctctaag taaacaacag 961 | | |
| | tttctaacca tcaatggatt tcctaataat | | |
| | tattggggct ggggaggaga agatgatgac 1021 | | |
| | atttttaaca gattagtttt tagaggcatg | | |
| | tctatatctc gcccaaatgc tgtggtcggg 1081 | | |
| | aggtgtcgca tgatccgcca ctcaagagac | | |
| | aagaaaaatg aacccaatcc tcagaggttt 1141 | | |
| | gaccgaattg cacacacaaa ggagacaatg | | |
| | ctctctgatg gtttgaactc actcacctac 1201 | | |
| | caggtgctgg atgtacagag atacccattg | | |
| | tatacccaaa tcacagtgga catcgggaca 1261 | | |
| | ccgagctagc gttttggtac acggataaga | | |
| | gacctgaaat tagccaggga cctctgctgt 1321 | | |
| | gtgtctctgc caatctgctg ggctggtccc | | |
| | tctcattttt accagtctga gtgacagctc 1381 | | |
| | cccttggctc atcattcaga tggctttcca | | |
| | gatgaccagg acaggtggga tattttgccc 1441 | | |
| | ccaacttggc tcggcatgtg aattcttagc | | |
| | tctgcaaggt gtttatgcct ttgcgggttt 1501 | | |
| | cttgatgtgt tcgcagtgtc acccaagagt | | |
| | cagaactgta gacatcccaa aatttggtgg 1561 | | |
| | ccgtggaaca cattcccggt gatagaattg | | |
| | ctaaattgtc gtgaaatagg ttagaatttt 1621 | | |
| | tctttaaatt atggttttct tattcgcgaa | | |
| | aattcggaga gtgctgctaa aattggattg 1681 | | |
| | gtgtcatctt tttggtagtt | | |
| | gtaatttaacagaaaaacac aaaatttcaa | | |
| | ccattcttaa 1741 tgttacgtcc tccccccacc | | |
| | cccttctttc agtggtatgc aaccactgca | | |
| | atcaatgtgt 1801 catatgtctt ttcttagcaa | | |
| | aaggatttaa aacttgagcc ctggaccttt | | |
| | tgcctatgtg 1861 tgtggattcc agggcaactc | | |
| | tagcatcaga gcaaaagcct tgggtttctc | | |
| | gcattcagtg 1921 gcctatctcc agattgtctg | | |
| | atttctgaat gtaaagttgt tgtgtttttt | | |
| | tttaaatagt 1981 aggtttgtag tattttaaag | | |
| | aaagaacaga tcgagttcta attatgatct | | |
| | agcttgattt 2041 tgtgttgatc caaatttgca | | |
| | tagctgttta atgttaagtc atgacaattt | | |
| | atttttcttg 2101 gcatgctatg taaacttgaa | | |
| | tttcctaagt atttttattc tggtgtttta | | |
| | aatatgggga 2161 ggggtattga gcatttttta | | |
| | gggagaaaaa taaatatatg ctgtagtggc | | |
| | cacaaatagg 2221 cctatgattt agctggcagg | | |
| | ccaggttttc tcaagagcaa aatcaccctc | | |
| | tggccccttg 2281 gcaggtaagg cctcccggtc | | |
| | agcattatcc tgccagacct cggggaggat | | |
| | acctgggaga 2341 cagaagcctc tgcacctact | | |
| | gtgcagaact ctccacttcc ccaaccctcc | | |
| | ccaggtgggc 2401 agggcggagg gagcctcagc | | |
| | ctccttagac tgacccctca ggcccctagg | | |
| | ctggggggtt 2461 gtaaataaca gcagtcaggt | | |
| | tgtttaccag ccctttgcac ctccccaggc | | |
| | agagggagcc 2521 tctgttctgg tgggggccac | | |
| | ctccctcaga ggctctgcta gccacactcc | | |
| | gtggcccacc 2581 ctttgttacc agttcttcct | | |
| | ccttcctctt ttcccctgcc tttctcattc | | |
| | cttccttcgt 2641 ctcccttttt gttcctttgc | | |
| | ctcttgcctg tcccctaaaa cttgactgtg | | |
| | gcactcaggg 2701 tcaaacagac tatccattcc | | |
| | ccagcatgaa tgtgcctttt aattagtgat | | |
| | ctagaaagaa 2761 gttcagccgc acccacaccc | | |
| | caactccctc ccaagaactt cggtcctaaa | | |
| | gcctcctgtt 2821 ccacctcagg ttttcacagg | | |
| | tgctcacacc acagttgagg ctcacacaca | | |
| | ggtctgtctg 2881 tcacaaaccc acctctgttg | | |
| | ggagctattg agccacctgg gatgagatga | | |
| | cacaagacac 2941 tcctaccact gagcgccttt | | |
| | gtccaggtgc cagcctgggc tcaggttcca | | |
| | agactcagct 3001 gcctaatccc agggttgagc | | |
| | cttgtgctcg tgtcggaccc caaaccactg | | |
| | ccctcctggt 3061 accagccctc agtgtggagg | | |
| | ctgagctggt gcctggcccc agtcttatct | | |
| | gtgcctttac 3121 tgctttgcgc atctcagatg | | |
| | ctaacttggt tctttttcca gaaggctttg | | |
| | tattggttaa 3181 aaattatttt ctattgcaga | | |
| | gagcagctgt gactcatgca aaaagtattt | | |
| | tctctgtcag 3241 atccccactc tataccaagg | | |
| | atattattaa aactagaaat gactgcattg | | |
| | agagggagtt 3301 gtgggaaata agaagaatga | | |
| | aagcctctct ttctgtccgc agatcctgac | | |
| | ttttccaaag 3361 tgccttaaaa gaaatcagac | | |
| | aaatgccctg agtggtaact tctgtgttat | | |
| | tttactctta 3421 aaaccaaact ctaccttttc | | |
| | ttggttacct 3481 tctcattcat gtcaagtatg | | |
| | tggttcattc ttagaaccaa gggaaatact | | |
| | gctcccccca 3541 tttgctgacg tagtgctctc | | |
| | atgggctcac ctgggcccaa ggcacagcca | | |
| | gggcacagtt 3601 aggcctggat gtttgcctgg | | |
| | tccgtgagat gccgcgggtc ctgtttcctt | | |
| | actggggatt 3661 tcagggctgg gggttcaggg | | |
| | agcatttcct tttcctggga gttatgtacc | | |
| | gcgaagtgtg 3721 tcatgtgccg tgcccttttc | | |
| | tgtttctgtg tatcctattg ctggtgactc | | |
| | tgtgtgaact 3781 ggcctttggg aaagatcaga | | |
| | gaggcagagg tggcacagga cagtaaagga | | |
| | gatgctgtgc 3841 tgcctacagc ctggacaggg | | |
| | tctctgctgt actgccaggg gcgggggctc | | |
| | tgcatagcca 3901 ggatgacgcc tttcatgtcc | | |
| | cagagacctg ttgtgctgtg tattttgatt | | |
| | tcctgtgtat 3961 gcaaatgtgt gtatttacca | | |
| | ttgtgtaggg ggctgtgtct gatcttggtg | | |
| | ttcaaaacag 4021 aactgtattt ttgcctttaa aattaaataa | | |
| | tataacgtga ataaatgacc ctaactttgt | | |
| α-1,4 | cgcgccgccc gcccgccgcc gctggagcta | NM_017436 | Seq ID No.4 |
| galactosyl | gagatggatt tgcagccgct gcaagtgtgt 61 | | |
| transferase | ggaagggccg tgttcgtgtt ggcaaagaag | | |
| | gtcggctgct gagccagggc gtgtctcccg 121 | | |
| | gaggcctgtg ggctgccagg atccccacct | | |
| | ctctgcaatg ggctgcccag gctgaccagc 181 | | |
| | cggttcctgc tggaagctcc tggtctgatc | | |
| | tggggatacc atgtccaagc cccccgacct 241 | | |
| | cctgctgcgg ctgctccggg gcgccccaag | | |
| | gcagcgggtc tgcaccctgt tcatcatcgg 301 | | |
| | cttcaagttc acgtttttcg tctccatcat | | |
| | gatctactgg cacgttgtgg gagagcccaa 361 | | |
| | ggagaaaggg cagctctata acctgccagc | | |
| | agagatcccc tgccccacct tgacaccccc 421 | | |
| | caccccaccc tcccacggcc ccactccagg | | |
| | caacatcttc ttcctggaga cttcagaccg 481 | | |
| | gaccaacccc aacttcctgt tcatgtgctc | | |
| | ggtggagtcg gccgccagaa ctcaccccga 541 | | |
| | atcccacgtg ctggtcctga tgaaagggct | | |
| | tccgggtggc aacgcctctc tgccccggca 601 | | |
| | cctgggcatc tcacttctga gctgcttccc | | |
| | gaatgtccag atgctcccgc tggacctgcg 661 | | |
| | ggagctgttc cgggacacac ccctggccga | | |
| | ctggtacgcg gccgtgcagg ggcgctggga 721 | | |
| | gccctacctg ctgcccgtgc tctccgacgc | | |
| | ctccaggatc gcactcatgt ggaagttcgg 781 | | |
| | cggcatctac ctggacacgg acttcattgt | | |
| | tctcaagaac ctgcggaacc tgaccaacgt 841 | | |
| | gctgggcacc cagtcccgct acgtcctcaa | | |
| | cggcgcgttc ctggccttcg agcgccggca 901 | | |
| | cgagttcatg gcgctgtgca tgcgggactt | | |
| | cgtggaccac tacaacggct ggatctgggg 961 | | |
| | tcaccagggc ccgcagctgc tcacgcgggt | | |
| | cttcaagaag tggtgttcca tccgcagcct 1021 | | |
| | ggccgagagc cgcgcctgcc gcggcgtcac | | |
| | caccctgccc cctgaggcct tctaccccat 1081 | | |
| | cccctggcag gactggaaga agtactttga | | |
| | ggacatcaac cccgaggagc tgccgcggct 1141 | | |
| | gctcagtgcc acctatgctg tccacgtgtg | | |
| | gaacaagaag agccagggca cgcggttcga 1201 | | |
| | ggccacgtcc agggcactgc tggcccagct | | |
| | gcatgcccgc tactgcccca cgacgcacga 1261 | | |
| | ggccatgaaa atgtacttgt gaggggcccg | | |
| | ccaggtcacc tccccaacct gctcctgatg 1321 | | |
| | gggcactggg ccgcccttcc cggggaggca | | |
| | agattgaggg cccgggagag ggaggcccga 1381 | | |
| | gctgccaccg ggcttaggca ggctgttgag | | |
| | gagctgtggg agcaggccca gtgggaggct 1441 | | |
| | gtggacaccc cgaggacagt gtcctgtctc | | |
| | gaggcagggc tgacacatgg tgccatagcc 1501 | | |
| | agcggagggc gctcagtgag tgccccgggc | | |
| | cttctagaca acaggcagga aggatgaacc 1561 | | |
| | tcagggcacc cccaggtggt gcggaaagcc | | |
| | aggcagttgg gacagaggtg cccacgaggg 1621 | | |
| | cagaggccgg tgctaagggg atggggaaga | | |
| | agggacaaga ttcccagaga ggagaggagg 1681 | | |
| | ctgttggtag gaaagtggca gggctggggg | | |
| | agacccagcc ccaagggtcc ggggcggagg 1741 | | |
| | atgctttgtt cttttctggt tttggttcct | | |
| | ctttcgcggg gggtggggga ggtcaacagg 1801 | | |
| | gactgagtgg ggcagaggcc cagaagtgcc | | |
| | agcctgggga gccgtttggg ggcagcccct 1861 | | |
| | tctgcccacc ccatccttct tcctctccag | | |
| | agatgccagg ggggcgtgta tgctctaccc 1921 | | |
| | cttccctcag acaggggctg ggtggggagg | | |
| | ctctttaggc tcaggagaag cattttaaag 1981 | | |
| | aaacccccac cctgccgccc gcattataaa | | |
| | cacaggagaa taatcaatag aataaaagtg 2041 | | |
| | accgactgtc aaaaaaaaaa aaaaa | | |
| β-1,4 N- | tggatcacag tctccatcga ctgactcagg | NM-022860 | Seq ID No.5 |
| acetylgalactosa | atgcggctgg accgccgggc cctctatgcg | | |
| minyl- | 61 ctagttctgc tgcttgcctg cgcctcgctg | | |
| transferase | ggtctcctgt acgccagcac ccgagacgcg 121 | | |
| | ccaggtctcc cgaaccctct ggcattgtgg | | |
| | tcacccccac aaggtccccc gaggctcgat 181 | | |
| | ctgctagacc ttgccactga gcctcgctac | | |
| | gcacacatcc cagtcaggat caaggagcaa 241 | | |
| | gtggtggggc tgctggctca gaacaattgc | | |
| | agttgtgagt ccagcggagg acgctttgcc 301 | | |
| | ttgccgttcc tgaggcaggt ccgggcgatt | | |
| | gacttcacta aagcctttga cgccgaggag 361 | | |
| | ctgagggctg tttctatctc cagagagcag | | |
| | gaataccagg ccttccttgc aaggagccgg 421 | | |
| | tccctggctg accagctgct gatagcccct | | |
| | gccaactccc ccttacagta tcccctgcag 481 | | |
| | ggtgtggagg ttcagcccct caggagcatc | | |
| | ctggtgccag ggctaagtct gcaggaagct 541 | | |
| | tctgttcagg aaatatatca ggtgaacctg | | |
| | attgcttccc ttggcacctg ggatgtggca 601 | | |
| | ggggaagtaa caggggtgac tctcactgga | | |
| | gaggggcagt cggacctcac ccttgccagc 661 | | |
| | ccaattctgg ataaactcaa ccgacagctg | | |
| | caactggtga cttacagcag ccggagctac 721 | | |
| | caagccaaca cagcagacac agtccggttc | | |
| | tccaccaagg gacatgaagt ggccttcacc 781 | | |
| | atcctcataa gacatcctcc caacccccgg | | |
| | ctgtacccac catcatccct accccaagga 841 | | |
| | gcccagtaca acatcagtgc tctggttacc | | |
| | gttgccacca agacctttct tcgttatgat 901 | | |
| | cggctacggg cactcattgc cagcatcaga | | |
| | cgcttttacc ctacggtcac catagtaatc 961 | | |
| | gctgacgaca gcgacaaacc ggagcgaatt | | |
| | agcgaccccc atgtggagca ctatttcatg 1021 | | |
| | cccttcggca agggttggtt tgcaggtcgg | | |
| | aacctggcgg tgtcccaagt aaccaccaaa 1081 | | |
| | tacgtgctgt gggtggacga cgactttgtc | | |
| | ttcacggcgc gcacgcggct ggagaagctt 1141 | | |
| | gtggatgtcc tggagaggac gcccctggac | | |
| | ttggttgggg gcgcggtgcg ggagatctcg 1201 | | |
| | ggctacgcta ccacctaccg acagctgcta | | |
| | agtgtggagc cgggcgcccc aggctttggg 1261 | | |
| | aactgcctcc ggcaaaagca gggcttccac | | |
| | cacgagctcg ctggctttcc aaactgcgtg 1321 | | |
| | gtcaccgacg gcgtagtcaa cttcttcctg | | |
| | gcgcgcacag ataaagtgcg ccaggtgggc 1381 | | |
| | tttgacccac gcctcaaccg ggtggctcat | | |
| | ctggaattct tcctggatgg tcttggttcc 1441 | | |
| | cttcgagttg gctcctgctc tgatgttgtt | | |
| | gtggatcatg cgtcaaaggt gaagctgcct 1501 | | |
| | tggacatcaa aggatccagg ggctgaactt | | |
| | tatgcccgtt accgttaccc gggatcactg 1561 | | |
| | gaccaaagtc aggtggccaa acatcgactg | | |
| | ctcttcttca aacaccggct acagtgcatg 1621 | | |
| | accgccgagt aacgtctgat ttgggccttc | | |
| | acactgtcag gctgggcctg cctcctccct 1681 | | |
| | gccaggaatt tccagcaacc accccccccc | | |
| | aatccctgag caccccactg atgaacaccc 1741 | | |
| | tggcttcccg accctctcca ccaatctgat | | |
| | tcctaacagg ggcttgtcct ggtgacaccc 1801 | | |
| | ttcctttctg tgagtgacca gaggccagat | | |
| | ggagccatat cctcccccac agccagtgcc 1861 | | |
| | aagtcctccc caaccccact cctatggggc | | |
| | aggaaatggg gaggttcact ttccaagtgc 1921 | | |
| | caaagagccc agacggactc taagaccctc | | |
| | aagtggaaac actctcacct cctgaggtgg 1981 | | |
| | gcagggaaac tcccaatttg caaccccagg | | |
| | gacatgcacc ccaccccagc tctggatcca 2041 | | |
| | gcaccatgtg tcccggctcc aacatacccc | | |
| | tacagaaagc actgtgactg tagttctgtg 2101 | | |
| | gggctggtga acacacggtg gaagccaaaa | | |
| | aaaaaaaaaa aaaaaaaaaa gggggggggg 2161 | | |
| | ggatcc | | |
| α-1,4 N- | tttttaaatt ttgcatttga cttaaagtgc | NM_020474 | Seq ID No.6 |
| acetylgalactosa | catgagaaaa tttgcatact gcaaggtggt 61 | | |
| minyl- | cctagccacc tccttgattt gggtactctt | | |
| transferase | ggatatgttc ctgctgcttt acttcagtga 121 | | |
| | atgcaacaaa tgtgatgaaa aaaaggagag | | |
| | aggacttcct gctggagatg ttctagagcc 181 | | |
| | agtacaaaag cctcatgaag gtcctggaga | | |
| | aatggggaaa ccagtcgtca ttcctaaaga 241 | | |
| | ggatcaagaa aagatgaaag agatgtttaa | | |
| | aatcaatcag ttcaatttaa tggcaagtga 301 | | |
| | gatgattgca ctcaacagat ctttaccaga | | |
| | tgttaggtta gaagggtgta aaacaaaggt 361 | | |
| | gtatccagat aatcttccta caacaagtgt | | |
| | ggtgattgtt ttccacaatg aggcttggag 421 | | |
| | cacacttctg cgaactgtcc atagtgtcat | | |
| | taatcgctca ccaagacaca tgatagaaga 481 | | |
| | aattgttcta gtagatgatg ccagtgaaag | | |
| | agactttttg aaaaggcctt tagagagtta 541 | | |
| | tgtgaaaaaa ctaaaagtac cagttcatgt | | |
| | aattcgaatg gaacaacgtt ctggattgat 601 | | |
| | cagagctaga ttaaaaggag ctgctgtgtc | | |
| | taaaggccaa gtgatcacct tcctggatgc 661 | | |
| | ccattgtgag tgtacagtgg gatggctgga | | |
| | gcctctcttg gccaggatca aacatgacag 721 | | |
| | gagaacagtg gtgtgtccca tcatcgatgt | | |
| | gatcagtgat gatacttttg agtacatggc 781 | | |
| | aggctctgat atgacctatg gtgggttcaa | | |
| | ctggaagctc aattttcgct ggtatcctgt 841 | | |
| | tccccaaaga gaaatggaca gaaggaaagg | | |
| | tgatcggact cttcctgtca ggacacctac 901 | | |
| | catggcagga ggcctttttt caatagacag | | |
| | agattacttt caggaaattg gaacatatga 961 | | |
| | tgctggaatg gatatttggg gaggagaaaa | | |
| | cctagaaatt tcctttagga tttggcagtg 1021 | | |
| | tggaggaact ttggaaattg ttacatgctc | | |
| | acatgttgga catgtgtttc ggaaagctac 1081 | | |
| | accttacacg tttccaggag gcacagggca | | |
| | gattatcaat aaaaataaca gacgacttgc 1141 | | |
| | agaagtgtgg atggatgaat tcaagaattt | | |
| | cttctatata atttctccag gtgttacaaa 1201 | | |
| | ggtagattat ggagatatat cgtcaagagt | | |
| | tggtctaaga cacaaactac aatgcaaacc 1261 | | |
| | tttttcctgg tacctagaga atatatatcc | | |
| | tgattctcaa attccacgtc actatttctc 1321 | | |
| | attgggagag atacgaaatg tggaaacgaa | | |
| | tcagtgtcta gataacatgg ctagaaaaga 1381 | | |
| | gaatgaaaaa gttggaattt ttaattgcca | | |
| | tggtatgggg ggtaatcagg ttttctctta 1441 | | |
| | tactgccaac aaagaaatta gaacagatga | | |
| | cctttgcttg gatgtttcca aacttaatgg 1501 | | |
| | cccagttaca atgctcaaat gccaccacct | | |
| | aaaaggcaac caactctggg agtatgaccc 1561 | | |
| | agtgaaatta accctgcagc atgtgaacag | | |
| | taatcagtgc ctggataaag ccacagaaga 1621 | | |
| | ggatagccag gtgcccagca ttagagactg | | |
| | caatggaagt cggtcccagc agtggcttct 1681 | | |
| | tcgaaacgtc accctgccag aaatattctg | | |
| | agaccaaatt tacaaaaaaa cgaaaaaaat 1741 | | |
| | aaggattgac tgggctacct cagcatacat | | |
| | ttctgccaca ttcttaagta gcaaaaaagg 1801 | | |
| | aaaagtgctt tcctcctctg caggatgtaa | | |
| | ggtttatcag ccattaaaac ttagacttct 1861 | | |
| | ctagcttttc actagctgtg aaccagcctt | | |
| | cctgtccatg gacgtgaaac tgcatagtaa 1921 | | |
| | tgagactgtg cacactgatg tttacaagat | | |
| | tgaaagagtc tttctccgaa aatcatggta 1981 | | |
| | aagaatactg agacaatgaa aaaaaatcaa | | |
| | caaaatatgc tttctggaga actgtacctt 2041 | | |
| | ctatggtttg cttgcacatc agtagtttct | | |
| | gctgaacgtg ctgtcataat gaagagattt 2101 | | |
| | ccaagatttt ttttcctgat tagaacgggt | | |
| | agccagtata ttaaatattg atagaaaaat 2161 | | |
| | aaaagaactg gaaccagatt cagaatcttg | | |
| | aaaacaacat tttttacaac aaacaaaaaa 2221 | | |
| | actatattaa acagggttta aaggaaaatt | | |
| | aaaacagaac tatgaagaag tacaatttgt 2281 | | |
| | tatagtatag tatcaaattt ctatatagat | | |
| | tttatacctc agtggggaaa aataactgat 2341 | | |
| | tccaatgaca ttcattttgt tttcatctgt | | |
| | gatagtcatg gatgctttta ttttccttgg 2401 | | |
| | ggtgctgaaa ttgagctgaa aaaaaaaggc | | |
| | tctttgaata tagttttaat ttctctctac 2461 | | |
| | agtttttttt gtttggtttg tgggctgttg | | |
| | gaattgtaat ttttaattgc cttctaaaaa 2521 | | |
| | atggaaattt aacaatgtct gatctcagct | | |
| | gaacaaatta gatgtttcag ttgctcttgg 2581 | | |
| | gtcaactggc ttacagattt acatgtgcac | | |
| | acacacacaa atttcttatc acattttcga 2641 | | |
| | cttcttcact tgacctaact gattatgcga | | |
| | aatacccaag attcatgcta ctgttccaca 2701 | | |
| | tttgttttca cagcaataaa tcttcagttc | | |
| | tgttgtttat gattccactt aacaaggggc 2761 | | |
| | ctgcaaatgt gatttattat ttgggtattt | | |
| | ggagataata catttgaggg ttttttggaa 2821 | | |
| | aacctttttc actccatact caaatatgct | | |
| | tcattgtcaa atgcatattt aaattaaatt 2881 | | |
| | attgaattgt aatgtttatc tgctgctttt | | |
| | tttaaataaa atttgactga aaatgtttaa 2941 | | |
| | ttggcatttt ttaatgactt acccaagaaa | | |
| | agtgcagcta ttattccata ttaataggct 3001 | | |
| | tgcatttctt ttcctaaatc ttatttaggc | | |
| | taaatcagtt ttattgtcct ctgatttttt 3061 | | |
| | ttaataccac agaaatcacc tgagtgtcaa | | |
| | ttgaaaagtt gtcaattaaa aggtaacctt 3121 | | |
| | ttaactctcg taggaggaat ctcattaaga | | |
| | catttttcct gatatgtaga gcagtctgtt 3181 | | |
| | ggcaaaaatg catatatttt ctttcatatt | | |
| | tgtaaaatta tatttaatgg aattcttttc 3241 | | |
| | tttgattatc aaggactttc actgcaggca | | |
| | gtgctatttc ttgtgcctaa gaatgtttcc 3301 | | |
| | aaaagtcgca tcgctaatga | | |
| | tatttgccaagttgagtgta cacaaagttt | | |
| | ctcatatcct 3361 gttcaagtta atcaacatca | | |
| | aacacatggg gatgctttag ggtgagtcta | | |
| | taatacaaaa 3421 tgcataaacc atgtccccag | | |
| | gaaatttgaa aggaagcaag tgctgaatgg | | |
| | aatttttttc 3481 cttttccatg agctgtgtta | | |
| | attctatctc cagtaggcct aatgcttgaa | | |
| | ataagcaaga 3541 tgtctaatca ataaattatt | | |
| | ttcatgctca gaatttcagg tttttgtact | | |
| | ccagcatagc 3601 ttggtcttat ttcttactgt | | |
| | atgaaagctt aacagcaatg tgatttaagg | | |
| | ttttgtttta 3661 aatgggagat gtaagtgatt | | |
| | taattcatgg gtacttttag aacctgatag | | |
| | ataatcccat 3721 tgcctttatt tttctaatta | | |
| | aagaatccta aatactttga aaatacaaaa tattcctg | | |
| β-1,6 N- | attaactggg ttttcctatt tatctatcct | BD230936 | Seq ID No.7 |
| acetylglucosam | ctcgcattac ttctctgagt cagagcctct 61 | | |
| ine transferase | tctctctaag tcacgggaac tgcccttgct | | |
| | acttgtgacc tgccctttac tcagcagttt 121 | | |
| | ttgttctggg aagccctggg attctgctaa | | |
| | tacctatcac tgtaggtgct gaagggaaac 181 | | |
| | agatgaagaa catgacctca aggagcttcc | | |
| | tgtcaatgag aagaccaagc tgacgcctgg 241 | | |
| | caaagatatt aaagaggagc ctgaaactgt | | |
| | tccttggaca tcttatgaat gtcagaaaat 301 | | |
| | accttttgga gggttagaag atcaggggac | | |
| | atggttgttc acatttgctg ccacggaaca 361 | | |
| | ccgccagtct tcacttggaa acagaatcac | | |
| | gccttgtgaa gagatcatcc ctaagcagga 421 | | |
| | gagaagctac taaaggattg tgtcctcctc | | |
| | caccttccct gtgctcggtc tccacctgtc 481 | | |
| | tcccattctg tgacgatggt tcaatggaag | | |
| | agactctgcc agctgcatta cttgtgggct 541 | | |
| | ctgggctgct atatgctgct gccactgtggctctgaaac | | |
| | tttctttcag gttgaagtgt 601 gactctgacc | | |
| | acttgggtct ggagtccagg gaatctcaaa | | |
| | gccagtactg taggaatatc 661 ttgtataatt | | |
| | tcctgaaact tccagcaaag aggtctatca | | |
| | actgttcagg ggtcacccga 721 ggggaccaag | | |
| | aggcagtgct tcaggctatt ctgaataacc | | |
| | tggaggtcaa gaagaagcga 781 gagcctttca | | |
| | cagacaccca ctacctctcc ctcaccagag | | |
| | actgtgagca cttcaaggct 841 gaaaggaagt | | |
| | tcatacagtt cccactgagc aaagaagagg | | |
| | tggagttccc tattgcatac 901 tctatggtga | | |
| | ttcatgagaa gattgaaaac tttgaaaggc | | |
| | tactgcgagc tgtgtatgcc 961 cctcagaaca | | |
| | tatactgtgt ccatgtggat gagaagtccc | | |
| | cagaaacttt caaagaggcg 1021 gtcaaagcaa | | |
| | ttatttcttg cttcccaaat gtcttcatag | | |
| | ccagtaagct ggttcgggtg 1081 gtttatgcct | | |
| | cctggtccag ggtgcaagct gacctcaact | | |
| | gcatggaaga cttgctccag 1141 agctcagtgc | | |
| | cgtggaaata cttcctgaat acatgtggga | | |
| | cggactttcc tataaagagc 1201 aatgcagaga | | |
| | tggtccaggc tctcaagatg ttgaatggga | | |
| | ggaatagcat ggagtcagag 1261 gtacctccta | | |
| | agcacaaaga aacccgctgg aaatatcact | | |
| | ttgaggtagt gagagacaca 1321 ttacacctaa | | |
| | ccaacaagaa gaaggatcct cccccttata | | |
| | atttaactat gtttacaggg 1381 aatgcgtaca | | |
| | ttgtggcttc ccgagatttc gtccaacatg | | |
| | ttttgaagaa ccctaaatcc 1441 caacaactga | | |
| | ttgaatgggt aaaagacact tatagcccag | | |
| | atgaacacct ctgggccacc 1501 cttcagcgtg | | |
| | cacggtggat gcctggctct gttcccaacc | | |
| | accccaagta cgacatctca 1561 gacatgactt | | |
| | ctattgccag gctggtcaag tggcagggtc | | |
| | atgagggaga catcgataag 1621 ggtgctcctt | | |
| | atgctccctg ctctggaatc caccagcggg | | |
| | ctatctgcgt ttatggggct 1681 ggggacttga | | |
| | attggatgct tcaaaaccat cacctgttgg | | |
| | ccaacaagtt tgacccaaag 1741 gtagatgata | | |
| | atgctcttca gtgcttagaa gaatacctac | | |
| | gttataaggc catctatggg 1801 actgaacttt | | |
| | gagacacact atgagagcgt tgctacctgt | | |
| | ggggcaagag catgtacaaa 1861 catgctcaga | | |
| | acttgctggg acagtgtggg tgggagacca | | |
| | gggctttgca attcgtggca 1921 tcctttagga | | |
| | taagagggct gctattagat tgtgggtaag | | |
| | tagatctttt gccttgcaaa 1981 ttgctgcctg | | |
| | ggtgaatgct gcttgttctc tcacccctaa | | |
| | ccctagtagt tcctccacta 2041 actttctcac | | |
| | taagtgagaa tgagaactgc tgtgataggg | | |
| | agagtgaagg agggatatgt 2101 ggtagagcac | | |
| | ttgatttcag ttgaatgcct gctggtagct | | |
| | tttccattct gtggagctgc 2161 cgttcctaat | | |
| | aattccaggt ttggtagcgt ggaggagaac | | |
| | tttgatggaa agagaacctt 2221 cccttctgta | | |
| | ctgttaactt aaaaataaat agctcctgat | | |
| | tcaaagtatt acctctactt 2281 tttgcctagt | | |
| | atgccagaaa taatataaat ctaaacaga | | |
| β-1,6 N- | aacagggcag gagtgagtgg agtatgttgc | AF401652 | Seq ID No.8 |
| acetylglucosam | aaaataagaa ctcagagaaa cgagtgagtt 61 | | |
| ine transferase | tggaaaaaag acttacagat tttgacggtc | | |
| | tcttgacatt tcacccttct ttgaggcatg 121 | | |
| | cctttatcaa tgcgttacct cttcataatt | | |
| | tctgtctcta gtgtaattat ttttatcgtc 181 | | |
| | ttctctgtgt tcaattttgg gggagatcca | | |
| | agcttccaaa ggctaaatat ctcagaccct 241 | | |
| | ttgaggctga ctcaagtttg cacatctttt | | |
| | atcaatggaa aaacacgttt cctgtggaaa 301 | | |
| | aacaaactaa tgatccatga gaagtcttct | | |
| | tgcaaggaat acttgaccca gagccactac 361 | | |
| | atcacagccc ctttatctaa ggaagaagct | | |
| | gactttccct tggcatatat aatggtcatc 421 | | |
| | catcatcact ttgacacctt tgcaaggctc | | |
| | ttcagggcta tttacatgcc ccaaaatatc 481 | | |
| | tactgtgttc atgtggatga | | |
| | aaaagcaacaactgaattta aagatgcggt | | |
| | agagcaacta 541 ttaagctgct tcccaaacgc | | |
| | ttttctggct tccaagatgg aacccgttgt | | |
| | ctatggaggg 601 atctccaggc tccaggctga | | |
| | cctgaactgc atcagagatc tttctgcctt | | |
| | cgaggtctca 661 tggaagtacg ttatcaacac | | |
| | ctgtgggcaa gacttccccc tgaaaaccaa | | |
| | caaggaaata 721 gttcagtatc tgaaaggatt | | |
| | taaaggtaaa aatatcaccc caggggtgct | | |
| | gcccccagct 781 catgcaattg gacggactaa | | |
| | atatgtccac caagagcacc tgggcaaaga | | |
| | gctttcctat 841 gtgataagaa caacagcgtt | | |
| | gaaaccgcct cccccccata atctcacaat | | |
| | ttactttggc 901 tctgcctatg tggctctatc | | |
| | aagagagttt gccaactttg ttctgcatga | | |
| | cccacgggct 961 gttgatttgc tccagtggtc | | |
| | caaggacact ttcagtcctg atgagcattt | | |
| | ctgggtgaca 1021 ctcaatagga ttccaggtgt | | |
| | tcctggctct atgccaaatg catcctggac | | |
| | tggaaacctc 1081 agagctataa agtggagtga | | |
| | catggaagac agacacggag gctgccacgg | | |
| | ccactatgta 1141 catggtattt gtatctatga | | |
| | aaacggagac ttaaagtggc tggttaattc | | |
| | accaagcctg 1201 tttgctaaca agtttgagct | | |
| | taatacctac ccccttactg tggaatgcct | | |
| | agaactgagg 1261 catcgcgaaa gaaccctcaa | | |
| | tcagagtgaa actgcgatac aacccagctg | | |
| | gtatttttga 1321 gctattcatg agctactcat | | |
| | gactgaaggg aaactgcagc t | | |
| β-1,3 N- | gcggtaaatc cgggcttgcg gccgctggcg | AF029893 | Seq ID No.9 |
| acetylglucosam | tagtctgtgg ccgggtggtc gttgctgcgc 61 | | |
| inyl- | gccccgagcc ccgagagcca tgcagatgtc | | |
| transferase | ctacgccatc cggtgcgcct tctaccagct 121 | | |
| | gctgctggcc gcgctcatgc tggtggcgat | | |
| | gctgcagctg ctctacctgt cgctgctgtc 181 | | |
| | cggactgcac gggcaggagg agcaagacca | | |
| | atattttgag ttctttcccc cgtccccacg 241 | | |
| | gtccgtggac caggtcaagg cgcagctccg | | |
| | caccgcgctg gcctctggag gcgtcctgga 301 | | |
| | cgctagcggc gattaccgcg tctacagggg | | |
| | cctgctgaag accaccatgg accccaacga 361 | | |
| | tgtgatcctg gccacgcacg ccagcgtgga | | |
| | caacctgctg cacctgtcgg gtctgctgga 421 | | |
| | gcgctgggag ggcccgctgt ccgtgtcggt | | |
| | gttcgcggcc accaaggagg aggcgcagct 481 | | |
| | ggccacggtg ctggcctacg cgctgagcag | | |
| | ccactgcccc gacatgcgcg ccagggtcgc 541 | | |
| | catgcacctc gtgtgcccct cgcgttacga | | |
| | ggcagccgtg cccgaccccc gggagccggg 601 | | |
| | ggagtttgcc ctgctgcggt cctgccagga | | |
| | ggtctttgac aagctagcca gggtggccca 661 | | |
| | gcccgggatt aattatgcgc tgggcaccaa | | |
| | tgtctcctac cccaataacc tgctgaggaa 721 | | |
| | tctggctcgt gagggggcca | | |
| | actatgccctggtgatcgat gtggacatgg | | |
| | tgcccagcga 781 ggggctgtgg agaggcctgc | | |
| | gggaaatgct ggatcagagc aaccagtggg | | |
| | gaggcaccgc 841 gctggtggtg cctgccttcg | | |
| | aaatccgaag agcccgccgc atgcccatga | | |
| | acaaaaacga 901 gctggtgcag ctctaccagg | | |
| | ttggcgaggt gcggcccttc tattatgggt | | |
| | tgtgcacccc 961 ctgccaggca cccaccaact | | |
| | attcccgctg ggtcaacctg ccggaagaga | | |
| | gcttgctgcg 1021 gcccgcctac gtggtacctt | | |
| | ggcaggaccc ctgggagcca ttctacgtgg | | |
| | caggaggcaa 1081 ggtgcccacc ttcgacgagc | | |
| | gctttcggca gtacggcttc aaccgaatca | | |
| | gccaggcctg 1141 cgagctgcat gtggcggggt | | |
| | ttgattttga ggtcctgaac gaaggtttct | | |
| | tggttcataa 1201 gggcttcaaa gaagcgttga | | |
| | agttccatcc ccaaaaggag gctgaaaatc | | |
| | agcacaataa 1261 gatcctatat cgccagttca | | |
| | aacaggagtt gaaggccaag taccccaact | | |
| | ctccccgacg 1321 ctgctgagcc cttccctccc | | |
| | ctaatctgag aagtcagcct cttggctcct | | |
| | caggccacca 1381 tttaggcctg actggggtaa | | |
| | gaaatgtcgc tccactttac agaggtagct | | |
| | gtggtgttga 1441 aacactggac ttggatatgg | | |
| | ggtgctggga tcgattccta gctttaccac | | |
| | taactagctg 1501 tgtggccttg agtaaatccc | | |
| | gttacctctc tgagcctcgg ttaccctgtc | | |
| | tgtaaaaagg 1561 gaggtgagaa tacctacctc | | |
| | acggaactgt tgggaggctc agatgagatg | | |
| | ctatatgtga 1621 aaacattctg taagcttcgt | | |
| | acaaatgtga agtattaata ttatcgcagt | | |
| | attattgttg 1681 ttattattat tgttattatt | | |
| | aacaatcttg ggtgggtagt aggagagcaa | | |
| | aaagtatgaa 1741 tgggatggag ctaagaagtc | | |
| | tgaatactta atgaaatgga ctttttggaa | | |
| | agaaatcaga 1801 tgaaggcata aaatttagtt | | |
| | cttagctctt gaacagaagc ctaaaattcc | | |
| | tggttctctc 1861 gggcttcgc cttcaagggt | | |
| | tctggaggag ggaagggtct gcaggttcca | | |
| | tgggtgacag 1921 cctgagatct gtcccttcaa | | |
| | cgggctgggc tgggtatgtg cctaccgatg | | |
| | acaatgtgta 1981 aataaatgcg tgttcacacc | | |
| | cacaaaaaaa a | | |
| GalNAcT6 | atgaggctcc tccgcagacg ccacatgccc | NM_007210 | Seq ID No. 10 |
| (UDP-N-acetyl- | ctgcgcctgg ccatggtggg ctgcgccttt 61 | | |
| α-D- | gtgctcttcc tcttcctcct gcatagggat | | |
| galactosamine: | gtgagcagca gagaggaggc cacagagaag 121 | | |
| Polypeptide N- | ccgtggctga agtccctggt gagccggaag | | |
| Acetylgalactos | gatcacgtcc tggacctcat gctggaggcc 181 | | |
| aminyltransfer | atgaacaacc ttagagattc aatgcccaag | | |
| ase-T3) | ctccaaatca gggctccaga agcccagcag 241 | | |
| | actctgttct ccataaacca gtcctgcctc | | |
| | cctgggttct ataccccagc tgaactgaag 301 | | |
| | cccttctggg aacggccacc acaggacccc | | |
| | aatgcccctg gggcagatgg aaaagcattt 361 | | |
| | cagaagagca agtggacccc cctggagacc | | |
| | caggaaaagg aagaaggcta taagaagcac 421 | | |
| | tgtttcaatg cctttgccag cgaccggatc | | |
| | tccctgcaga ggtccctggg gccagacacc 481 | | |
| | cgaccacctg agtgtgtgga ccagaagttc | | |
| | cggcgctgcc ccccactggc caccaccagc 541 | | |
| | gtgatcattg tgttccacaa cgaagcctgg | | |
| | tccacactgc tgcgaacagt gtacagcgtc 601 | | |
| | ctacacacca cccctgccat cttgctcaag | | |
| | gagatcatac tggtggatga tgccagcaca 661 | | |
| | gaggagcacc taaaggagaa gctggagcag | | |
| | tacgtgaagc agctgcaggt ggtgagggtg 721 | | |
| | gtgcggcagg aggagcggaa ggggttgatc | | |
| | accgcccggc tgctgggggc cagcgtggca 781 | | |
| | caggcggagg tgctcacgtt cctggatgcc | | |
| | cactgtgagt gcttccacgg ctggctggag 841 | | |
| | cccctcctgg ctcgaatcgc tgaggacaag | | |
| | acagtggtgg tgagcccaga catcgtcacc 901 | | |
| | atcgacctta atacttttga gttcgccaag | | |
| | cccgtccaga ggggcagagt ccatagccga 961 | | |
| | ggcaactttg actggagcct gaccttcggc | | |
| | tgggaaacac ttcctccaca tgagaagcag 1021 | | |
| | aggcgcaagg atgaaacata ccccatcaaa | | |
| | tccccgacgt ttgctggtgg cctcttctcc 1081 | | |
| | atccccaagt cctactttga gcacatcggt | | |
| | acctatgata atcagatgga gatctgggga 1141 | | |
| | ggggagaacg tggaaatgtc cttccgggtg | | |
| | tggcagtgtg ggggccagct ggagatcatc 1201 | | |
| | ccctgctctg tcgtaggcca tgtgttccgg | | |
| | accaagagcc cccacacctt ccccaagggc 1261 | | |
| | actagtgtca ttgctcgcaa tcaagtgcgc | | |
| | ctggcagagg tctggatgga cagctacaag 1321 | | |
| | aagattttct ataggagaaa tctgcaggca | | |
| | gcaaagatgg cccaagagaa atccttcggt 1381 | | |
| | gacatttcgg aacgactgca gctgagggaa | | |
| | caactgcact gtcacaactt ttcctggtac 1441 | | |
| | ctgcacaatg tctacccaga gatgtttgtt | | |
| | cctgacctga cgcccacctt ctatggtgcc 1501 | | |
| | atcaagaacc tcggcaccaa ccaatgcctg | | |
| | gatgtgggtg agaacaaccg cggggggaag 1561 | | |
| | cccctcatca tgtactcctg ccacggcctt | | |
| | ggcggcaacc agtactttga gtacacaact 1621 | | |
| | cagagggacc ttcgccacaa catcgcaaag | | |
| | cagctgtgtc tacatgtcag caagggtgct 1681 | | |
| | ctgggccttg ggagctgtca ttcactggcaagaatagcc | | |
| | aggtccccaa ggacgaggaa 1741 tgggaattgg | | |
| | cccaggatca gctcatcagg aactcaggat | | |
| | ctggtacctg cctgacatcc 1801 caggacaaaa | | |
| | agccagccat ggccccctgc aatcccagtg | | |
| | acccccatca gttgtggctc 1861 tttgtctagg | | |
| | acccagatca tccccagaga gagcccccac | | |
| | aagctcctca ggaaacagga 1921 ttgctgatgt | | |
| | ctgggaacct gatcaccagc ttctctggag | | |
| | gccgtaaaga tggatttcta 1981 aacccactgg | | |
| | gtggcaaggc aggaccttcc taatccttgc | | |
| | aacaacattg ggcccatttt 2041 ctttccttca | | |
| | caccgatgga agagaccatt aggacatata | | |
| | tttagcctag cgttttcctg 2101 ttctagaaat | | |
| | agaggctccc aaagtaggga aggcagctgg | | |
| | gggagggttc agggcagcaa 2161 tgctgagttc | | |
| | aagaaaagta cttcaggctg ggcacagtgg | | |
| | ctcatgcctg aaatcctagc 2221 actttgggaa | | |
| | gacaatgtgg gagaatggct tgagcccagg | | |
| | agttcaagac cggcctgagc 2281 aacatagtga | | |
| | ggatcccatc tctacgccca ccctcccccc | | |
| | ggcaaaaaaa aaagctgggt 2341 atggtggctt | | |
| | atgcctgtag tcgcagctac tcagaaggct | | |
| | gaggtgggag gattgcttgt 2401 tccccggagg | | |
| | ttgaagctac agtgagcctt gattgtgtca | | |
| | ctgcactcca gcctgggcaa 2461 caggtaagac | | |
| | tctgtctcaa aaaaaaaaca aaaaagaaga | | |
| | agaaaagtac ttctacagcc 2521 atgtcctatt | | |
| | ccttgatcat ccaaagcacc tgcagagtcc | | |
| | agtgaaatga tatattctgg 2581 ctgggcacag | | |
| | tggctcacac ctgtaatcct agcactttgg | | |
| | gaggccaagg caggtggatc 2641 acctgaggtc | | |
| | agaagtttga aaccagcctg gactacatgg | | |
| | tgaaactcca tctctactaa 2701 aagtacaaaa | | |
| | attagctggg catgatggca cgcacctgca | | |
| | gtcccagcta cttgggaggc 2761 tgaggcagga | | |
| | gaatcactcg aacccaggag gcagaggttg | | |
| | cagtgagcca agacagcacc 2821 attgcacccc | | |
| | agcctgagca acaagagcga aactccatct | | |
| | caggaaaaaa aaaaaaaaaa 2881 a | | |
| β-1,3 N- | attcccacct cctccagaag ccccgcccac | NM_030765 | Seq ID No. 11 |
| acetylglucosam | tcccgagccc cgagagctcc gcgcacctgg 61 | | |
| inyl- | gcgccatccg ccctggctcc gctgcacgag | | |
| transferase 4 | ctccacgccc gtaccccggc gtcacgctca 121 | | |
| | gcccgcggtg ctcgcacacc tgagactcat | | |
| | ctcgcttcga ccccgccgcc gccgccgccc 181 | | |
| | ggcatcctga gcacggagac agtctccagc | | |
| | tgccgttcat gcttcctccc cagccttccg 241 | | |
| | cagcccacca gggaaggggc ggtaggagtg | | |
| | gccttttacc aaagggaccg gcgatgctct 301 | | |
| | gcaggctgtg ctggctggtc tcgtacagct | | |
| | tggctgtgct gttgctcggc tgcctgctct 361 | | |
| | tcctgaggaa ggcggccaagccgcaggagaccccacggc | | |
| | ccaccagcct ttctgggctc 421 ccccaacacc | | |
| | ccgtcacagc cggtgtccac ccaaccacac | | |
| | agtgtctagc gcctctctgt 481 ccctgcctag | | |
| | ccgtcaccgt ctcttcttga cctatcgtca | | |
| | ctgccgaaat ttctctatct 541 tgctggagcc | | |
| | ttcaggctgt tccaaggata ccttcttgct | | |
| | cctggccatc aagtcacagc 601 ctggtcacgt | | |
| | ggagcgacgt gcggctatcc gcagcacgtg | | |
| | gggcagggtg gggggatggg 661 ctaggggccg | | |
| | gcagctgaag ctggtgttcc tcctaggggt | | |
| | ggcaggatcc gctcccccag 721 cccagctgct | | |
| | ggcctatgag agtagggagt ttgatgacat | | |
| | cctccagtgg gacttcactg 781 aggacttctt | | |
| | caacctgacg ctcaaggagc tgcacctgca | | |
| | gcgctgggtg gtggctgcct 841 gcccccaggc | | |
| | ccatttcatg ctaaagggag atgacgatgt | | |
| | ctttgtccac gtccccaacg 901 tgttagagtt | | |
| | cctggatggc tgggacccag cccaggacct | | |
| | cctggtggga gatgtcatcc 961 gccaagccct | | |
| | gcccaacagg aacactaagg tcaaatactt | | |
| | catcccaccc tcaatgtaca 1021 gggccaccca | | |
| | ctacccaccc tatgctggtg ggggaggata | | |
| | tgtcatgtcc agagccacag 1081 tgcggcgcct | | |
| | ccaggctatc atggaagatg ctgaactctt | | |
| | ccccattgat gatgtctttg 1141 tgggtatgtg | | |
| | cctgaggagg ctggggctga gccctatgca | | |
| | ccatgctggc ttcaagacat 1201 ttggaatccg | | |
| | gcggcccctg gaccccttag acccctgcct | | |
| | gtataggggg ctcctgctgg 1261 ttcaccgcct | | |
| | cagccccctc gagatgtgga ccatgtgggc | | |
| | actggtgaca gatgaggggc 1321 tcaagtgtgc | | |
| | agctggcccc ataccccagc gctgaagggt | | |
| | gggttgggca acagcctgag 1381 agtggactca | | |
| | gtgttgattc tctatcgtga tgcgaaattg | | |
| | atgcctgctg ctctacagaa 1441 aatgccaact | | |
| | tggtttttta actcctctca ccctgttagc | | |
| | tctgattaaa aacactgcaa 1501 cccaaaaaaa | | |
| | aaaaaaaaaa aaaaaaaaaa aaaaaaaaaa | | |
| | aaaaaaaaaa aaaa | | |

α-1,3 galactosyltransferase (α-1,3-GT)

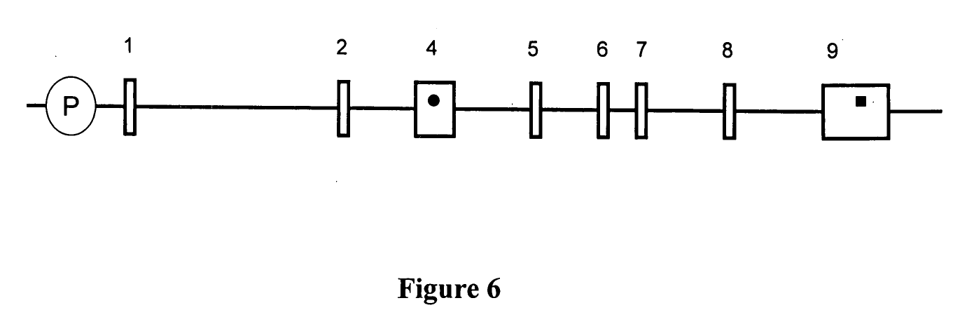

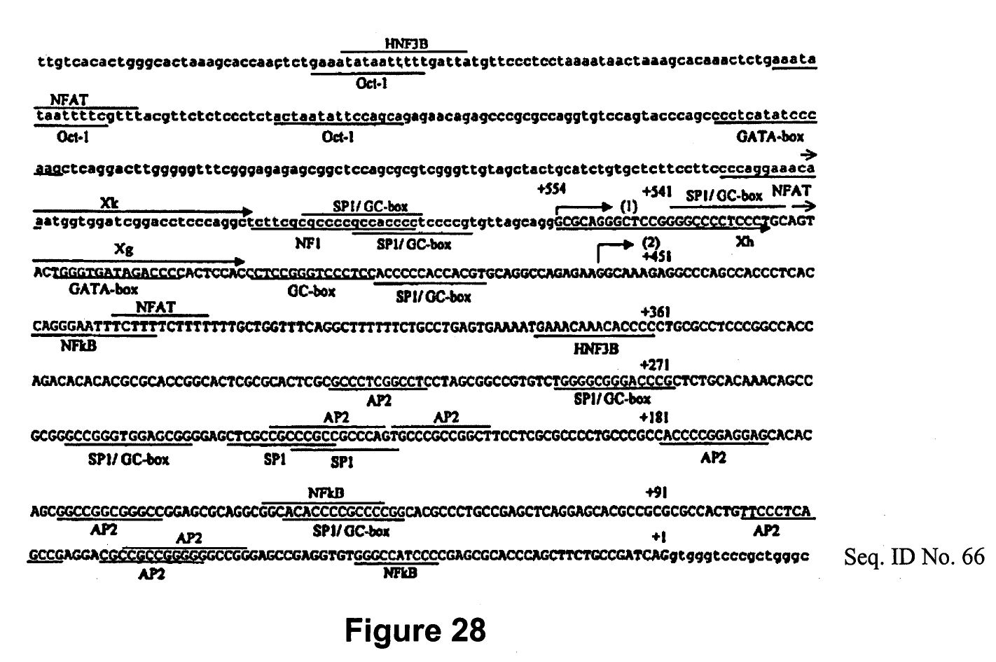

In one embodiment of the present invention, α-1,3-GT genomic sequence can be used to design constructs that target the α-1,3-GT gene. The genomic organization of the α-1,3-GT gene is provided in FIG. 6. The genomic sequence of the porcine α-1,3-GT is provided below in Table 4. In other embodiments of the present invention, the promoter sequence of the α-1,3-GT gene can be utilized, the promoter for the porcine α-1,3-GT gene is provided in FIG. 28.

TABLE 4
Genomic Sequence for alpha-1,3-galactosyltransferase

| | | |
|---|---|---|
| aggcctaaac ctagaactcc tgaccctgaa gctaaggaat ataatcttga aggtgttttc | Intron 1 | Seq. ID No. 12 |
| cagtcagtag aataacacag agtttccaca catgcgtggg tctctttcta ggttgcttat | | |
| tctgttccat tggtccaata aaccatcctg gcgctaatgc tatactgagt tcactgcgtt | | |
| tcatggtctg tcttggtatc tggtggaaca agagcccaac tctcccctcc ctgctttgtc | | |
| aagactgcct tggttatatc tggccccttc ccgctgctgt ccaaatttta agaatagctg | | |
| gccaagctcc cccaaaactc tgttggcatt tgtcttgagt ttataggttg atgcatggag | | |
| aattgttgcc ttcgtgatgc tgatgctttc cagtgctcac tcgggggtct ctttccttcc | | |
| acctaaagac ttctgcacat ggttctgctt gggtcactct tccccaagcc ttcacctagt | | |
| gaactcctcc tcctcctggt ctcagggtct cctgcaccct tatttcttcc ttagagccct | | |
| gatcacaatg gtcctgaaat cactcattgc gtgggtcttn gtgacagata gtaggtccca | | |
| gtaaatatct gttaaaagaa tgaaggaagt ttaggtagga aggtcttcgg gacctggagc | | |
| accttggcca tagttagagg gatggtgacc agaggtactt aacttgcctg tgccttggct | | |
| ttcttcctac aaaaccggga tgtgatcaga atgtgtataa gatgaagtga gctcagctag | | |
| gccgtgaggc aagtggagca aagcctggca agggatcaga gctacttgtt tacctgccct | | |
| gcccttctgc tcagtgaatc ttcagtcctg cactcctgtg atgctcctgg aggctccaac | | |
| actctttccc cagcagtgat cccgtcttga ctccacctct cctatgaact agtcacctta | | |
| tttctactca gcatatgaca caaatgagtc tcaggaagaa tgactcataa ggccttaaac | | |
| ctagaactcc tgaccctgaa gctaaggaat ataatcttga aggtgttttc cagtcagtag | | |
| aattgctagt tagatttggg gagctacata gttctcaaaa gaaaacaaaa cttccggacc | | |
| cgccgtgtta atttgaatta tttttatctt attgttactg aaataggtat aaacctagaa | | |
| ctaagaatga agtcctcatg ctcctagctc tgcacaccta ccatgatacc aaagcaaatc | | |
| ttttaagtag gtgcaattac agccacaaaa ccaataaaat ccaaattagc aacgttaaat | | |
| ttatgcaact gatgacatgg tgctgaaatc aaacctcttg cattgagtct aatggtagca | | |
| gagtgatgtt tttacatgtt tcattccctg tgtcatcatc ttttgatttt gatcctgatg | | |
| agctatcact tcagccatgg tcagaattac cgtcataatt ttcactaaaa aaaaaaccca | | |
| aaaaacacat ttattatcca atttgatggg ctgagcaatt taaacactgg atcctcaagt | | |
| gcaataatga caactgggaa atactttgct aacatcactc cttgtgtatt tatttactgc | | |
| atcattaaag acctagtgca agtgagttca ccgatgacaa taatggcgca gtttatgctt | | |
| ttgcaaagga tccattgttc ggattgtcat ggagctcctc attcctgagc taccctgtgg | | |
| ggctgatgat tcaactctcc caccctttag tccactgaac ccatcaggaa agttcattat | | |
| cccaagctcc aagatgtcac ttggctccct gcagcctctc tgcaaccgtc aagtattcaa | | |
| tcagatctct gttcttttca aatcaggatg aaacagttaa aattatacat cacactcagg | | |
| ttctgtgcca ttttcatgtc acaattccaa tgccttaaaa tatttaagaa actaatttct | | |
| tagtctctga agtcccgtgg tgaatgatcc tggcaaaagc aagttctgaa ttttgcagca | | |
| gtaaaataga tggtccggga ccccaaggag tcttgtaaag gctgagtgag ggcagccgga | | |
| tgtgcctaca ccagctcatc agaagtgaac tgttgtcaca ctgggcacta aagcaccaac | | |
| tctgaaatat aatttttgat tatgttccct cctaaaataa ctaaagcaca aactctgaaa | | |
| tataattttc gtttacgttc tctccctcta ctaatattcc agcagagaac agagcccgcg | | |
| ccaggtgtcc agtacccagc ccctcatatc cgaagctcag gacttggggg tttcgggaga | | |
| gagcggctcc agcgcgtcgg gttgtagcta ctgcatctgt gctcttcctt ccccaggaaa | | |
| caaatggtgg atcggacctc ccaggctctt cgcgccccgc cacccctccc cgtgttagca | | |
| gg | | |
| gcgcaggg ctccggggcc cctccctgca gtactgggtg atagacccca ctccaccctc | Exon 1 | Seq. ID No. 13 |
| cgggtccctc cacccccacc acgtgcaggc cagagaaggc aaagaggccc agccaccctc | | |
| accagggaat ttcttttctt tttttgctgg tttcaggctt ttttctgcct gagtgaaaat | | |
| gaaacaaaca ccccctgcgc ctcccggcca ccagacacac acgcgcaccg gcactcgcgc | | |
| actcgcgccc tcggcctcct agcggccgtg tctggggcgg gacccgctct gcacaaacag | | |
| ccgcgggccg ggtggagcgg ggagctcgcc gcccgccgcc cagtgcccgc cggcttcctc | | |
| gcgcccctgc ccgccacccc ggaggagcac acagcggccg gcgggccgga gcgcaggcgg | | |
| cacaccccgc cccggcacgc cctgccgagc tcaggagcac gccgcgcgcc actgttccct | | |
| cagccgagga cgccgccggg gggccgggag ccgaggtgtg ggccatcccc gagcgcaccc | | |
| agcttctgcc gatcag | | |
| gtgg gtcccgctgg gcgctgcccg agcccctgga ggccgcgagt | Intron 2 | Seq. ID No. 14 |
| cccgcccggc ccggggctgc gggcgccgtg gaggcagcgc ggggagagga caggccaccg | | |
| cgccggccct gccctgttgc tgccctgccg tgtccccgct tttgttctcg tcgttacctc | | |
| tgtgctcaac tctgaccccg tctctgtccc catcttgtcg ggcctgaggg gctgcgggct | | |
| tccacggggt ccgccggatg gaggcgggag aggggaggct cggggcgcgc agaggaggag | | |
| gactgcccgg gaagtctcga aaggagggag gggtctgtct cccaatgtgg ggcaggggag | | |
| gcggaggcct ccctcgcccg ggactaggtg ggaagaggat gcctccgcaa gagggaacct | | |
| gagagtgaag tggggggcac agaaaccctg aacgcacaga gagggagaag tcggggaact | | |
| cagagagcgg aggaccgaac ccgaaacccg gccgggggaa actttggaac gccgaaactt | | |
| tggcggcgaa aaaggccgct gtatcgggtg acaggaagca aagggtcctt cagactttaa | | |
| gccacacgtt ccaggaggga gggaggcgcg gagaccgtct gcgggcgccg ctcctccccc | | |
| caggaaagac aagagacccg gacggttgct tttgtggttt tgcttgtcgt cgtttgccct | | |
| cctcttggcc cctgagcggg ccttgtcgcc ttgttcttgt gcttggaaat gggtgggtct | | |
| cggagcgctg gacgtgcggg gaccgggggg gtgggggcga ggaggagtcg gggccgggac | | |
| gcctcctagc tggcaaaccc ttttccaggg agaatccgtt tccacaaacc tgaaatagag | | |
| agactgctgg aagtaaggaa atgccaagtg cgaagaggtt gtgtgtgtgt | | |
| gtggtggggggggatgtgga tgcttt | | |
| aaaatctgat tttgatctga tttggctagt ttatcacagt ccatccttac ctggtcaaat | | |
| tcacatactt ctgctgcctg cctggctcct gtaggctttc actcagcatt aattcagcaa | | |
| atatttactg aacatctgat agatgtcaaa tactgttcca ggtaccagga aagcccagaa | | |
| gtgaccaaga cagaagacaa gtgctccctc ccacccccca aagagcttgg gttctagtgg | | |
| aatctggttc atgaccctct tcttgttctg cctccgttag catccccagc ttggtctgac | | |
| ttcaccacca ccaggggtgt acaaggctga ggtgggacag actcacagaa agacctcaaa | | |
| cttgtcttcc attccagggc tgctgactca taccatacga ctctgtaagt ttcttccctg | | |
| atcttcagtt ccctttctta taacttgggg cttgtaatat ttcacctact tagcctctat | | |
| gttatgtggc ttttgtggat ggcagtgggc tctaaacggg gcgtgggtgt gaccttgacg | | |
| gaagatgagc ttatcacgtg ttcaaaaagc agtcctgctt tgaggcaggg agctgactta | | |
| cctgactttg aggttctctc tgctgaggaa agagtgagaa cttctgtggg gggtcggggg | | |
| caagggtacc ccctggcacc tactgcccaa ttgtgaataa ggagcaggtg cctctttctc | | |
| acctccatct ggggtacttg gcctgaggaa ggggtgagaa ggaccaagag agggtaggaa | | |
| tagagcggtt tccttgggtg gggaaatcct ccagtcacct gtgctggtgc tcaagcccag | | |
| gctgtcatca gtacccgggc ctcgcccttc cgtgggagcg cctcacatct ccccagctgt | | |
| caacaaagcc agcttctttc ttctctagga agagtctgac ctatagagct tgaaggactg | | |
| acatgagccc cagagaggga cttcctggtg tgcaggagga gggctgaggc tcaggatgga | | |
| tgcttgcaga ggcaggagtg cttcagcatg gctttggtgg agtctgtcct ggagttacct | | |
| ggggcagagg cagatctcaa gatgattagc aatgtactgg cctggaaaga gtcatcatga | | |
| tttcattttt ccagctcttc tcaaggaaat agacttatag atgcaacctc tcttgactgc | | |
| cgttatttat tatgtgggct tttgccaaga tcgtttcagc tctgatactc acaggcgtgt | | |
| gtggggggca gtacttaaca gtaacggaaa cgtcgtgcca ggaacccttc cctccgtacc | | |
| tttccccacc tgcagggtta catggtcaaa atgactattt gatacacaaa tgtaaactcc | | |
| aaggagctgc agcctcggat taatagaaca gcagagacgg acaatgattg agcacctcaa | | |
| gcacttttcc gggcgtgtct ccttacttct tgcaatattg ggtaatacgt atctctagac | | |
| acttaccatg tgccagctac catccagctg ctgttgttcc cattgtgcag ccgtagaaac | | |
| agagacacag agaggttaag cacattgccc aggatcgcat atgggcaggc ctgggactcg | | |
| aactccggca gcctgggccc agagtccaca ttcataacca cggtgctcta ggcccctcac | | |
| ccaccccgag cggtggggat tataattatc ctcaccacac ggaagaggaa accaactaaa | | |
| ctgctccatc actcacaagt gacagcaaga atgtcttata cctgccttaa acgtatttag | | |
| gattaaaagt gacagctgca acctttgtat ctgtagcact ttttgccaag aacacttaat | | |
| cctccctctc ccacagggtg ggaatccgga cctttgtgtt tctcagctgg aaggggtctg | | |
| gggcatgaag ccgggaccct tcacacctgg gctgcagctg ctgagccgca gctccaaggc | | |
| cctgcactcc tctgcagggg acatggcaga tggacaggct ctgaatgctg gctgtcatct | | |
| gacaggccta tggactgtta gggctggaag gggccttggg gaacattgag tgatgagatt | | |
| agtcggcctg gctgggctgg gaaacgtgcc aaactcctac ctggatggcc actggcctcc | | |
| tttgatcagc agacctgagg ctcacttgct acagttccct gcctctccat gaaggaatgg | | |
| ccggaagtac atgcttcctt gttttgagag tctgggcatc agggtatgtc ggagaaggag | | |
| gaaggtcatg tcggatcctc tggaagttga attttctgcc ttccaagttt gcatactctg | | |
| tcgtgctctg attcatgaac ctggagcctc taattccacg aacctgtagg gtgttcccca | | |
| gaggcagctc aggaggaagg gcagcatcag acccaccagc cggcaacttt gagcaagtca | | |
| cagaggctcc cagtgcctcc ctcccttccc tgacccgggg cgggtgagcc tgaggatttg | | |
| ctgagttaaa ggagagaggc tgctttgtaa actggaaggt ggcaaccatg atgggtgctt | | |
| gctttttttt gttgttgttg ttttgttttt ttgtcttttt gccttttcta gggccgctcc | | |
| tgcagcatat ggaggttccc agcaggctag gggtcaagtt ggagctgtag ctgccagcct | | |
| acgccagagc cacagcaacg tgggatctga gccgcgtctg caacctacac cgcagttcac | | |
| ggcaacactg gatccttaac ccactgagcg aggccaggga ttggacccgc aacctcatgg | | |
| ttcctagtca gatttgttaa ccactgagcc tcgatgggaa ctcctgggtg cttgcttctt | | |
| gaaaggacca gtttatctta gcccagttcc tgagcctcca aatgctgtga actttccctc | | |
| ccagttgacc acagtccagc tgcctgcatc atttaatgtg aaagatcttc cctgagtccg | | |
| tacttaggtg ctctgtggtg cttggtattg gggcgttgaa cccaagagaa ggaaaaaacg | | |
| gggtctatcc acgaccctgt ggccctgaga ccctgtagac tcaggggaag tcagaattcc | | |
| caagagaagg cagcttccag caggaagatt tctgtgcatc tttgttttta acacacacac | | |
| tgaaagggaa tgtttgtgag gcattttccc aaggtggaca cacctgcata accactacct | | |
| ggctcgagaa acaacatgac aagccccccc ccctccccca gcagctctct gagcctcccc | | |
| ttcccagtct ctaccactcc cactctgact tctggcacca cagattggtt ttgtcttttt | | |
| tttttttttg tctttttagg gctacacttg gggcatatgg aagttcccag gctaggggtc | | |
| caattggagc tgtggctgtt ggcctacacc acagccacag caacatggga tccgagccgc | | |
| atctgcaacc tacaccacag ctggtggcaa tactggatcc ttaacccact gagtgaggcc | | |
| agggatcgaa cttgcattct cgtacatact ggtcagattt gtttctgctg agccaccatg | | |
| ggaactccct ggttttgtct attttttttt ttttttttgt cttttttgcc atttcttggg | | |
| ccgctcttgc ggcatatgga ggttcccagg ctaagggtcc aatcggagcc gtagccccag | | |
| cctacgccag agccacagca acgtgggatc cgagccgagt ctgcaaccta caccacagct | | |
| cgcggcaacg ccagatccct taacccactg agcaaggcca gggaccgaac ccgcaacctc | | |
| atggttctta gtcggattcg ttaaccactg cgccacgacg ggaactcccg gttttgtcta | | |
| tttttgaacg ttaaataaat gcaagcatcc agggctgctt tgactcagta ccatgtgtga | | |
| gatttaccct gttgatgtca gcagctgtgg ctggttcctt ctcacggatg tgtgtgaccc | | |
| tcacctggac cacacctgat ctggctgatg atgggccttg gggtttttcc agcttttggt | | |
| cccaggtcac gtctctgttt gaacttaaat gcacttgctt tcaggtatta atctggggcg | | |
| gaatgactgg aacatgaggt gtggttggtt cagctttagt acatgccagc agggaggatt | | |
| tcagtagttt attaagcaga tcttgaagac tgtggtcaac tagctcatgc cccacaggag | | |
| ggggcggtga atttcttccc cagaacagga gtgacaagct aaattaggca tccatccgct | | |
| ggaagctgag ggggcagttc ttggctcctt tctgtcaggt ttcggcccct tctccttagt | | |
| ctggggtttc taggctctac tcccaggaag tgtctggggc cacttgggaa caatgggtgg | | |
| gggggctctg agcccctact tacttcattt ccctccttca gccaaagccc cctgtgtcct | | |
| ctgttttaca tagtggggtt ctgagaatga cttcattttt tttttttttt tttttaaagc | | |
| tttagctgtt gcgacattta caaatccact gctgtgaggt ctcttccagg taggaaattg | | |
| tattttggga gcaggaggtg ggtgtgggga gggttaagca ttattcagcc aaagagttgg | | |
| gttgggcctc agtgaccttt tgaagttctt atagcttggc | | |
| ttgccatgca ggagatctca gaacattcta taaaaatagt gttcaaacag aacaacttct gaagcctaaa | Exon 1A | Seq. ID No. 15 |
| ggatgcgaacaagaggctcg gaag | | |
| gtagca tttcaacggg agttttgagg atgctctcct ttagccaccc | Intron 3 | Seq. ID No. 16 |
| ctctccattt tctgccccct tctttttaaa ttctccattg gctgtccctg ctagttgtca | | |
| tttggggtgg tttgggttca gaatggttct cattttcgcc gaggagtggg tgatgtgggc | | |
| ggcctgtgtg tctctcccaa gggtggtggc tgtccctcct ccaccaccag gcctagtttg | | |
| gacctgtagt ttcgcttagt gaaggaggcc gggccgatcc tgggccggag agagacgtct | | |
| ctgccttggc atgcagctct gagtcaacag gcctgataaa cagcccactt cccagggcga | | |
| gcaaggagga acaaggcccc tggctgctgt gggatccgtc tgcgctcctc ttcgtgaaac | | |
| cgctgtttat tcttttgaca g | | |
| gagttggaa cgcagcacct tcccttcctc ccagccctgc | Exon 2 | Seq. ID No. 17 |
| ctccttctgc agagcagagc tcactagaac ttgtttcgcc ttttactctg gggggagaga | | |
| agcagaggat gag | | |
| gtacgtg aaacgttgaa atgatttacc tccgctttgc tggggtcacc | Intron 4 | Seq. ID No. 18 |
| gggggggtgg gtatcatgag ctggctgcag cgtggagaga ggagcccccc tctccccctg | | |
| acttcttgct gctcccccca gttgttctga aagaagacaa agtcctccag tccccggcat | | |
| cggatctagg agtgggagct ggcaggatgc tggctcagtc actgttggtt ctgctttcgt | | |
| tggctgcccg gcaggacctc acggggtgtg gctacagcct ggggttctct gtgtgggcca | | |
| cacagtgcca ttgtggggcc aggaggacga gtctcaggcc cgggacctgt gctgggggcg | | |
| gacatagtgc cctctcaggg cagcaccgat ccttcatgta cctcgcccta tttctcttgg | | |
| aaaaactctt gcaccatgat ttctgagcca ggcagcaagg agaagctggc tggatccagg | | |
| cttcagattt ttgaagggga ttcaagaaag gggcctacaa gatgtccctc cgagaacagg | | |
| tctgtgatgg ctggagcgac agctgtgaaa aaaataagtg gaaagagcct tcggtgcggt | | |
| actccccccc cacccctgcc ccccaaatta taccatgttt cttccaacag ggagcatttc | | |
| cctgtaatgc aagccaattt aaattcttga gggtgcacat tttggtttta tttcaactga | | |
| ttattagtgt agaggaglat aagataacat ttctttaaaa accatcaaca caaacccatc | | |
| actcgtgatt caattgttta ggagaggagg gaactccgcc tcgtatacca aatacagtct | | |
| gctctcggtg cagcgtgcag tcccagcaag gccctctcct cgaactcaca cagctcttgt | | |
| ctccagcggc ttccttccca tgtcttggct aggctgggct ttcttagtaa ccccaaaggc | | |
| ggagaatcaa attcacagat tttttttttc tggatattta gatcttgtat tttaagccac | | |
| actatttata aggctcagag atacatttaa actctgacta gggcttctta taaaagtgat | | |
| atctggaaag aaggtctggc tttaacagag taagggtcag accccccctt ttcccattaa | | |
| tgactccagg aatgctctgg aagactgaag tggaggcaaa gaaggacttg aatttgcatg | | |
| acctgatctt gaatccaggc taaatttttc ctggctgtgc gcctttaggt gggtcattta | | |
| cctcccctaa ttctcaggtg gctcacttca tcatctattc ttttactgag gcagagaggt | | |
| ccctctacca ccaggttgaa tgagctcagt gacctctgaa aactccaaag tgctgcacag | | |
| atcaaggtgg tatgaggtag aagaggaagg gaaaaaggaa tgagtaggat caaagaaaga | | |
| aggagtgaaa agaagcagag tggagagaca gagccaacac aaggatctgg gtaccacttc | | |
| tggattaggg tcagggctta gaagatgaca ttgatggttg ggtctttttc actacacaga | | |
| gaatagagct gaccattaga cttggcccgg agccagtcat tgtgaaagaa atcaatattc | | |
| agattatcat gacaactacc atttgtgtaa ttttaattca caggatcact ttttctggcc | | |
| cacgaggttg aaataagaat ggctggtcag attgactggg gcggtccgac tggcctgtgc | | |
| ttgagagttg accatgagct ccctgccatc tagcgtgtat gtcacccaga cttttaactc | | |
| accatctgga ctgaccctcg agaacttgat gccatttgag agcacccaag gggtccagag | | |
| gaccttatca aatcctctga ctcctctgtg caggctgttg gccagcttat actccttccc | | |
| atccaacgtg atgttccttt ggcaatttgc tttgccaccc tgccaaccac tgctccaaag | | |
| tagggatgct tttggaggta cccttccaat tcagcaaagc caagcaccac atctgaggct | | |
| ctgccttgcc tgtctttgac ctccagggcc gtgatggtgc agcccgagga gatgatttcc | | |
| actcccagtg ttgttcagcc cgaggagatg atttccaatt cccagttggt ctgcttgcag | | |
| ctggaatttt tccatgttcc ttgcccccaa ggggagttct ccaaacacag atcttgtaac | | |
| tgaaaccatg aggaaagctt ggggtgtgta ggtgctccag gtccttcaaa cgccccatct | | |
| tttggcagtt tcttgctcag gtgggtccag ccagagtcct ggagaattca gctctttgat | | |
| cctggctgga gtggggggtg caccaccagg tgattgtgag gtctggatcg tgacctgtga | | |
| gcagggagcc aagtagcatc atgttcagct ccttctcctt gggatcaaag tgagaggctc | | |
| caaggagctc agcaaggtct acctggatgg ggcaggttgc tcctaggacc caggtaggtg | | |
| cggggagcag ggtcagtacc tgggctccac ctgcagcccc aggacaggca cccaggctgg | | |
| aacgattccc ccaggcaggg gcagcacctc acctggagga agcatttggg ccttgcccac | | |
| tccacacccc aggcctgcct gggggcctga cccggaggct tctgggtgaa gtggcctgag | | |
| ggctcaacac attttgtggg caatcctatc tcttttttta tttttatttt tttatttttt | | |
| gctttttagg gccgtacccg ctgcatatag aagtttcctg gctaggggtc aaatcggagc | | |
| tacagctgcc agcctacacc acagccacag caacacagga tccaagccgc gtctgtgacc | | |
| tacaccacag ctcatggcaa tgccggatcc ttaacccact gagcgaggcc agggatcgaa | | |
| cccgcaacct catggttcct agtcagattc atttccgttg cgtcatgacg gaaactctgg | | |
| caatcctatc ttttgatcac cacttctagg aatctgtggc cactgcagca agttgagctc | | |
| cagtgaacct gtcctcataa aaggagcctt cagctctgtg gctgccttct catacaggtc | | |
| ttggctcatt caggggaagt taagcccaca ggacatgttt caaaggacgg gaaatgcact | | |
| gggttttagc acagtctgca cgaggcccgg gagtgggggt gcaagtggtt tcttttggaa | | |
| accgctgcag gggctgagtt gtgggagtgg cccaggagca gagagaaatg gcaaacgcct | | |
| tggcaggagg gcctgtggga tggtgggagg gctcaggtgg aactgggccc gctgggttca | | |
| cctgatcctc tgagggctgg ggcccaggtg gtgctgaggt ggttacactc tcccttataa | | |
| gacaggatgc tagtgctctc taggctctaa tcctgtgctc tccctcttcc atgagaaatg | | |
| tagaagcaac ccccactttt cctatttggt gggtaagata gtcaaccacc aatcttgaga | | |
| attagagagt tttgaaaatt ctgtgacaaa cacatccgtg aagggctttt agaccacatg | | |
| ggctgccaaa tgcctcattt taatccagag agaaaaataa aattgtttt | | |
| aattttccct tctccttttc ttttcccagg | | |
| agaaaataat gaatgtcaaa ggaagagtgg | Exon 4 | Seq. ID No. 19 |
| ttctgtcaat gctgcttgtc tcaactgtaa tggttgtgtt ttgggaatac atcaaca | | |
| ggtaattatgaaa catgatgaaa tgatgttgat gaaagtctcc tctaatctcc tagttatcag | Intron 5 | Seq. ID No. 20 |
| ccaagtcacc agcttgcatt aaaagtagga ttcactgaca ccgtaaagaa agcattccag | | |
| aagcttttaa ggactctaag ccttcatttt tctttttttt tttcctatct tcgacttggt | | |
| tgctaggaag cttagagcaa agtattgtgc ttaaatgctt gcattttcct tggccttcat | | |
| tttttttaaa acattttttc ttattaaagt atagctgatt tatagtagcc ttcatctgat | | |
| atgatttatc ccctggtgtt aaatcctggc ttttgttaga tgccatggga tcttggcaat | | |
| ttgctcaaac tcattttgcc aatatcttag ctatgaagta aaaataaagt taaagatttt | | |
| gttctcacag agtggctggg atgaccaaag tcatgtgaaa acacccgagt gactaaaatg | | |
| tttctctgtt tcgttttgtt ttgttttgat tcttgtattg ttttcctatt tatcgtaacc | | |
| acactttctt cataagccat ttcaagcact tcctgaaagt agatggactt taagtttctt | | |
| ggacttccag ttgtggcgca gtgcaaacaa atctgactag tatccatgag gatgcatctt | | |
| cgatccctgg ccttgctcag tgggttaagg atctggtgct gctgtgacct gtggtgtagg | | |
| tcacagaggc ggctcagatt ccaagttgct gtggctgtgg cgtaggccgg cagctacagc | | |
| tccaattaga cccctagcct gggaacttcc acatgccgca gggtgcaacc ccaaaagata | | |
| aatgaataaa taaataaata tgcgaccttc ctttcttggg gcccttgcat gtttttctct | | |
| ctgttaggca cactcttgct aatccctctt cactgggcct cctatgtatc cttcagaact | | |
| cagctaaaac atcatcccct cccctgggga gccttcgagg tcttcctgtt aagtgctcct | | |
| atgctttctt ggagttttga agtcctataa tgatgtgttt atcaaaatag ggtccaccct | | |
| ccctgccagc ttctttacac cacagacaca tggtgtctgt ttcagtcaac actgtatgtc | | |
| tggcacttga catgtaacgc atgctcagca ggtatttgtt gaatgaatgg aggcggtctg | | |
| ctagagtcgt catatattta ctgatcccgt cttgtaggat ggtctcactg cttttgttag | | |
| cttaagaagt accttttttt tttttttttt tttaatggcc acacccatgg catatagaaa | | |
| ttccacgaag gaaggaagaa agaaagaaag aaagaaggaa attcctgggt cagggattga | | |
| atccaagcca caggtgcaac ctgagctgca gttgcggcaa caccacatct tttaacccac | | |
| tgtgctgggc cagggatcat acctgtgcat ctacagcgac ccaagccacg gcagtcagat | | |
| tgccttttct aggtgcggca tatggaggtt cccaggctag gtgtcgaatc agagctgtag | | |
| acgccggcct aaaccacggc cacagcaaca caggatccaa gccttgtctg tgacctacac | | |
| cacagctcaa cggcaacgtt ggatccttaa cccgttgagc gaggccaggg attgaacccg | | |
| caacctcatg gttcttagtt ggattcgtta accactgagc catgatggga actcctgcag | | |
| tcagattctt aacccaccat gccacagcag gaactcctag aagtgccctt tgaggctact | | |
| ctgtagacag ctttgagcca gcgaggcaag acctgttttt ctggaggaag ataaatcctg | | |
| ggtgagggat gggtgggctg tggtcttcct gggacccatc tctggagcct ctctccctca | | |
| gcaaagccac cttggacaat aagagctgcc atctattttt tttttcttta aactaagatt | | |
| tgatattttc cagagacctc cctcccaccg ttcgatctga gtaattctga aatgacgaga | | |
| gccccgtgat atcatttttt cgatctcgaa ggtggaaacc tgggagtagc cacaacccag | | |
| gctctcagct cagcctaggg tttcaatgat aatgattgca aaatagcttt tctctgcgtt | | |
| ccaagtaaca tgatatgttt ttatttccat t | | |
| tgcttttag cccagaaggt tctttgttctggatatacca gtcaaa | Exon 5 | Seq. ID No. 21 |
| gtaa gtgctttgaa ttccaaatat ctctaggtca ccttccatgt | Intron 6 | Seq. ID No. 22 |
| gaccctggtg gccctacagt ccattcttaa catggcaggt ggtgacgcac ttgtggtcct | | |
| aggtggagga gagggatggg gttccagggg tctgagctgt acttctccag cccctagact | | |
| tgcctttcta gagcatgagt tgtgtttttc ctttgcttct catcaagtat ctatctcttt | | |
| aagtgatgtt gtttggagaa cattcctgcc ttgctcataa aaaagaatca gagtagatat | | |
| tatccattat gctacctact acatgtggta taaagaccct tgcccagaaa ttttgccaag | | |
| acaaaggatt aggaagaaag gctgggtgtc ctgataaact aagtgtgtgt attattatta | | |
| tttaatatta ttactaatac tgggtgattt aagggactcc taaggccttc aatttttcct | | |
| tttttctttt tttttcccta atcttccgac ctttggtttg cctaa | | |
| tttctaaaaa atgtttgtca tctttttcat ttctta | | |
| gaaa cccagaagtt ggcagcagtg ctcagagggg ctggtggttt ccgagctggt t | Exon 6 | Seq. ID No. 23 |
| taacaatgg gtaagactgg gaaacggcca | Exon 7 | Seq. ID No. 24 |
| tctgtgtatc tgctcaaggc tgtagagtcc aaataaaatg gtttcacagc catgaccttc | | |
| atgaccttct ccagtcgcgt cgtccttctg gcttattgga cattctggca catgggtcac | | |
| cctccctgcc ttcctcagct tgttttccgt ttgtacgtag g | | |
| actcacagt taccacgaag aagaagacgc tataggcaac gaaaaggaac aaagaaaaga | Exon 7 | Seq. ID No. 25 |
| agacaacaga ggagagcttc cgctagtgga ctggtttaat cctga | | |
| gtaag aaaagaagcg ttgccctatt tcagtaaatc ca | Intron 8 | Seq. ID No. 26 |
| agcagaacag ggggacggaa gtacatacac gttgtacagg tacgatcccc aaagggccac | | |
| cagggcagcc cgcagaggca cttgggccag agcctcctgt ccttccccca gaagatgccg | | |
| caatgtcaca ccaccagctg actggggcta aaatacagtc aggattcaag gccagtccca | | |
| caagccatga ctgacccatg ttcccccaga ctgtcgtacc ttagcaaagc catcctgact | | |
| ctatgttttg tcaccag | | |
| gaa acgcccagag gtcgtgacca taaccagatg gaaggctcca | Exon 8 | Seq. ID No. 27 |
| gtggtatggg aaggcactta caacagacgt cttagataat tattatgcca aacagaaaat | | |
| taccgtgggc ttgacggttt ttgct | | |
| gtcgg aaggtaggtg ttgctaataa aactggcctt | Intron 9 | Seq. ID No. 28 |
| gagtttttcc ccttccacta tcagaggatg ggtgaggggc ccctgggttt acagaggctg | | |
| ttcatgtcat gtctgaatta gtggagagga gaatggtgtc acagggccat tttagactcc | | |
| cttctgctga ggtccccaaa ggctaagaat aaaactagtc agagggtcaa ctctttccca | | |
| cctcagggtg aggggcttgg gttgcaggga agaaaatctg ctatacccac tgcacccaaa | | |
| gtcgacagta cacccacagc cacctccacc ctgacctcca cggccctctg tggaaattcc | | |
| tgcaatgccc agagcagctg aaaacacatg ttctctctgc ctggttggct tccaagagtg | | |
| agagaggaag gagcagggct gagcatgccc agecaccctg ccagaatcac cagtcaggta | | |
| agccactcca cctccccaaa gctgaatgac tgaatggtgg agagtagctg ggaatgttac | | |
| agcaacagac gtctctcatc caggatgggg aaaaatcatt cctttcctaa actgcaaaat | | |
| acagactaga tgataatagc atattgtctc ctctagaaat cccagaggtt acatttaccc | | |
| cattcttctt tatttcag | | |
| at acattgagca ttacttggag gagttcttaa tatctgcaaa | Exon 9 | Seq. ID No. 29 |
| tacatacttc atggttggcc acaaagtcat cttttacatc atggtggatg atatctccag | | |
| gatgcctttg atagagctgg gtcctctgcg ttcctttaaa gtgtttgaga tcaagtccga | | |
| gaagaggtgg caagacatca gcatgatgcg catgaagacc atcggggagc acatcctggc | | |
| ccacatccag cacgaggtgg acttcctctt ctgcatggac gtggatcagg tcttccaaaa | | |
| caactttggg gtggagaccc tgggccagtc ggtggctcag ctacaggcct ggtggtacaa | | |
| ggcacatcct gacgagttca cctacgagag gcggaaggag tccgcagcct acattccgtt | | |
| tggccagggg gatttttatt accacgcagc catttttggg ggaacaccca ctcaggttct | | |
| aaacatcact caggagtgct tcaagggaat cctccaggac aaggaaaatg acatagaagc | | |
| cgagtggcat gatgaaagcc atctaaacaa gtatttcctt ctcaacaaac ccactaaaat | | |
| cttatcccca gaatactgct gggattatca tataggcatg tctgtggata ttaggattgt | | |
| caagatagct tggcagaaaa aagagtataa tttggttaga aat | | |
| aacatct gactttaaat | 3′UTR | Seq. ID No. 30 |
| tgtgccagca gttttctgaa tttgaaagag tattactctg gctacttctc cagagaagta | | |
| gcacctaatt ttaactttta aaaaaatact aacaaaatac caacacagta agtacatatt | | |
| attcttcctt gcaactttga gccttgtcaa atgggggaat gactctgtgg taatcagatg | | |
| taaattccca atgatttctt atctgttctg ggttgagggg gtatatacta ttaactgaac | | |
| caaaaaaaaa attgtcatag gcaaagaaaa agtcagagac actctacatg tcatactgga | | |
| gaaaagtatg caaagggaag tgtttggcaa caaaataaga ttgggagggg tcgtcctctt | | |
| gattttagcg tcttcctgtc tctgctaagt ctaaagcaac agagttgctt tgcagcagga | | |
| gatcagagtc taccttagca atcctcagat gatttcaaca gcagaggact tcaggttatt | | |
| tgaagtccat gtccttttcg catcagggtt ttgtttggct tctgcgcagg atactgatca | | |
| agattcccaa tgtgaatgtt ggagttacag ggaatccgaa tgaaccaatg ggagctcagc | | |
| acgaaataaa agcacagctt ctaagtaagt ttgccatgaa gtagcgaaga cagattggaa | | |
| agagaggggg ctgatcactg tggggcaatg ccatttctaa gagacacagg gcatggagtt | | |
| ggcatgtaca tacagcttgg atccaggcac tgaatgggag gcaatgagag tggctccagc | | |
| ctcctcaacc atatgacaac tagagcagca ctgtcttaga agatgcttct tgctttggcc | | |
| aagtcatatt cagtctgcca gactctggaa cttgtgtcta caaatccttg ctcagaggaa | | |
| gtggatgatg tcagagtgga cagaggccta cattgggttg aagtgacttc ctagaccttg | | |
| gcttcatgac aatcaggcat cagcaagccc tgctgccacc tgctctaact ctcagagtcc | | |
| ctcagcccat catgggcaac ttgagagcca ccgtcaagga gtggactaga ggaaaagcct | | |
| gcttatcagg gaacctctca tttcccctgc cccagctgca ctactgaagt gtaactgccg | | |
| gacatgttta ataaagtggt taattgattt tatatcaaag tagagaggat ggcaatggga | | |
| gacccagtcc tcatgactaa acagcttttc aatccctttc tctaagaaaa gctatgagat | | |
| cttacatgta atttaaagtt aagcagtttg gtgtaaagga agttaggagg caatatttac | | |
| atctgcaggt atgtgatata cttttgcttg tgttccagtt taggtcattt gtgtccattt | | |
| tcaaatgatt tacttgaaga gccattgcac tgacttgatg ttcagcacga tgggcttctt | | |
| tgataaaatg aaacctacat tttctctact gtttccctgg gcctcctact cttcaattct | | |
| tgctaaaaat ttttgcaacc cagcaaaata actcaacaaa ataacccaac aaaataactc | | |
| aacaaaaatc ctggagaagt agtcttgtaa aagaaaaagg aaatcacaag tcaattagga | | |
| ctcttgtttc tctataacgc aagtttatgg aatccattct ggagtgcaga gacttcatgg | | |
| tgcaagttcc aaactacaga aatgattcgt tctcaaagat taaagaaaag gactgatatt | | |
| tccttttgaa ggaatcttga tttttaaaaa aaaaatcatt taaatttaaa tttcaaatgg | | |
| acaaattcaa gatcttatta atagttcaat attaaaaaat aaaaattcct gatttaaaat | | |
| taaataaatt attttctcag tatattctgg tctggtcatg gattgtggct tttttcccaa | | |
| agatgttcag aactgtcatt taca | | |

Isogloboside 3 Synthase (iGb3 Synthase)

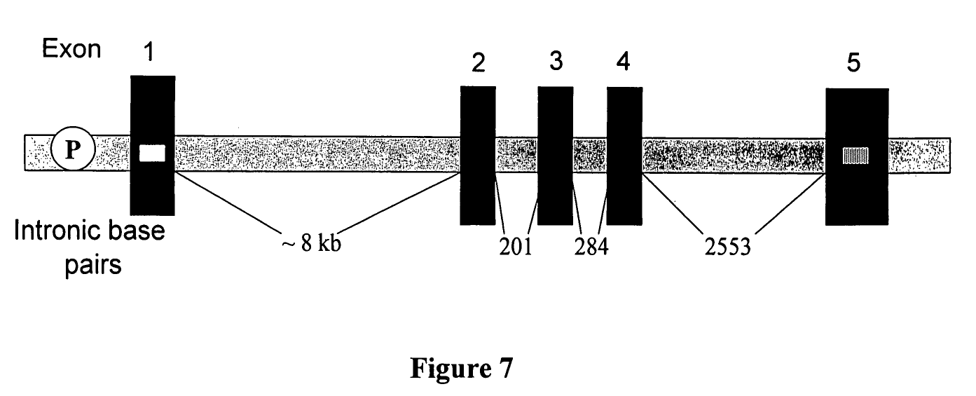

In one embodiment of the present invention, iGb3 synthase genomic sequence can be used to design constructs that target the iGb3 synthase gene. The genomic organization of the iGb3 synthase gene is provided in FIG. 7. The genomic sequence of the porcine iGb3 synthase is provided below in Table 5. In other embodiments of the present invention, the promoter sequence of the iGb3 synthase gene can be utilized.

TABLE 5

| GENOMIC SEQUENCE OF PORCINE iGb3 SYNTHASE GENE | | |
|---|---|---|
| ccttgttcaaccctttagcagggattaactcaacatccaggacagccctccaaagtaggtgttcttagga | Intron 1 | Seq. ID No. 31 |
| cccacctttctagatgaggaaactcaggtgcggaggtccagaaccttgcctgaggtcagacagctaaga | | |
| agtggtggcctgggattcgaacccagggggtcttgctccagcagtcttgcuctcaccctaggggtccag | | |
| tctgtctagaaacaccagcacccagcaggggtgaggagagatggaagagatccccccagaggagctt | | |
| attcaaattcttcatttttgggcccttctggaaaacagccaaccacgctccaatcctaaagtactcctcctct | | |
| gagccagcaaaggggctggtacctctgctggaggtacctggcttggggactaagagccaccatagac | | |
| acagagtccctgagcacaggtggccctccgtgcagcccagcaatgcatctctaagccccagagagctc | | |
| tcaactcctagcttccaagccacaaacttccctgcatccctctcagactctcccctgcccaaggtcagtcc | | |
| tacacactgcctggacgaagcgccccaccccctaatggttactgtcacttgagtgtgcctactgggaaaa | | |
| gcaaagaattaaacatctaaatgctcatcaaaagggacctgggtgaggtaaagtgatgccccctcccgt | | |
| caatggcatgttaggcagctggaaaaaggggtgaggaagcgcttcaaaaataggaagttccccattgtg | | |
| gctcagggggaaacaaaccccgccttgtaccccatgaggatacgggttcgatccccggcctcgctcag | | |
| tgggttaaggatccggtgtcgctgtgagctgcagtgtcagttgcaggcatggctcgagtcctgcgttgcc | | |
| gtggctggggcataggccagcagctgcagctctgatttagcccctagcctgggaacctccacatgccat | | |
| aggtgcggccctaaaaagcaaaaaaaaaaaaaaaaaaaaagagagagagagagagagagatggaa | | |
| taaactcaaagacataatggtcagtggaaaatacaaggcaaggaagagcatatcagcaggctaccgtg | | |
| tgtgggaggaaaagcacaggaagagaaggagagagcgcatttgctaccgtatttacatttgcctgcata | | |
| tacacgactgtccccatgcagaggaacaggaaagactgcactgtctatactctctaggacctttgaatgtc | | |
| tgccatgtgcacagagtaatacatagtcaaagcaaataaaatgaaacattaaattatatactttcccat | | |
| atatatgtatatatgtggaaattacacacacacacatatatattttgtgttgctaatgtccctccctactccccg | | |
| cccacccag | | |
| GGCCTGGAAGAGAATCCTCTGGTGGTTTGATCCTACTTTGCACTTT | Exon 2 | Seq. ID No. 32 |
| GACCTCTTAGGGGTGCTCGTGTLTGGCCTCCGTGGTGTCAG | | |
| gtacaacccccttcccctagtgctcaagatgggaccagcaggggagggttaaagtggctctttcccagt | Intron 2 | Seq. ID No. 33 |
| gcctccttaagggatagagagtgctggctctctcctgcacaagtgtccttgcgggctctcccccttgtaag | | |
| gagcaaagccacagggctcctgagcaggctgacacccctcactgctgcccccatcccccag | | |
| GCATGTGGAAGTCCTTGTCCCCGTGGGTGTCTGGCCTTTTGACC | Exon 3 | Seq. ID No. 34 |
| AGAACACCCCTGGTGGGAGACAACTCCACGGGTCCCCTGCATC | | |
| CTTG | | |
| gtaaggagctgccatctccaggatctctgggcctccagcaccccacccccaagtccctgccctcctcgc | Intron 3 | Seq. ID No. 35 |
| atcccccaccctggcagggctaggcgctccaccccagggccccagcaggttacacatctcgaaatacc | | |
| ctgctggatctggggtagagagttctagggcagggcctgggtgtgacccacttgcaagtccctggggc | | |
| ccaggcctggggaggtgacagtgaccacgcacgaagcaggtggataatggacgaatccctccatccc | | |
| tgccctggctag | | |
| GGCGCGGCCTGAAGTGCTGACCTGCAGCTCCTGGGGGGGCCCC | Exon 4 | Seq. ID No. 36 |
| ATTATATGGGACGGCACCTTGGAGCCAGATGTGGGGCAGCAAG | | |
| AGGCTACCCAGCAGAACCTCACCATTGGCCTGACGGTGCTTGC | | |
| TGTGGGCAG | | |
| gtaaggcctgggaggcgagcagtgctgtccaagcgaagggttgggaggggcgtgcatgtgaagcag | Intron 4 | Seq. ID No. 37 |
| ggcgtggggtgccccattctccggggccacagcatcccaagcggaagcagaaggcaaagacagcac | | |
| ctcctgggcaagactccaagggtgaggcaggaccgacccctccttcccttcctccctggacaccagca | | |
| ccatggagcccagccagcgcaggcagccgggggctcaggaccatgtcctggaaggaacctggctag | | |
| tggtgagaaaacaatggagtttttcaggcgaaagtgagaagaggtgagaactgggtaagtagagggga | | |
| tgacccagctgcagtgagcgccccgcccccatggaggtcagtggctcaggcgcaggttagggaggg | | |
| aggaagattcaccaagcaagtctgatggtgggactggggccgggggacggagggctcttgcaaggg | | |
| agtggatctgggctgagtaaagagaaacgtgaagaaatggggatgcaacagtaacgaacctgactag | | |
| gacccatgaggacccgggttcaatccctggcctcgctcagtgggttaaggatccagcgttgccgtgact | | |
| gtggagtagtcgcagacatggttcggatcccgagttgctgtggctgtggcgtaggtgggcagttgcagc | | |
| tccagcctgacccctagactgggaacttccatatgccgggggtgcgcccccccaaaaaaagaaaggg | | |
| ggatgttgagagtggcagggtcagcaggccagagggctcagtgagggaggactatggggggtggta | | |
| tcaggaagcgggctggaaggacggggctgctgagggggacgagtgaggccgcagtttgggaggga | | |
| aggcagactgatgatgagcaagctgagggagaggtcatgggggcaggtggctcaggagagggaag | | |
| gacagactctctccaggagaggaggccaatcgaggaagtgagaggcccccaggtatggaggaggaa | | |
| cctggaatggtaggtggagaactcacaagggtgctggtctccccatctcccgattagggatggcgggg | | |
| ggtccaagctgggtactcactttccagtagtgatgcaaatgggactcctggctgagagtggcacttagat | | |
| cctatagtcctaaggctcagagaggtagagttcaggacaatttaagggagcgtttaataatggaagaagc | | |
| tgctttcgggaggcagtaaaaagctttgcatcccggaaaagatatccaaaagtatctgatgaattcagctc | | |
| ctccaaatgactcctctctgtccctcacaccctagacgggagaaagccaggaggacccctgggaggcc | | |
| agggtgcaaagaggaccaaggtggacggaactgctggcctctccagggccttgatgtccccacttccg | | |
| ttctggatgctgagtagggtgttcccataccagccctctgggtccagaaattccagagtcttgagatccaa | | |
| attccaaggttctatgagtccaacactctgggatgctgaggcttccaaggtctctcattccagttttcacagt | | |
| tccaccaggaatagaacaagtgcaggtaaagctatgggctccactgccaagcagggttcaaatcctgg | | |
| cttcatacctaccagctgtgtgcgagggtgcatgagttcctaaagctcttggagactgtttcctcaccagg | | |
| aaacggaactaataatggtgaggattaaatgagataatacacattactttgaacactctcacatgataaatg | | |
| ttcaaaaagatcaggcattattattattattttagaaccttaggatcccaaagtctgttcatacagtttccagta | | |
| ttctggatgtctcgattatctgtgtaaggaatcactacaaacgcagtagctgaaggcagttcactattatcat | | |
| agctcatgactttgtggctcaagaattccgactgctcagcagcaaaggttcatcacttctctcaaacagct | | |
| gggtctcctgtgagacagccgcctgaggaagactggcagggtgcctctccatggctagcttgggttctc | | |
| tcactctgtggcagtatcggagttccaggacttcttatgcgaagggtcagagctctaaagggacagagg | | |
| ctaacgcgcgggtcttcccaaggcccagcatggcatcccttccttgtgcctctattgatcaaaggggtcc | | |
| gggagagccgagttcaagggaagggacacaggggctctaggggcagggctggcaaacaatggaca | | |
| attgttatgattattatttaccacaccttccgcatgaggaagttcttgggccaggattccaacccaggccag | | |
| ggatcaaacccgtgacccaagccacagtagtaacaacgccagatccttaacttgctgagccaccaagg | | |
| aactccaattggcaattaattttaatttgcctccaacggggactgccctttccggagttcctgggcctgggg | | |
| tcgcagggtcaccagaacggacatgggggcggctgggaagggcgcagtgaccagctgactcggac | | |
| ggcccgctccgcag | | |
| GTACCTGGAGAAGTACCTGGCACACTTGGTGGAGACAGCAGA | Exon 5 | Seq. ID No. 38 |
| GCAGCACTTCATGGTGGGCCAGTGGGTCGCGTACTACGTGTTC | | |
| AGCGAGCGCCCTGCAGCCATGCGCCGCGTGCTGCTGGGGCCCG | | |
| ACCGTGGGCTAGGGATGGAGCACTTGGGGCGTGAGCGGCGCT | | |
| GGCAGGACGTGTCCATGGCGCGCATGCGCGGGGTGCACCCGG | | |
| CGCTCGGGGGGCGCGTGGGCCACGGGGCGTGCTTCGTGTCTG | | |
| CATGGACGTGGATCAGCACTTGAGTGGGGCCTTCGGGGCGGAG | | |
| GGGCTGGCCGAGTCGGTGGCGCAGGTGCACGCCTGGCACTAGG | | |
| GCTGGCCGCGGTGGCTGCTGGCCTTTTGAGCGTGACACGCGCTC | | |
| GGCCGCCGTGGTGGGCGCGGGCGAGGGCGACGTCTACTACCAT | | |
| GCGGCCGTGTTCGGGGGCAGCGTGGGCGCGCTGCGGCGTCTG | | |
| ACGGCGCACTGCGCCCGGGGCCTGCGGGGGGACCGCTCGGGC | | |
| GGCCTAGAGGGGCGCTGGCACGACAAGAGCCAGGTCAATAAG | | |
| TTCTTCTGGCTGCACAAGGCCACCAAGCTGCTGTCGCCTGAGT | | |
| TTTGCTGGAGCGCGGATGTTTGGCCGGTGGGCTGAGATGCACTG | | |
| CCCGGGCCTGGTCTGGGGGCCCAAGGAGTATGCCCTGCTGCAA | | |
| AGCTAGCAATGGCGGTGAGGGCCCTTCTGGAAGCAGCGGGGC | | |
| ACTGGGGGTGGGGGGAGACTGGGTGAACGCCTCGGCCGCTGGG | | |
| GCATGGCTGCAGGAAGCTGGGGCTTTTGGGACGTGGCTGCCGG | | |
| AGGAGGATGAGCCATCCCTTTTCCATCGAGACCCGGGCACCTCC | | |
| AGCTGCGTGGAGACCATTCACCTCTGACCTTACTGAGTTGAGC | | |
| GGAGGGCGTCTGAAGAGATGTTTTTAGCCCCTTCCCGATATCCG | | |
| CTACGCTTTATATGGTACTGAGGCGGCAAAAGGGAACATGATG | | |
| GCCCGAGGACCCAGAGGATCTATGAGTCAGCCTGTGAGGTCA | | |
| GCAGCTGGAGAGGAAGACTGACCCTCAGGGCAAATACATCTG | | |
| CTTCTAGGCAGAAGCCGCAGATGAAGAAAGTCAGTGGCATCC | | |
| GGTTCGCTGACTTTTGCTGGTT | | |

PCT publication No. WO 05/04769 by the University of Pittsburgh provides porcine isolgloboside 3 synthase protein, cDNA, genomic organization and regulatory regions. In addition WO 05/04769 also describes porcine animals, tissue and organs as well as cells and cell lines derived from such animals, tissue and organs, which lack expression of functional porcine iGb3 synthase, for use in in research and in medical therapy, including xenotransplantation. WO 05/04769 is incorporated by reference in its entirety.

Forssman Synthase (FSM Synthase)

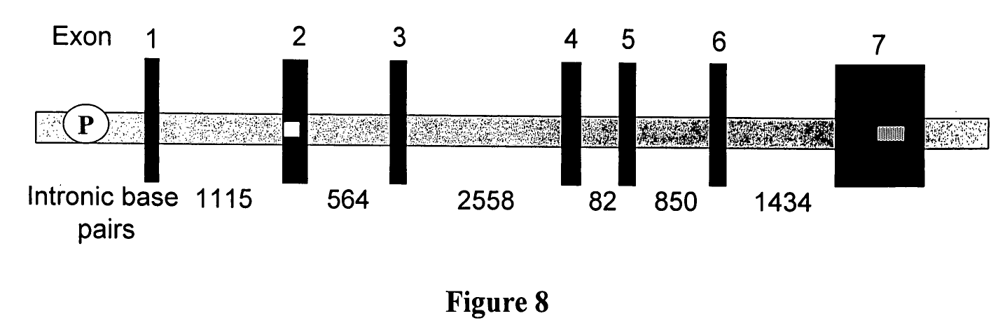

In one embodiment of the present invention, FSM synthase genomic sequence can be used to design constructs that target the FSM synthase gene. The genomic organization of the FSM synthase gene is provided in FIG. 8. The genomic sequence of the porcine FSM synthase is provided below in Tables 6 and 7. In other embodiments of the present invention, the promoter sequence of the FSM synthase gene can be utilized.

TABLE 6
GENOMIC SEQUENCE OF PORCINE FSM SYNTHETASE GENE

| | | |
|---|---|---|
| TGAATTCTAGCTCCGTCTGCCTACGCTGGTCCGACCGCAAGGG | exon 1 | Seq. ID No. 39 |
| Gtgagtctgcagccggtaaggacaatcgcgctccctccgctgcgcctt | intron 1 | Seq. ID No. 40 |
| gtccctgccccgcgcccagccggaggaagagcgccgcgagtccccagc | | |
| ccgcagtggtagtcgagatgtgtgtcttcggccccaggctcctgggtg | | |
| cagatccccggctggggcggaccgagctcggccctggctgtgagtcgg | | |
| cagagcgtccccggcggcctgggccccgcgggagggagaatctcgcgg | | |
| agccaactgtcgaggggggccttggaggacgcttcgccccaaaccggg | | |
| atgggaaaactgaggtctgtagagggagggagagggattgggaacggc | | |
| cttgcagaggccaccgaatgagcagggccaaagccccagaactctggc | | |
| ccggggatctttgacctcgagcggatccccacagagcggccaggggtc | | |
| cggtgctcactgcttactgtgacacaaccctcccggtacatcagggag | | |
| tgcgtattgcgtcttgtcccctgcaccaagccccctctagccgaggag | | |
| gaccccgacgctgtggcggagcggggacgagagtgacttgcccaagat | | |
| tatcgccgagcgggtgcgagctgaagctcgttcctgcggtccccggga | | |
| gagtccaggctgccgcctcctggagcaacgccctgctgccacccctgc | | |
| ccctgctccccgcccggggggatcgcggccgcccctcgctgcgcagca | | |
| tcccgcttcccaggcccggcgtgtccccgctgtgccggctcagagctt | | |
| aatttcggcgtcctcattgtctccctggggaatccctctccaagatca | | |
| gcccaagcgctgttgccctggtccggaggatggccgcccttcgctcgc | | |
| cgcaggagtttgggagggagacctgagagccaaggcaggggaccggtc | | |
| cttggggcacggctgcaggcttcgggtgagcaatgagcctctgtcccc | | |
| gggtcaacttgccagaactgccccatctgggcctagggtccagcagga | | |
| tgagaagatgacctggaatccacagtcccctagcggggctgcccgggg | | |
| gagggcggagcagcaaggctggggcaactatcctccagataaggagca | | |
| ttcctttgcag | | |
| GTCTCCTCCGGACCCCGAAGACACAAGCTCAGAGCCTGACGGCCCCTG | exon 2 | Seq. ID No. 41 |
| AGAGAGGTGGGCGGATCCGCCAAGTCACACCCAGGCTCTGCAGGTGCT | | |
| CAGGCCCAGACGCTGCACCCAGAGATGCGCTGCCGCAGACTAGCCCTG | | |
| GGCCTGGGGTTCGGCCTGCTGGTGGGCGTGGCCCTCTGCTCTCTGTG | | |
| gtgagcatgccccgtggagccctccggccccacccgactcctccctct | intron 2 | Seq. ID No. 42 |
| ctcagcatctcaacccccaagcctgacccttcactgaactcccagggc | | |
| tctcatccgcctctcctgacacacctgtccttctggcgccgtaagaga | | |
| tgaactagtctggacttacggattttgctttgcactggctctttcctc | | |
| tgcctggactattcttctagccatgttaacgaggaactccagtttatg | | |
| ctccaaaattcaccccaatgtgttctttctgcgaagttcctggccccc | | |
| ccacccccaccccccacccccgccccttgtgtgcagggtctggcatca | | |
| ggaacattcctgccccaggaatgaagggctgcatggctctataataac | | |
| tgtgttgccacagaccgggggctttgccatccacggttcgccagaccc | | |
| aaggagtgattggtggggtgggggtgggggtcccaggtgcacccctgg | | |
| gggccttcattcccactaacatggaccaagtgggttttcagcctcagg | | |
| ttcaaagtcgagtcagccagtgttcttccctcccag | | |
| GCTGTATGTGGAGAACGTGCCGCCGCCGGTCTATATCCCCTATTACCT | exon 3 | Seq. ID No. 43 |
| CCCCTGCCCTGAGATCTT | | |
| gtgagtatgagacggggagaatgggcgagatgggaggggtttttaagg | intron 3 | Seq. ID No. 44 |
| ccgctttgcaggttcttacattctcagctcaggattctgatcagtgtg | | |
| attaaacagtgaggcaatttatgaacggctgcaaatgtggagtaaaaa | | |
| ctcccctgtttcagtcccgaggggtgccctttggcatgttgtgtggct | | |
| ctgagcctcacttgctgcacgtgtaaaagggggcgatagatggtacct | | |
| gtgaccgtgctggtgtcacccctggcacataggaggtgcccaggaaag | | |
| agtgcttttaggacaagacctttttgctcaatttggtgttctgcgtgg | | |
| attcgaggaacaaggtgcccagtctctcccacatggcaaggctgactt | | |
| tttgacagctaagtgtgacacagatcaagtgtgatgtaggttgggaca | | |
| gtcccgagggtgcatctggccccctggtcttttgctgtccatgacagc | | |
| agaaggaaagtaaagcatgcatcgcaagggaagttcctgtcgtggctc | | |
| agtggaaatggatctgacgcgtatccatgaggatgcaggttcgatccc | | |
| tggcctcactcagtgggttaaggatccggtgttgccgtgagctgtggt | | |
| gtagattgcagacacgactcggatctggcatggctgtggctgtggtgt | | |
| aggccaggggctacagctccccggaacctccatatgctgcgggtgcgg | | |
| ccctaaaaagacaaccaaaaaaagcatgcatcacagggagttccctgg | | |
| tagtctagtggttaggattcagtgcttatgttctaaaaaagcagaaag | | |
| gctgcttgcttttgaaaacagttgtgaccacaatgtttttggattttt | | |
| atcctgtttccccggatttggccttatttttggcatctggtcaccatt | | |
| attttattctaacctgggtctgggccccctgaacccctttcccaccaa | | |
| caactttgaagcatttaggtggtttccaggtgcccagcgttctaaatt | | |
| agtttgtaatgagcagctctggacataaagctttttcccgcctaaaga | | |
| tcctttcatctggtatgttcctgagccaaaggatatggctgggttctc | | |
| atccgcttgctctccagagggaccagaccgtcccacactcacgctcat | | |
| ccccgcacccctacgcacccccgccccagcagctgcgccgccgctggg | | |
| ctaggactggacataccagctgtcatgagaaacaaaacccaaaccacc | | |
| tcgctgattggagagatgggaaatgcagtctggtgtaaattacgcttc | | |
| tttgatttgttcggggccctcatttcccccaggcctttccatgaattg | | |
| aattctgcctccatgaacttgccctctcacctccttccctcccgggcc | | |
| tctttgctgtcctctgtccccacccttgtatttgctacctcttttttt | | |
| ttttttttttttttttttttccttttgccatttcttggccgctccccc | | |
| gacatatggaggttcccaggctaggggtcgaatcggactgtagccacc | | |
| agcctacgccagagccacagcaacatgggatccaagccccgtctgcga | | |
| cctacaccacagttcacggcaacgccagatccttaacccacgagtgag | | |
| gacggggatcgaacccgccacctcatggttcctagtcggattcatcaa | | |
| tcactgagccacaacgggaactccagtatttgctacatcttgctactt | | |
| ttttttttctttctagtttgtctacctcttggttcttctgagggtttg | | |
| tgtgtgtgtgttgtgatagattgaggctggagatttgtgactttattt | | |
| aatgtttagttatgtatgtatttattggccacacccacggcatatgga | | |
| agttcccaggcgaggggttgaatcggagccccagctgccagcctacac | | |
| cacagccacagcaacacaggatccgagctgcgtctgtgacctataccc | | |
| cagctcacggcagcgctggatccttaactcactgagtgagaccaggga | | |
| tcgaacctgcgtcctcatggatactagtcgggtttgttaccactgagc | | |
| cacgacgggaactcccgaggatagtctttatataaggtcagctggtgt | | |
| cggcgttactcacatgtgcaaaatacagaccttcacagccgtgcctgg | | |
| attgatggccgtgtaactgggtcccacaaccacccatcaccgtgggct | | |
| caggttaagcaactcgcccaggctagaaagtggcagaaccgggcttac | | |
| tgggcctttgcagcttctcagtccttctacccaatgcccaggcccttc | | |
| cagagcaacatgtttgcaagagagacagaaaaagactttggagacaag | | |
| tggtaccgggtttgaatcacagcaaccccggacagaccgcctctgtag | | |
| aagcccagcccctgcagtgggggaggtctaagagagtctgcgtggagc | | |
| ctggtggggagggggtacctgtcccgtgggggggttcatcttggcttc | | |
| cctgccgagcatccctgcccccggccccggcactaatggctgtgtctc | | |
| gcctctcccaccag | | |
| CAACATGAAGCTCCAGTACAAGGGGGTGAAGCCATTCCAGCCCGTGGC | exon 4 | Seq. ID No. 45 |
| ACA | | |
| gtaagcagactgtcacttcccccttggtggcccccgggggtgggggcg | intron 4 | Seq. ID No. 46 |
| gcctccccttaccaccggcccttcttggttgcag | | |
| GTCCCAGTACCCTCAGCCCAAGCTGCTTGAGCCAAA | exon 5 | Seq. ID No.47 |
| gtaggtgtcaattaggggcggggcacagaagggagactcctggggcgg | intron 5 | Seq. ID No. 48 |
| aggtgggggggacagagcgctgattgacaagttggggtggtggagggg | | |
| tcaggtggccttgggagccgggtggtctggcacctgggctccagtcca | | |
| gccctgtcactagctgtgtggcctacccaactgctctgagcttttcct | | |
| gcgtgggtggatagtaatacccccacctggagcgttcccgctgtggct | | |
| cagcaggtgaaggacccagtgaggtctccgtgaggatgcgggctccat | | |
| ccctggcctcgctcagtgggttaaggacctggcgtggctgcaagctgt | | |
| gccacaggtcgcatatgcggctcagggctggtgtggctgtggctgtgg | | |
| cgtaggccgaagctgcagctccagttctccacccctggcccgggaact | | |
| tccatgcgccacaggtacggccatactgataataataacaataatagt | | |
| aataatgataatacccacctcataggaggttacagggcccgacgagat | | |
| ggtgtttgcaaaacgcagggcactgtgcctgcgccctacggggtgccc | | |
| gacccaccgttaataatggtatcaatgactcccgtttCtgaggCactt | | |
| ggcagacaccagaaatgccaggcctttccagaccctggacgcctggtc | | |
| ctcccgaccatgctgagaagtagctgttactacccacactttccacgt | | |
| gaggctcctggagcccagagacaggagtgaagctgcccagggccacac | | |
| agcacaggaggcaggaccaggatgagactgaggctttcacaaggggag | | |
| cgtctcagcccccacggcctcctgtgctgccag | | |
| GCCCTCAGAGCTCCTGACGCTCACGTCCTGGTTGGCACCCATCGTCTC | exon 6 | Seq. ID No. 49 |
| CGAGGGCACCTTCGACCCTGAGCTTCTTCATCACATCTACCAGCCACT | | |
| GAACCTGACCATCGGGCTCACGGTGTTTGCCGTGGGGAA | | |
| gtgagtcgtgggctgggcgtggggagggtgggtatagattctgaaccc | intron 6 | Seq. ID No. 50 |
| caggaatgtatggtctggggacagacaggaccccgcccaggcaccagg | | |
| gaggccctgagccaggtgctgagcaggtgggaagcacagggtcgagcg | | |
| tgatggttgcaggggggcttcctggaggaagggggtctggctctggca | | |
| gcgaagcaggggagcggcccaggtgagagatcgatggcacctttgtca | | |
| ggagacaccttgtccccttaccccttctgcttcccctgagccgcccag | | |
| gcaggtggggagggatagaaagccccccaaccacctcccataaatggg | | |
| ggtccctggtcgggccacacgcaggtcaagagacctgggcagagcagc | | |
| ccggcccccaggagcctctctccaacacgccctcccccggcgggcccg | | |
| ctgccctctgttcagcctgttctcccctctcctccctcagcctgcctg | | |
| gcatttcctaaattaaccgccacctggcagcttccctcggggaccctt | | |
| tctgggagtcctgagagaggggccctaatggggtcctaatgcccaaag | | |
| cgctgtccagatgctggatggctcagcgggggtcaagaccccccctcc | | |
| cccgccaccccagcccagtcagcacccagcatcacaccttccctcgat | | |
| gcagccactcaccgcctgtgtctataagatgggtgtgtggtccctgcc | | |
| tcctagggagttgacgaggcctgaaggagtcccttaaaacaggagtcc | | |
| cttagaacactgcctggcacttagtaagtgctcaataaaagttagctc | | |
| aggagttccctggtagcctagcggttaaggtcctggtgttgtcactgc | | |
| tgtggcgcggattggctccctggactgagaacttccacatgttgtggg | | |
| tgcggggaaaaagaaagttagctctggagttcccatcgtgactcagtg | | |
| gttaatgaatctgactagcatccatgaggacgcaggttcgatcccagg | | |
| cctcgctcagtgagttaaggatccgacattgccatgagctgtggtgta | | |
| ggtcgcagacacggctcggatctggcatgactgtggctgtggcgtagg | | |
| ccgtcggctacagctctgattggacccctagcctggaaacctccatat | | |
| gccgtgggtgcagccctcaaaagacaaacaaaaaaggttagctcagtc | | |
| tgtgaatgtaagactcctcgagggtcagcctaggacggtcttaagagg | | |
| ctggtgctgtgagtgtgggaatttgacaagtaaggactcggaggagcc | | |
| tcttgagccgggaagctgggaggtggaccccagcctggccgaccctgg | | |
| gctctgtgccccgtgtggtgccagcccgtggtggggactcaggcagtg | | |
| gccctgctgaggcggtggtggccactgggctctcgtccacag | | |
| GTACACCCAGTTCGTCCAGCGCTTCCTGGAGTCGGCCGAGCGCTTCTT | exon 7 | Seq. ID No. 40 |
| CATGCAGGGCTACCGGGTGCACTACTACATCTTTACCAGCGACCCCGG | | |
| GGCCGTTCCTGGGGTCCCGCTGGGCCCGGGCCGCCTCCTCAGCGTCAT | | |
| CGCCATCCGGAGACCCTCCCGCTGGGAGGAGGTCTCCACACGCCGGAT | | |
| GGAGGCCATCAGCCAGCACATTGCCGCCAGGGCGCACCGGGAGGTCGA | | |
| CTACCTCTTCTGCCTCAGCGTGGACATGGTGTTCCGGAACCCATGGGG | | |
| CCCCGAGACCTTGGGGGACCTGGTGGCTGCCATTCACCCGGGCTACTT | | |
| CGCCGCGCCCCGCCAGCAGTTCCCCTACGAGCGCCGGCATGTTTCTAC | | |
| CGCCTTCGTGGCGGACAGCGAGGGGGACTTCTATTATGGTGGGGCGGT | | |
| CTTCGGGGGGCGGGTGGCCAGGGTGTACGAGTTCACCCAGGGCTGCCA | | |
| CATGGGCATCCTGGCGGACAAGGCCAATGGCATCATGGCGGCCTGGCA | | |
| GGAGGAGAGCCACCTGAACCGCCGCTTCATCTCCCACAAGCCCTCCAA | | |
| GGTGCTGTCCCCCGAGTACCTCTGGGATGACCGCAGGCCCCAGCCCCC | | |
| CAGCCTGAAGCTGATCCGCTTTTCCACACTGGACAAAGACACCAACTG | | |
| GCTGAGGAGCTGACAGCACAGCCGGGGCTGCTGTGCATGCGGGGGGAC | | |
| CCCAAGCCCTGCCCCCAGCTCGCCCCAGCAGCGCCTCCTCACCCGGAC | | |
| GCCTCACTTCCCAAGCCTTCTGTGAAACCAGCCCTGCGCTGCCTACCT | | |
| CTCAGGCTGCCAGCAGACTCCGAGGCCTGTGTAAACTGTGAAGGGCTG | | |
| TGCCCTTGTGAGAACACACAGCCTGTGAGCCAGAAACGGTCAGACGGG | | |
| AGGAGACGGACCAGAGGTAGAAGAAGACGGGACCCGCAGTCCTCACCC | | |
| AGCCCACGTGCCTTTGGGGTGGGCGCTGGAGGGTCAGCCCTGCCCAGT | | |
| GCCTGACGTCCCGCCCACCCCCCTTTTGTGGCCGTTTGTACCTCTGAC | | |
| ACATGAGAGAGGTATCCTGGACCCCTGTCCTCTGGCTGCAGGGGCCCC | | |
| GGGGACTGTTCTGTCCCCCTGCCACAAGGAGCCAGTACCTCACTCAGG | | |
| ACCCCGACCGAGCCTTCGAAATGGACCCCGCCTGGGCTCTCTCGTTCC | | |
| ACGTCCAGCCCACCTCTGCAGTGGACCACGCTCCCTGGTGCCCACCGC | | |
| CTCCTTTGCAAGGGGGTTTGGGCAGCTTTTTAATACAGGTGGCATGTG | | |
| CTCAGCCCTAACC | | |

PCT Publication No. WO 04/108904 to Univerity of Pittsburgh provides the full length cDNA sequence, peptide sequence, and genomic organization of the porcine CMP-Neu5Ac hydroxylase gene. In addition, this publication provides porcine animals, tissues, and organs, as well as cells and cell lines derived from such animals, tissue, and organs, which lack expression of functional CMP-Neu5Ac hydroxylase, which can be used in research and medical therapy, including xenotransplantation. WO 04/108904 is incorporated by reference in its entirety.

c. Hexosamine Synthesis Pathway

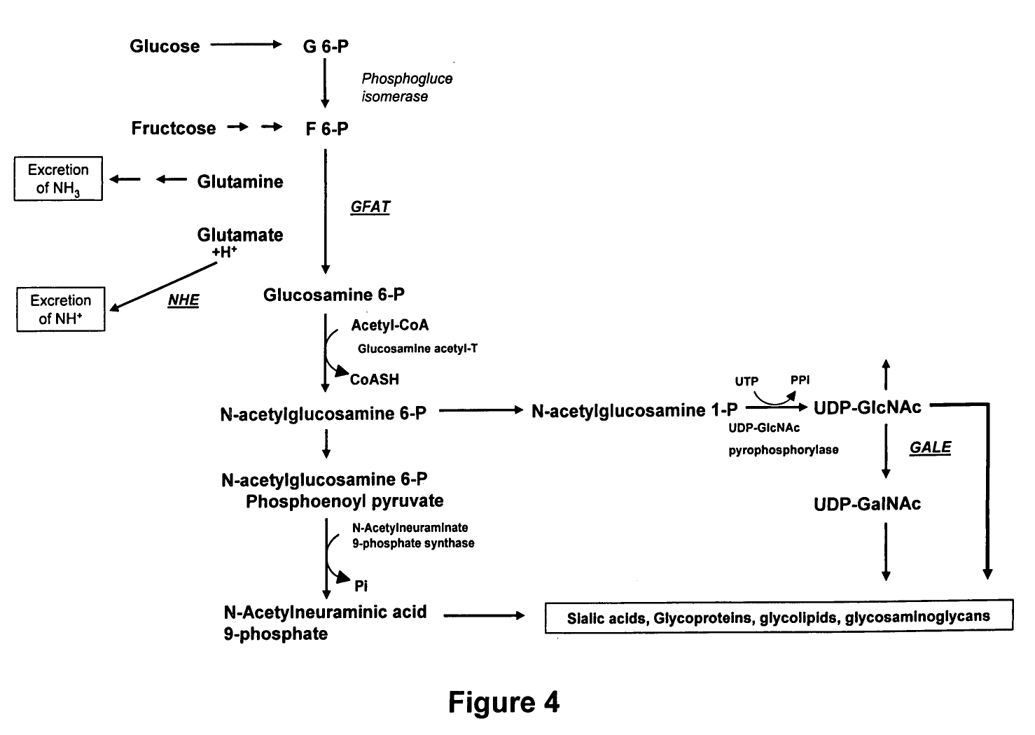

In the hexosamine pathway, N-acetylated sugars are produced in the coupling reaction with glutamine and the rate-limiting enzyme glutamine:fructose-6-phosphate amidotransferase (GFAT). In the reaction, galactose is 1) phosphorylated at C1 by ATP in a reaction catalyzed by galactokinase to produce galactose-1-phosphate; 2) galactose-1-phosphate uridyl transferase transfers the uridyl group of UDP-glucose to galactose-1-phosphate to yield glucose-1-phosphate and UDP-galactose by the reversible cleavage of UDP-glucose's pyrophosphoryl bond, 3) glucose 1-phosphate is converted to fructose-6-phosphate by the enzyme phosphoglucoisomerase, 4) fructose-6-phosphate is then converted to glucosamine 6-phosphate with the concomitant conversion of glutamine to glutamate by glucosamine:fructose-6-phosphate amindotransferase (GFAT), which is the rate limiting step for hexosamine synthesis, 7) glucosamine 6-phosphate is then rapidly converted through a series of steps to produce UDP-GlcNac, UDP-GalNAc, and sialic acid (See, for example, FIGS. 1A, 2, 4). Proteins associated with the hexosamine pathway include, but are not limited to, glutamine: fructose-6-phosphate amidotransferase (GFAT), the sodium-calcium exchanger (NCX) and the sodium-hydrogen exchanger (NHE).

In one embodiment, sugar metabolic processes are modified by genetically altering the expression of proteins associated with the hexosamine synthesis pathway and corresponding byproducts. Proteins associated with hexosamine synthesis that can be utilized for compensation in the present invention include, but are not limited to, phosphoglucomutase, phosphogluco-isomerase, glutamine:fructose-6-phosphate amidotransferase (GFAT), glucosamine-phosphate N-acetyl transferase, phosphoacetylglucosamine mutase, UDP-GlcNAc pyrophosphorylase, UDP-GlcNAc 4-epimerase, glucosamine kinase, and sodium hydrogen exchangers (NHE), including NHE-1, NHE-2, NHE-3, NHE-4, NHE-5, NHE-6, NHE-regulatory cofactor 1, NHE-regulatory cofactor 2, solute carrier family proteins such as SLC9 and related isoforms, and related homologs and isoforms.

TABLE 7
cDNA encoding Proteins involved in the Hexosamine Pathway

| Protein | | | |
|---|---|---|---|
| Associated | | Correspond- | |
| with | | ing | |
| Sugar | | Assession | Sequence |
| Metabolism | cDNA Sequence | Number | Identifier |
| glutamine- | ggtggcggag cccgggaggc ggagaaggct gtcgttgcct | BC045641 | Seq ID No. 51 |
| fructose- | tggccgtcgc atccccgagg 61 gagtcgtgtc | | |
| 6-phosphate | ggcgccaccc cggcccccga gcccgcagat tgcccaccga | | |
| amidotrans- | agctcgtgtg 121 tgcacccccg atcccgccag | | |
| ferase (GFAT) | ccactcgccc ctggcctcgc gggccgtgtc tccggcatca | | |
| | 181 tgtgtggtat atttgcttac ttaaactacc | | |
| | atgttcctcg aacgagacga gaaatcctgg 241 | | |
| | agaccctaat caaaggcctt cagagactgg agtacagagg | | |
| | atatgattct gctggtgtgg 301 gatttgatgg | | |
| | aggcaatgat aaagattggg aagccaatgc ctgcaaaatc | | |
| | cagcttatta 361 agaagaaagg aaaagttaag | | |
| | gcactggatg aagaagttca caagcaacaa gatatggatt | | |
| | 421 tggatataga atttgatgta caccttggaa | | |
| | tagctcatac ccgttgggca acacatggag 481 | | |
| | aacccagtcc tgtcaatagc cacccccagc gctctgataa | | |
| | aaataatgaa tttatcgtta 541 ttcacaatgg | | |
| | aatcatcacc aactacaaag acttgaaaaa gtttttggaa | | |
| | agcaaaggct 601 atgacttcga atctgaaaca | | |
| | gacacagaga caattgccaa gctcgttaag tatatgtatg | | |
| | 661 acaatcggga aagtcaagat accagcttta | | |
| | ctaccttggt ggagagagtt atccaacaat 721 | | |
| | tggaaggtgc ttttgcactt gtgtttaaaa gtgttcattt | | |
| | tcccgggcaa gcagttggca 781 caaggcgagg | | |
| | tagccctctg ttgattggtg tacggagtga acataaactt | | |
| | tctactgatc 841 acattcctat actctacaga | | |
| | acaggcaaag acaagaaagg aagctgcaat ctctctcgtg | | |
| | 901 tggacagcac aacctgcctt ttcccggtgg | | |
| | aagaaaaagc agtggagtat tactttgctt 961 | | |
| | ctgatgcaag tgctgtcata gaacacacca atcgcgtcat | | |
| | ctttctggaa gatgatgatg 1021 ttgcagcagt | | |
| | agtggatgga cgtctttcta tccatcgaat taaacgaact | | |
| | gcaggagatc 1081 accccggacg agctgtgcaa | | |
| | acactccaga tggaactcca gcagatcatg aagggcaact | | |
| | 1141 tcagttcatt tatgcagaag gaaatatttg | | |
| | agcagccaga gtctgtcgtg aacacaatga 1201 | | |
| | gaggaagagt caactttgat gactatactg tgaatttggg | | |
| | tggtttgaag gatcacataa 1261 aggagatcca | | |
| | gagatgccgg cgtttgattc ttattgcttg tggaacaagt | | |
| | taccatgctg 1321 gtgtagcaac acgtcaagtt | | |
| | cttgaggagc tgactgagtt gcctgtgatg gtggaactag | | |
| | 1381 caagtgactt cctggacaga aacacaccag | | |
| | tctttcgaga tgatgtttgc tttttcctta 1441 | | |
| | gtcaatcagg tgagacagca gatactttga tgggtcttcg | | |
| | ttactgtaag gagagaggag 1501 ctttaactgt | | |
| | ggggatcaca aacacagttg gcagttccat atcacgggag | | |
| | acagattgtg 1561 gagttcatat taatgctggt | | |
| | cctgagattg gtgtggccag tacaaaggct tataccagcc | | |
| | 1621 agtttgtatc ccttgtgatg tttgccctta | | |
| | tgatgtgtga tgatcggatc tccatgcaag 1681 | | |
| | aaagacgcaa agagatcatg cttggattga aacggctgcc | | |
| | tgatttgatt aaggaagtac 1741 tgagcatgga | | |
| | tgacgaaatt cagaaactag caacagaact ttatcatcag | | |
| | aagtcagttc 1801 tgataatggg acgaggctat | | |
| | cattatgcta cttgtcttga aggggcactg aaaatcaaag | | |
| | 1861 aaattactta tatgcactct gaaggcatcc | | |
| | ttgctggtga attgaaacat ggccctctgg 1921 | | |
| | ctttggtgga taaattgatg cctgtgatca tgatcatcat | | |
| | gagagatcac acttatgcca 1981 agtgtcagaa | | |
| | tgctcttcag caagtggttg ctcggcaggg gcggcctgtg | | |
| | gtaatttgtg 2041 ataaggagga tactgagacc | | |
| | attaagaaca caaaaagaac gatcaaggtg ccccactcgg | | |
| | 2101 tggactgctt gcagggcatt ctcagcgtga | | |
| | tccctttaca gttgctggct ttccaccttg 2161 | | |
| | ctgtgctgag aggctatgat gttgatttcc cacggaatct | | |
| | tgccaaatct gtgactgtag 2221 agtgaggaat | | |
| | atctatacaa aatgtacgaa actgtatgat taagcaacac | | |
| | aagacacctt 2281 ttgtatttaa aaccttgatt | | |
| | taaaatatca ccacttgaag ccttttttta gtaaatcctt | | |
| | 2341 atttatatat cagttataat tattccactc | | |
| | aatatgtgat ttttgtgaag ttacctctta 2401 | | |
| | cattttccca gtaatttgtg gaggactttg aataatggaa | | |
| | tctatattgg aatctgtatc 2461 agaaagattc | | |
| | tagctattat tttctttaaa gaatgctggg tgttgcattt | | |
| | ctggaccctc 2521 cacttcaatc tgagaagaca | | |
| | atatgtttct aaaaattggt acttgtttca ccatacttca | | |
| | 2581 ttcagaccag tgaaagagta gtgcatttaa | | |
| | ttggagtatc taaagccagt ggcagtgtat 2641 | | |
| | gctcatactt ggacagttag ggaagggttt gccaagtttt | | |
| | aagagaagat gtgatttatt 2701 ttgaaatttg | | |
| | tttctgtttt gtttttaaat caaactgtaa aacttaaaac | | |
| | tgaaaaattt 2761 tattggtagg atttatatct | | |
| | aagtttggtt agccttagtt tctcagactt gttgtctatt | | |
| | 2821 atctgtaggt ggaagaaatt taggaagcga | | |
| | aatattacag tagtgcattg gtgggtctca 2881 | | |
| | atccttaaca tatttgcaca attttatagc acaaacttta | | |
| | aattcaagct gctttggaca 2941 actgacaata | | |
| | tgattttaaa tttgaagatg ggatgtgtac atgttgggta | | |
| | tcctactact 3001 ttgtgttttc atctcctaaa | | |
| | agtggttttt atttccttgt atctgtagtc ttttattttt | | |
| | 3061 taaatgactg ctgaatgaca tattttatct | | |
| | tgttctttaa aatcacaaca cagagctgct 3121 | | |
| | attaaattaa tattgatata ttcaaaaaaa aaaaaaaaaa | | |
| NHE (sodium- | atgggcctgg ggcctgcctg ggtcacacag ccttgcctgg | XM_062645 | Seq ID No. 52 |
| hydrogen | tcactgactc ccagcctgat 61 gcggaattac | | |
| exchanger) | tctcctcaag agcaccctgc ctaggtcggc ggtgctgctg | | |
| | gtccccgggc 121 agaggaggcg tgggcggctc | | |
| | cgggaccacg gagcctggtg acgcggcgct cccctgcccg | | |
| | 181 ggtcgggttg cccaggcgcc gccgcggcgg | | |
| | ctgctgctgc tgctgccgct gctgctgggt 241 | | |
| | aggggacttc gagtaacggc cgaggcctcg gcctcctcct | | |
| | ctggggcggc ggtcgagaac 301 agcagcgcca | | |
| | tggaggagct cgtcactgag aaggaggcgg aagagagcca | | |
| | ccggccagac 361 agtgtgagcc tgctcacctt | | |
| | catcctgctg ctcacgctgg ccatcctcac catatggctc | | |
| | 421 ttcaagtact gccgggtgca ctttctgcat | | |
| | gagaccgggc tggccatgat ctgtgggctc 481 | | |
| | atcgttgggg tgatcctgag gtatggtacc cctggcacca | | |
| | ggggccgtga caaattactc 541 aattgcactc | | |
| | aagaagatca ggccttcagc actttagtag tggatgtcag | | |
| | cggtaaattc 601 ttcgaataca ccctgaaaag | | |
| | agaaatcagc cctggcaaga tcaacagcgt aaagcagaat | | |
| | 661 gacatgctag ggaaggtaac attcgaccca | | |
| | taggtatttt tcaacattct tctgcctcca 721 | | |
| | gttattttcc atgctggata cagcttaaag agacactttt | | |
| | ttagaaatct tgggtcactc 781 cttcttgggg | | |
| | actgctgttt cgtgcttccg tattggaaat ctcaggtatg | | |
| | gtatggtgaa 841 gctcatgagg attatgagac | | |
| | agctctcaga taaattttac tacacacatt gtctcttttt | | |
| | 901 tagagcaatc atctctgcca ctgacccagt | | |
| | gactgtgctg gtgatatcaa tgaattgcat 961 | | |
| | gcagacatgg atctttatgt acttctgttt ggagagagca | | |
| | tcctaaatga cgttgttatg 1021 ttgtactttc | | |
| | ctcatctatt gttggctacc agccagcagg actgaacttc | | |
| | aactcacgcc 1081 tttgatgctg ctgccttttt | | |
| | aaagtcagtt ggcatttttc taggtatatt tagtggctgt | | |
| | 1141 tttaccatgg gagctgtgac tggtgttgtg | | |
| | actgctttag tgaccaagtt taccaaactg 1201 | | |
| | gactgctttc ccctgctgga gacggcgctc ttcttcctca | | |
| | tgtcctggag cacgtttctc 1261 ttggcagaag | | |
| | cttgcggatt tacaggcgtt gtagctgtcc ttttctgtgg | | |
| | aatcacacaa 1321 gctcattaca ccttcaacaa | | |
| | tctgtcggtg gaatcaagaa gtcgaagcaa gcagctcttt | | |
| | 1381 gaggcagaga acttcatctt ctcctgcatg | | |
| | atcctggcgc tatttacctt ccagaagcac 1441 | | |
| | gttttcagcc ctgttttcat cattggagct tttgttgctg | | |
| | tcttcctggg cagagccgcc 1501 catatctacc | | |
| | cgctctcttt cttcctcagc ttgggcagaa ggcataagat | | |
| | tggctggaat 1561 tttcaacaca cgatgatgtt | | |
| | ttcaggcctc aggggagcaa tggcatttgc gttggccatc | | |
| | 1621 tgtgacacgg catcctatgc tcgccagatg | | |
| | acgttcccca ccacgccttt catcgtgttc 1681 | | |
| | ttcaccatct ggatcattgg aggaggcacg acacccatgt | | |
| | tgtcatggct taatatcaga 1741 gttagcatca | | |
| | aggagccctc caaagaggac cacaacgaac accaccgaca | | |
| | gtacttcaga 1801 gttggtgttg accctgatca | | |
| | agatccacca cccaacaatg acagctttca agtcttacaa | | |
| | 1861 ggggacagcc cagattctgc cagaggaaac | | |
| | tggacaaaac aggagagcac atggatattc 1921 | | |
| | aggcggtggt acagctttga tcacaattac ctgaagccca | | |
| | tcctcacaca cagcggctcc 1981 ccgctaacca | | |
| | ccactctccc gcctggtgga gacacagcgg ctccccgcta | | |
| | accaccactc 2041 tcctgcctgg tgtagacaaa | | |
| | gcggctcccc gccaaccacc actctcccgc ctggtgtagc | | |
| | 2101 ttgctagctt gatgtctgac cagtccccag | | |
| | gtgtacgata accaagagcc actgagagag 2161 | | |
| | ggaaactctg attttattct gactgaaggc gacctcacat | | |
| | tgacctatgg ggacagcaca 2221 gtgactgcaa | | |
| | atggcttctc aggttcccac actgcctcca cgagtctgga | | |
| | gggcagctgg 2281 agaatgaaga gcagctcaga | | |
| | ggaagtgctg gagcaggacg tgggaatggg aaaccagaag | | |
| | 2341 gtttcgagcc agggtacccg cctagtgttt | | |
| | cctctggaag ataatgtttg actttccctg 2401 | | |
| | caaaccctgg cacgatgggg taggctccca atggggtgag | | |
| | gatggcttca agccctaatg 2461 ttgcttgagg | | |
| | tggggcagtg actagattga attaactctt ctattttatt | | |
| | ggggtctgaa 2521 gttattgtaa cacttaaaat | | |
| | ttaactcatg atgcagatgg tgaggcaaaa gtgtctctaa | | |
| | 2581 attcagacaa atgtagacct atttctactt | | |
| | tttttcacac agtagtgcgc tgtttcagag 2641 | | |
| | ttaaacaaac aaaaaaatag cat | | |

The tables above represent cDNA sequences for certain mammalian galactosyltransferases as well as proteins involved in sugar catabolism, sugar chain synthesis and the hexosamine pathway (Tables 1-7). These cDNA sequences can be inserted into vectors for expression in host cells.

cDNAs can be prepared by a variety of methods, including cloning, synthetic or enzymatic methods known in the art. cDNAs can be synthesized, in whole or in part, using chemical methods well known in the art (see, for example, Caruthers et al. (1980) Nucleic Acids Symp. Ser. (7)215-233). Alternatively, cDNAs can be produced enzymatically, recombinantly or can be cloned from any mammalian cell or cDNA library.

d. Other Proteins Involved in Sugar Metabolism

In other embodiments, additional proteins associated with sugar metabolism can be used according to the present invention, such proteins include, but are not limited to: Ribulose-phosphate 3-epimerase (Enzyme Classification No. (EC) 5.1.3.1); UDP-glucose 4-epimerase (EC5.1.3.2); Aldose 1-epimerase (EC5.1.3.3); L-ribulose-phosphate 4-epimerase (EC5.1.3.4); UDP-arabinose 4-epimerase (EC5.1.3.5); UDP-glucuronate 4-epimerase (EC5.1.3.6); UDP-N-acetylglucosamine 4-epimerase (EC5.1.3.7); N-acylglucosamine 2-epimerase (EC5.1.3.8); N-acylglucosamine-6-phosphate 2-epimerase (EC5.1.3.9); CDP-abequose epimerase (EC5.1.3.10); Cellobiose epimerase (EC5.1.3.11); UDP-glucuronate 5′-epimerase (EC5.1.3.12); dTDP-4-dehydrorhamnose 3,5-epimerase (EC5.1.3.13); UDP-N-acetylglucosamine 2-epimerase (EC5.1.3.14); Glucose-6 phosphate 1-epimerase (EC5.1.3.15); UDP-glucosamine epimerase (EC5.1.3.16); Heparosan-N-sulfate-glucuronate 5-epimerase (EC5.1.3.17); GDP-mannose 3,5-epimerase (EC5.1.3.18); Chondroitin-glucuronate 5-epimerase (EC5.1.3.19); ADP-glyceromanno-heptose 6-epimerase (EC5.1.3.20); Maltose epimerase (EC5.1.3.21); Triosephosphate isomerase (EC5.3.1.1); Arabinose isomerase (EC5.3.1.3); L-arabinose isomerase (EC5.3.1.4); Xylose isomerase (EC5.3.1.5); Ribose 5-phosphate epimerase (EC5.3.1.6); Mannose isomerase (EC5.3.1.7); Mannose-6-phosphate isomerase (EC5.3.1.8); Glucose-6-phosphate isomerase (EC5.3.1.9); Glucuronate isomerase (EC5.3.1.12); Arabinose-5-phosphate isomerase (EC5.3.1.13); L-rhamnose isomerase (EC5.3.1.14); D-lyxose ketol-isomerase (EC5.3.1.15); 1-(5-phosphoribosyl)-5-[(5-phosphoribosylamino)methylideneamino] (EC5.3.1.16); 4-deoxy-L-threo-5-hexosulose-uronate ketol-isomerase (EC5.3.1.17); Ribose isomerase (EC5.3.1.20); Corticosteroid side-chain-isomerase (EC5.3.1.21); Hydroxypyruvate isomerase (EC5.3.1.22); 5-methylthioribose-1-phosphate isomerase (EC5.3.1.23); Phosphoribosylanthranilate isomerase (EC5.3.1.24); L-fucose isomerase (EC5.3.1.25); galactose-6-phosphate isomerase (EC5.3.1.26); Phosphoglycerate mutase (EC5.4.2.1); Phosphoglucomutase (EC5.4.2.2); Phosphoacetylglucosamine mutase (EC5.4.2.3); Bisphosphoglycerate mutase (EC5.4.2.4); Phosphoglucomutase (glucose-cofactor (EC5.4.2.5); Beta-phosphoglucomutase (EC5.4.2.6); Phosphopentomutase (EC5.4.2.7); Phosphomannomutase.(EC5.4.2.8); Phosphoenolpyruvate mutase (EC5.4.2.9); Phosphoglucosamine mutase (EC5.4.2.10); Maltose alpha-D-glucosyltransferase (EC5.4.99.16); Transketolase (EC2.2.1.1); Transaldolase.(EC2.2.1.2); Glucosamine N-acetyltransferase (EC2.3.1.3); Glucosamine 6-phosphate N-acetyltransferase (EC2.3.1.4); Maltose O-acetyltransferase (EC2.3.1.79); Phosphorylase (EC2.4.1.1); Dextrin dextranase (EC2.4.1.2); Amylosucrase (EC2.4.1.4); Dextransucrase (EC2.4.1.5); Sucrose phosphorylase (EC2.4.1.7); Maltose phosphorylase (EC2.4.1.8); Inulosucrase.(EC2.4.1.9); Levansucrase (EC2.4.1.10); Glycogen (starch) synthase (EC2.4.1.11); Cellulose synthase (UDP-forming) (EC2.4.1.12); Sucrose synthase (EC2.4.1.13); Sucrose-phosphate synthase (EC2.4.1.14); Alpha,alpha-trehalose-phosphate synthase (UDP-forming)(EC2.4.1.15); Chitin synthase (EC2.4.1.16); UDP-glucuronosyltransferase (EC2.4.1.17); 1,4-alpha-glucan branching enzyme (EC2.4.1.18); Cyclomaltodextrin glucanotransferase (EC2.4.1.19); Cellobiose phosphorylase (EC2.4.1.20); Starch (bacterial glycogen) synthase (EC2.4.1.21); Lactose synthase (EC2.4.1.22); Sphingosine beta-galactosyltransferase (EC2.4.1.23); 1,4-alpha-glucan 6-alpha-glucosyltransferase (EC2.4.1.24); 4-alpha-glucanotransferase.(EC2.4.1.25); Dna alpha-glucosyltransferase (EC2.4.1.26); Dna beta-glucosyltransferase (EC2.4.1.27); Glucosyl-DNA beta-glucosyltransferase (EC2.4.1.28); Cellulose synthase (GDP-forming) (EC2.4.1.29); 1,3-beta-oligoglucan phosphorylase (EC2.4.1.30); Laminaribiose phosphorylase (EC2.4.1.31); Glucomannan 4-beta-mannosyltransferase (EC2.4.1.32); Alginate synthase (EC2.4.1.33); 1,3-beta-glucan synthase (EC2.4.1.34); Phenol beta-glucosyltransferase (EC2.4.1.35); Alpha,alpha-trehalose-phosphate synthase (GDP-forming) (EC2.4.1.36); Glycoprotein-fucosylgalactoside alpha-galactosyltransferase (EC2.4.1.37); Beta-N-acetylglucosaminyl-glycopeptide beta-1,4-galactosyltransferase (EC2.4.1.38); Steroid N-acetylglucosaminyltransferase (EC2.4.1.39); Glycoprotein-fucosylgalactoside alpha-N-acetylgalactosaminyltransferase (EC2.4.1.40); Polypeptide N-acetylgalactosaminyltransferase (EC2.4.1.41); Polygalacturonate 4-alpha-galacturonosyltransferase (EC2.4.1.43); Lipopolysaccharide galactosyltransferase (EC2.4.1.44); 2-hydroxyacylsphingosine 1-beta-galactosyltransferase (EC2.4.1.45); 1,2-diacylglycerol 3-beta-galactosyltransferase (EC2.4.1.46); N-acylsphingosine galactosyltransferase (EC2.4.1.47); Heteroglycan alpha-mannosyltransferase (EC2.4.1.48); Cellodextrin phosphorylase (EC2.4.1.49); Procollagen galactosyltransferase (EC2.4.1.50); Poly(glycerol-phosphate) alpha-glucosyltransferase (EC2.4.1.52); Poly(ribitol-phosphate) beta-glucosyltransferase (EC2.4.1.53); Undecaprenyl-phosphate mannosyltransferase (EC2.4.1.54); Lipopolysaccharide N-acetylglucosaminyltransferase (EC2.4.1.56); Phosphatidyl-myo-inositol alpha-mannosyltransferase (EC2.4.1.57); Lipopolysaccharide glucosyltransferase I (EC2.4.1.58); Abequosyltransferase (EC2.4.1.60); Ganglioside galactosyltransferase (EC2.4.1.62); Linamarin synthase (EC2.4.1.63); Alpha,alpha-trehalose phosphorylase (EC2.4.1.64); 3-galactosyl-N-acetylglucosaminide 4-alpha-L-fucosyltransferase (EC2.4.1.65); Procollagen glucosyltransferase (EC2.4.1.66); Galactinol-raffinose galactosyltransferase (EC2.4.1.67); Glycoprotein 6-alpha-L-fucosyltransferase (EC2.4.1.68); Galactoside 2-alpha-L-fucosyltransferase (EC2.4.1.69); Poly(ribitol-phosphate) N-acetylglucosaminyltransferase (EC2.4.1.70); Arylamine glucosyltransferase (EC2.4.1.71); Lipopolysaccharide glucosyltransferase (EC2.4.1.73); Glycosaminoglycan galactosyltransferase (EC2.4.1.74); UDP-galacturonosyltransferase (EC2.4.1.75); Phosphopolyprenol glucosyltransferase (EC2.4.1.78); Galactosylgalactosylglucosylceramide beta-D-acetyl-(EC2.4.1.79); Ceramide glucosyltransferase (EC2.4.1.80); Flavone 7-O-beta-glucosyltransferase (EC2.4.1.81); Galactinol-sucrose galactosyltransferase (EC2.4.1.82); Dolichyl-phosphate beta-D-mannosyltransferase (EC2.4.1.83); Cyanohydrin beta-glucosyltransferase (EC2.4.1.85); Glucosaminylgalactosylglucosylceramide beta-galactosyltransferase (EC2.4.1.86); Beta-galactosyl-N-acetylglucosaminylglycopeptide alpha-1,3-(EC2.4.1.87); Globoside alpha-N-acetylgalactosaminyltransferase (EC2.4.1.88); N-acetyllactosamine synthase (EC2.4.1.90); Flavonol 3-O-glucosyltransferase (EC2.4.1.91); (N-acetylneuraminyl)-galactosylglucosylceramide (EC2.4.1.92). Inulin fructotransferase (depolymerizing) (EC2.4.1.93); Protein N-acetylglucosaminyltransferase (EC2.4.1.94); Bilirubin-glucuronoside glucuronosyltransferase (EC2.4.1.95); Sn-glycerol-3-phosphate 1-galactosyltransferase (EC2.4.1.96); 1,3-beta-glucan phosphorylase (EC2.4.1.97); Sucrose 1F-fructosyltransferase (EC2.4.1.99); 1,2-beta-fructan 1F-fructosyltransferase (EC2.4.1.100); Alpha-1,3-mannosyl-glycoprotein 2-beta-N-(EC2.4.1.101); Beta-1,3-galactosyl-O-glycosyl-glycoprotein beta-1,6-N-(EC2.4.1.102); Alizarin 2-beta-glucosyltransferase (EC2.4.1.103); O-dihydroxycoumarin 7-O-glucosyltransferase (EC2.4.1.104); Vitexin beta-glucosyltransferase (EC2.4.1.105); Isovitexin beta-glucosyltransferase (EC2.4.1.106); Dolichyl-phosphate-mannose-protein mannosyltransferase (EC2.4.1.109); tRNA-queuosine beta-mannosyltransferase (EC2.4.1.110); Coniferyl-alcohol glucosyltransferase (EC2.4.1.111); Alpha-1,4-glucan-protein synthase (UDP-forming) (EC2.4.1.112); Alpha-1,4-glucan-protein synthase (ADP-forming) (EC2.4.1.113); 2-coumarate O-beta-glucosyltransferase (EC2.4.1.114); Anthocyanidin 3-O-glucosyltransferase (EC2.4.1.115); Cyanidin-3-rhamnosylglucoside 5-O-glucosyltransferase (EC2.4.1.116); Dolichyl-phosphate beta-glucosyltransferase (EC2.4.1.117); Cytokinin 7-beta-glucosyltransferase (EC2.4.1.118); Dolichyl-diphosphooligosaccharide-protein glycosyltransferase (EC2.4.1.119); Sinapate 1-glucosyltransferase (EC2.4.1.120); Indole-3-acetate beta-glucosyltransferase (EC2.4.1.121); Glycoprotein-N-acetylgalactosamine 3-beta-galactosyltransferase (EC2.4.1.122); Inositol 1-alpha-galactosyltransferase (EC2.4.1.123); N-acetyllactosamine 3-alpha-galactosyltransferase (EC2.4.1.124); Sucrose-1,6-alpha-glucan 3(6)-alpha-glucosyltransferase (EC2.4.1.125); Hydroxycinnamate 4-beta-glucosyltransferase (EC2.4.1.126); Monoterpenol beta-glucosyltransferase (EC2.4.1.127); Scopoletin glucosyltransferase (EC2.4.1.128); Peptidoglycan glycosyltransferase (EC2.4.1.129); Dolichyl-phosphate-mannose-glycolipid alpha-mannosyltransferase (EC2.4.1.130); Glycolipid 2-alpha-mannosyltransferase (EC2.4.1.131); Glycolipid 3-alpha-mannosyltransferase (EC2.4.1.132); Xylosylprotein 4-beta-galactosyltransferase [(EC2.4.1.133-]); Galactosylxylosylprotein 3-beta-galactosyltransferase (EC2.4.1.134); Galactosylgalactosylxylosylprotein 3-beta-glucuronosyltransferase (EC2.4.1.135); Gallate 1-beta-glucosyltransferase (EC2.4.1.136); Sn-glycerol-3-phosphate 2-alpha-galactosyltransferase (EC2.4.1.137); Mannotetraose 2-alpha-N-acetylglucosaminyltransferase (EC2.4.1.138); Maltose synthase (EC2.4.1.139); Alternansucrase (EC2.4.1.140); N-acetylglucosaminyldiphosphodolichol N-acetylglucosaminyltransferase (EC2.4.1.141); Chitobiosyldiphosphodolichol beta-mannosyltransferase (EC2.4.1.142); Alpha-1,6-mannosyl-glycoprotein 2-beta-N-(EC2.4.1.143); Beta-1,4-mannosyl-glycoprotein 4-beta-N-acetylglucosaminyltransferase (EC2.4.1.144); Alpha-1,3-mannosyl-glycoprotein 4-beta-N-acetylglucosaminyltransferase (EC2.4.1.145); Beta-1,3-galactosyl-O-glycosyl-glycoprotein beta-1,3-N-[(EC2.4.1.146-]); Acetylgalactosaminyl-O-glycosyl-glycoprotein beta-1,3-N-(EC2.4.1.147); Acetylgalactosaminyl-O-glycosyl-glycoprotein beta-1,6-N-(EC2.4.1.148); N-acetyllactosaminide beta-1,3-N-acetylglucosaminyltransferase (EC2.4.1.149); N-acetyllactosaminide beta-1,6-N-acetylglucosaminyltransferase (EC2.4.1.150); N-acetyllactosaminide alpha-1,3-galactosyltransferase (EC2.4.1.151); 4-galactosyl-N-acetylglucosaminide 3-alpha-L-fucosyltransferase (EC2.4.1.152); Dolichyl-phosphate alpha-N-acetylglucosaminyltransferase (EC2.4.1.153); Globotriosylceramide beta-1,6-N-acetylgalactosaminyltransferase (EC2.4.1.154); Alpha-1,6-mannosyl-glycoprotein 6-beta-N-(EC2.4.1.155); Indolylacetyl-myo-inositol galactosyltransferase (EC2.4.1.156); 1,2-diacylglycerol 3-glucosyltransferase (EC2.4.1.157); 13-hydroxydocosanoate 13-beta-glucosyltransferase (EC2.4.1.158); Flavonol-3-O-glucoside L-rhamnosyltransferase (EC2.4.1.159); Pyridoxine 5′-O-beta-D-glucosyltransferase (EC2.4.1.160); Oligosaccharide 4-alpha-D-glucosyltransferase (EC2.4.1.161); Aldose beta-D-fructosyltransferase (EC2.4.1.162); Beta-galactosyl-N-acetylglucosaminylgalactosyl-glucosylceramide (EC2.4.1.163); Galactosyl-N-acetylglucosaminylgalactosyl-glucosylceramide beta-1,6-(EC2.4.1.164); N-acetylneuraminylgalactosylglucosylceramide beta-1,4-N-(EC2.4.1.165); Raffinose-raffinose alpha-galactosyltransferase (EC2.4.1.166); Sucrose 6(F)-alpha-galactosyltransferase (EC2.4.1.167); Xyloglucan 4-glucosyltransferase (EC2.4.1.168); Xyloglucan 6-xylosyltransferase (EC2.4.1.169); Isoflavone 7-O-glucosyltransferase (EC2.4.1.170); Methyl-ONN-azoxymethanol glucosyltransferase (EC2.4.1.171); Salicyl-alcohol glucosyltransferase (EC2.4.1.172); Sterol glucosyltransferase (EC2.4.1.173); Glucuronylgalactosylproteoglycan 4-beta-N-(EC2.4.1.174); Glucuronosyl-N-acetylgalactosaminyl-proteoglycan 4-beta-N-(EC2.4.1.175); Gibberellin beta-glucosyltransferase (EC2.4.1.176); Cinnamate glucosyltransferase (EC2.4.1.177); Hydroxymandelonitrile glucosyltransferase (EC2.4.1.178); Lactosylceramide beta-1,3-galactosyltransferase (EC2.4.1.179); Lipopolysaccharide N-acetylmannosaminouronosyltransferase (EC2.4.1.180); Hydroxyanthraquinone glucosyltransferase (EC2.4.1.181); Lipid-A-disaccharide synthase (EC2.4.1.182); Alpha-1,3-glucan synthase (EC2.4.1.183); Galactolipid galactosyltransferase (EC2.4.1.184); Flavonone 7-O-beta-glucosyltransferase (EC2.4.1.185); Glycogenin glucosyltransferase (EC2.4.1.186); N-acetylglucosaminyldiphosphoundecaprenol N-acetyl-beta-D-(EC2.4.1.187); N-acetylglucosaminyldiphosphoundecaprenol glucosyltransferase (EC2.4.1.188); Luteolin 7-O-glucoronosyltransferase (EC2.4.1.189); Luteolin-7-O-glucuronide 7-O-glucuronosyltransferase (EC2.4.1.190); Luteolin-7-O-diglucuronide 4′-O-glucuronosyltransferase (EC2.4.1.191); Nuatigenin 3-beta-glucosyltransferase (EC2.4.1.192); Sarsapogenin 3-beta-glucosyltransferase (EC2.4.1.193); 4-hydroxybenzoate 4-O-beta-D-glucosyltransferase (EC2.4.1.194); Thiohydroximate beta-D-glucosyltransferase (EC2.4.1.195); Nicotinate glucosyltransferase (EC2.4.1.196); High-mannose-oligosaccharide beta-1,4-N-acetyl-glucosaminyltransferase (EC2.4.1.197); Phosphatidylinositol N-acetylglucosaminyltransferase (EC2.4.1.198); Beta-mannosylphosphodecaprenol-mannooligosaccharide (EC2.4.1.199); Inulin fructotransferase (depolymerizing, difructofuranose-(EC2.4.1.200); Alpha-1,6-mannosyl-glycoprotein 4-beta-N-acetylglucosaminyltransferase (EC2.4.1.201); 2,4-dihydroxy-7-methoxy-2H-1,4-benzoxazin-3(4H)-one (EC2.4.1.202); Trans-zeatin O-beta-D-glucosyltransferase (EC2.4.1.203); Zeatin O-beta-D-xylosyltransferase (EC2.4.1.204); Galactogen 6-beta-galactosyltransferase (EC2.4.1.205); Lactosylceramide 1,3-N-acetyl-beta-D-glucosaminyl-transferase (EC2.4.1.206); Xyloglucan:xyloglucosyl transferase (EC2.4.1.207); Diglucosyl diacylglycerol (DGlcDAG) synthase (EC2.4.1.208); Cis-p-coumarate glucosyltransferase (EC2.4.1.209); Limonoid glucosyltransferase (EC2.4.1.210); 1,3-beta-galactosyl-N-acetylhexosamine phosphorylase (EC2.4.1.211); Hyaluronan synthase (EC2.4.1.212); Glucosylglycerol-phosphate synthase (EC2.4.1.213); Glycoprotein 3-alpha-L-fucosyltransferase (EC2.4.1.214); Cis-zeatin O-beta-D-glucosyltransferase (EC2.4.1.215); Trehalose 6-phosphate phosphorylase (EC2.4.1.216); Mannosyl-3-phosphoglycerate synthase (EC2.4.1.217); Hydroquinone glucosyltransferase (EC2.4.1.218); Vomilenine glucosyltransferase (EC2.4.1.219); Indoxyl-Udpg glucosyltransferase (EC2.4.1.220); Peptide-O-fucosyltransferase (EC2.4.1.221); O-fucosylpeptide 3-beta-N-acetylglucosaminyltransferase (EC2.4.1.222); Glucuronyl-galactosyl-proteoglycan 4-alpha-N-(EC2.4.1.223); Glucuronosyl-N-acetylglucosaminyl-proteoglycan 4-alpha-N-(EC2.4.1.224); N-acetylglucosaminyl-proteoglycan 4-beta-glucuronosyltransferase (EC2.4.1.225); N-acetylgalactosaminyl-proteoglycan 3-beta-glucuronosyltransferase (EC2.4.1.226); Undecaprenyldiphospho-muramoylpentapeptide beta-N-(EC2.4.1.227); Lactosylceramide 4-alpha-galactosyltransferase (EC2.4.1.228); Beta-galactosamide alpha-2,6-sialyltransferase (EC2.4.99.1); Monosialoganglioside sialyltransferase (EC2.4.99.2); Alpha-N-acetylgalactosaminide alpha-2,6-sialyltransferase (EC2.4.99.3); Beta-galactoside alpha-2,3-sialyltransferase (EC2.4.99.4); Galactosyldiacylglycerol alpha-2,3-sialyltransferase (EC2.4.99.5); N-acetyllactosaminide alpha-2,3-sialyltransferase (EC2.4.99.6); (Alpha-N-acetyl-neuraminyl-2,3-beta-galactosyl-1,3)-N-(EC2.4.99.7); Alpha-N-acetyl-neuraminide alpha-2,8-sialyltransferase (EC2.4.99.8); Lactosylceramide alpha-2,3-sialyltransferase (EC2.4.99.9); Neolactotetraosylceramide alpha-2,3-sialyltransferase (EC2.4.99.10); Lactosylceramide alpha-2,6-N-sialyltransferase (EC2.4.99.11); Hexokinase (EC2.7.1.1); Glucokinase (EC2.7.1.2); Ketohexokinase (EC2.7.1.3); Fructokinase (EC2.7.1.4); Rhamnulokinase (EC2.7.1.5); Galactokinase (EC2.7.1.6); Mannokinase (EC2.7.1.7); Glucosamine kinase (EC2.7.1.8); Phosphoglucokinase (EC2.7.1.10); 6-phosphofructokinase (EC2.7.1.11); Gluconokinase (EC2.7.1.12); Dehydogluconokinase (EC2.7.1.13); Sedoheptulokinase (EC2.7.1.14); Ribokinase (EC2.7.1.15); L-ribulokinase (EC2.7.1.16); Xylulokinase (EC2.7.1.17); Phosphoribokinase (EC2.7.1.18); Phosphoribulokinase (EC2.7.1.19); Ribosylnicotinamide kinase (EC2.7.1.22); NAD(+) kinase (EC2.7.1.23); Riboflavin kinase (EC2.7.1.26); Erythritol kinase (EC2.7.1.27); Triokinase (EC2.7.1.28); Glycerone kinase (EC2.7.1.29); Glycerol kinase (EC2.7.1.30); Glycerate kinase (EC2.7.1.31); Phosphorylase kinase (EC2.7.1.38); Pyruvate kinase (EC2.7.1.40); Glucose-1-phosphate phosphodismutase (EC2.7.1.41); Riboflavin phosphotransferase (EC2.7.1.42); Glucuronokinase (EC2.7.1.43); Galacturonokinase (EC2.7.1.44); 2-dehydro-3-deoxygluconokinase (EC2.7.1.45); L-arabinokinase (EC2.7.1.46); D-ribulokinase (EC2.7.1.47); Uridine kinase (EC2.7.1.48); Hydroxymethylpyrimidine kinase (EC2.7.1.49); Hydroxyethylthiazole kinase (EC2.7.1.50); L-fuculokinase (EC2.7.1.51); Fucokinase (EC2.7.1.52); L-xylulokinase (EC2.7.1.53); D-arabinokinase (EC2.7.1.54); Allose kinase (EC2.7.1.55); 1-phosphofructokinase (EC2.7.1.56); 2-dehydro-3-deoxygalactonokinase (EC2.7.1.58); N-acetylglucosamine kinase (EC2.7.1.59); N-acylmannosamine kinase (EC2.7.1.60); Acyl-phosphate-hexose phosphotransferase (EC2.7.1.61); Phosphoramidate-hexose phosphotransferase (EC2.7.1.62); Polyphosphate-glucose phosphotransferase (EC2.7.1.63); Inositol 3-kinase (EC2.7.1.64); Scyllo-inosamine kinase (EC2.7.1.65); Undecaprenol kinase (EC2.7.1.66); 1-phosphatidylinositol 4-kinase (EC2.7.1.67); 1-phosphatidylinositol-4-phosphate 5-kinase (EC2.7.1.68); Protein-N(pi)-phosphohistidine-sugar phosphotransferase (EC2.7.1.69); Protamine kinase (EC2.7.1.70); Shikimate kinase (EC2.7.1.71); Streptomycin 6-kinase (EC2.7.1.72); Inosine kinase (EC2.7.1.73); Diphosphate-glycerol phosphotransferase (EC2.7.1.79); Alkylglycerone kinase (EC2.7.1.84); Beta-glucoside kinase (EC2.7.1.85); Nadh kinase (EC2.7.1.86); Diphosphate-fructose-6-phosphate 1-phosphotransferase (EC2.7.1.90); Sphinganine kinase (EC2.7.1.91); 5-dehydro-2-deoxygluconokinase (EC2.7.1.92); Alkylglycerol kinase (EC2.7.1.93); Acylglycerol kinase (EC2.7.1.94); [Pyruvate dehydrogenase(lipoamide)] kinase (EC2.7.1.99); 5-methylthioribose kinase (EC2.7.1.100); Tagatose kinase (EC2.7.1.101); Hamamelose kinase (EC2.7.1.102); 6-phosphofructo-2-kinase (EC2.7.1.105); Glucose-1,6-bisphosphate synthase (EC2.7.1.106); Diacylglycerol kinase (EC2.7.1.107); Phosphoenolpyruvate-glycerone phosphotransferase (EC2.7.1.121); Xylitol kinase (EC2.7.1.122); Tetraacyldisaccharide 4′-kinase (EC2.7.1.130); Phosphatidylinositol 3-kinase (EC2.7.1.137); Ceramide kinase (EC2.7.1.138); Glycerol-3-phosphate-glucose phosphotransferase (EC2.7.1.142); Tagatose-6-phosphate kinase (EC2.7.1.144); 4-(cytidine 5′-diphospho)-2-C-methyl-D-erythritol kinase (EC2.7.1.148); 1-phosphatidylinositol-5-phosphate 4-kinase (EC2.7.1.149); 1-phosphatidylinositol-3-phosphate 5-kinase (EC2.7.1.150); Phosphatidylinositol-4,5-bisphosphate 3-kinase (EC2.7.1.153); Phosphatidylinositol-4-phosphate 3-kinase (EC2.7.1.154); Ribose-phosphate pyrophosphokinase (EC2.7.6.1); UTP-glucose-1-phosphate uridylyltransferase (EC2.7.7.9); UTP-hexose-1-phosphate uridylyltransferase (EC2.7.7.10); UTP-xylose-1-phosphate uridylyltransferase (EC2.7.7.11); UDP-glucose-hexose-1-phosphate uridylyltransferase (EC2.7.7.12); Mannose-1-phosphate guanylyltransferase (EC2.7.7.13); Mannose-1-phosphate guanylyltransferase (GDP) (EC2.7.7.22); UDP-N-acetylglucosamine pyrophosphorylase (EC2.7.7.23); Glucose-1-phosphate thymidylyltransferase (EC2.7.7.24); Glucose-1-phosphate adenylyltransferase (EC2.7.7.27); Nucleoside-triphosphate-hexose-1-phosphate nucleotidyltransferase (EC2.7.7.28); Hexose-1-phosphate guanylyltransferase (EC2.7.7.29); Fucose-1-phosphate guanylyltransferase (EC2.7.7.30); Glucuronate-1-phosphate uridylyltransferase (EC2.7.7.44); Alpha-amylase (EC3.2.1.1); Beta-amylase (EC3.2.1.2); Glucan 1,4-alpha-glucosidase (EC3.2.1.3); Cellulase (EC3.2.1.4); Endo-1,3(4)-beta-glucanase (EC3.2.1.6); Inulinase (EC3.2.1.7); Endo-1,4-beta-xylanase (EC3.2.1.8); Oligosaccharide alpha-1,6-glucosidase (EC3.2.1.10); Dextranase (EC3.2.1.11); Chitinase (EC3.2.1.14); Polygalacturonase (EC3.2.1.15); Lysozyme (EC3.2.1.17); Exo-alpha-sialidase (EC3.2.1.18); Alpha-glucosidase (EC3.2.1.20); Beta-glucosidase (EC3.2.1.21); Alpha-galactosidase (EC3.2.1.22); Beta-galactosidase (EC3.2.1.23); Alpha-mannosidase (EC3.2.1.24); Beta-mannosidase (EC3.2.1.25); Beta-fructofuranosidase (EC3.2.1.26); Alpha,alpha-trehalase (EC3.2.1.28); Beta-glucuronidase (EC3.2.1.31); Xylan endo-1,3-beta-xylosidase (EC3.2.1.32); Amylo-alpha-1,6-glucosidase (EC3.2.1.33); Hyaluronoglucosaminidase (EC3.2.1.35); Hyaluronoglucuronidase (EC3.2.1.36); Xylan 1,4-beta-xylosidase (EC3.2.1.37); Beta-D-fucosidase (EC3.2.1.38); Glucan endo-1,3-beta-D-glucosidase (EC3.2.1.39); Alpha-L-rhamnosidase (EC3.2.1.40); Pullulanase (EC3.2.1.41); GDP-glucosidase (EC3.2.1.42); Beta-L-rhamnosidase (EC3.2.1.43); Fucoidanase (EC3.2.1.44); Glucosylceramidase (EC3.2.1.45); Galactosylceramidase (EC3.2.1.46); Galactosylgalactosylglucosylceramidase (EC3.2.1.47); Sucrose alpha-glucosidase (EC3.2.1.48); Alpha-N-acetylgalactosaminidase (EC3.2.1.49); Alpha-N-acetylglucosaminidase (EC3.2.1.50); Alpha-L-fucosidase (EC3.2.1.51); Beta-N-acetylhexosaminidase (EC3.2.1.52); Beta-N-acetylgalactosaminidase (EC3.2.1.53); Cyclomaltodextrinase (EC3.2.1.54); Alpha-L-arabinofuranosidase (EC3.2.1.55); Glucuronosyl-disulfoglucosamine glucuronidase (EC3.2.1.56); Isopullulanase (EC3.2.1.57); Glucan 1,3-beta-glucosidase (EC3.2.1.58); Glucan endo-1,3-alpha-glucosidase (EC3.2.1.59); Glucan 1,4-alpha-maltotetrahydrolase (EC3.2.1.60); Mycodextranase (EC3.2.1.61); Glycosylceramidase (EC3.2.1.62); 1,2-alpha-L-fucosidase (EC3.2.1.63); 2,6-beta-fructan 6-levanbiohydrolase (EC3.2.1.64); Levanase (EC3.2.1.65); Quercitrinase (EC3.2.1.66); Galacturan 1,4-alpha-galacturonidase (EC3.2.1.67); Isoamylase (EC3.2.1.68); Glucan 1,6-alpha-glucosidase (EC3.2.1.70); Glucan endo-1,2-beta-glucosidase (EC3.2.1.71); Xylan 1,3-beta-xylosidase (EC3.2.1.72); Licheninase (EC3.2.1.73); Glucan 1,4-beta-glucosidase (EC3.2.1.74); Glucan endo-1,6-beta-glucosidase (EC3.2.1.75); L-iduronidase (EC3.2.1.76); Mannan 1,2-(1,3)-alpha-mannosidase (EC3.2.1.77); Mannan endo-1,4-beta-mannosidase (EC3.2.1.78); Fructan beta-fructosidase (EC3.2.1.80); Agarase (EC3.2.1.81); Exo-poly-alpha-galacturonosidase (EC3.2.1.82); Kappa-carrageenase (EC3.2.1.83); Glucan 1,3-alpha-glucosidase (EC3.2.1.84); *6-phospho-beta-galactosidase (EC3.2.1.85); 6-phospho-beta-glucosidase (EC3.2.1.86); Capsular-polysaccharide endo-1,3-alpha-galactosidase (EC3.2.1.87); Beta-L-arabinosidase (EC3.2.1.88); Arabinogalactan endo-1,4-beta-galactosidase (EC3.2.1.89); Cellulose 1,4-beta-cellobiosidase (EC3.2.1.91); Peptidoglycan beta-N-acetylmuramidase (EC3.2.1.92); Alpha,alpha-phosphotrehalase (EC3.2.1.93); Glucan 1,6-alpha-isomaltosidase (EC3.2.1.94); Dextran 1,6-alpha-isomaltotriosidase (EC3.2.1.95); Mannosyl-glycoprotein endo-beta-N-acetylglucosamidase (EC3.2.1.96); Glycopeptide alpha-N-acetylgalactosaminidase (EC3.2.1.97); Glucan 1,4-alpha-maltohexaosidase (EC3.2.1.98); Arabinan endo-1,5-alpha-L-arabinosidase (EC3.2.1.99); Mannan 1,4-beta-mannobiosidase (EC3.2.1.100); Mannan endo-1,6-beta-mannosidase (EC3.2.1.101); Blood-group-substance endo-1,4-beta-galactosidase (EC3.2.1.102); Keratan-sulfate endo-1,4-beta-galactosidase (EC3.2.1.103); Steryl-beta-glucosidase (EC3.2.1.104); Strictosidine beta-glucosidase (EC3.2.1.105); (EC3.2.1.105); Mannosyl-oligosaccharide glucosidase (EC3.2.1.106); Protein-glucosylgalactosylhydroxylysine glucosidase (EC3.2.1.107); Lactase (EC3.2.1.108); Endogalactosaminidase (EC3.2.1.109); Mucinaminylserine mucinaminidase (EC3.2.1.110); 1,3-alpha-L-fucosidase (EC3.2.1.111); 2-deoxyglucosidase (EC3.2.1.112); Mannosyl-oligosaccharide 1,2-alpha-mannosidase (EC3.2.1.113); Mannosyl-oligosaccharide 1,3-1,6-alpha-mannosidase (EC3.2.1.114); Branched-dextran exo-1,2-alpha-glucosidase (EC3.2.1.115); Glucan 1,4-alpha-maltotriohydrolase (EC3.2.1.116); Amygdalin beta-glucosidase (EC3.2.1.117); Prunasin beta-glucosidase (EC3.2.1.118); Vicianin beta-glucosidase (EC3.2.1.119); Oligoxyloglucan beta-glycosidase (EC3.2.1.120); Polymannuronate hydrolase (EC3.2.1.121); Maltose-6′-phosphate glucosidase (EC3.2.1.122); Endoglycosylceramidase (EC3.2.1.123); 3-deoxy-2-octulosonidase (EC3.2.1.124); Raucaffricine beta-glucosidase (EC3.2.1.125); Coniferin beta-glucosidase (EC3.2.1.126); 1,6-alpha-L-fucosidase (EC3.2.1.127); Glycyrrhizinate beta-glucuronidase (EC3.2.1.128); Endo-alpha-sialidase (EC3.2.1.129); Glycoprotein endo-alpha-1,2-mannosidase (EC3.2.1.130); Xylan alpha-1,2-glucuronosidase (EC3.2.1.131); Chitosanase (EC3.2.1.132); Glucan 1,4-alpha-maltohydrolase (EC3.2.1.133); Difructose-anhydride synthase (EC3.2.1.134); Neopullulanase (EC3.2.1.135); Glucuronoarabinoxylan endo-1,4-beta-xylanase (EC3.2.1.136); Mannan exo-1,2-1,6-alpha-mannosidase (EC3.2.1.137); Anhydrosialidase (EC3.2.1.138); Alpha-glucosiduronase (EC3.2.1.139); Lacto-N-biosidase (EC3.2.1.140); 4-alpha-D-{(1->4)-alpha-D-glucano}trehalose trehalohydrolase (EC3.2.1.141); Limit dextrinase (EC3.2.1.142); Poly(ADP-ribose) glycohydrolase (EC3.2.1.143); 3-deoxyoctulosonase (EC3.2.1.144); Galactan 1,3-beta-galactosidase (EC3.2.1.145); Beta-galactofuranosidase (EC3.2.1.146); Thioglucosidase (EC3.2.1.147); Ribosylhomocysteinase (EC3.2.1.148.); Beta-primeverosidase (EC3.2.1.149); D-glutamyltransferase (EC2.3.2.1); Glucosamine N-acetyltransferase (EC2.3.1.3.); Glucosamine 6-phosphate N-acetyltransferase (EC2.3.1.4); Glycine N-acyltransferase (EC2.3.1.13); Glutamine N-phenylacetyltransferase (EC2.3.1.14); Glycerol-3-phosphate O-acyltransferase (EC2.3.1.15); Glutamate N-acetyltransferase (EC2.3.1.35); N-acetylneuraminate 4-O-acetyltransferase (EC2.3.1.44); N-acetylneuraminate 7-O(or 9-O)-acetyltransferase (EC2.3.1.45); Maltose O-acetyltransferase (EC2.3.1.79); Aminoglycoside N(3′)-acetyltransferase (EC2.3.1.81); Galactosylacylglycerol O-acyltransferase (EC2.3.1.141); Glycoprotein O-fatty-acyltransferase (EC2.3.1.142); Beta-glucogallin-tetrakisgalloylglucose O-galloyltransferase (EC2.3.1.143); Glucosamine-1-phosphate N-acetyltransferase (EC2.3.1.157); Formaldehyde transketolase (EC2.2.1.3); Acetoin-ribose-5-phosphate transaldolase (EC2.2.1.4); galactose-6-sulfurylase (EC2.5.1.5); UDP-N-acetylglucosamine 1-carboxyvinyltransferase (EC2.5.1.7); Glutamine-pyruvate aminotransferase (EC2.6.1.15); Glutamine-fructose-6-phosphate transaminase (isomerizing) (EC2.6.1.16); dTDP-4-amino-4,6-dideoxy-D-glucose aminotransferase (EC2.6.1.33); UDP-4-amino-2-acetamido-2,4,6-trideoxyglucose aminotransferase (EC2.6.1.34); Oximinotransferase (EC2.6.3.1); Ribose-phosphate pyrophosphokinase (EC2.7.6.1); Phosphomannan mannosephosphotransferase (EC2.7.8.9); CDP-ribitol ribitolphosphotransferase (EC2.7.8.14); UDP-N-acetylglucosamine-dolichyl-phosphate (EC2.7.8.15); CDP-diacylglycerol-inositol 3-phosphatidyltransferase (EC2.7.8.11); CDP-glycerol glycerophosphotransferase (EC2.7.8.12); UDP-N-acetylglucosamine-lysosomal-enzyme (EC2.7.8.17); UDP-galactose-UDP-N-acetylglucosamine galactosephosphotransferase (EC2.7.8.18); UDP-glucose-glycoprotein glucosephosphotransferase (EC2.7.8.19); Phosphatidylglycerol-membrane-oligosaccharide glycerophosphotransferase (EC2.7.8.20); Membrane-oligosaccharide glycerophosphotransferase (EC2.7.8.21); 1-alkenyl-2-acylglycerol cholinephosphotransferase (EC2.7.8.22); Pyruvate, phosphate dikinase (EC2.7.9.1); Pyruvate, water dikinase (EC2.7.9.2); Alpha-glucan, water dikinase (EC2.7.9.4); [Heparan sulfate]-glucosamine 3-sulfotransferase 2 (EC2.8.2.29); [Heparan sulfate]-glucosamine 3-sulfotransferase 3 (EC2.8.2.30); Keratan sulfotransferase (EC2.8.2.21); Arylsulfate sulfotransferase (EC2.8.2.22); [Heparan sulfate]-glucosamine 3-sulfotransferase 1 (EC2.8.2.23); Triglucosylalkylacylglycerol sulfotransferase (EC2.8.2.19); Protein-tyrosine sulfotransferase (EC2.8.2.20); Chondroitin 6-sulfotransferase (EC2.8.2.17); UDP-N-acetylgalactosamine-4-sulfate sulfotransferase (EC2.8.2.7); Aryl sulfotransferase (EC2.8.2.1.); Alcohol sulfotransferase (EC2.8.2.2); Arylamine sulfotransferase (EC2.8.2.3); Galactosylceramide sulfotransferase (EC2.8.2.11); Glycerol dehydrogenase (EC1.1.1.6); Glycerol-3-phosphate dehydrogenase (NAD+) (EC1.1.1.8); D-xylulose reductase (EC1.1.1.9); L-xylulose reductase (EC1.1.1.10); Galactitol 2-dehydrogenase (EC1.1.1.16); Mannitol-1-phosphate 5-dehydrogenase (EC1.1.1.17); Glucuronate reductase (EC1.1.1.19); Glucuronolactone reductase (EC1.1.1.20); Aldehyde reductase (EC1.1.1.21); UDP-glucose 6-dehydrogenase (EC1.1.1.22); Shikimate 5-dehydrogenase (EC1.1.1.25); Glycolate reductase (EC1.1.1.26); L-lactate dehydrogenase (EC1.1.1.27); D-lactate dehydrogenase (EC1.1.1.28); Glycerate dehydrogenase (EC1.1.1.29); 6-phosphogluconate 2-dehydrogenase (EC1.1.1.43); Phosphogluconate dehydrogenase (decarboxylating) (EC1.1.1.44); L-gulonate 3-dehydrogenase (EC1.1.1.45); L-arabinose 1-dehydrogenase (EC1.1.1.46); Glucose 1-dehydrogenase (EC1.1.1.47); D-galactose 1-dehydrogenase (EC1.1.1.48); Glucose-6-phosphate 1-dehydrogenase (EC1.1.1.49); Lactaldehyde reductase (NADPH) (EC1.1.1.55); Ribitol 2-dehydrogenase (EC1.1.1.56); Fructuronate reductase (EC1.1.1.57); Tagaturonate reductase (EC1.1.1.58); Gluconate 5-dehydrogenase (EC1.1.1.69); Glycerol dehydrogenase (NADP+) (EC1.1.1.72); L-xylose 1-dehydrogenase (EC1.1.1.113); Apiose 1-reductase (EC1.1.1.114); Ribose 1-dehydrogenase (NADP+) (EC1.1.1.115); D-arabinose 1-dehydrogenase (EC1.1.1.116); D-arabinose 1-dehydrogenase (NAD(P)+) (EC1.1.1.117); Glucose 1-dehydrogenase (NAD+) (EC1.1.1.118); Glucose 1-dehydrogenase (NADP+) (EC1.1.1.119); galactose 1-dehydrogenase (NADP+) (EC1.1.1.120); Aldose 1-dehydrogenase (EC1.1.1.121); D-threo-aldose 1-dehydrogenase (EC1.1.1.122); Sorbose 5-dehydrogenase (NADP+) (EC1.1.1.123); Fructose 5-dehydrogenase (NADP+) (EC1.1.1.124); 2-deoxy-D-gluconate 3-dehydrogenase (EC1.1.1.125); 2-dehydro-3-deoxy-D-gluconate 6-dehydrogenase (EC1.1.1.126); 2-dehydro-3-deoxy-D-gluconate 5-dehydrogenase (EC1.1.1.127); L-idonate 2-dehydrogenase (EC1.1.1.128); L-threonate 3-dehydrogenase (EC1.1.1.129); 3-dehydro-L-gulonate 2-dehydrogenase (EC1.1.1.130); Mannuronate reductase (EC1.1.1.131); GDP-mannose 6-dehydrogenase (EC1.1.1.132); dTDP-4-dehydrorhamnose reductase (EC1.1.1.133); dTDP-6-deoxy-L-talose 4-dehydrogenase (EC1.1.1.134); GDP-6-deoxy-D-talose 4-dehydrogenase (EC1.1.1.135); UDP-N-acetylglucosamine 6-dehydrogenase (EC1.1.1.136); Ribitol-5-phosphate 2-dehydrogenase (EC1.1.1.137); Mannitol 2-dehydrogenase (NADP+) (EC1.1.1.138); Sorbitol-6-phosphate 2-dehydrogenase (EC1.1.1.140); Glycerol 2-dehydrogenase (EC1.1.1.156); UDP-N-acetylmuramate dehydrogenase (EC1.1.1.158); L-rhamnose 1-dehydrogenase (EC1.1.1.173); D-xylose 1-dehydrogenase (EC1.1.1.175); Glycerol-3-phosphate 1-dehydrogenase (NADP+) (EC1.1.1.177); D-xylose 1-dehydrogenase (NADP+) (EC1.1.1.179); L-glycol dehydrogenase (EC1.1.1.185); dTDP-galactose 6-dehydrogenase (EC1.1.1.186); GDP-4-dehydro-D-rhamnose reductase (EC1.1.1.187); Aldose-6-phosphate reductase (EC1.1.1.200); Mannose-6-phosphate 6-reductase (EC1.1.1.224); N-acylmannosamine 1-dehydrogenase (EC1.1.1.233); N-acetylhexosamine 1-dehydrogenase (EC1.1.1.240); D-arabinitol 2-dehydrogenase (EC1.1.1.250); Galactitol-1-phosphate 5-dehydrogenase (EC1.1.1.251); Mannitol dehydrogenase (EC1.1.1.255); Glycerol-1-phosphate dehydrogenase [NAD(P)] (EC1.1.1.261); dTDP-4-dehydro-6-deoxyglucose reductase (EC1.1.1.266); GDP-L-fucose synthase EC1.1.1.271); Glucose oxidase (EC1.1.3.4); Hexose oxidase (EC1.1.3.5); galactose oxidase (EC1.1.3.9); Pyranose oxidase (EC1.1.3.10); L-sorbose oxidase EC1.1.3.11); Glycerol-3-phosphate oxidase (EC1.1.3.21); Xanthine oxidase (EC1.1.3.22); L-galactonolactone oxidase (EC1.1.3.24); Cellobiose oxidase (EC1.1.3.25); N-acylhexosamine oxidase (EC1.1.3.29); D-arabinono-1,4-lactone oxidase EC1.1.3.37); D-mannitol oxidase (EC1.1.3.40); Xylitol oxidase EC1.1.3.41); Gluconate 2-dehydrogenase (acceptor) (EC1.1.99.3); Dehydrogluconate dehydrogenase (EC1.1.99.4); Glycerol-3-phosphate dehydrogenase EC1.1.99.5); Lactate-malate transhydrogenase EC1.1.99.7); Glucose dehydrogenase (acceptor) (EC1.1.99.10); Fructose 5-dehydrogenase (EC1.1.99.11); Sorbose dehydrogenase (EC1.1.99.12); Glucoside 3-dehydrogenase (EC1.1.99.13); Glucose dehydrogenase (pyrroloquinoline-quinone) (EC1.1.99.17); Cellobiose dehydrogenase (EC1.1.99.18); Glucose-fructose oxidoreductase (EC1.1.99.28); Glutamate dehydrogenase (EC1.4.1.2); Glutamate dehydrogenase (NAD(P)+) (EC1.4.1.3); Glutamate dehydrogenase (NADP+) (EC1.4.1.4); ADP-ribose pyrophosphatase EC3.6.1.13); Monosaccharide-transporting ATPase (EC3.6.3.17); Oligosaccharide-transporting ATPase (EC3.6.3.18); Maltose-transporting ATPase (EC3.6.3.19); Glycerol-3-phosphate-transporting ATPase (EC3.6.3.20); Phosphoketolase EC4.1.2.9); Fructose-bisphosphate aldolase (EC4.1.2.13); L-fuculose-phosphate aldolase (EC4.1.2.17); Rhamnulose-1-phosphate aldolase EC4.1.2.19); Fructose-6-phosphate phosphoketolase (EC4.1.2.22); Tagatose-bisphosphate aldolase EC4.1.2.40); UDP-glucose 4,6-dehydratase (EC4.2.1.76); Hyaluronate lyase (EC4.2.2.1); Pectate lyase (EC4.2.2.2); Poly(beta-D-mannuronate) lyase (EC4.2.2.3); Chondroitin Abc lyase (EC4.2.2.4); Chondroitin Ac lyase (EC4.2.2.5); Oligogalacturonide lyase (EC4.2.2.6); Heparin lyase (EC4.2.2.7); Heparitin-sulfate lyase (EC4.2.2.8); Exopolygalacturonate lyase (EC4.2.2.9); Pectin lyase (EC4.2.2.10); Poly(alpha-L-guluronate) lyase (EC4.2.2.11); Xanthan lyase (EC4.2.2.12); Exo-(1,4)-alpha-D-glucan lyase (EC4.2.2.13); Glucuronan lyase (EC4.2.2.14); Phosphoglycerate mutase (EC5.4.2.1); Phosphoglucomutase (EC5.4.2.2); Phosphoacetylglucosamine mutase (EC5.4.2.3); Bisphosphoglycerate mutase (EC5.4.2.4); Phosphoglucomutase (glucose-cofactor) (EC5.4.2.5); Beta-phosphoglucomutase (EC5.4.2.6); Phosphopentomutase (EC5.4.2.7); Phosphomannomutase EC5.4.2.8.), Phosphoenolpyruvate mutase (EC5.4.2.9); Phosphoglucosamine mutase EC5.4.2.10); UDP-galactopyranose mutase EC5.4.99.9); Isomaltulose synthase (EC5.4.99.11); (1,4)-alpha-D-glucan 1-alpha-D-glucosylmutase (EC5.4.99.15); Maltose alpha-D-glucosyltransferase (EC5.4.99.16), and all related homologs and isoforms.

II. Vectors and Constructs to Modify Sugar Metabolic Pathway Genes

Another aspect of the present invention provides nucleic acid constructs that contain cDNA encoding galactose transport-related proteins as described above. In one embodiment, the proteins can be associated with sugar catabolism, such as GALE, the hexosamine pathway, such as GFAT and/or NHE. In another embodiment, the proteins can be associated with sugar chain synthesis, such as β-1,3-GT, β-1,4-GT, α-1,4-GT, α-1,4-GalNAcT, β-1,4-GalNAcT, β-1,3-GlcNAcT and/or β-1,6-GlcNAcT. These cDNA sequences encoding these proteins can be derived from any prokaryote or eukaryote. The nucleic acid sequences encoding for the protein can be derived from, for example, mammals including, but not limited to, humans, pigs, sheep, goats, cows (bovine), deer, mules, horses, monkeys and other non-human primates, dogs, cats, rats, mice, rabbits and, birds including, but not limited to, chickens, turkeys, ducks, geese, canaries, and the like, reptiles, fish, amphibians, worms including C. elegans, and insects including but not limited to, Drosophila, Trichoplusa, and Spodoptera.

Nucleic acid contructs or vectors are provided that contains at least one cDNA sequence encoding a galactose transport-related protein as described above. At least one, two, three, four, five, or ten separate nucleic acid sequences encoding for different proteins can be cloned into a vector.

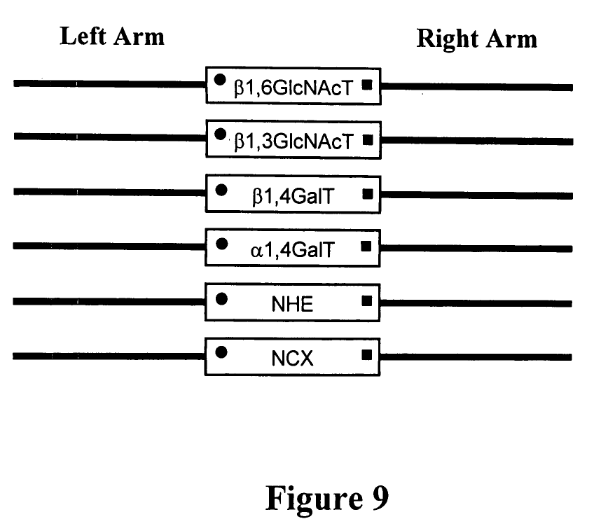

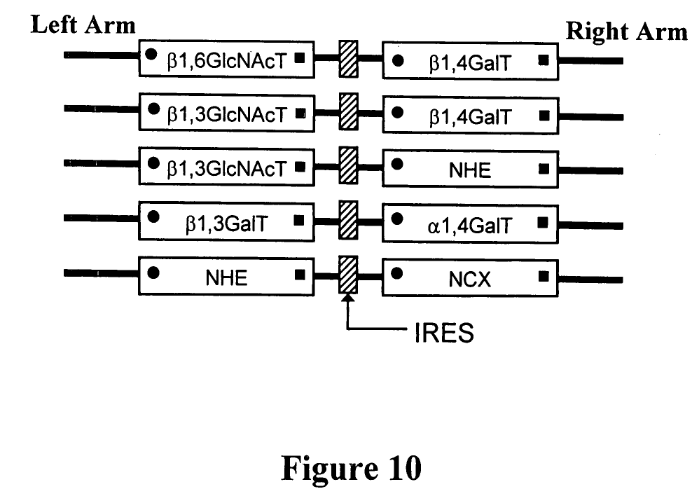

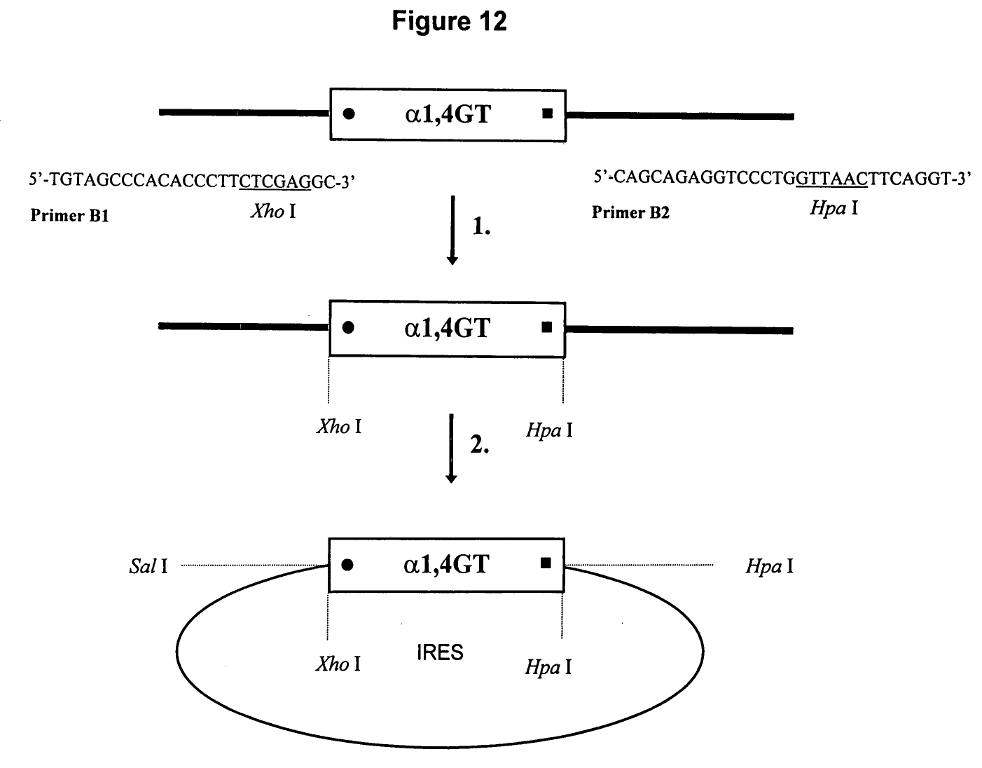

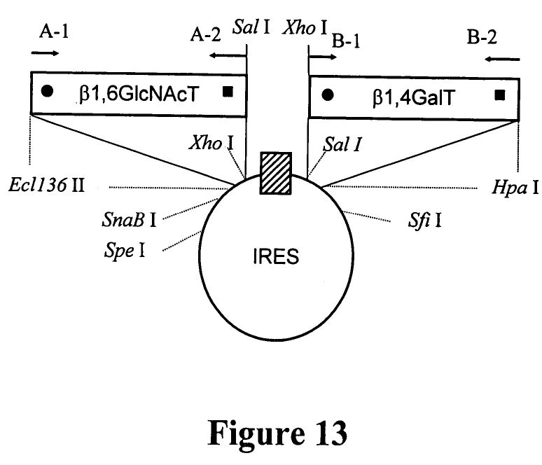

The construct can contain a single cassette encoding a single galactose transport-related protein, double cassettes encoding two galactose transport-related proteins, or multiple cassettes encoding more than two galactose transport-related proteins. Constructs can further contain one, or more than one, internal ribosome entry site (IRES). (See, for example, FIGS. 9-13).

In one embodiment, the nucleic acid construct contains a single cassette encoding a galactose transport-related protein, such as GALE, GFAT, NHE, NCX, β-1,3-GT, β-1,4-GT, α-1,4-GT, α-1,4-GalNAcT, β-1,4-GalNAcT, β-1,3-GlcNAcT and β-1,6-GlcNAcT (see, for example, FIG. 9). In another embodiment, the nucleic acid construct contains more than one cassette encoding the same galactose transport-related protein. In still another embodiment, the nucleic acid construct contains more than one cassette encoding more than one galactose transport-related protein in combination. Such combination include, but are not limited to, β-1,6-GlcNAcT and β-1,4-GT, β1,3-GlcNAcT and β-1,4-GT, β-1,3-GlcNAcT and NHE, β1,3-GT and α-1,4-GT, and NHE and NCX (see, for example, FIG. 10).

Nucleic Acid Contructs/Vectors

The term “vector,” as used herein, refers to a nucleic acid molecule (preferably DNA) that provides a useful biological or biochemical property to an inserted nucleic acid. “Expression vectors” according to the invention include vectors that are capable of enhancing the expression of one or more nucleic acid sequences encoding for a protein that has been inserted or cloned into the vector, upon transformation of the vector into a cell. The terms “vector” and “plasmid” are used interchangeably herein. Examples of vectors include, phages, autonomously replicating sequences (ARS), centromeres, and other sequences which are able to replicate or be replicated in vitro or in a cell, or to convey a desired nucleic acid segment to a desired location within a cell of an animal. Expression vectors useful in the present invention include chromosomal-, episomal- and virus-derived vectors, e.g., vectors derived from bacterial plasmids or bacteriophages, and vectors derived from combinations thereof, such as cosmids and phagemids. A vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, selectable markers, etc. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575), TA Cloning® brand PCR cloning (Invitrogen Corp., Carlsbad, Calif.)) can also be applied to clone a nucleic acid into a vector to be used according to the present invention. The vector can further contain one or more selectable markers to identify cells transformed with the vector, such as the selectable markers and reporter genes described herein. In addition, the sugar metabolic associated protein containing expression vector is assembled to include a cloning region and a poly(U)-dependent PolIII transcription terminator.

In accordance with the invention, any vector can be used to construct the sugar metabolic associated protein containing expression vectors of the invention. In addition, vectors known in the art and those commercially available (and variants or derivatives thereof) can, in accordance with the invention, be engineered to include one or more recombination sites for use in the methods of the invention. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts.

Other vectors of interest include viral origin vectors (Ml 3 vectors, bacterial phage λ vectors, adenovirus vectors, and retrovirus vectors), high, low and adjustable copy number vectors, vectors which have compatible replicons for use in combination in a single host (pACYC184 and pBR322) and eukaryotic episomal replication vectors (pCDM8).

Vectors of interest include prokaryotic expression vectors such as pcDNA II, pSL301, pSE280, pSE380, pSE420, pTrcHisA, B, and C, pRSET A, B, and C (Invitrogen, Corp.), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and pProEx-HT (Invitrogen, Corp.) and variants and derivatives thereof. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI10, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and pYES2, pAC360, pBlueBacHis A, B, and C, pVL1392, pBlueBacIII, pCDM8, pcDNA1, pZeoSV, pcDNA3 pREP4, pCEP4, and pEBVHis (Invitrogen, Corp.) and variants or derivatives thereof.

Other vectors that can be used include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen) and variants or derivatives thereof. Viral vectors can also be used, such as lentiviral vectors (see, for example, WO 03/059923; Tiscornia et al. PNAS 100:1844-1848 (2003)).

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZα, pGAPZ, pGAPZα, pBlueBac4.5, pBlueBacHis2, pMelBac, pSinRep5, pSinHis, pIND, pIND(SP1), pVgRXR, pcDNA2.1, pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; λ ExCell, λ gt11, pTrc99A, pKK223-3, pGEX-1λT, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32LIC, pET-30LIC, pBAC-2 cp LIC, pBACgus-2 cp LIC, pT7Blue-2 LIC, pT7Blue-2, λSCREEN-1, λBlueSTAR, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, pβgal-Basic, pβgal-Control, pβgal-Promoter, pβgal-Enhancer, pCMV, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, λgt10, λgt11, pWE15, and TriplEx from Clontech; Lambda ZAP II, pBK-CMV, pBK-RSV, pBluescript II KS+/−, pAD-GALA, pBD-GAL4 Cam, pSurfscript, Lambda FIX II, Lambda DASH, Lambda EMBL3, Lambda EMBLA, SuperCos, pCR-Scrigt Amp, pCR-Script Cam, pCR-Script Direct, pBS +/−, pBC KS+/−, pBC SK+/−, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMC1neo, pMC1neo Poly A, pOG44, pOG45, pFRTβGAL, pNEOβGAL, pRS403, pRS404, pRS405, pRS406, pRS413, pRS414, pRS415, and pRS416 from Stratagene.

Two-hybrid and reverse two-hybrid vectors of interest include pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof. Another aspect of the present invention provides nucleic acid constructs that contain cDNA encoding galactose transport-related proteins, such as those associated with sugar catabolism, such as GALE, the hexosamine pathway, such as GFAT and/or NHE, or sugar chain synthesis, such as α-1,3-GT, β-1,4-GT, α-1,4-GT, α-1,4-GalNAcT, β-1,4-GalNAcT, β-1,3-GlcNAcT and/or α-1,6-GlcNAcT. These cDNA sequences can be derived from any prokaryotic or eukaryotic nucleic acid sequence that encodes for a galactose transport-related protein. The construct can contain a single cassette encoding a single galactose transport-related protein (see, for example, FIG. 9), double cassettes (see, for example, FIG. 10) encoding two galactose transport-related proteins, or multiple cassettes encoding more than two galactose transport-related proteins. Constructs can further contain one, or more than one, internal ribosome entry site (IRES). The construct can also contain a promoter operably linked to the nucleic acid sequence encoding galactose transport-related proteins, or, alternatively, the construct can be promoterless. The nucleic acid constructs can further contain nucleic acid sequences that permit random or targeted insertion into a host genome.

In one embodiment, the nucleic acid construct contains a single cassette encoding a galactose transport-related protein, such as GALE, GFAT, NHE, NCX, 1-1,3-GT, β-1,4-GT, α-1,4-GT, α-1,4-GalNAcT, β-1,4-GalNAcT, β-1,3-GlcNAcT and β-1,6-GlcNAcT (see, for example, FIG. 9). In another embodiment, the nucleic acid construct contains more than one cassette encoding the same galactose transport-related protein. In still another embodiment, the nucleic acid construct contains more than one cassette encoding more than one galactose transport-related protein in combination. Such combination include, but are not limited to, β-1,6-GlcNAcT and β-1,4-GT, β-1,3-GlcNAcT and β-1,4-GT, >1,3-GlcNAcT and NHE, β-1,3-GT and α-1,4-GT, and NHE and NCX (see, for example, FIG. 10).

Nucleic acid constructs useful for targeted insertion of the galactose transport-related cDNA can include 5′ and 3′ recombination arms for homologous recombination. In one embodiment, targeting vectors are provided wherein homologous recombination in somatic cells can be rapidly detected. These targeting vectors can be transformed into mammalian cells to target a gene via homologous recombination. In one embodiment, the targeting vectors can target a gene associated with galactose transport. In another embodiment, the targeting construct can target a house keeping gene. In a further embodiment, the targeting construct can target a galactose transport-related gene that has been rendered inactive. In another embodiment, the targeting construct can target a galactose transport-related gene or a housekeeping gene so as to be in reading frame with the upstream sequence, which can allow it to be expressed under the control of the endogenous promoter of the galactose transport-related or housekeeping gene. In an alternate embodiment, the targeting construct can be constructed to render the galactose transport-related gene inactive, i.e., it can be used to knock-out the gene. In another embodiment, the targeting construct also contains a selectable marker gene. Cells can be transformed with the constructs using the methods of the invention and are selected by means of the selectable marker and then screened for the presence of recombinants.

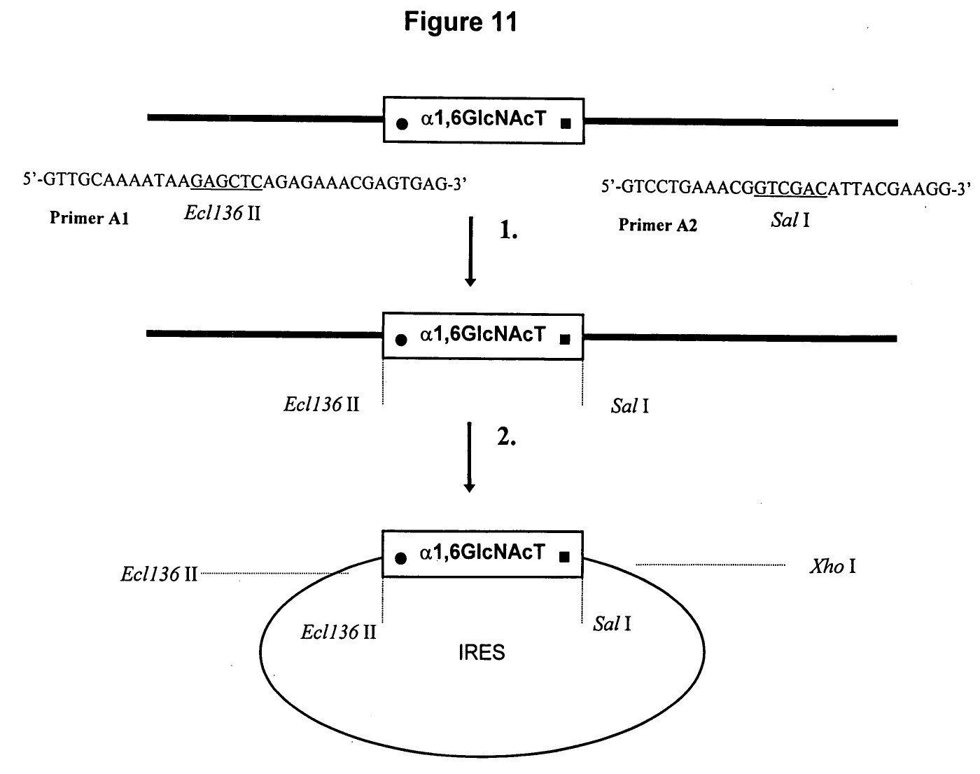

In other embodiments of the invention, galactose transport-related cDNAs (such as those described above) can be cloned and inserted into vectors (see, for eample, FIGS. 11, 12 and 13). cDNA sequences can be isolated from cells and then cloned into the vector using restriction enzymes. In another embodiment, the cDNA sequences can be synthesized and then cloned into vectors. Restriction enzyme cloning into vectors can be accomplished using blunt-end cloning or sticky-end cloning. Restriction enzymes can create staggered, single strand cuts, double strand, or blunt end cuts. Restriction enzymes useful for cloning into vectors include, but are not limited to, Type 1 restriction enzymes, Type 2 restriction enzymes, Type 3 restriction enzymes, Sal I, Xho I, Sfi I, Spe I, SnaB I, Hpa I, Ecl136II, and those listed in the tables below.

TABLE 8

| | | | |
|---|---|---|---|
| Restric- | | Ends of | |
| tion | | DNA Sequence | Cleaved |
| Enzyme | Source | Recognized | Molecule |

| | | | |
|---|---|---|---|
| EcoRI | Escherchia | 5′GAATTC | 5′AATTC - G |
| | coli | 3′CTTAAG | G - CTTAA5′ |
| BamHI | Bacillus | 5′GGATCC | 5′GATCC - G |
| | amylolique- | 3′CCTAGG | G - CCTAG5′ |
| | faciens | | |
| HindIII | Haemophilus | 5′AAGCTT | 5′ACCTT - A |
| | influenzae | 3′TTCGAA | A - TTCGA5′ |
| MstII | Microcoleus | 5′CCTNAGG | 5′CTNAGG - C |
| | species | 3′GGANTCC | G - GGANTC5′ |
| TaqI | Thermus | 5′TCGA | 5′CGA - T |
| | aquaticus | 3′AGCT | T - AGC5′ |
| NotI | Nocardia | 5′GCGGCCGC | 5′GGCCGC - GC |
| | otitidis | 3′CGCCGGCG | CG - CGCCGGC5′ |
| AluI* | Arthrobacter | 5′AGCT | 5′AG - CT |
| | luteus | 3′TCGA | TC - GA5′ |

*=blunt ends

TABLE 9

| | | Target sequence |
|---|---|---|
| | Organism from | (cut at *) |
| Enzyme | which derived | 5′→3′ |
| Ava I | Anabaena variabilis | C* C/T C G A/G G |
| Bam HI | Bacillus amyloliquefaciens | G* G A T C C |
| Bgl II | Bacillus globigii | A* G A T C T |
| Eco RI | Escherichia coli RY 13 | G* A A T T C |
| Eco RII | Escherichia coli R245 | * C C A/T G G |
| Hae III | Haernophilus aegyptius | G G * C C |
| Hha I | Haemophilus haemolyticus | G C G * C |
| Hind III | Haemophilus inflenzae Rd | A * A G C T T |
| Hpa I | Haemophilus parainflenzae | G T T * A A C |
| Kpn I | Klebsiella pneumoniae | G G T A C * C |
| Mbo I | Moraxella bovis | *G A T C |
| Mbo I | Moraxella bovis | *G A T C |
| Pst I | Providencia stuartii | C T G C A * G |
| Sma I | Serratia marcescens | C C C * G G G |
| SstI | Streptomyces stanford | G A G C T * C |
| Sal I | Streptomyces albus G | G * T C G A C |
| Taq I | Thermophilus aquaticus | T * C G A |
| Xma I | Xanthamonas malvacearum | C * C C G G G |

Promoters

In one aspect of the present invention, nucleic acid contructs or vectors are provided that contain at least one cDNA sequence encoding a galactose transport-related protein and at least one promoter. At least one, two, three, four, five, or ten separate nucleic acid sequences encoding for different proteins can be cloned into a vector. The promoter can be operably linked to the nucleic acid sequence encoding galactose transport-related proteins. The promoter can be an exogenous or endogenous promoter.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter” denotes a segment of DNA which contains sequences capable of providing promoter functions (i.e., the functions provided by a promoter element). For example, the long terminal repeats of retroviruses contain promoter functions. The promoter may be “endogenous” or “exogenous” or “heterologous.” An “endogenous” promoter is one which is associated with a given gene in the genome. An “exogenous” or “heterologous” promoter is one which is placed in juxtaposition to a gene by means of genetic manipulation (i.e., molecular biological techniques such as cloning and recombination) such that transcription of that gene is directed by the linked promoter. Promoters can also contain enhancer activities.

a. Endogenous Promoters

In one embodiment, the operably linked promoter of the sugar metabolic associated protein containing vector is an endogenous promoter. In one aspect of this embodiment, the endogenous promoter can be any unregulated promoter that allows for the continual transcription of its associated gene.

In another aspect, the promoter can be a constitutively active promoter. More preferably, the endogenous promoter is associated with a housekeeping gene. Non limiting examples of housekeeping genes whose promoter can be operably linked to the sugar metabolic associated protein include the conserved cross species analogs of the following housekeeping genes; mitochondrial 16S rRNA, ribosomal protein L29 (RPL29), H3 histone, family 3B (H3.3B) (H₃F₃B), poly(A)-binding protein, cytoplasmic 1 (PABPC1), HLA-B associated transcript-1 (D6S81E), surfeit 1 (SURF1), ribosomal protein L8 (RPL8), ribosomal protein L38 (RPL38), catechol-O-methyltransferase (COMT), ribosomal protein S7 (RPS7), heat shock 27 kD protein 1 (HSPB1), eukaryotic translation elongation factor 1 delta (guanine nucleotide exchange protein) (EEF1D), vimentin (VIM), ribosomal protein L41 (RPL41), carboxylesterase 2 (intestine, liver) (CES2), exportin 1 (CRM1, yeast, homolog) (XPO1), ubiquinol-cytochrome c reductase hinge protein (UQCRH), Glutathione peroxidase 1 (GPX1), ribophorin II (RPN2), Pleckstrin and Sec7 domain protein (PSD), human cardiac troponin T, proteasome (prosome, macropain) subunit, beta type, 5 (PSMB5), cofilin 1 (non-muscle) (CFL1), seryl-tRNA synthetase (SARS), catenin (cadherin-associated protein), beta 1 (88 kD) (CTNNB1), Duffy blood group (FY), erythrocyte membrane protein band 7.2 (stomatin) (EPB72), Fas/Apo-1, LIM and SH3 protein 1 (LASP1), accessory proteins BAP31/BAP29 (DXS1357E), nascent-polypeptide-associated complex alpha polypeptide (NACA), ribosomal protein L18a (RPL18A), TNF receptor-associated factor 4 (TRAF4), MLN51 protein (MLN51), ribosomal protein L11 (RPL11), Poly(rC)-binding protein 2 (PCBP2), thioredoxin (TXN), glutaminyl-tRNA synthetase (QARS), testis enhanced gene transcript (TEGT), prostatic binding protein (PBP), signal sequence receptor, beta (translocon-associated protein beta) (SSR2), ribosomal protein L3 (RPL3), centrin, EF-hand protein, 2 (CETN2), heterogeneous nuclear ribonucleoprotein K (HNRPK), glutathione peroxidase 4 (phospholipid hydroperoxidase) (GPX4), fusion, derived from t(12;16) malignant liposarcoma (FUS), ATP synthase, H+ transporting, mitochondrial F0 complex, subunit c (subunit 9), isoform 2 (ATP5G2), ribosomal protein S26 (RPS26), ribosomal protein L6 (RPL6), ribosomal protein S18 (RPS18), serine (or cysteine) proteinase inhibitor, clade A (alpha-1 antiproteinase, antitrypsin), member 3 (SERPINA3), dual specificity phosphatase 1 (DUSP1), peroxiredoxin 1 (PRDX1), epididymal secretory protein (19.5 kD) (HE1), ribosomal protein S8 (RPS8), translocated promoter region (to activated MET oncogene) (TPR), ribosomal protein L13 (RPL13), SON DNA binding protein (SON), ribosomal prot L19 (RPL19), ribosomal prot (homolog to yeast S24), CD63 antigen (melanoma 1 antigen) (CD63), protein tyrosine phosphatase, non-receptor type 6 (PTPN6), eukaryotic translation elongation factor 1 beta 2 (EEF1B2), ATP synthase, H+ transporting, mitochondrial F0 complex, subunit b, isoform 1 (ATP5F1), solute carrier family 25 (mitochondrial carrier; phosphate carrier), member 3 (SLC25A3), tryptophanyl-tRNA synthetase (WARS), glutamate-ammonia ligase (glutamine synthase) (GLUL), ribosomal protein L7 (RPL7), interferon induced transmembrane protein 2 (1-8D) (IFITM2), tyrosine 3-monooxygenase/tryptophan 5-monooxygenase activation protein, beta polypeptide (YWHAB), Casein kinase 2, beta polypeptide (CSNK2B), ubiquitin A-52 residue ribosomal protein fusion product 1 (UBA52), ribosomal protein L13a (RPL13A), major histocompatibility complex, class I, E (HLA-E), jun D proto-oncogene (JUND), tyrosine 3-monooxygenase/tryptophan 5-monooxygenase activation protein, theta polypeptide (YWHAQ), ribosomal protein L23 (RPL23), Ribosomal protein S3 (RPS3), ribosomal protein L17 (RPL17), filamin A, alpha (actin-binding protein-280) (FLNA), matrix Gla protein (MGP), ribosomal protein L35a (RPL35A), peptidylprolyl isomerase A (cyclophilin A) (PPIA), villin 2 (ezrin) (VIL2), eukaryotic translation elongation factor 2 (EEF2), jun B proto-oncogene (JUNB), ribosomal protein S2 (RPS2), cytochrome c oxidase subunit VIIc (COX7C), heterogeneous nuclear ribonucleoprotein L (HNRPL), tumor protein, translationally-controlled 1 (TPT1), ribosomal protein L31 (RPL31), cytochrome c oxidase subunit VIIa polypeptide 2 (liver) (COX7A2), DEAD/H (Asp-Glu-Ala-Asp/His) box polypeptide 5 (RNA helicase, 68 kD) (DDX5), cytochrome c oxidase subunit VIa polypeptide 1 (COX6A1), heat shock 90 kD protein 1, alpha (HSPCA), Sjogren syndrome antigen B (autoantigen La) (SSB), lactate dehydrogenase B (LDHB), high-mobility group (nonhistone chromosomal) protein 17 (HMG17), cytochrome c oxidase subunit VIc (COX6C), heterogeneous nuclear ribonucleoprotein A1 (HNRPA1), aldolase A, fructose-bisphosphate (ALDOA), integrin, beta 1 (fibronectin receptor, beta polypeptide, antigen CD29 includes MDF2, MSK12) (ITGB1), ribosomal protein S11 (RPS1), small nuclear ribonucleoprotein 70 kD polypeptide (RN antigen) (SNRP20), guanine nucleotide binding protein (G protein), beta polypeptide 1 (GNB1), heterogeneous nuclear ribonucleoprotein A1 (HNRPA1), calpain 4, small subunit (30K) (CAPN4), elongation factor TU (N-terminus)/X03689, ribosomal protein L32 (RPL32), major histocompatibility complex, class II, DP alpha 1 (HLA-DPA1), superoxide dismutase 1, soluble (amyotrophic lateral sclerosis 1 (adult)) (SOD1), lactate dehydrogenase A (LDHA), glyceraldehyde-3-phosphate dehydrogenase (GAPD), Actin, beta (ACTB), major histocompatibility complex, class II, DP alpha (HLA-DRA), tubulin, beta polypeptide (TUBB), metallothionein 2A (MT2A), phosphoglycerate kinase 1 (PGK1), KRAB-associated protein 1 (TIF1B), eukaryotic translation initiation factor 3, subunit 5 (epsilon, 47 kD) (EIF3S5), NADH dehydrogenase (ubiquinone) 1 alpha subcomplex, 4 (9 kD, MLRQ) (NDUFA4), chloride intracellular channel 1 (CLIC1), adaptor-related protein complex 3, sigma 1 subunit (AP3S1), cytochrome c oxidase subunit IV (COX4), PDZ and LIM domain 1 (elfin) (PDLIM1), glutathione-5-transferase like; glutathione transferase omega (GSTTLp28), interferon stimulated gene (20 kD) (ISG20), nuclear factor I/B (NFIB), COX10 (yeast) homolog, cytochrome c oxidase assembly protein (heme A: farnesyltransferase), conserved gene amplified in osteosarcoma (OS4), deoxyhypusine synthase (DHPS), galactosidase, alpha (GLA), microsomal glutathione S-transferase 2 (MGST2), eukaryotic translation initiation factor 4 gamma, 2 (EIF4G2), ubiquitin carrier protein E2-C (UBCH10), BTG family, member 2 (BTG2), B-cell associated protein (REA), COP9 subunit 6 (MOV34 homolog, 34 kD) (MOV34-34 KD), ATX1 (antioxidant protein 1, yeast) homolog 1 (ATOX1), acidic protein rich in leucines (SSP29), poly(A)-binding prot (PABP) promoter region, selenoprotein W, 1 (SEPW1), eukaryotic translation initiation factor 3, subunit 6 (48 kD) (EIF3S6), carnitine palmitoyltransferase I, muscle (CPT1B), transmembrane trafficking protein (TMP21), four and a half LIM domains 1 (FHL1), ribosomal protein S28 (RPS28), myeloid leukemia factor 2 (MLF2), neurofilament triplet L prot/U57341, capping protein (actin filament) muscle Z-line, alpha 1 (CAPZA1), 1-acylglycerol-3-phosphate O-acyltransferase 1 (lysophosphatidic acid acyltransferase, alpha) (AGPAT1), inositol 1,3,4-triphosphate 5/6 kinase (ITPK1), histidine triad nucleotide-binding protein (HINT), dynamitin (dynactin complex 50 kD subunit) (DCTN-50), actin related protein 2/3 complex, subunit 2 (34 kD) (ARPC2), histone deacetylase 1 (HDAC1), ubiquitin B, chitinase 3-like 2 (CHI3L2), D-dopachrome tautomerase (DDT), zinc finger protein 220 (ZNF220), sequestosome 1 (SQSTM1), cystatin B (stefin B) (CSTB), eukaryotic translation initiation factor 3, subunit 8 (110 kD) (EIF3S8), chemokine (C-C motif) receptor 9 (CCR9), ubiquitin specific protease 11 (USP11), laminin receptor 1 (67 kD, ribosomal protein SA) (LAMR1), amplified in osteosarcoma (OS-9), splicing factor 3b, subunit 2, 145 kD (SF3B2), integrin-linked kinase (ILK), ubiquitin-conjugating enzyme E2D 3 (homologous to yeast UBC4/5) (UBE2D3), chaperonin containing TCP1, subunit 4 (delta) (CCT4), polymerase (RNA) II (DNA directed) polypeptide L (7.6 kD) (POLR2L), nuclear receptor co-repressor 2 (NCOR2), accessory proteins BAP31/BAP29 (DXS1357E, SLC6A8), 13 kD differentiation-associated protein (LOC55967), Tax1 (human T-cell leukemia virus type I) binding protein 1 (TAX1BP1), damage-specific DNA binding protein 1 (127 kD) (DDB1), dynein, cytoplasmic, light polypeptide (PIN), methionine aminopeptidase; eIF-2-associated p67 (MNPEP), G protein pathway suppressor 2 (GPS2), ribosomal protein L21 (RPL21), coatomer protein complex, subunit alpha (COPA), G protein pathway suppressor 1 (GPS1), small nuclear ribonucleoprotein D2 polypeptide (16.5 kD) (SNRPD2), ribosomal protein S29 (RPS29), ribosomal protein S10 (RPS10), ribosomal proteinS9 (RPS9), ribosomal protein S5 (RPS5), ribosomal protein L28 (RPL28), ribosomal protein L27a (RPL27A), protein tyrosine phosphatase type IVA, member 2 (PTP4A2), ribosomal prot L36 (RPL35), ribosomal protein L10a (RPL10A), Fc fragment of IgG, receptor, transporter, alpha (FCGRT), maternal G10 transcript (G110), ribosomal protein L9 (RPL9), ATP synthase, H+ transporting, mitochondrial F0 complex, subunit c (subunit 9) isoform 3 (ATP5G3), signal recognition particle 14 kD (homologous Alu RNA-binding protein) (SRP14), mutL (E. coli) homolog 1 (colon cancer, nonpolyposis type 2) (MLH1), chromosome 1 q subtelomeric sequence D1S553./U06155, fibromodulin (FMOD), amino-terminal enhancer of split (AES), Rho GTPase activating protein 1 (ARHGAP1), non-POU-domain-containing, octamer-binding (NONO), v-raf murine sarcoma 3611 viral oncogene homolog 1 (ARAF1), heterogeneous nuclear ribonucleoprotein A1 (HNRPA1), beta 2-microglobulin (B2M), ribosomal protein S27a (RPS27A), bromodomain-containing 2 (BRD2), azoospermia factor 1 (AZF1), upregulated by 1,25 dihydroxyvitamin D-3 (VDUP1), serine (or cysteine) proteinase inhibitor, clade B (ovalbumin), member 6 (SERPINB6), destrin (actin depolymerizing factor) (ADF), thymosin beta-10 (TMSB10), CD34 antigen (CD34), spectrin, beta, non-erythrocytic 1 (SPTBN1), angio-associated, migratory cell protein (AAMP), major histocompatibility complex, class I, A (HLA-A), MYC-associated zinc finger protein (purine-binding transcription factor) (MAZ), SET translocation (myeloid leukemia-associated) (SET), paired box gene(aniridia, keratitis) (PAX6), zinc finger protein homologous to Zfp-36 in mouse (ZFP36), FK506-binding protein 4 (59 kD) (FKBP4), nucleosome assembly protein 1-like 1 (NAP1L1), tyrosine 3-monooxygenase/tryptophan 5-monooxygenase activation protein, zeta polypeptide (YWHAZ), ribosomal protein S3A (RPS3A), ADP-ribosylation factor 1, ribosomal protein S19 (RPS19), transcription elongation factor A (SII), 1 (TCEA1), ribosomal protein S6 (RPS6), ADP-ribosylation factor 3 (ARF3), moesin (MSN), nuclear factor of kappa light polypeptide gene enhancer in B-cells inhibitor, alpha (NFKBIA), complement component 1, q subcomponent binding protein (C1QBP), ribosomal protein S25 (RPS25), clusterin (complement lysis inhibitor, SP40,40, sulfated glycoprotein 2, testosterone-repressed prostate message 2, apolipoprotein J) (CLU), nucleolin (NCL), ribosomal protein S16 (RPS16), ubiquitin-activating enzyme E1 (A1S9T and BN75 temperature sensitivity complementing) (UBE1), lectin, galactoside-binding, soluble, 3 (galectin 3) (LGALS3), eukaryotic translation elongation factor 1 gamma (EEF1G), pim-1 oncogene (PIM1), S100 calcium-binding protein A10 (annexin II ligand, calpactin I, light polypeptide (p11)) (S100A10), H2A histone family, member Z (H2AFZ), ADP-ribosylation factor 4 (ARF4) (ARF4), ribosomal protein L7a (RPL7A), major histocompatibility complex, class II, DQ alpha 1 (HLA-DQA1), FK506-binding protein 1A (12 kD) (FKBP1A), CD81 antigen (target of antiproliferative antibody 1) (CD81), ribosomal protein S15 (RPS15), X-box binding protein 1 (XBP1), major histocompatibility complex, class II, DN alpha (HLA-DNA), ribosomal protein S24 (RPS24), leukemia-associated phosphoprotein p18 (stathmin) (LAP18), myosin, heavy polypeptide 9, non-muscle (MYH9), casein kinase 2, beta polypeptide (CSNK2B), fucosidase, alpha-L-1, tissue (FUCA1), diaphorase (NADH) (cytochrome b-5 reductase) (DIA1), cystatin C (amyloid angiopathy and cerebral hemorrhage) (CST3), ubiquitin C (UBC), ubiquinol-cytochrome c reductase binding protein (UQCRB), prothymosin, alpha (gene sequence 28) (PTMA), glutathione S-transferase pi (GSTP1), guanine nucleotide binding protein (G protein), beta polypeptide 2-like 1 (GNB2L1), nucleophosmin (nucleolar phosphoprotein B23, numatrin) (NPM1), CD3E antigen, epsilon polypeptide (TiT3 complex) (CD3E), calpain 2, (m/Il) large subunit (CAPN2), NADH dehydrogenase (ubiquinone) flavoprotein 2 (24 kD) (NDUFV2), heat shock 60 kD protein 1 (chaperonin) (HSPD1), guanine nucleotide binding protein (G protein), alpha stimulating activity polypeptide 1 (GNAS1), clathrin, light polypeptide (Lca) (CLTA), ATP synthase, H+ transporting, mitochondrial F1 complex, beta polypeptide, calmodulin 2 (phosphorylase kinase, delta) (CALM2), actin, gamma 1 (ACTG1), ribosomal protein S17 (RPS17), ribosomal protein, large, P1 (RPLP1), ribosomal protein, large, P0 (RPLP0), thymosin, beta 4, X chromosome (TMSB4X), heterogeneous nuclear ribonucleoprotein C (C1/C2) (HNRPC), ribosomal protein L36a (RPL36A), glucuronidase, beta (GUSB), FYN oncogene related to SRC, FGR, YES (FYN), prothymosin, alpha (gene sequence 28) (PTMA), enolase 1, (alpha) (ENO1), laminin receptor 1 (67 kD, ribosomal protein SA) (LAMR1), ribosomal protein S14 (RPS14), CD74 antigen (invariant polypeptide of major histocompatibility complex, class II antigen-associated), esterase D/formylglutathione hydrolase (ESD), H3 histone, family 3A (H₃F₃A), ferritin, light polypeptide (FTL), Sec23 (S. cerevisiae) homolog A (SEZ23A), actin, beta (ACTB), presenilin 1 (Alzheimer disease 3) (PSEN1), interleukin-1 receptor-associated kinase 1 (IRAK1), zinc finger protein 162 (ZNF162), ribosomal protein L34 (RPL34), beclin 1 (coiled-coil, myosin-like BCL2-interacting protein) (BECN1), phosphatidylinositol 4-kinase, catalytic, alpha polypeptide (PIK4CA), IQ motif containing GTPase activating protein 1 (IQGAP1), signal transducer and activator of transcription 3 (acute-phase response factor) (STAT3), heterogeneous nuclear ribonucleoprotein F (HNRPF), putative translation initiation factor (SUI1), protein translocation complex beta (SEC61B), ras homolog gene family, member A (ARHA), ferritin, heavy polypeptide 1 (FTH1), Rho GDP dissociation inhibitor (GDI) beta (ARHGDIB), H2A histone family, member O (H2AFO), annexin A11 (ANXA1), ribosomal protein L27 (RPL27), adenylyl cyclase-associated protein (CAP), zinc finger protein 91 (HPF7, HTF10) (ZNF91), ribosomal protein L18 (RPL18), farnesyltransferase, CAAX box, alpha (FNTA), sodium channel, voltage-gated, type I, beta polypeptide (SCN1B), calnexin (CANX), proteolipid protein 2 (colonic epithelium-enriched) (PLP2), amyloid beta (A4) precursor-like protein 2 (APLP2), Voltage-dependent anion channel 2, proteasome (prosome, macropain) activator subunit 1 (PA28 alpha) (PSME1), ribosomal prot L12 (RPL12), ribosomal protein L37a (RPL37A), ribosomal protein S21 (RPS21), proteasome (prosome, macropain) 26S subunit, ATPase, 1 (PSMC1), major histocompatibility complex, class II, DQ beta 1 (HLA-DQB1), replication protein A2 (32 kD) (RPA2), heat shock 90 kD protein 1, beta (HSPCB), cytochrome c oxydase subunit VIII (COX8), eukaryotic translation elongation factor 1 alpha 1 (EEF1A1), SNRPN upstream reading frame (SNURF), lectin, galactoside-binding, soluble, 1 (galectin 1) (LGALS1), lysosomal-associated membrane protein 1 (LAMP1), phosphoglycerate mutase 1 (brain) (PGAM1), interferon-induced transmembrane protein 1 (9-27) (IFITM1), nuclease sensitive element binding protein 1 (NSEP1), solute carrier family 25 (mitochondrial carrier, adenine nucleotide translocator), member 6 (SLC25A6), ADP-ribosyltransferase (NAD+; poly (ADP-ribose) polymerase) (ADPRT), leukotriene A4 hydrolase (LTA4H), profilin 1 (PFN1), prosaposin (variant Gaucher disease and variant metachromatic leukodystrophy) (PSAP), solute carrier family 25 (mitochondrial carrier; adenine nucleotide translocator), member 5 (SLC25A5), beta-2 microglobulin, insulin-like growth factor binding protein 7, Ribosomal prot S13, Epstein-Barr Virus Small Rna-Associated prot, Major Histocompatibility Complex, Class I, C X58536), Ribosomal prot S12, Ribosomal prot L10, Transformation-Related prot, Ribosomal prot L5, Transcriptional Coactivator Pc4, Cathepsin B, Ribosomal prot L26, “Major Histocompatibility Complex, Class I X12432”, Wilm S Tumor-Related prot, Tropomyosin Tm30 nm Cytoskeletal, Liposomal Protein S4, X-Linked, Ribosomal prot L37, Metallopanstimulin 1, Ribosomal prot L30, Heterogeneous Nuclear Ribonucleoprot K, Major Histocompatibility Complex, Class I, E M21533, Major Histocompatibility Complex, Class I, E M20022, Ribosomal protein L30 Homolog, Heat Shock prot 70 Kda, “Myosin, Light Chain/U02629”, “Myosin, Light Chain/U02629”, Calcyclin, Single-Stranded Dna-Binding prot Mssp-1, Triosephosphate Isomerase, Nuclear Mitotic Apparatus prot 1, prot Kinase Ht31 Camp-Dependent, Tubulin, Beta 2, Calmodulin Type I, Ribosomal prot S20, Transcription Factor Btf3b, Globin, Beta, Small Nuclear RibonucleoproteinPolypeptide CAlt. Splice 2, Nucleoside Diphosphate Kinase Nm23-H2s, Ras-Related C3 Botulinum Toxin Substrate, activating transcription factor 4 (tax-responsive enhancer element B67) (ATF4), prefoldin (PFDN5), N-myc downstream regulated (NDRG1), ribosomal protein L14 (RPL14), nicastrin (KIAA0253), protease, serine, 11 (IGF binding) (PRSS11), KIAA0220 protein (KIAA0220), dishevelled 3 (homologous to Drosophila dsh) (DVL3), enhancer of rudimentary Drosophila homolog (ERH), RNA-binding protein gene with multiple splicing (RBPMS), 5-aminoimidazole-4-carboxamide ribonucleotide formyltransferase/IMP cyclohydrolase (ATIC), KIAA0164 gene product (KIAA0164), ribosomal protein L39 (RPL39), tyrosine 3 monooxygenase/tryptophan 5-monooxygenase activation protein, eta polypeptide (YWHAH), Ornithine decarboxylase antizyme 1 (OAZ1), proteasome (prosome, macropain) 26S subunit, non-ATPase, 2 (PSMD2), cold inducible RNA-binding protein (CIRBP), neural precursor cell expressed, developmentally down-regulated 5 (NEDD5), high-mobility group nonhistone chromosomal protein 1 (HMG1), malate dehydrogenase 1, NAD (soluble) (MDH1), cyclin I (CCNI), proteasome (prosome, macropain) 26S subunit, non-ATPase, 7 (Mov34 homolog) (PSMD7), major histocompatibility complex, class I, B (HLA-B), ATPase, vacuolar, 14 kD (ATP6S14), transcription factor-like 1 (TCFL1), KIAA0084 protein (KIAA0084), proteasome (prosome, macropain) 26S subunit, non-ATPase, 8 (PSMD8), major histocompatibility complex, class I, A (HIA-A), alanyl-tRNA synthetase (AARS), lysyl-tRNA synthetase (KARS), ADP-ribosylation factor-like 6 interacting protein (ARL61P), KIAA0063 gene product (KIAA0063), actin binding LIM protein 1 (ABLIM), DAZ associated protein 2 (DAZAP2), eukaryotic translation initiation factor 4A, isoform 2 (EIF4A2), CD151 antigen (CD151), proteasome (prosome, macropain) subunit, beta type, 6 (PSMB6), proteasome (prosome, macropain) subunit, beta type, 4 (PSMB4), proteasome (prosome, macropain) subunit, beta type, 2 (PSMB2), proteasome (prosome, macropain) subunit, beta type, 3 (PSMB3), Williams-Beuren syndrome chromosome region 1 (WBSCR1), ancient ubiquitous protein 1 (AUP1), KIAA0864 protein (KIAA0864), neural precursor cell expressed, developmentally down-regulated 8 (NEDD8), ribosomal protein L4 (RPL4), KIAA0111 gene product (KIAA0111), transgelin 2 (TAGLN2), Clathrin, heavy polypeptide (Hc) (CLTC, CLTCL2), ATP synthase, H+ transporting, mitochondrial F1complex, gamma polypeptide 1 (ATP5C1), calpastatin (CAST), MORF-related gene X (KIA0026), ATP synthase, H+ transporting, mitochondrial F1 complex, alpha subunit, isoform 1, cardiac muscle (ATP5A1), phosphatidylserine synthase 1 (PTDSS1), anti-oxidant protein 2 (non-selenium glutathione peroxidase, acidic calcium-independent phospholipase A2) (KIAA0106), KIAA0102 gene product (KIAA0102), ribosomal protein S23 (RPS23), CD164 antigen, sialomucin (CD164), GDP dissociation inhibitor 2 (GDI2), enoyl Coenzyme A hydratase, short chain, 1, mitochondrial (ECHS1), eukaryotic translation initiation factor 4A, isoform 1 (EIF4A1), cyclin D2 (CCND2), heterogeneous nuclear ribonucleoprotein U (scaffold attachment factor A) (HNRPU), APEX nuclease (multifunctional DNA repair enzyme) (APEX), ATP synthase, H+ transporting, mitochondrial F0 complex, subunit c (subunit 9), isoform 1 (ATP5G1), myristoylated alanine-rich protein kinase C substrate (MARCKS, 80K-L) (MACS), annexin A2 (ANXA2), similar to S. cerevisiae RER1 (RER1), hyaluronoglucosaminidase 2 (HYAL2), uroplakin 1A (UPK1A), nuclear pore complex interacting protein (NPIP), karyopherin alpha 4 (importin alpha 3) (KPNA4), ant the gene with multiple splice variants near HD locus on 4p16.3 (RES4-22).

In addition, the endogenous promoter can be a promoter associated with the expression of tissue specific or physiologically specific genes, such as heat shock genes.

In an alternative embodiment, the endogenous promoter can be a promoter for the genes encoding the proteins associated with the sugar metabolic pathway. In one preferred embodiment, the promoter is selected from the group consisting of the endogenous promoter for the α1,3 galactosyltransferase gene (see, for example, FIG. 28), the iGb3 synthase, or FSM synthase (GenBank Accession No._{—}039206).

b. Exogenous Promoters

In another embodiment, the promoter can be an exogenous promoter, such as a constitutively active viral promoter. Non-limiting examples of promoters include the RSV LTR, the SV40 early promoter, the CMV IE promoter, the adenovirus major late promoter, Srα-promoter (a very strong hybrid promoter composed of the SV40 early promoter fused to the R/U5 sequences from the HTLV-I LTR), the Epstein Barr viral promoter, and the Hepatitis B promoter.

Expression of the Vectors in Host Cells

The present invention also provides for methods that allow for the expression vectors to enter the host cells. Techniques that can be used to allow the DNA construct entry into the host cell include calcium phosphate/DNA coprecipitation, microinjection of DNA into the nucleus, electroporation, bacterial protoplast fusion with intact cells, transfection, or any other technique known by one skilled in the art. The DNA can be single or double stranded, linear or circular, relaxed or supercoiled DNA. For various techniques for transfecting mammalian cells, see, for example, Keown et al., Methods in Enzymology Vol. 185, pp. 527-537 (1990).

a. Transient Expression

In one aspect of the present invention, expression of the nucleic acid constructs encoding for proteins associated with the sugar metabolic pathway in a cell is transient. In one embodiment, transient expression vectors are provided that contain cDNA encoding a sugar metabolism-related protein operably linked to a promoter, such as, but not limited to those promoters described above. Transient expression can result from an expression vector that does not insert into the genome of the cell. Alternatively, transient expression can be from the direct insertion of RNA molecules into the cell.

RNA molecules encoding proteins associated with the sugar metabolic pathway can be made through the well-known technique of solid-phase synthesis. Equipment for such synthesis is sold by several vendors including, for example, Applied Biosystems (Foster City, Calif.). Other methods for such synthesis that are known in the art can additionally or alternatively be employed. It is well-known to use similar techniques to prepare oligonucleotides such as the phosphorothioates and alkylated derivatives. By way of non-limiting example, see, for example, U.S. Pat. Nos. 4,517,338, and 4,458,066; Lyer R P, et al., Curr. Opin. Mol Ther. 1:344-358 (1999); and Verma S, and Eckstein F., Annual Rev. Biochem. 67:99-134 (1998).

RNA directly inserted into a cell can include modifications to either the phosphate-sugar backbone or the nucleoside. For example, the phosphodiester linkages of natural RNA can be modified to include at least one of a nitrogen or sulfur heteroatom. The RNA encoding a protein associated with the sugar metabolic pathway can be produced enzymatically or by partial/total organic synthesis. The constructs can be synthesized by a cellular RNA polymerase or a bacteriophage RNA polymerase (e.g., T3, T7, SP6). If synthesized chemically or by in vitro enzymatic synthesis, the RNA can be purified prior to introduction into a cell or animal. For example, RNA can be purified from a mixture by extraction with a solvent or resin, precipitation, electrophoresis, chromatography or a combination thereof as known in the art. Alternatively, the RNA construct can be used without, or with a minimum of purification to avoid losses due to sample processing. The RNA molecules can be dried for storage or dissolved in an aqueous solution. The solution can contain buffers or salts to promote annealing, and/or stabilization of the duplex strands. Examples of buffers or salts that can be used in the present invention include, but are not limited to, saline, PBS, N-(2-Hydroxyethyl)piperazine-N′-(2-ethanesulfonic acid) (HEPES®), 3-(N-Morpholino)propanesulfonic acid (MOPS), 2-bis(2-Hydroxyethylene)amino-2-(hydroxymethyl)-1,3-propanediol (bis-TRIS®), potassium phosphate (KP), sodium phosphate (NaP), dibasic sodium phosphate (Na2HPO4), monobasic sodium phosphate (NaH2PO4), monobasic sodium potassium phosphate (NaKHPO₄), magnesium phosphate (Mg3(PO4)2.4H₂O), potassium acetate (CH3COOH), D(+)-α-sodium glycerophosphate (HOCH2CH(OH)CH2OPO3Na2) and other physiologic buffers known to those skilled in the art. Additional buffers for use in the invention include, a salt M-X dissolved in aqueous solution, association, or dissociation products thereof, where M is an alkali metal (e.g., Li+, Na+, K+, Rb+), suitably sodium or potassium, and where X is an anion selected from the group consisting of phosphate, acetate, bicarbonate, sulfate, pyruvate, and an organic monophosphate ester, glucose 6-phosphate or DL-α-glycerol phosphate.

b. Stable Expresssion

The nucleic acid constructs can further contain nucleic acid sequences that permit insertion into a host genome, i.e. “knocked-in” to the host genome. In one embodiment, the nucleic acid construct can be randomly integrated into the host genome. Alternatively, the nucleic acid construct can be inserted via targeted insertion into the host genome. In an another embodiment, the nucleic acid sequences encoding the protein can be cloned into a promoterless vector, and inserted into the genome of a cell, wherein the promoterless vector is under the control of a promoter associated with an endogenous gene. Nucleic acid constructs useful for targeted insertion of the galactose transport-related cDNA include 5′ and 3′ recombination arms for homologous recombination.

1. Random Insertion

Genomic Insertion of the nucleic acid contruct encoding for a protein associated with sugar metabolism can be accomplished using any known methods of the art. In one embodiment, the vector is inserted into a genome randomly using a viral based vector. Insertion of the virally based vector occurs at random sites consistent with viral behavior (see, for example, Daley et al. (1990) Science 247:824-830; Guild et al. (1988) J Virol 62:3795-3801; Miller (1992) Curr Topics MicroBiol Immunol 158:1-24; Samarut et al. (1995) Methods Enzymol 254:206-228). Non limiting examples of viral based vectors include Moloney murine leukemia retrovirus, the murine stem cell virus, vaccinia viral vectors, Sindbis virus, Semliki Forest alphavirus, EBV, ONYX-15, adenovirus, or lentivirus based vectors (see, for example, Hemann M T et al. (2003) Nature Genet. 33:396400; Paddison & Hannon (2002) Cancer Cell 2:17-23; Brummelkamp T R et al. (2002) Cancer Cell 2:243-247; Stewart S A et al. (2003) RNA 9:493-501; Rubinson D A et al. (2003) Nature Genen. 33:401-406; Qin X et al. (2003) PNAS USA 100:183-188; Lois C et al. (2002) Science 295:868-872).

2. Targeted Insertion

One embodiment of the invention which allows transfer of the nucleic acid sequences encoding proteins associated with sugar metabolism to the genome while also limiting the amount of the expression vector that is also transferred to a fragment that is not significant, is the method of recombinational cloning, see, for example, U.S. Pat. Nos. 5,888,732 and 6,277,608.

Recombinational cloning (see, for example, U.S. Pat. Nos. 5,888,732 and 6,277,608) describes methods for moving or exchanging nucleic acid segments using at least one recombination site and at least one recombination protein to provide chimeric DNA molecules. One method of producing these chimeric molecules which is useful in the methods of the present invention to produce the nucleic acid sequences encoding proteins associated with sugar metabolism expression vectors comprises: combining in vitro or in vivo, (a) one or more nucleic acid molecules comprising the one or more nucleic acid sequences encoding proteins associated with sugar metabolism of the invention flanked by a first recombination site and a second recombination site, wherein the first and second recombination sites do not substantially recombine with each other, (b) one or more expression vector molecules comprising a third recombination site and a fourth recombination site, wherein the third and fourth recombination sites do not substantially recombine with each other, and (c) one or more site specific recombination proteins capable of recombining the first and third recombinational sites and/or the second and fourth recombinational sites, thereby allowing recombination to occur, so as to produce at least one cointegrate nucleic acid molecule which comprises the one or more nucleic acid sequences encoding proteins associated with sugar metabolism.

Recombination sites and recombination proteins for use in the methods of the present invention, include, but are not limited to those described in U.S. Pat. Nos. 5,888,732 and 6,277,608, such as, Cre/loxP, Integrase (λInt, Xis, IHF and FIS)/att sites (attB, attP, attL and attR), and FLP/FRT. Members of a second family of site-specific recombinases, the resolvase family (e.g., gd, Tn3 resolvase, Hin, Gin, and Cin) are also known and can be used in the methods of the present invention. Members of this highly related family of recombinases are typically constrained to intramolecular reactions (e.g., inversions and excisions) and can require host-encoded factors. Mutants have been isolated that relieve some of the requirements for host factors (Maeser and Kahnmann Mol. Gen. Genet. 230:170-176 (1991)), as well as some of the constraints of intramolecular recombination.

Other site-specific recombinases similar to λint and similar to P1 Cre that are known in the art and that will be familiar to one of ordinary skill can be substituted for Int and Cre. In many cases the purification of such other recombinases has been described in the art. In cases when they are not known, cell extracts can be used or the enzymes can be partially purified using procedures described for Cre and Int.

The family of enzymes, the transposases, have also been used to transfer genetic information between replicons and can be used in the methods of the present invention to transfer nucleic acid sequences encoding proteins associated with sugar metabolism. Transposons are structurally variable, being described as simple or compound, but typically encode the recombinase gene flanked by DNA sequences organized in inverted orientations. Integration of transposons can be random or highly specific. Representatives such as Tn7, which are highly site-specific, have been applied to the in vivo movement of DNA segments between replicons (Lucklow et al., J. Virol. 67:45664579 (1993)). For example, Devine and Boeke (Nucl. Acids Res. 22:3765-3772 (1994)) disclose the construction of artificial transposons for the insertion of DNA segments, in vitro, into recipient DNA molecules. The system makes use of the integrase of yeast TY1 virus-like particles. The nucleic segment of interest is cloned, using standard methods, between the ends of the transposon-like element TY1. In the presence of the TY1 integrase, the resulting element integrates randomly into a second target DNA molecule.

Additional recombination sites and recombination proteins, as well as mutants, variants and derivatives thereof, for example, as described in U.S. Pat. Nos. 5,888,732, 6,277,608 and 6,143,557 can also be used in the methods of the present invention.

Following the production of an expression vector containing one or more nucleic acid sequences encoding proteins associated with sugar metabolism flanked by recombination proteins, the nucleic acid sequences encoding proteins associated with sugar metabolism can be transferred to the genome of a target cell via recombinational cloning. In this embodiment, the recombination proteins flanking the nucleic acid sequences encoding proteins associated with sugar metabolism are capable of recombining with one or more recombination proteins in the genome of the target cell. In combination with one or more site specific recombination proteins capable of recombining the recombination sites, the nucleic acid sequences encoding proteins associated with sugar metabolism is transferred to the genome of the target cell without transferring a significant amount of the remaining expression vector to the genome of the target cell. The recombination sites in the genome of the target cell can occur naturally or the recombination sites can be introduced into the genome by any method known in the art. In either case, the recombination sites flanking the one or more nucleic acid sequences encoding proteins associated with sugar metabolism in the expression vector must be complementary to the recombination sites in the genome of the target cell to allow for recombinational cloning.

Another embodiment of the invention relates to methods to produce a non-human transgenic or chimeric animal comprising crossing a male and female non-human transgenic animal produced by any one of the methods of the invention to produce additional transgenic or chimeric animal offspring. By crossing transgenic male and female animals that both contain the one or more nucleic acid sequences encoding proteins associated with sugar metabolism in their genome, the progeny produced by this cross also contain the nucleic acid sequences encoding proteins associated with sugar metabolism in their genome. This crossing pattern can be repeated as many times as desired.

In another embodiment, the insertion is targeted to a specific gene locus through homologous recombination. Homologous recombination provides a precise mechanism for targeting defined modifications to genomes in living cells (see, for example, Vasquez K M et al. (2001) PNAS USA 98(15):8403-8410). A primary step in homologous recombination is DNA strand exchange, which involves a pairing of a DNA duplex with at least one DNA strand containing a complementary sequence to form an intermediate recombination structure containing heteroduplex DNA (see, for example, Radding, C. M. (1982) Ann. Rev. Genet. 16: 405; U.S. Pat. No. 4,888,274). The heteroduplex DNA can take several forms, including a three DNA strand containing triplex form wherein a single complementary strand invades the DNA duplex (see, for example, Hsieh et al. (1990) Genes and Development 4: 1951; Rao et al., (1991) PNAS 88:2984)) and, when two complementary DNA strands pair with a DNA duplex, a classical Holliday recombination joint or chi structure (Holliday, R. (1964) Genet. Res. 5: 282) can form, or a double-D loop (“Diagnostic Applications of Double-D Loop Formation” U.S. Pat. No. 5,273,881). Once formed, a heteroduplex structure can be resolved by strand breakage and exchange, so that all or a portion of an invading DNA strand is spliced into a recipient DNA duplex, adding or replacing a segment of the recipient DNA duplex. Alternatively, a heteroduplex structure can result in gene conversion, wherein a sequence of an invading strand is transferred to a recipient DNA duplex by repair of mismatched bases using the invading strand as a template (see, for example, Genes, 3rd Ed. (1987) Lewin, B., John Wiley, New York, N.Y.; Lopez et al. (1987) Nucleic Acids Res. 15: 5643). Whether by the mechanism of breakage and rejoining or by the mechanism(s) of gene conversion, formation of heteroduplex DNA at homologously paired joints can serve to transfer genetic sequence information from one DNA molecule to another.

A number of papers describe the use of homologous recombination in mammalian cells. Illustrative of these papers are Kucherlapati et al. (1984) Proc. Natl. Acad. Sci. USA 81:3153-3157; Kucherlapati et al. (1985) Mol. Cell. Bio. 5:714-720; Smithies et al. (1985) Nature 317:230-234; Wake et al. (1985) Mol. Cell. Bio. 8:2080-2089; Ayares et al. (1985) Genetics 111:375-388; Ayares et al. (1986) Mol. Cell. Bio. 7:1656-1662; Song et al. (1987) Proc. Natl. Acad. Sci. USA 84:6820-6824; Thomas et al. (1986) Cell 44:419428; Thomas and Capecchi, (1987) Cell 51: 503-512; Nandi et al. (1988) Proc. Natl. Acad. Sci. USA 85:3845-3849; and Mansour et al. (1988) Nature 336:348-352; Evans and Kaufman, (1981) Nature 294:146-154; Doetschman et al. (1987) Nature 330:576-578; Thoma and Capecchi, (1987) Cell 51:503-512; Thompson et al. (1989) Cell 56:316-321.

Cells useful for homologous recombination include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc.

The vector construct containing the nucleic acid sequence encoding for a protein associated with sugar metabolism can comprise a full or partial sequence of one or more exons and/or introns of the gene targeted for insertion, a full or partial promoter sequence of the gene targeted for insertion, or combinations thereof. In one embodiment of the invention, the construct comprises a first nucleic acid sequence region homologous to a first nucleic acid sequence region of the gene targeted for insertion, a second nucleic acid sequence containing the nucleic acid sequence encoding a protein associated with the sugar metabolic pathway and a third nucleic acid sequence region homologous to a second nucleic acid sequence region of the gene targeted for insertion. The vector can contain a promoter operably linked to the second nucleic acid sequence encoding for a protein associated with sugar metabolism. Alternatively, the vector can be promoterless, and driven by the associated targeted gene's promoter. The orientation of the vector construct should be such that the first nucleic acid sequence is upstream of the third nucleic acid sequence and the second nucleic acid region containing the nucleic acid sequence encoding for the protein associated with the sugar metabolic pathway should be there between.

A nucleic acid sequence region(s) can be selected so that there is homology between the vector construct sequence(s) and the gene targeted for insertion. Preferably, the construct sequences are isogonics sequences with respect to the region targeted for insertion. The nucleic acid sequence region of the construct may correlate to any region of the gene provided that it is homologous to the gene. A nucleic acid sequence is considered to be “homologous” if it is at least about 90% identical, preferably at least about 95% identical, or most preferably, about 98% identical to the nucleic acid sequence. Furthermore, the 5′ and 3′ nucleic acid sequences flanking the nucleic acid sequence encoding for a protein associated with the sugar metabolic pathway should be sufficiently large to provide complementary sequence for hybridization when the construct is introduced into the genomic DNA of the target cell. For example, homologous nucleic acid sequences flanking the nucleic acid sequence encoding for a protein associated with the sugar metabolic pathway should be at least about 500 bp, preferably, at least about 1 kilobase (kb), more preferably about 24 kb, and most preferably about 34 kb in length. In one embodiment, both of the homologous nucleic acid sequences flanking the nucleic acid sequence encoding for a protein associated with the sugar metabolic pathway of the construct should be at least about 500 bp, preferably, at least about 1 kb, more preferably about 2-4 kb, and most preferably about 3-4 kb in length.

In another embodiment, the vector is inserted into a single allele of a housekeeping gene. Non limiting examples of targeted housekeeping genes include, but are not limited to, those describes above.

In an alternative embodiment, the vector can be inserted into a host gene associated with xenotransplantation rejection in a host. In one particular embodiment, the gene the vector is inserted into is selected from the group consisting of the α1,3-galactosyltransferase gene, the Forsmann synthestase gene, and the iGb3 synthase gene.

Methods for generating gene constructs for use in generating “knock-in” and “knockout” mammals and the techniques for generating the mammals are known to those of skill in the art, and may be found, for example, in Sambrook et al., 1989, Molecular Cloning, A Laboratory Manual, 3.sup.rd ed., Cold Spring Harbor Laboratory; Yoo et al., 2003, Neuron, 37: 383; Watase et al., 2002, Neuron, 34:905; Lorenzetti et al., 2000, Human Molecular Genetics, 9:779; and Lin et al., 2001, Human Molecular Genetics, 10: 137.

a. Promoter Trap

In an alternative embodiment, a nucleic acid construct encoding for a protein associated with the sugar metabolic pathway lacking an operably linked promoter can be inserted into an endogenous gene via a promoter trap strategy. The insertion allows expression of a promoterless vector to be driven by the endogenous gene's associated promoter. This ‘promoter trap’ gene targeting construct may be designed to contain a sequence with homology to an endogenous gene's 3′ intron sequence upstream of the start codon, the upstream intron splice acceptor sequence comprising the AG dinucleotide splice acceptor site, a Kozak consensus sequence, a promoterless vector containing nucleic acid sequence encoding for a protein associated with the sugar metabolic process, including a stop codon, a polyA termination sequence, a splice donor sequence comprising a dinucleotide splice donor site from a intron region downstream of the start codon, and a sequence with 5′ sequence homology to the downstream intron. It will be appreciated that the method may be used to target the exon containing the start codon within the targeted gene.

In one embodiment, the vector is inserted into an exon containing the start codon of a housekeeping gene. Preferably, the vector is inserted into a single allele of the housekeeping gene.

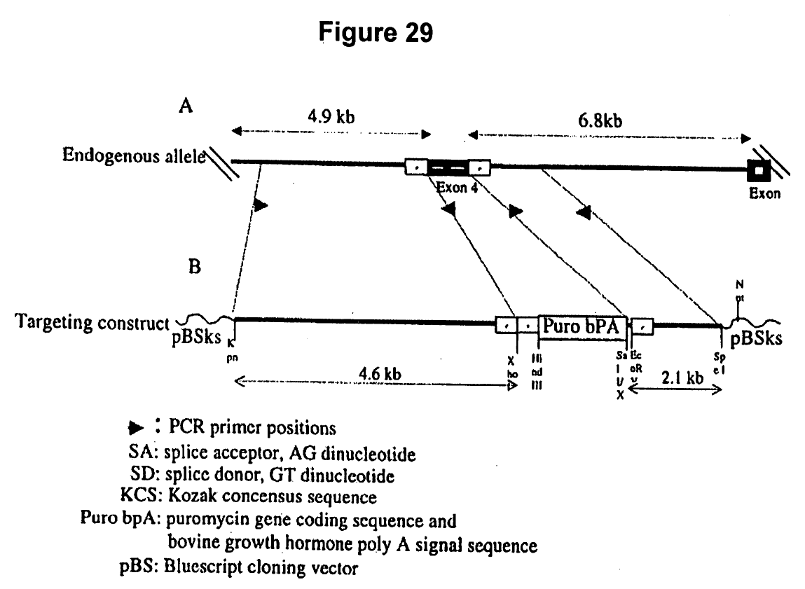

In an alternative embodiment, the vector is inserted into the α1,3-galactosyltransferase gene utilizing a promoter trap strategy. In a more particular embodiment, the vector is inserted into exon 4 of the porcine α1,3-galactosyltransferase gene. (See, for example, FIG. 29, and PCT Publication No. WO 01/23541).

In an alternative embodiment, the vector is inserted into the Forsmann synthetase gene utilizing a promoter trap strategy. In a more particular embodiment, the vector is inserted into exon 2 of the porcine Forsmann Synthetase gene in a promoter trap strategy.

In still another embodiment, the vector is inserted into the isoGloboside 3 synthase gene utilizing a promoter trap strategy. More particularly, the vector is inserted into exon 1 of the porcine isoGloboside 3 synthase gene.

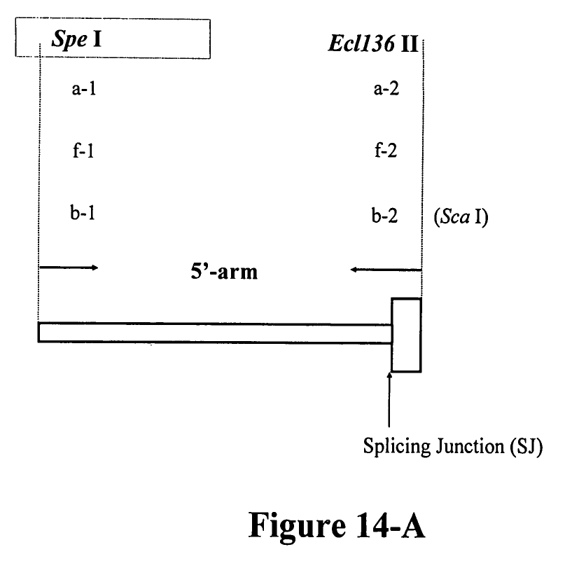

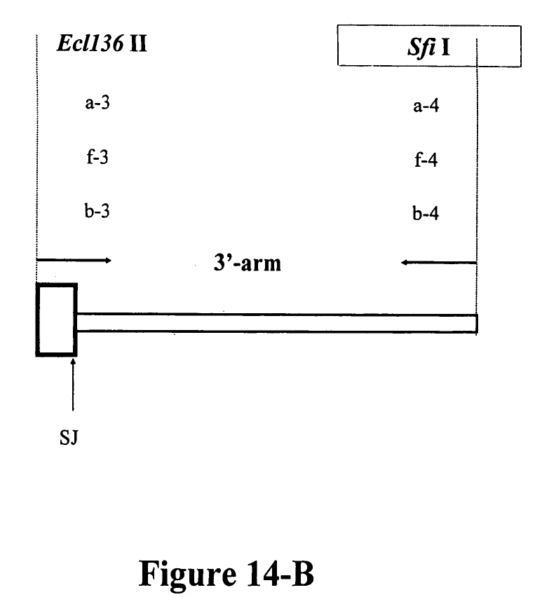

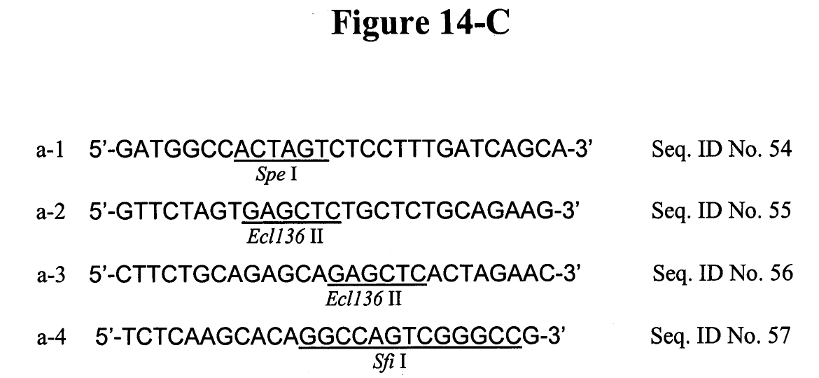

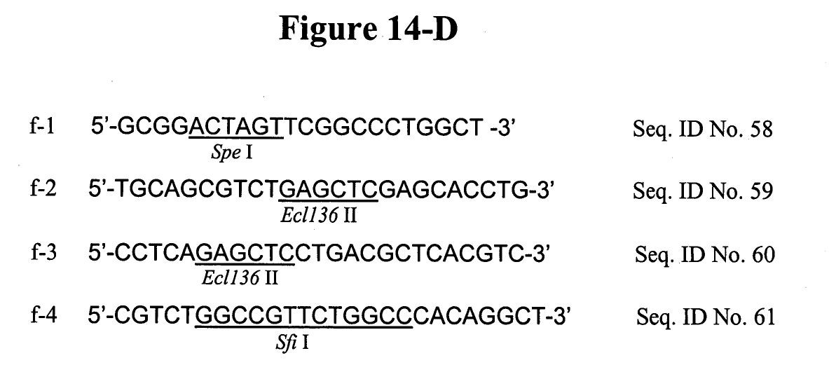

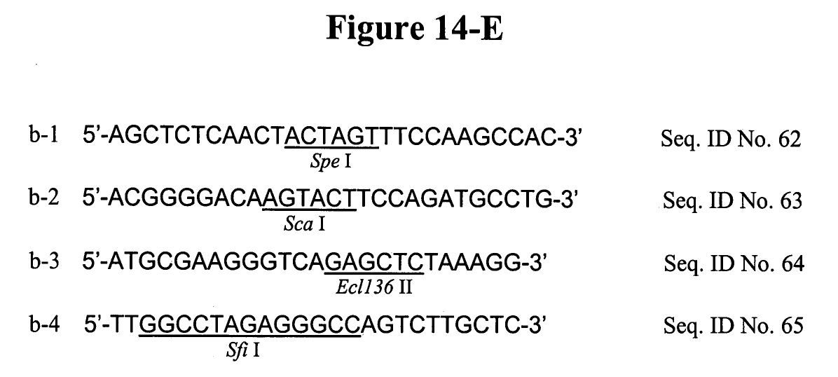

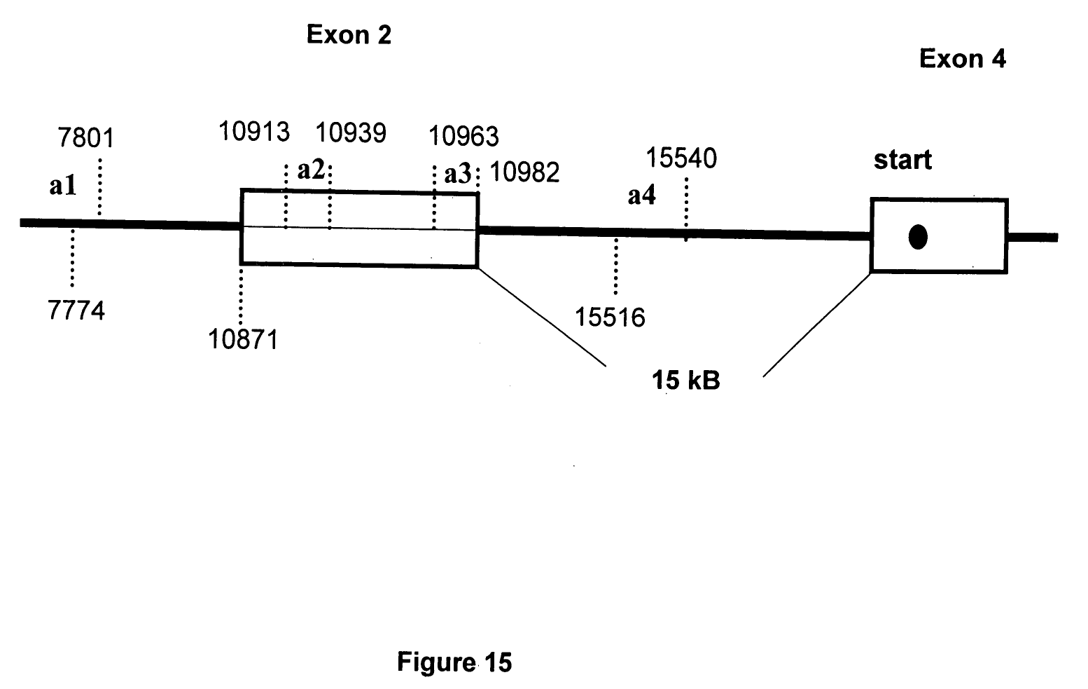

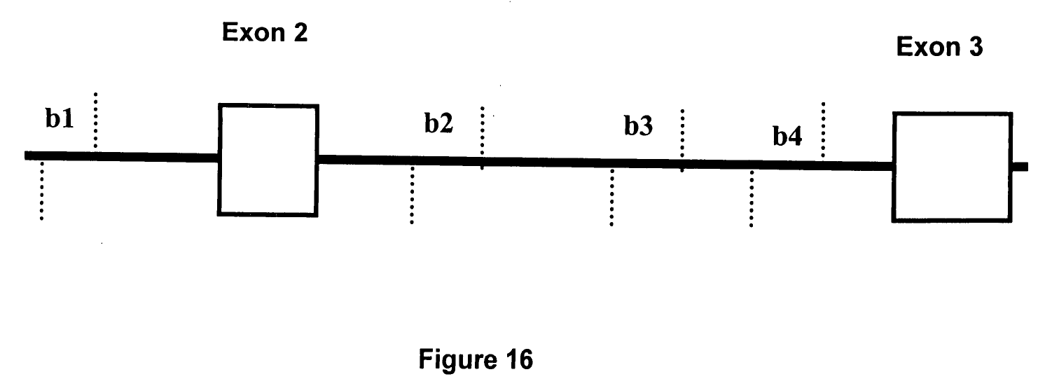

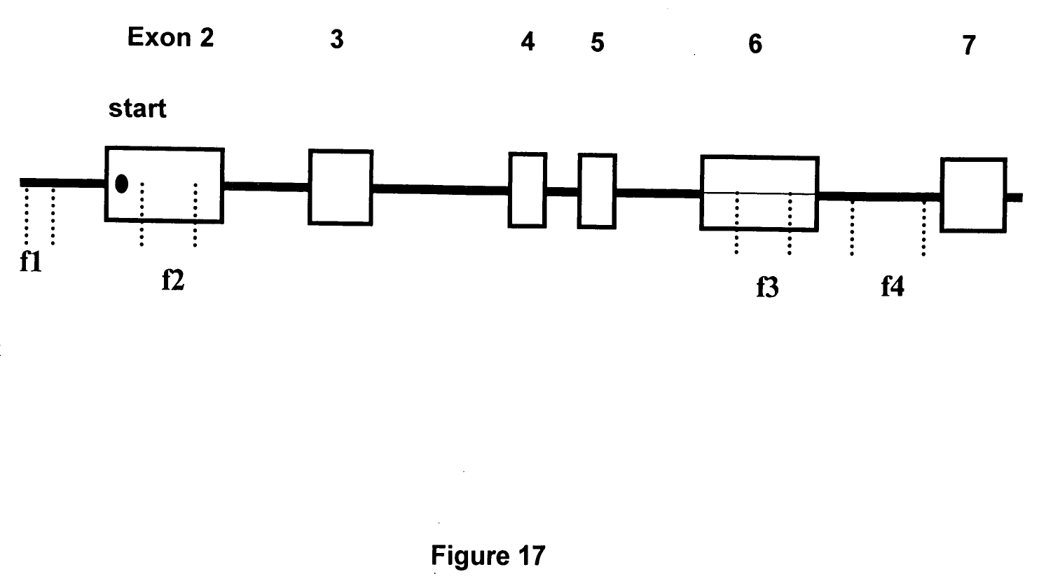

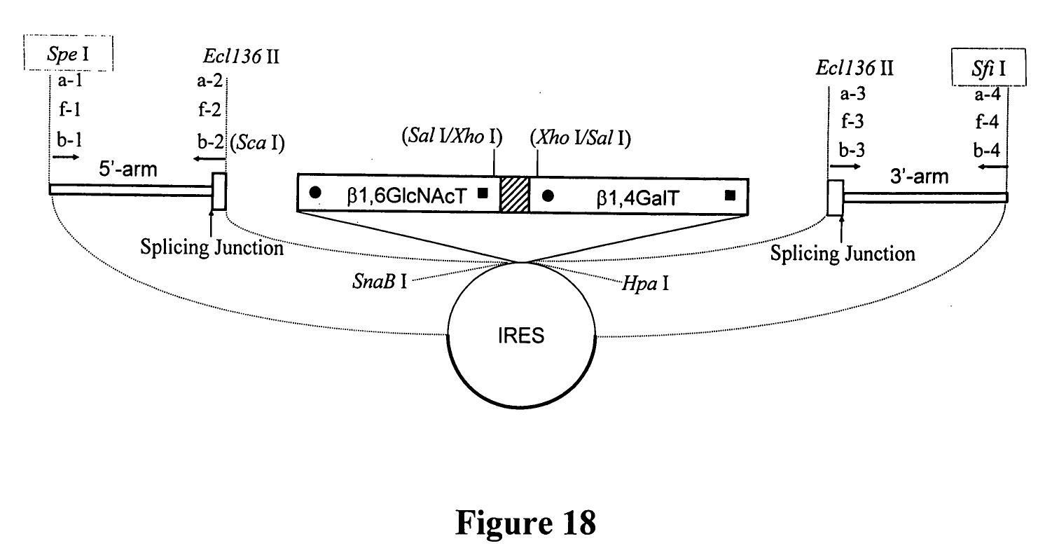

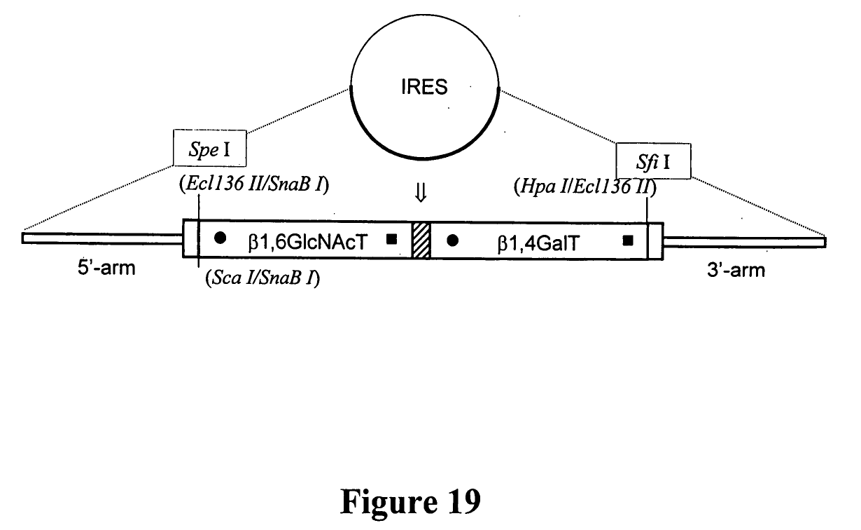

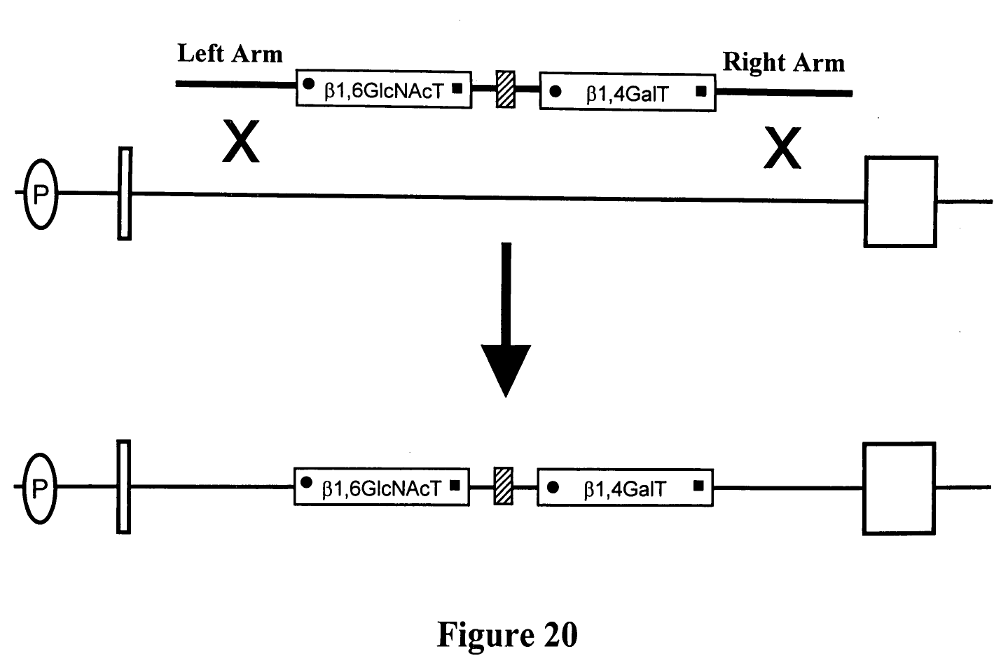

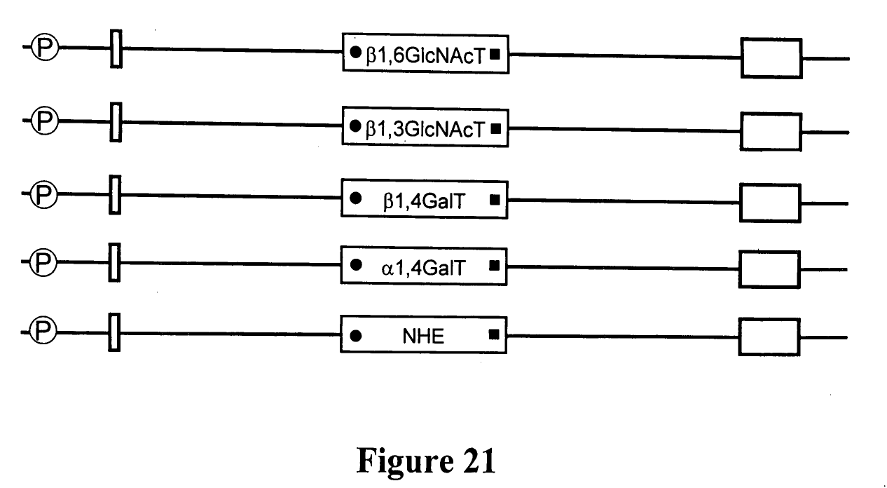

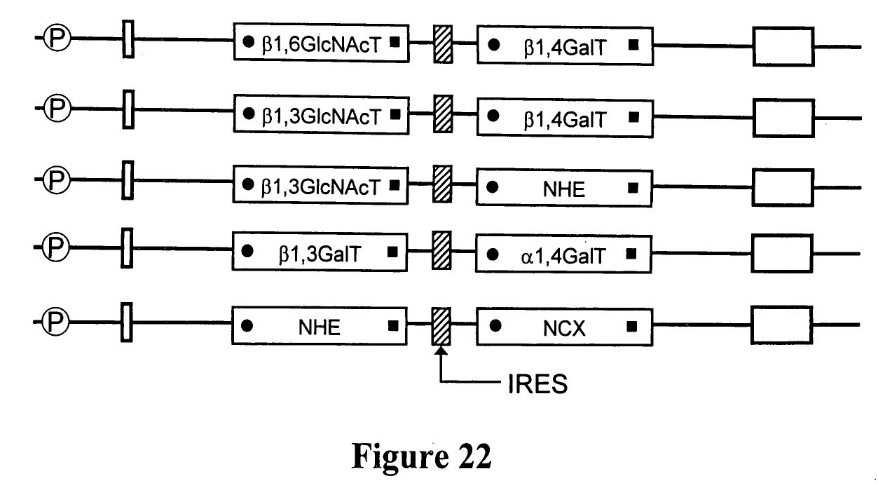

Specific embodiments of the present invention provide methods to produce a cell which has at least one additional protein associated with sugar catabolism, such as GALE, the hexosamine pathway, such as GFAT and/or NHE, or sugar chain synthesis, such as β-1,3-GT, β-1,4-GT, α-1,4-GT, α-1,4-GalNAcT, β-1,4-GalNAcT, β-1,3-GlcNAcT and/or β1,6-GlcNAcT inserted into a cell that already lacks functional expression of α1,3-galactosyltransferase, iGb3 synthase, Forssman synthetase, or a gene associated with xenotransplant rejection. In one embodiment, the nucleic acid construct is transiently transfected into the cell. In another embodiment, the nucleic acid construct is inserted into the genome of the cell via random or targeted insertion. In a further embodiment, the contruct is inserted via homologous recombination into a targeted genomic sequence within the cell such that it is under the control of an endogenous promoter. In a specific embodiment, the nucleic acid construct is inserted into the α1,3-galactosyltransferase genomic sequence, iGb3 synthase genomic sequence, Forssman synthetase genomic sequence, or a xenotransplant rejection-associated genomic sequence via homologous recombination such that the galactose transport-related cDNA is under the control of the α-1,3-GT, iGb3 synthase or FSM promoter (see, for example, FIGS. 7-22).

In one embodiment, cells are provided that lack functional expression of the alpha-1,3-galactosyltransferase (α-1,3-GT) gene, which have at least one additional protein associated with galactose transport, such as sugar catabolism associated proteins, such as GALE, hexosamine pathway associated proteins, such as GFAT and/or NHE, or sugar chain synthesis associated proteins, such as β-1,3-GT, β-1,4-GT, α-1,4-GT, α-1,4-GalNAcT, β-1,4-GalNAcT, β-1,3-GlcNAcT and/or β-1,6-GlcNAcT inserted into their genome. These sugar-related proteins from any known prokaryote or eukaryote, such as humans or porcine, can be inserted into the genome via random or targeted insertion, or expressed transiently. These proteins can be under the control of the endogenous α-1,3-GT promoter or a constitutively active promoter, such as a housekeeping gene promoter or viral promoter.

In an alternate embodiment, cells are provided that lack functional expression of the isoGloboside 3 (iGb3) synthase gene, which have at least one additional protein associated with galactose transport, such as sugar catabolism associated proteins, such as GALE, hexosamine pathway associated proteins, such as GFAT and/or NHE, or sugar chain synthesis associated proteins, such as β-1,3-GT, β-1,4-GT, α-1,4-GT, α-1,4-GalNAcT, β-1,4-GalNAcT, β-1,3-GlcNAcT and/or β-1,6-GlcNAcT inserted into their genome. These sugar-related proteins from any known prokaryote or eukaryote, such as humans or porcine, can be inserted into the genome via random or targeted insertion, or expressed transiently. These proteins can be under the control of the endogenous iGb3 synthase promoter or a constitutively active promoter, such as a housekeeping gene promoter or viral promoter.

In another embodiment, cells are provided that lack functional expression of the Forssman (FSM) synthetase gene, which have at least one additional protein associated with galactose transport, such as sugar catabolism associated proteins, such as GALE, hexosamine pathway associated proteins, such as GFAT and/or NHE, or sugar chain synthesis associated proteins, such as β-1,3-GT, β-1,4-GT, α-1,4-GT, α-1,4-GalNAcT, β-1,4-GalNAcT, β-1,3-GlcNAcT and/or β-1,6-GlcNAcT inserted into their genome. These sugar-related proteins from any known prokaryote or eukaryote, such as humans or porcine, can be inserted into the genome via random or targeted insertion, or expressed transiently. These proteins can be under the control of the endogenous Forssman synthetase promoter or a constitutively active promoter, such as a housekeeping gene promoter or a viral promoter.

III. Production of Genetically Modified Animals

The present invention provides animals, as well as tissues, organs and cells derived from such animals that have deficiencies in sugar metabolism, which have been genetically modified to compensate for the metabolic deficiency. This modification serves to decrease the accumulation of toxic metabolites in the cell caused by the metabolic deficiency. Such animals, tissues, organs and cells can be used in research and in medical therapy, including in xenotransplantation. In addition, methods are provided to produce such animals, organs, tissues, and cells. Furthermore, methods are provided for reducing toxic metabolite accumulation in animals, tissues, organs, and cells, which have metabolic deficiencies.

In one aspect of the invention, animals, as well as tissues, organs and cells derived therefrom, are provided in which at least one allele of a gene involved in galactose transport has been inactivated, which have been genetically modified to express at least one additional protein that can transport galactose out of the cell to compensate for this deficiency. Proteins involved in galactose transport include: proteins involved in: sugar catabolism, such as, but not limited to, galactokinase (GALK), galactose-1-phosphate uridyl transferase (GALT) and UDP-galactose-4-epimerase (GALE); the hexosamine pathway, such as, but not limited to, glutamine: fructose-6-phosphate amidotransferase (GFAT), the sodium-calcium exchanger (NCX) and the sodium-hydrogen exchanger (NHE); sugar chain synthesis, such as, but not limited to, β-1,3-galactosyltransferase (β-1,3-GT), 1-1,4-galactosyltransferase (1-1,4-GT), α-1,4-galactosyltransferase (α-1,4-GT), α-1,3-galactosyltransferase (α-1,3-GT), IsoGlobide 3 synthase (iGb3), Forssman synthase (FSM), N-acetylgalactosaminyltransferases (GalNAcT), and N-acetylglucosaminyltransferases (GlcNAc-T), such as β-1,6 GlcNac-T.

Any non-human transgenic animal can be produced by any one of the methods of the present invention including, but not limited to, non-human mammals including, but not limited to, pigs, sheep, goats, cows (bovine), deer, mules, horses, monkeys, apes, and other non-human primates, dogs, cats, rats, mice, rabbits, birds including, but not limited to chickens, turkeys, ducks, geese, canaries, and the like, reptiles, fish, amphibians, worms including C. elegans, and insects including, but not limited to, Drosophila, Trichoplusa, and Spodoptera.

The present invention also provides animal that have nucleic acid sequences encoding proteins associated with sugar metabolism inserted in its genome. In one embodiment, the animal is capable of expressing the product of the inserted sequence within the majority of its cells. In another embodiment, the animal is capable of expressing the product of the inserted sequence in virtually all of its cells. Since the sequence is incorporated into the genome of the animal, the nucleic acid insert will be inherited by subsequent generations, thus allowing these generations to also produce the product of the inserted nucleic acid sequence within their cells.

Another aspect of the present invention provides methods to produce a transgenic animal from a cell which has at least one galactose transport-related protein associated with sugar catabolism, such as GALE, the hexosamine pathway, such as GFAT and/or NHE, or sugar chain synthesis, such as β-1,3-GT, 1-1,4-GT, α-1,4-GT, α-1,4-GalNAcT, 13-1,4-GalNAcT, 1-1,3-GlcNAcT and/or 1-1,6-GlcNAcT transfected into a cell that already lacks functional expression of α1,3-galactosyltransferase, iGb3 synthase, Forssman synthetase, or a gene associated with xenotransplant rejection. Cells which have at least one sugar-related protein associated with sugar catabolism transfected into a cell that already lacks functional expression of α1,3-galactosyltransferase, iGb3 synthase, Forssman synthetase, or a gene associated with xenotransplant rejection can be used as donor cells to provide the nucleus for nuclear transfer into enucleated oocytes to produce cloned, transgenic animals. Alternatively, insertions containing nucleic acid sequence encoding for sugar-related proteins can be created in embryonic stem cells lacking functional expression of α1,3-galactosyltransferase, iGb3 synthase, Forssman synthetase, or a gene associated with xenotransplant rejection, which are then used to produce offspring. The methods of the invention are particularly suitable for the production of transgenic mammals (e.g. mice, rats, sheep, goats, cows, pigs, rabbits, dogs, horses, mules, deer, cats, monkeys and other non-human primates and the like), birds (particularly chickens, ducks, geese and the like), fish, reptiles, amphibians, worms (e.g. C. elegans), insects (including but not limited to, Drosophila spp., Trichoplusa spp., and Spodoptera spp.) and the like. While any species of animal can be produced, in a specific embodiment the animals are transgenic pigs.

In one aspect of the present invention, an animal can be prepared by a method in accordance with any aspect of the present invention. The genetically modified animals can be used as a source of tissues and/or organs for human transplantation therapy. An animal embryo prepared in this manner or a cell line developed therefrom can also be used in cell-transplantation therapy. In one embodiment, the animal utilized is a pig. Accordingly, there is provided in a further aspect of the invention a method of therapy comprising the administration of genetically modified animal cells which have at least one galactose transport-related protein associated with sugar catabolism transfected into a cell that already lacks functional expression of α1,3-galactosyltransferase, iGb3 synthase, Forssman synthetase, or a gene associated with xenotransplant rejection to a patient, wherein the cells have been prepared from an embryo or animal. This aspect of the invention can include the use of such cells in medicine, e.g. cell-transplantation therapy, and also the use of cells derived from such embryos in the preparation of a cell or tissue graft for transplantation. The cells can be organized into tissues or organs, for example, heart, lung, liver, kidney, pancreas, corneas, nervous (e.g. brain, central nervous system, spinal cord), skin, or the cells can be islet cells, blood cells (e.g. haemocytes, i.e. red blood cells, leucocytes) or haematopoietic stem cells or other stem cells (e.g. bone marrow). In a specific embodiment, the animal utilized is a pig.

Another aspect of the present invention includes methods for modifying sugar metabolic processes within a cell by inserting a nucleic acid construct encoding at least one galactose transport-related protein associated with sugar catabolism, such as GALE, the hexosamine pathway, such as GFAT and/or NHE, or sugar chain synthesis, such as β-1,3-GT, β-1,4-GT, α-1,4-GT, α-1,4-GalNAcT, >1,4-GalNAcT, β-1,3-GlcNAcT and/or β-1,6-GlcNAcT. In one embodiment, the nucleic acid construct is inserted into a cell that lacks functional expression of a galactose transport-related protein. In a more particular embodiment, the inserted construct encodes for a galactose transport-related protein that is different from the galactose transport-related protein that is lacking functional expression.

In an alternative aspect of the present invention, methods for modifying sugar metabolism in animals, tissues, organs, or cells lacking functional expression of a particular galactose transport-related protein are provided wherein dietary intake of sugars is restricted. In one embodiment, animals, tissues, organs, or cells lacking functional expression of α1,3-galactosyltransferase, iGb3 synthase, or Forssman synthetase, are fed a diet reduced in galactose and lactose. In a more particular embodiment, animals, tissues, organs, or cells lacking functional expression of α1,3-galactosyltransferase are fed a diet lacking galactose and lactose.

In one embodiment of the present invention, non-human transgenic animals are produced via the process of nuclear transfer. Production of non-human transgenic animals which express one or more nucleic acid sequences encoding for proteins associated with sugar metabolism via nuclear transfer comprises: (a) identifying the proteins associated with sugar metabolism to be used to compensate for the aberrant, abnormal, or absent expression of an other protein associated with sugar metabolism; (b) preparing one or more expression vectors containing one or more nucleic acid sequences encoding for proteins associated with sugar metabolism, (c) inserting the one or more expression vectors into the genome of a nuclear donor cell; (e) transferring the genetic material of the nuclear donor cell to an acceptor cell; (f) transferring the acceptor cell to a recipient female animal; and (g) allowing the transferred acceptor cell to develop to term in the female animal. See, for example, U.S. Patent Publication No. 2002/0012260.

Methods on the generation of genetically modified somatic cells for use in nuclear transfer can be found in WO 00/51424 to PPL Therapeutics, Inc. In addition, U.S. Pat. No. 6,872,868 to Ohio Universiry describes methods for the transgenic expression of proteins in animals.

The term nuclear donor cell is used to describe any cell which serves as a donor of genetic material to an acceptor cell. Examples of cells which can be used as nuclear donor cells include any somatic cell of an animal species in the embryonic, fetal, or adult stage. As used herein, the term “embryonic” refers to all concepts of an animal embryo, such as an oocyte, egg, zygote, or an early embryo. As used herein, the term “fetal” refers to an unborn animal, post embryonic stage, after it has attained the particular form the animal species. As used herein, the term “adult” cell refers to an animal or animal cell which is born. Thus an animal and its cells are deemed “adult” from birth. Such adult animals, cover animals from birth onwards and thus include “babies” and “juveniles.”

Somatic nuclear donor cells can be obtained from a variety of different organs and tissues such as, but not limited to, skin, mesenchyme, lung, pancreas, heart, intestine, stomach, bladder, blood vessels, kidney, urethra, reproductive organs, and a diaggregated preparation of a whole or part of an embryo, fetus, or adult animal. In one embodiment of the invention, nuclear donor cells are selected from the group consisting of epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells or endothelial cells. In another embodiment, the somatic nuclear donor cell is an embryonic stem cell.

In another embodiment of the invention, the nuclear donor cells of the invention are germ cells of an animal. Any germ cell of an animal species in the embryonic, fetal, or adult stage can be used as a nuclear donor cell. In one embodiment, the nuclear donor cell is an embryonic germ cell.

Nuclear donor cells can be arrested in any phase of the cell cycle (G0, G1, G2, S, M) so as to ensure coordination with the acceptor cell. Any method known in the art can be used to manipulate the cell cycle phase. Methods to control the cell cycle phase include, but are not limited to, G0 quiescence induced by contact inhibition of cultured cells, G0 quiescence induced by removal of serum or other essential nutrient, G0 quiescence induced by senescence, G0 quiescence induced by addition of a specific growth factor; G0 or G1 quiescence induced by physical or chemical means such as heat shock, hyperbaric pressure or other treatment with a chemical, hormone, growth factor or other substance; S-phase control via treatment with a chemical agent which interferes with any point of the replication procedure; M-phase control via selection using fluorescence activated cell sorting, mitotic shake off, treatment with microtubule disrupting agents or any chemical which disrupts progression in mitosis. See, for example, Freshney, R. I,. “Culture of Animal Cells: A Manual of Basic Technique,” Alan R. Liss, Inc, New York (1983) for teachings regarding control of cell cycle phase.

Acceptor cells for use in the present invention include, but are not limited to: oocytes, fertilized zygotes, or two cell embryos. In all cases, the original genomic material of the acceptor cells must be removed. This process has been termed “enucleation.” The removal of genetic material via enucleation does not require that the genetic material of the acceptor cell be enclosed in a nuclear membrane, though it can be, or can partially be. Enucleation can be achieved physically by actual removal of the nucleus, pronuclei, or metaphase plate (depending on the acceptor cell) via mechanical aspiration, centrifugation followed by physical cutting of the cell, or aspiration. Enucleation can also be achieved functionally, such as by the application of ultra-violet radiation; chemically such as via treatment with topoisomerase inhibitors such as ectoposide; or via other enucleating influence.

Following removal of the genetic material from the acceptor cell, genetic material from the nuclear donor cell must be introduced. Various techniques can be used to introduce the genetic material of the nuclear donor cell to the acceptor cell. These techniques include, but are not limited to, cell fusion induced by chemical, viral, or electrical means; injection of an intact nuclear donor cell; injection of a lysed or damaged nuclear donor cell; and injection of the nucleus of a nuclear donor cell into an acceptor cell.

After the transfer of genetic material from the donor to acceptor cell, the acceptor cell must be stimulated to initiate development. In the case of a fertilized zygote, development has already been initiated by sperm entry at fertilization. When using oocytes as acceptor cells, activation must come from other stimuli, such as, application of a DC electric stimulus, treatment with ethanol, ionomycin, Inositol tris-phosphate, calcium ionophore, treatment with extracts of sperm, or any other treatment which induces calcium entry into the oocyte or release of internal calcium stores and results in initiation of development.

Following transfer of genetic material to the acceptor cells and initiation of development, the acceptor cells are then transferred to a recipient female via methods known in the art (see for example Robertson, E. J. “Teratocarcinomas and Embryonic Stem Cells: A Practical Approach” IRL Press, Oxford, England (1987)) and allowed to develop to term.

Nuclear transfer techniques or nuclear transplantation techniques are known in the art (Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, U.S. Pat. Nos. 4,944,384 and 5,057,420).

The present invention provides methods of producing a non-human transgenic animal that express one or more nucleic acid sequences encoding proteins associated with sugar metabolism through the genetic modification of totipotent embryonic cells. In one embodiment, the animals can be produced by: (a) identifying the proteins associated with sugar metabolism to be used to compensate for the aberrant, abnormal, or absent expression of an other protein associated with sugar metabolism; (b) preparing one or more expression vectors containing one or more nucleic acid sequences encoding for proteins associated with sugar metabolism; (c) inserting the one or expression vectors into the genomes of a plurality of totipotent cells of the animal species, thereby producing a plurality of transgenic totipotent cells; (e) obtaining a tetraploid blastocyst of the animal species; (f) inserting the plurality of totipotent cells into the tetraploid blastocyst, thereby producing a transgenic embryo; (g) transferring the embryo to a recipient female animal; and (h) allowing the embryo to develop to term in the female animal. The method of transgenic animal production described here by which to generate a transgenic animal, such as a mouse, is further described, for example, in U.S. Pat. No. 6,492,575.

In another embodiment, the totipotent cells can be embryonic stem (ES) cells. The isolation of ES cells from blastocysts, the establishing of ES cell lines and their subsequent cultivation are carried out by conventional methods as described, for example, by Doetchmann et al., J. Embryol. Exp. Morph. 87:2745 (1985); L1 et al., Cell 69:915-926 (1992); Robertson, E. J. “Tetracarcinomas and Embryonic Stem Cells: A Practical Approach,” ed. E. J. Robertson, IRL Press, Oxford, England (1987); Wurst and Joyner, “Gene Targeting: A Practical Approach,” ed. A. L. Joyner, IRL Press, Oxford, England (1993); Hogen et al., “Manipulating the Mouse Embryo: A Laboratory Manual,” eds. Hogan, Beddington, Costantini and Lacy, Cold Spring Harbor Laboratory Press, New York (1994); and Wang et al., Nature 336:741-744 (1992).

In a further embodiment of the invention, the totipotent cells can be embryonic germ (EG) cells. Embryonic Germ cells are undifferentiated cells functionally equivalent to ES cells, that is they can be cultured and transfected in vitro, then contribute to somatic and germ cell lineages of a chimera (Stewart et al., Dev. Biol. 161:626-628 (1994)). EG cells are derived by culture of primordial germ cells, the progenitors of the gametes, with a combination of growth factors: leukemia inhibitory factor, steel factor and basic fibroblast growth factor (Matsui et al., Cell 70:841-847 (1992); Resnick et al., Nature 359:550-551 (1992)). The cultivation of EG cells can be carried out using methods known to one skilled in the art, such as described in Donovan et al., “Transgenic Animals, Generation and Use,” Ed. L. M. Houdebine, Harwood Academic Publishers (1997).

Tetraploid blastocysts for use in the invention can be obtained by natural zygote production and development, or by known methods by electrofusion of two-cell embryos and subsequently cultured as described, for example, by James et al., Genet. Res. Camb. 60:185-194 (1992); Nagy and Rossant, “Gene Targeting: A Practical Approach,” ed. A. L. Joyner, IRL Press, Oxford, England (1993); or by Kubiak and Tarkowski, Exp. Cell Res. 157:561-566 (1985).

The introduction of the ES cells or EG cells into the blastocysts can be carried out by any method known in the art, for example, as described by Wang et al., EMBO J. 10:2437-2450 (1991).

A “plurality” of totipotent cells can encompass any number of cells greater than one. For example, the number of totipotent cells for use in the present invention can be about 2 to about 30 cells, about 5 to about 20 cells, or about 5 to about 10 cells. In one embodiment, about 5-10 ES cells taken from a single cell suspension are injected into a blastocyst immobilized by a holding pipette in a micromanipulation apparatus. Then the embryos are incubated for at least 3 hours, possibly overnight, prior to introduction into a female recipient animal via methods known in the art (see for example Robertson, E. J. “Teratocarcinomas and Embryonic Stem Cells: A Practical Approach” IRL Press, Oxford, England (1987)). The embryo can then be allowed to develop to term in the female animal.

In one embodiment of the invention, the methods of producing transgenic animals, whether utilizing nuclear transfer, embryo generation, or other methods known in the art, result in a transgenic animal comprising a genome that does not contain significant fragments of the expression vector used to transfer nucleic acid sequences encoding proteins associated with sugar metabolism. The term “significant fragment” of the expression vector as used herein denotes an amount of the expression vector that comprises about 10% to about 100% of the total original nucleic acid sequence of the expression vector. This excludes the nucleic acid sequences encoding proteins associated with sugar metabolism insert portion that was transferred to the genome of the transgenic animal. Therefore, for example, the genome of a transgenic animal that does NOT contain significant fragments of the expression vector used to transfer the nucleic acid sequences encoding proteins associated with sugar metabolism, can contain no fragment of the expression vector, outside of the sequence that contains the nucleic acid sequences encoding proteins associated with sugar metabolism. Similarly, the genome of a transgenic animal that does not contain significant fragments of the expression vector used to transfer the nucleic acid sequences encoding proteins associated with sugar metabolism can contain about 1%, about 2%, about 3%, about 4%, about 5%, about 6%, about 7%, about 8%, about 9%, or about 10% of the expression vector, outside of the sequence that contains the nucleic acid sequences encoding proteins associated with sugar metabolism. Any method which allows transfer of the nucleic acid sequences encoding proteins associated with sugar metabolism to the genome while also limiting the amount of the expression vector that is also transferred to a fragment that is not significant can be used in the methods of the present invention.

Certain aspects of the invention can be described in greater detail in the non-limiting Examples that follow.

### EXAMPLES

### Example 1

### The Effect of a Galactose-Rich Diet and Carbon Dioxide Exposure on α1,3GT Knockout Mice

To elucidate the underlying mechanism(s) of the galactosemia, as measured by the formation of early onset cataracts (EOC), in the α1,3GT-knock-out (KO) mouse, the influence of a) a galactose-rich diet and b) carbon dioxide (CO₂) exposure on the 129 SV α1,3GT was studied.

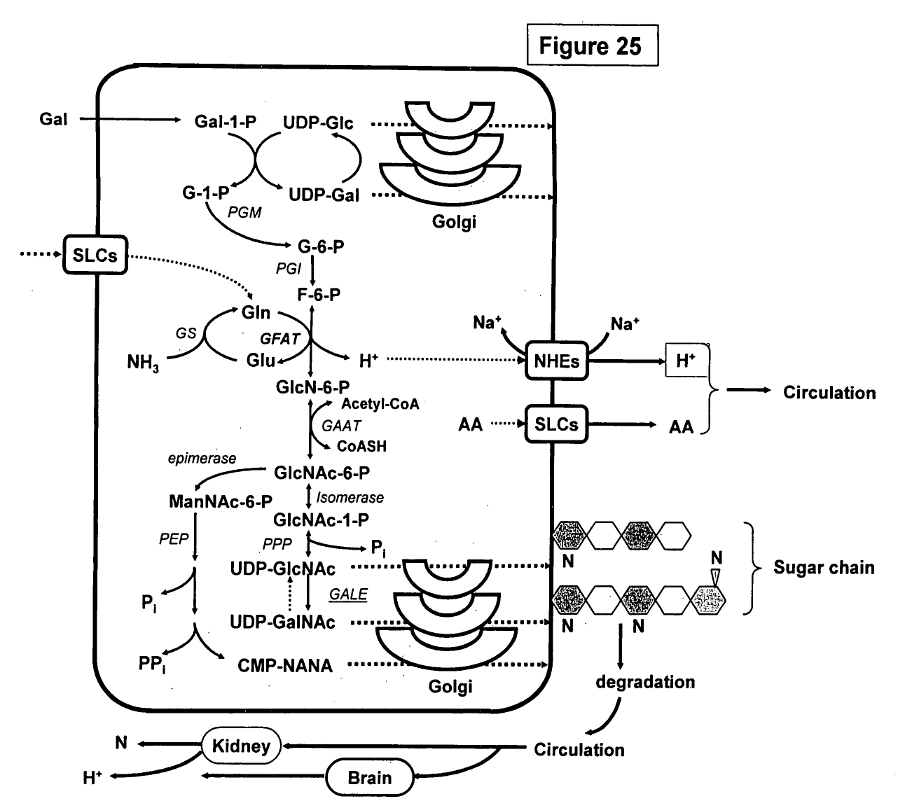

The α1,3GT-double knockout mice exhibited EOC soon after weaning, however, the EOC was slight, generally being of a pinhead size (FIG. 26-a). Wild type (WT) and the α1,3GT-double knockout mice were divided into 4 groups (n=10, each). Each group was fed either galactose-rich diet (40, 20, or 10% galactose) or normal diet (4.5% galactose). No cataract formation was observed in the WT mice even at the 40% diet level. The cataract size in the α1,3GT-double knockout mice remained the same regardless of the galactose concentration.

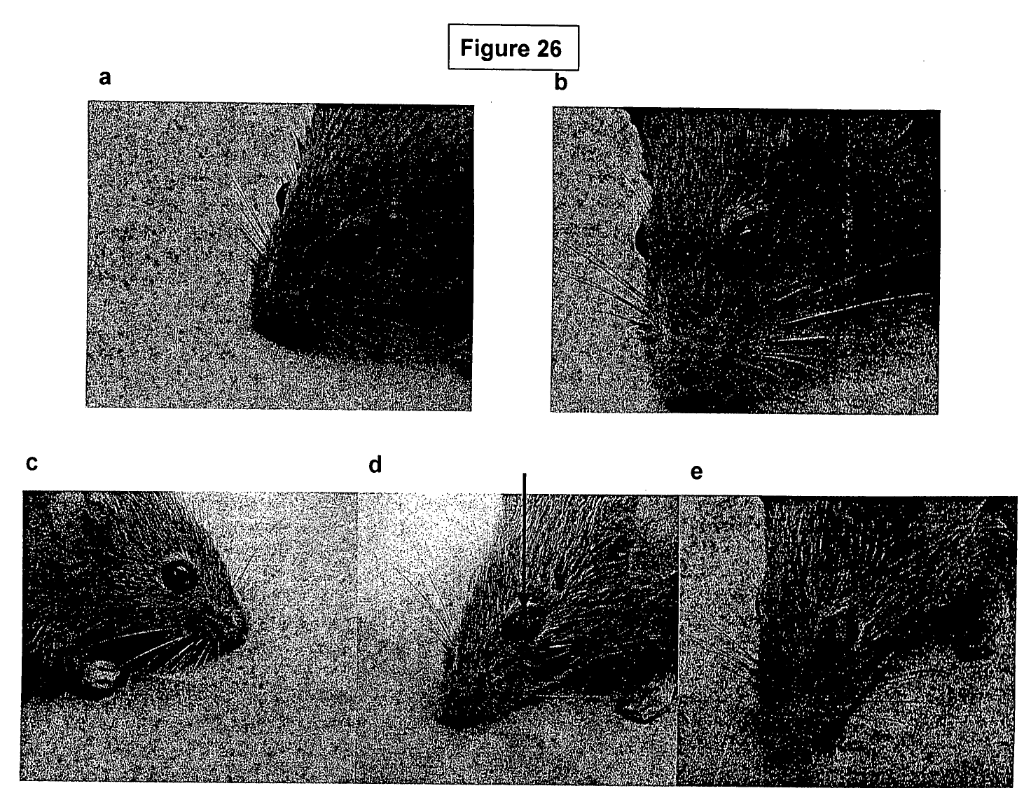

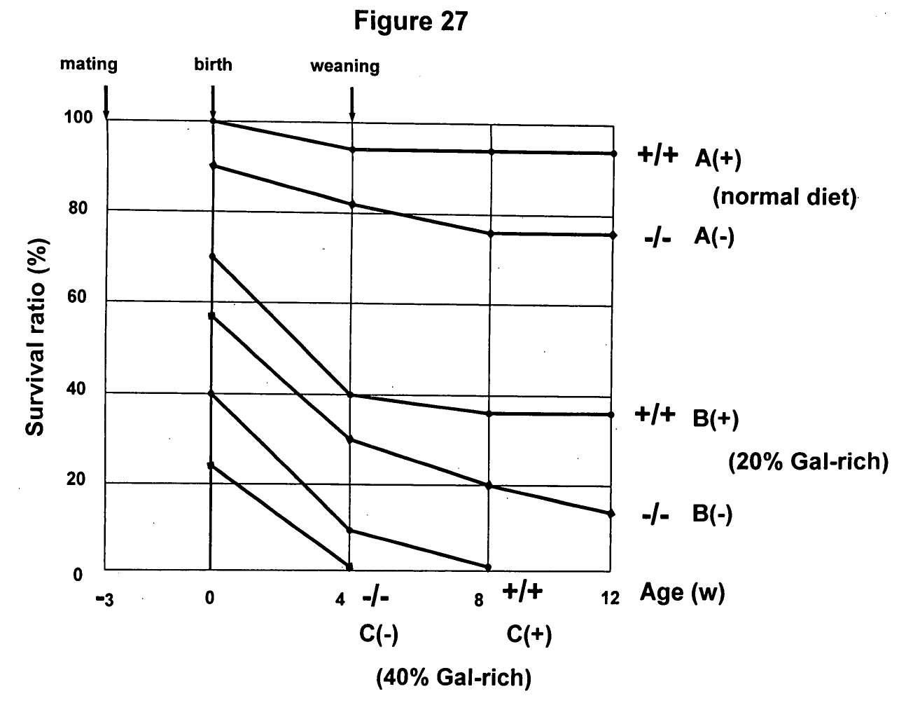

However, long term feeding of a galactose-rich diet resulted in systemic impairment. Both WT and α1,3GT-double knockout mice fed galactose-rich diets gradually appeared less healthy. The mice were visually less active, developed a harsher coat, continuous closed eyes and a rounded back posture, amongst other things. Increased water intake and polyuria were also noted. Fewer pups were born from both WT and α1,3GT double knockout mothers fed the 40% galactose-rich diet. Those pups, much smaller than the normal control, died before weaning, resulting in the production of no progeny in both WT and α1,3GT-knockout mice (FIG. 27).

In mice fed the 20% galactose-rich diet, litter sizes were smaller in both WT and α1,3GT double knockout mice than comparative controls. Approximately half of the progeny survived weaning, but no progeny of either mouse type produced next generation offspring while being fed the 20% galactose-rich diet. When the galactose-rich diet was replaced with the normal diet, the mice were able to thrive and reproduce next generation offspring. However, the litter size was still smaller in the α1,3GT double knockout than that of WT (FIG. 27). Thus, it was demonstrated that galactose-rich diet is toxic to the mouse in a dose-dependent manner.

b) Carbon Dioxide Exposure

The α1,3GT double knockout mice exposed to CO₂ (carbon dioxide), experienced prompt enlargement of cataract opacity (FIG. 26-b). Comparatively, no change was observed in the opacity of the lens of WT mice. Strikingly, when the exposure time was less than 15 second, the enlarged opacity gradually became smaller as spontaneous hyperventilation recovered under room air, and returned to the original size (FIG. 26-c). These animal experiments were run in triplicate with similar results.

The results of the galactose diet exposure experiment and carbon dioxide exposure experiment shed light on the role sugars and sugar chains play in cellular homeostasis. The enlargement of the cataract size in the α1,3GT double knockout mice in the presence of CO₂ followed by the reversal in its absence, and the compensation of loss of the α1,3Gal expression by enhanced expression of sialic moieties imply that the α1,3Gal expression is directly linked to galactose metabolism, sugar chain synthesis, hexosamine synthesis, and acid-base homeostasis.

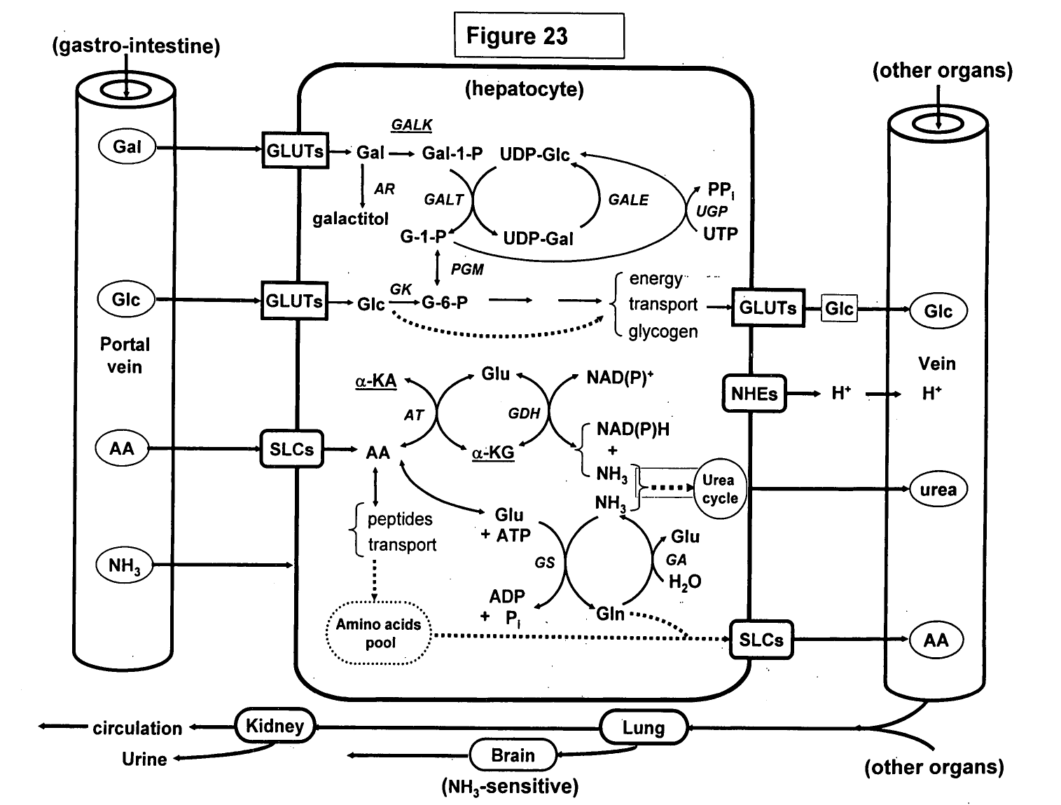

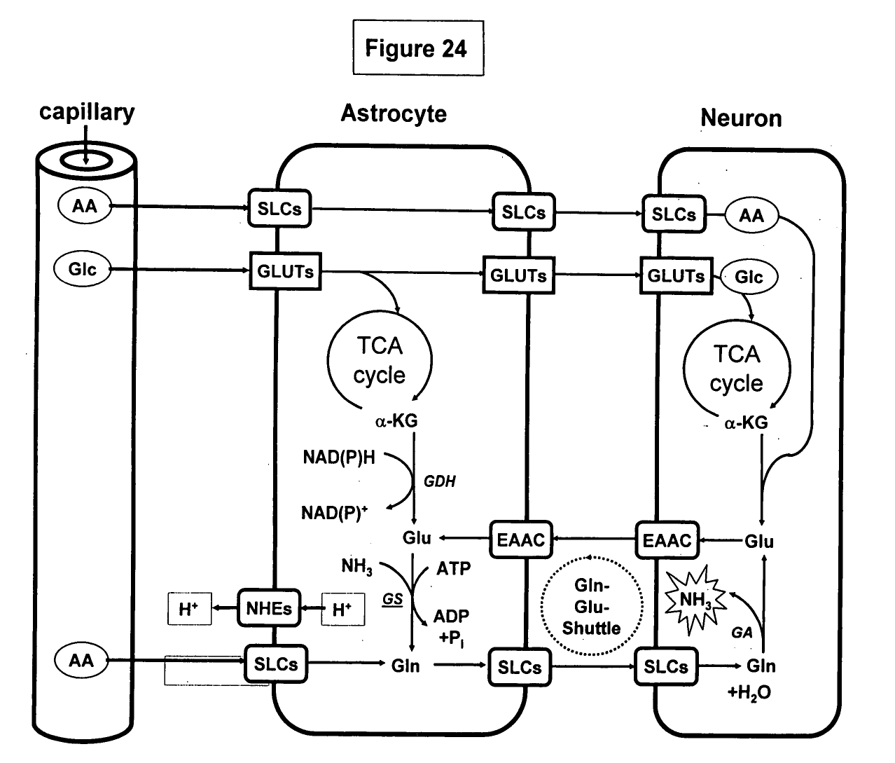

The NHE system in the α1,3GT double knockout mice must deal with the elevated level of hydrogen ion produced as a result of expressing sialic acids to compensate loss of the α1,3Gal expression, which in turn produces an intracellular acidosis-prone state. Because of this, α1,3GT double knockout mice were unable to promptly react against the extra-cellular respiratory acidosis produced by CO₂ inhalation. Normally, the extracellular acidotic state produced by inhalation of CO₂ is partially reduced through the intracellular import of hydrogen ions through the NHE system (see, for example, FIGS. 24 and 24). Because of the already increased intracellular hydrogen ion concentrations, the intracellular import is significantly reduced. This intracellular acidotic state likely accounted for the observation that the pinhead size of the EOC promptly enlarged with inhalation of carbon dioxide (FIG. 25).

### Example 2

### Evolution of α-1,3-GT in Higher Primates

The α1,3-galactosyltransferase (α1,3GT) gene (Blanken, W. M et al. J. Biol. Chem. 260, 12927-12934 (1985)) was inactivated 23 MYA, contemporaneous with higher primate emergence (Glazko, G. V. et al. Mol. Biol. Evol. 20, 424434 (2003)). Alignment of the active gene and unprocessed and processed α1,3GT pseudogenes of multiple αGal-positive and negative species allowed reconstruction of 4 protogenes thought to have been expressed successively between 56-23 MYA. Throughout this period, selection pressure on the enzyme's stem region favored expression for prevention of intra-Golgi UDP-galatose accumulation. α1,3GT inactivation apparently occurred when glycoconjugate enzyme(s) substituted for this housekeeping function, allowing other changes that powerfully propelled speciation. The inactivation was thereby causal in higher primate emergence.

The α1,3Gal epitope is expressed at the surface of cells of essentially all lower mammals and of the new world monkeys (NWM) that are grouped as platyrrhines (e.g. cebus and marmoset), but not in any of the higher primates (old world monkeys [OWM], apes, and humans) that are collectively termed catarrhines (Galili, U et al. J. Biol. Chem. 263, 17755-17762 (1988)). In turn, catarrhines secrete “natural” anti-αGal antibodies that cause immediate (hyperacute) rejection of tissues and organs transplanted from α1,3Gal-positive to these α1,3Gal-negative species (Good, A. H et al. Transplant. Proc. 24, 559-562 (1992)). The reciprocal relation of α1,3Gal epitope to cognate natural antibodies is similar to that of the A, B, and H antigens of the ABO histo-blood group system. Both the α1,3Gal and the ABH antigens are members of a large family of sugar chains whose biologic role(s) is poorly understood. The molecular basis for expression of the bovine α1,3Gal epitope and for expression of the human ABO system were described in 1989 (Joziasse, D. H et al. J. Biol. Chem. 264, 14290-14297 (1989)) and 1990 (Yamamoto, F et al. Nature 345, 229-233 (1990)), respectively.

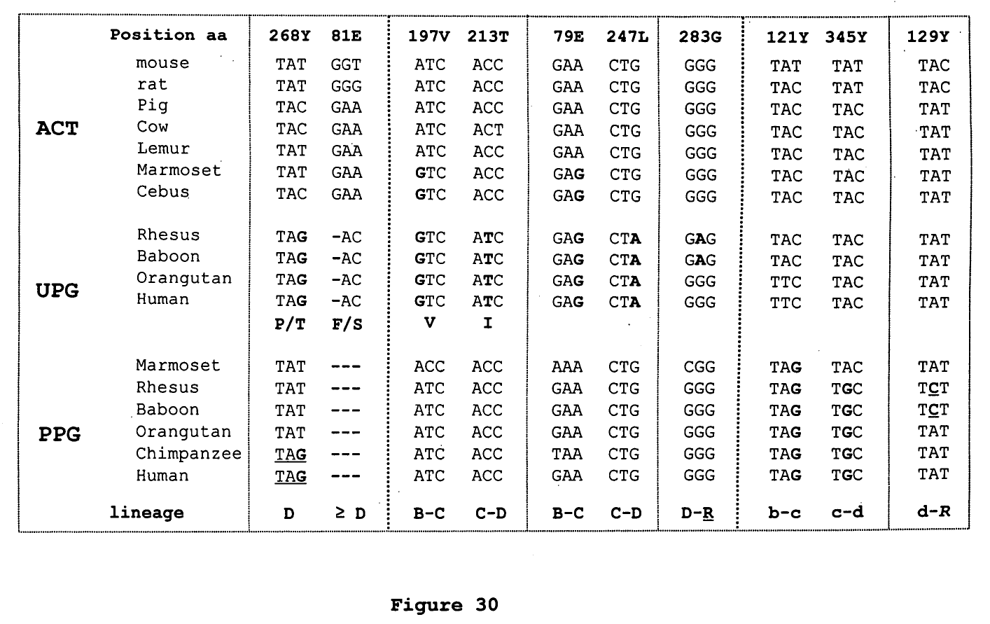

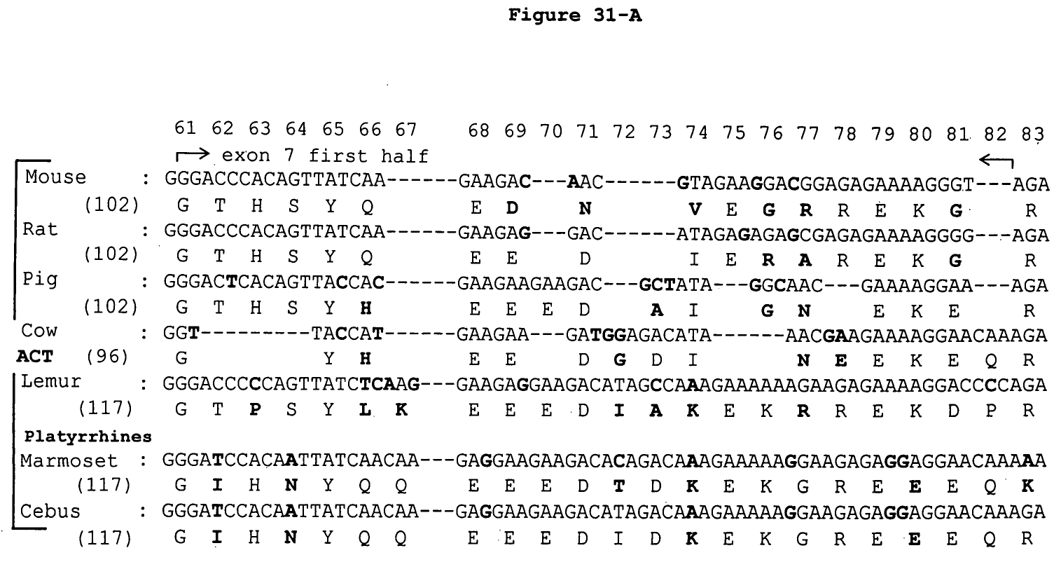

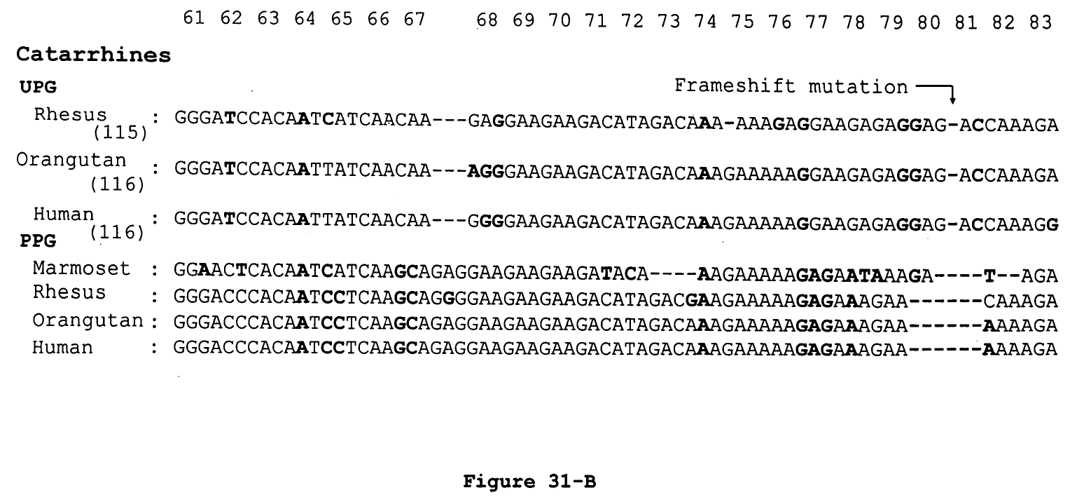

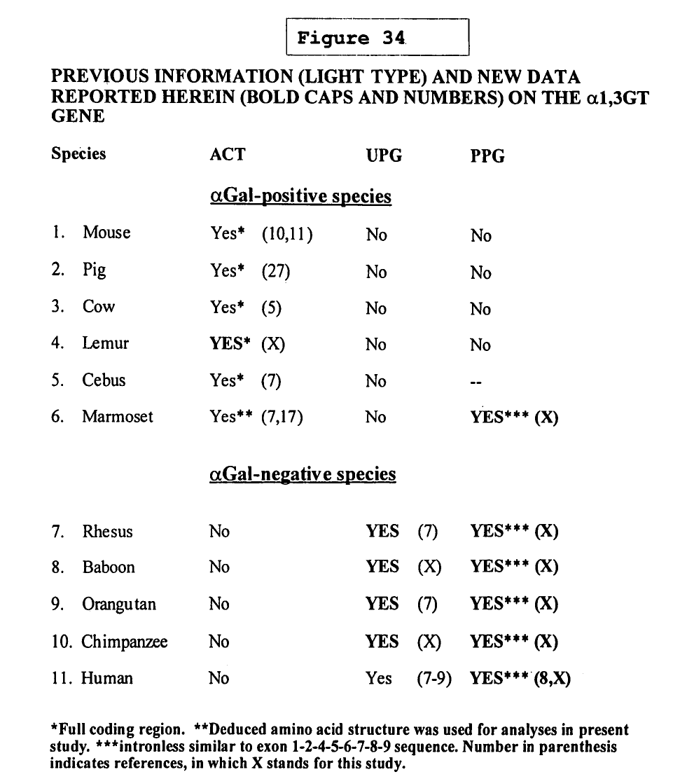

The molecular basis for the inactivation of the α1,3Gal antigen in catarrhines was not fully elucidated until 2002 (Koike, C et al. J. Biol. Chem. 277, 10114-20 (2002)). As early as 1991, however, short sequences (Joziasse, D. H et al. J. Biol. Chem. 266, 6991-6998 (1991), Larsen, R. D. et al. J. Biol. Chem. 266, 7055-7061 (1990)), of an inactivated α1,3GT gene (i.e. unprocessed pseudogene [UPG]) homologous to portions of the bovine (Joziasse, D. H et al. J. Biol. Chem. 264, 14290-14297 (1989)). Good, A. H et al. Transplant. Proc. 24, 559-562 (1992) and mouse Larsen, R. D. et al. Proc. Natl. Acad. Sci. USA. 86, 8227-8231 (1989). α1,3GT gene were found in human chromosome 9 (Shaper, N. L. et al. Genomics 12, 613-615 (1992)). In addition, a processed (intronless) pseudogene (Wilde, C. D. et al. Nature 297, 83-84 (1982)) [PPG] resembling the α1,3GT cDNA of α1,3Gal-positive species was demonstrated in human chromosome 12 (Wilde, C. D. et al. Nature 297, 83-84 (1982)) and termed HGT-2 (ref.8). Further progress was forestalled for nearly a decade until xenotransplantation-related studies led to the discovery of a variety of α1,3GT mRNA transcripts in the rhesus, orangutan, and human cDNA libraries (Koike, C et al. J. Biol. Chem. 277, 10114-20 (2002)). The full coding region and the exon-intron structure of the α1,3GT UPG in these 3 different species were then elucidated (FIG. 34). Multiple mutations that could have resulted in gene inactivation were identified, 2 of which were shared by all 3 species (Koike, C et al. J. Biol. Chem. 277, 10114-20 (2002)). The data suggest that baboon and chimpanzee UPG also share these mutations: position 81 E of exon 7 and 268Y of exon 9 (FIGS. 30 and 34).

The intronless α1,3GT PPG, which was an indispensable genetic marker for the alignment studies herein reported, has a nucleotide sequence similar to much of the major porcine transcript (FIG. 30). Presumably produced by a retrotransposon (Vanin, E. F. Annu. Rev. Genet. 19, 253-72 (1985)), this PPG was found in all 5 catarrhines studied and in the marmoset (a platyrrhine) (FIG. 34). It was not present, however, in the lemur (a prosimian) or in any other lower mammalian species examined. These findings, clearly demonstrate that the PPG was generated before inactivation of the α1,3GT source gene, rather than after as previously postulated (Larsen, R. D. et al. J. Biol. Chem. 266, 7055-7061 (1990), (Joziasse, D. H., Oriol, R. Bioch. Biophy. Acta. 1455, 403418 (1999)). A key element in the earlier hypothesis was the assumption that the TAG at 268Y in the human PPG (HGT-2) had been present throughout the entire platyrrhine-catarrhine period. Instead, this mutation in the PPG was found only in the late catarrhines (FIG. 30).

Using the full coding region of the marmoset as reference, the UPG and PPG of the 5 αGal-negative catarrhines and the PPG of the αGal-positive marmoset were aligned against the full coding region of the active α1,3GT gene of the different species (including lemur) shown in FIG. 30. Transition mutations (substitution between A and G, or C and T) and transversion mutations (substitutions other than transition. [15]) that corresponded to the marmoset cDNA coding region were determined, based on which lineage a given nucleotide did or did not mutate (FIG. 30). Deletion and addition mutations that could not be uniquely assigned were excluded from analysis (Casane, D. et al. J. Mol. Evol. 45, 216-26 (1997)). The ancestral nucleotide state was inferred for each polymorphic site with the generally accepted premise that the ancestral nucleotide was the one that required the minimum number of substitutions to account for the ultimate differences (Henion, T. R., Galili, U. Subcell Biochem. 32, 49-77 (1999)).

The alignment revealed a total of 16 homologous sequences, ranging from 1107-1131 bp in the 12 extant species (Joziasse, D. H et al. J. Biol. Chem. 264, 14290-14297 (1989), (Larsen, R. D. et al. Proc. Natl. Acad. Sci. USA. 86, 8227-8231 (1989)), (Koike, C et al. J. Biol. Chem. 277, 10114-20 (2002), (Henion, T. R., Galili, U. Subcell Biochem. 32, 49-77 (1999)). Most of the 1107-1131 bp variability was in exon 7: 102 bp in rodents and pig, 96 in cow, and 117 in the lemur, marmoset, and cebus. It was not previously recognized that almost all of the length variation was in the mutation-rich first half of this exon. The data showed this, and indicate that the mutation-rich first half of exon 7 corresponds with the stem region. The second half of exon 7 starting with 83K in the marmoset is as highly preserved as in exons 4, 8, and 9 and is the beginning of the catalytic domain. The findings explain the observation that splicing out exon 7 reduces gene activity >95% (Henion, T. R., Galili, U. Subcell Biochem. 32, 49-77 (1999)).

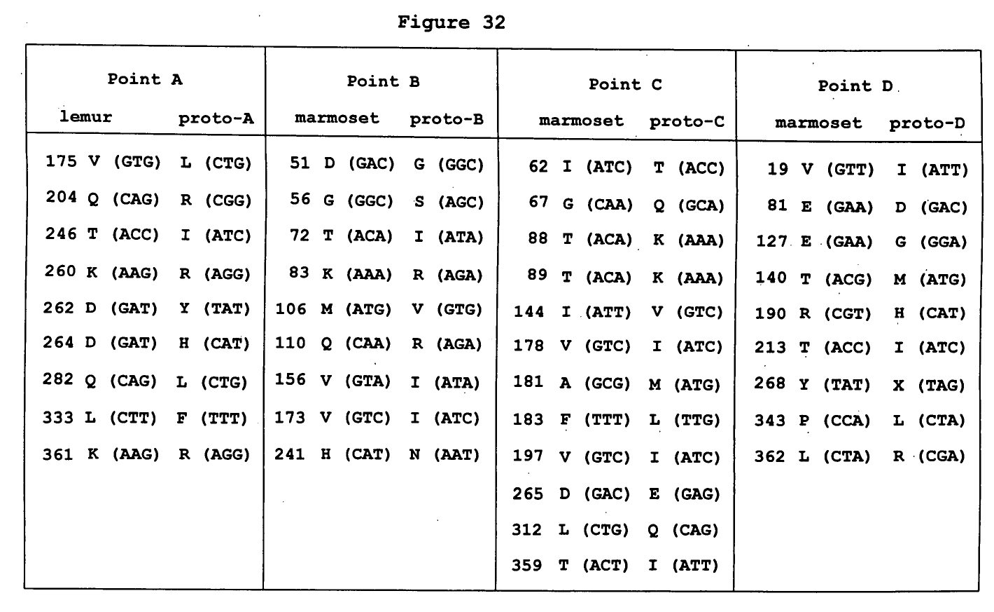

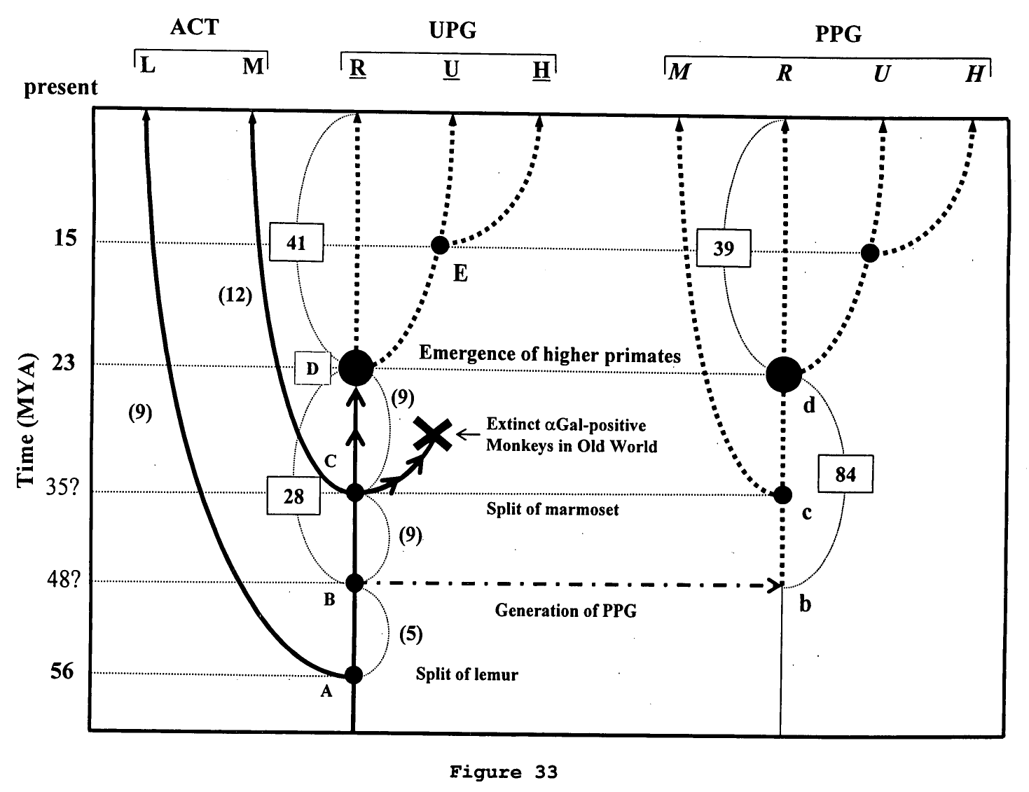

The alignment analysis allowed elucidation of 4 distinct α1,3GT cDNA sequences (i.e. protogenes) that could have been expressed in succeeding periods between the split of prosimians from a common mammalian lineage 56 MYA (Kumar, S., Hedges, B. Nature 392, 917-920 (1998), Bowen, G. J. et al. Science 295, 2062-2065 (2002)) and the emergence of higher primates (and α1,3GT inactivation) 23 MYA (Glazko, G. V. et al. Mol. Biol. Evol. 20, 424-434 (2003). Throughout this approximately 33 MY period and to the present day, the 16 key amino acids of exons 8 and 9 that have been described as essential for α1,3GT expression (Y147, W203, S207, R210, D233, D235, Q236, Q255, W258, W258, T267, W322, D324, E325 and W364 and H288 [20,21]) were identical to the amino acids of the catalytic domain of all modern α1,3Gal-positive mammals ((Joziasse, D. H et al. J. Biol. Chem. 264, 14290-14297 (1989), (Larsen, R. D. et al. Proc. Natl. Acad. Sci. USA. 86, 8227-8231 (1989)), (Koike, C et al. J. Biol. Chem. 277, 10114-20 (2002), (Henion, T. R., Galili, U. Subcell Biochem. 32, 49-77 (1999), Shetterly, S. et al. J Glycobiol. 11, 645-653 (2001)) including the lemur (data not shown). The non-synonymous mutations that occurred between the time of protogene A (56 MYA) and the present day lemur, and between protogene C (35 MYA) and the current marmoset (Glazko, G. V. et al. Mol. Biol. Evol. 20, 424434 (2003)), (Koike, C et al. J. Biol. Chem. 277, 10114-20 (2002)), (Henion, T. R., Galili, U. Subcell Biochem. 32, 49-77 (1999)), are shown in FIG. 32, and depicted graphically FIG. 33.

The 56 MYA (Kumar, S., Hedges, B. Nature 392, 917-920 (1998)), (Bowen, G. J. et al. Science 295, 2062-2065 (2002)) and 23 MYA (Glazko, G. V. et al. Mol. Biol. Evol. 20, 424-434 (2003)). used to anchor the chronology (protogenes A and D) are generally accepted, based on fossil and molecular evidence. There is less complete concensus that platyarrhines MYA (Glazko, G. V. et al. Mol. Biol. Evol. 20, 424434 (2003)), (Jones, S et al., The Cambridge Encyclopedia of Human Evolution. Cambridge University Press. Cambridge, UK. pp. 197-230 (1992)), (Napier, J. R., Napier, P. H. The natural history of the primates. The MIT Press, Cambridge, Mass. pp. 20-60 (1985)) emerged 35 MYA (protogene C). The demonstration of the α1,3GT PPG in the current marmoset but not in the lemur or any other lower mammal places generation of the PPG by protogene B between protogenes A and C. With the assumption that this occurred 48 MYA, the time intervals of events between Points A-B, B-C, C-D, and D—to present were estimated by analysis of mutation rates of the active α1,3GT gene and of the UPGs and PPGs (FIG. 33). The bold lines connote certain α1,3GT expression. Bold lines with arrows represent deduced expression.

Substitution mutations during the D-R period in the rhesus UPG numbered 41, essentially the same as in the rhesus PPG (n=39) (FIG. 32 d-R). In contrast, the mutations that preceded 23 MYA (B-D in FIG. 32) numbered 28 (of which 18 were non-synonymous), while the mutations in the PPG in the same earlier period (b-d in FIG. 32) totaled 84 (2.9 fold faster). Because nonfunctional sequences mutate much faster than functioning genes that are subject to selection pressure (Strachan, T., Read, A. Human Molecular Genetics. A John Wiley & Sons, Inc., New York, N.Y. pp. 241-273 (1996)), the mutation rates are congruent with the independently derived conclusion (Jones, S et al., The Cambridge Encyclopedia of Human Evolution. Cambridge University Press. Cambridge, UK. pp. 197-230 (1992)), (Napier, J. R., Napier, P. H. The natural history of the primates. The MIT Press, Cambridge, Mass. pp. 20-60 (1985)) that emergence of higher primates 23 MYA was contemporaneous with inactivation of the α1,3GT gene.

Importantly, it is emphasized that a change in the mutation rate of the PPG per se occurred at 23 MYA. Assuming that the PPG was generated 48 MYA, it underwent 84 mutations between 48-23 MYA (3.4/MY), 2-fold greater than the 39 mutations that occurred between 23 MYA and the present time (1.7/MY) (compare b-d with d-R, FIG. 32). The reduction by half of the PPG mutation rate would be even more pronounced if the PPG was generated later (e.g. to 35% if PPG generation occurred 40 MYA). The striking decrease in mutation is congruent with the lengthening of time between the production of offspring (generation time) and of ontogeny that is known to have occurred in higher primates after 23 MYA (L1, W.-H., Grauer, D. “Fundamentals of Molecular Evolution”, Sinauer, Sunderland, Mass., (1991)).

When the framework provided by the totality of the studies of the α1,3GT gene is transposed on what is known from fossil and molecular research (FIG. 33), it helps fill gaps in information of primate evolution from 56 MYA-present, and especially the 15 MY period preceding gene inactivation. In the fossil-based classical view, platyrrhines and early catarrhines were thought to have split from a common anthropoid lineage approximately 35 MYA (Jones, S et al., The Cambridge Encyclopedia of Human Evolution. Cambridge University Press. Cambridge, UK pp. 197-230 (1992)), (Napier, J. R., Napier, P. H. The natural history of the primates. The MIT Press, Cambridge, Mass. pp. 20-60 (1985)). The Oligopithecus, Propliopithecus, and Aegyptopithecus, whose fossil remains were identified in the Fayum deposits of Egypt and dated 30 MYA, were considered to be the immediate precursors of higher primates.

These primitive primates were diminutive (maximum estimated weight 6 kg) and had other features resembling present day NWM (Jones, S et al., The Cambridge Encyclopedia of Human Evolution. Cambridge University Press. Cambridge, UK. pp. 197-230 (1992)), (Napier, J. R., Napier, P. H. The natural history of the primates. The MIT Press, Cambridge, Mass. pp. 20-60 (1985)). The principal rationale for viewing them as higher primate precursors was the similarity of their dental formula to that of current catarrhines: i.e., 32 teeth and narrow nostril versus the 36 teeth and wide nostril of all platyrrhines except the marmoset (32 teeth). These extinct species could have been the short lived ancient anthropoid that presumably expressed the proto α1,3GT gene (Proto C) (X in FIG. 33). The findings also are consistent with the combined fossil and molecular evidence that dates the emergence of higher primates to 23 MYA (Glazko, G. V. et al. Mol. Biol. Evol. 20, 424-434 (2003)). The appearance of the Prohylobates tandyi and P. simosi of Wadi Moghara (Egypt) and Gebel Zeltan (Libya) at this time heralded the beginning of the Miocene radiation (Jones, S et al., The Cambridge Encyclopedia of Human Evolution. Cambridge University Press. Cambridge, UK. pp. 197-230 (1992)), (Napier, J. R., Napier, P. H. The natural history of the primates. The MIT Press, Cambridge, Mass. pp. 20-60 (1985)) that coincided with α1,3GT inactivation.

What caused (or permitted) α1,3GT inactivation? This has been attributed to selection pressure exerted by the threat of α1,3Gal-expressing micro- or macro-pathogens ((Glazko, G. V. et al. Mol. Biol. Evol. 20, 424434 (2003)), Joziasse, D. H et al. J. Biol. Chem. 266, 6991-6998 (1991)), Joziasse, D. H., Oriol, R. Bioch. Biophy. Acta. 1455, 403418 (1999)). The hypothesis is weakened by the fact that no examples of α1,3Gal-negative species are known to have appeared during the more than 125 million years of lower mammalian evolution (Ji, Q et al. Nature 416, 816-822 (2002)). Moreover, the alignment analyses do not lend support to the theory. Despite continuous nucleotide mutation, and especially that in the ostensible stem region of the gene, the remarkable homology of the catalytic domain suggests that selection pressure conspired until 23 MYA in favor of retention of α1,3Gal expression for reason(s) other than any potential immunologic advantage of inactivation.

The data suggest that expression of the α1,3GT gene acted as a physiologic constraint(s) (i.e. as a housekeeping gene [Strachan, T., Read, A. Human Molecular Genetics. A John Wiley & Sons, Inc., New York, N.Y. pp. 241-273 (1996); Koike, C et al. Transplant. 70, 1275-1283 (2000)]), and that the primary constraint was prevention of detrimental accumulation of intra-Golgi UPD-galactose. In this view, gene inactivation became consistent with survival in the wild only when other glycoconjugate enzyme(s) substituted efficiently for delivery of UPD-galactose to the cell membrane. The result was a different cell surface epitope(s) (e.g. ABH antigens). Although potentially important, any consequent immunologic advantage would have been fortuitous.

Survival after α1,3GT inactivation undoubtedly necessitated multiple other changes. A specific example was described by Zhang and Webb in their studies of the molecular basis for the loss 23 MYA of pheromone signal transduction pathways (Zhang, J., Webb, D. M.\. Proc. Natl. Acad. Sci. USA, 100, 8337-8341 (2003)). The authors suggested that the resulting reduced ability to detect pheromones would have profoundly altered the social-reproductive practices of higher primates and made these practices dependent on more discriminating vision (including color). Although Zhang and Webb did not associate involution of the vomeronasal organ with inactivation of the α1,3GT gene, Takami, Getchell and Getchell (Takami, S. et al. Cell Tissue Res. 280, 211-216 (1995)) previously had described in the rat a dense concentration of α1,3Gal epitopes in the organ's sensory neurons and extracellular mucoid components. Disappearance of α1,3Gal epitopes from the olfactory organ could explain why higher primates have only a vestigial vomeronasal apparatus.

Additional derivative changes after α1,3GT inactivation would have included the extension of generation time and increased body growth implicit in the results of the mutation rate analyses, as well as accelerated brain development. It is noteworthy that a similar but less dramatic chain of events with the arrival of modern humanoids 2.8 MYA has been associated by Chou and Varki et al (Chou, H et al. Proc. Natl. Acad. Sci. USA. 99, 11736-11741 (2002)) with inactivation of the gene encoding the enzyme CMAH (CMP-N-acetylneuraminic acid hydroxylase) responsible for synthesis of the glycoconjugate Neu5Gc (N-glycolyoneuraminic acid).

In summary, dynamic changes in the biochemistry and genetics of carbohydrate metabolism seem to have exerted a powerful force propelling speciation. Inactivation of the α1,3GT gene could have been causal in the dramatic evolutionary events that allowed the emergence of higher mammalian species and eventuated in the ascent of man.

Materials and Methods

Tissues Examined

Whole blood from the lemur (Lemur catta), marmoset (Callithrix jacchus), rhesus (Macaca Mullata), orangutan (Pongo pygmaeus) and chimpanzee (Pan paniscus) was kindly provided by the Pittsburgh Zoo (Pittsburgh, Pa.), University of Wisconsin-Madison (Madison, Wis.), or the Duke University Primate Research Center (Durham, N.C.). Human blood samples were obtained from normal adult volunteers.

Isolation of Nucleic Acids

To isolate high molecular weight genomic DNA from the respective samples, standard methods were employed. Total RNA was extracted from the samples with Trizol reagent (Gibco). Poly A+ RNA was separated from total RNA using the Dynabeads mRNA Purification Kit (Dynal, Oslo, Norway) according to the manufacturer's instructions.

Construction of GenomeWalker™ Libraries

GenomeWalker™ libraries for the respective species were constructed using the Universal GenomeWalker™ Library Kit (Clontech, Palo Alto, Calif.). Human processed α1,3GT pseudogene was obtained with GenomeWalker-PCR (GW-PCR). Gene-specific primers (Table A) were designed from the human PPG (i.e. the HGT-2 sequence [8]). For the marmoset, rhesus and orangutan counterparts of HGT-2, primers were designed from the exon 8 and exon 9 sequences of the unprocessed pseudogene of the respective species. For the lemur α1,3GT active gene, the human unprocessed gene primers were utilized. TaKaRa LA Taq (Takara Shuzo Co., Ltd., Shiga, Japan) enzyme was used for all PCR experiments. The PCR thermal cycling conditions, recommended by the manufacturer, were performed on a Perkin Elmer Gene Amp System 9600 or 9700 thermocycler.

Construction of the RACE and RT-PCR Libraries

To identify the 5′- and 3′-ends of the α1,3GT gene transcripts of the lemur, baboon, and chimpanzee, the Marathon™ RACE (rapid amplification of cDNA end) libraries (Clontech) were constructed from total RNA of the respective species in accordance with the manufacturer's specified protocol. SuperScript Preamplification System™ (Gibco) was used according to the manufacturer's instructions for the generation of first strand cDNA template for RT-PCR.

Subcloning and Sequencing of Amplified Products

PCR products amplified by the GW-PCR, RACE-PCR, and RT-PCR were subcloned into the pCR II™ vector provided with the Original TA Cloning™ Kit (Invitrogen, Carlsbad, Calif.). Automated fluorescent sequencing of cloned inserts was performed using an ABI 377 Automated DNA Sequence Analyzer (Applied Biosystems, Inc., Foster City, Calif.).

Sequence of Oligonucleotides Used as PCR Primers

Primer sequences used for identify the various genes are as follows.

Rhesus processed pseudogene:

(Seq ID No. 53)

Rpa: 5′-GGTGAGTGGATGGATGATGGGGAGGAG-3′,

(Seq ID No. 54)

Rpq: 5′-CAAGCTGATCTCGAACTCCTGACCTCACGTG-5′.
Orangutan processed pseudogene:

(Seq ID No. 55)

Upa: 5′-GTCAAAGGGGATACGTTTTTCCCGGCAG-3′,

(Seq ID No. 56)

Upq: 5′-ACCATAGATTCATTCTCTCATATTAGAGTGGTC-3′.
Human processed pseudogene:

(Seq ID No. 57)

Hpa: 5′-CTGCTAAGCTCAGGTGATGCACTGGGC-3′,

(Seq ID No. 58)

Hpq: 5′-GAATCAAGGGTATAGCCCCGTACAACCA-3′.
Lemur gene:

(Seq ID No. 59)

L9A: 5′-CATCATGCTGGACGACATCTCGAAGATGC-3′,

(Seq ID No. 60)

L9B: 5′-CAAGCCTGAGAAGAGGTGGCAGGACATC-3′,

(Seq ID No. 61)

L9P: 5′-GTATGCTGAGTTTACGCCTCTGATAGG-3′,

(Seq ID No. 62)

L9Q: 5′-GTAGCTGAGCCACTGACTGGCCGAG.

Alignment Analyses

Transition mutations (substitution between A and G, or C and T) and transversion mutations (substitutions other than transition) corresponding to the marmoset α1,3GT cDNA coding region were determined on the basis of which lineage a given nucleotide did or did not mutate. Other kinds of mutations (e.g. deletions or additions or those that could not be uniquely assigned) were excluded from this assignment analysis. The direction of the mutation and the ancestral nucleotide state were inferred for each polymorphic site. This required the assumption that the ancestral nucleotide is the one that requires the minimum number of substitutions to account for the nucleotide differences (Casane, D. et al. J. Mol. Evol. 45, 216-26 (1997).

The GenBank accession numbers used in this analysis were as follows: Processed α1,3GT pseudogene: Rhesus; AF521019, Orangutan; AF521020, Human; AF378672; Unprocessed α1,3GT pseudogene: Rhesus; AY026225-AY026237, Orangutan; AF456457, Human; AF378121-AF378123; and Active α1,3GT gene: Marmoset; AF384428, Cebus: AY034181, Lemur: AY126667.

This invention has been described with reference to its preferred embodiments. Variations and modifications of the invention, will be obvious to those skilled in the art from the foregoing detailed description of the invention. It is intended that all of these variations and modifications be included within the scope of this invention.

We claim:

1. A galactose deficient cell comprising a genetic modification that results in expression of a protein of a galactose metabolic pathway wherein the expression of the protein reduces the accumulation of a toxic galactose metabolite in the cell.

2. The cell of claim 1, wherein the genetic modification comprises transgenic expression of the protein.

3. The cell of claim 1, wherein the galactose metabolic pathway is selected from the group consisting of the sugar catabolic pathway, the hexosamine pathway and the sugar chain synthesis pathway.

4. The cell of claim 3, wherein the protein of the sugar catabolic pathway is selected from the group consisting of galactokinase (GALK), galactose-1-phosphate uridyl transferase (GALT) and UDP-galactose-4-epimerase (GALE).

5. The cell of claim 3, wherein the protein of the hexosamine pathway is selected from the group consisting of glutamine: fructose-6-phosphate amidotransferase (GFAT), the sodium-calcium exchanger (NCX) and the sodium-hydrogen exchanger (NHE).

6. The cell of claim 3, wherein the protein of the sugar chain synthesis pathway is selected from the group consisting of β1,3-galactosyltransferase (β-1,3-GT), β-1,4-galactosyltransferase (β-1,4-GT), α-1,4-galactosyltransferase (C-1,4-GT), N-acetylgalactosaminyltransferases (GalNAcT), and N-acetylglucosaminyltransferases (GlcNAc-T).

7. The cell of claim 1, wherein the galactose deficiency comprises inactivation of at least one allele of a gene, wherein the gene is selected from the group consisting of alpha-1,3-galactosyltransferase, Forssman synthetase and isoGloboside 3 synthase.

8. The cell of claim 7, wherein the galactose metabolic pathway is selected from the group consisting of the sugar catabolic pathway, the hexosamine pathway and the sugar chain synthesis pathway.

9. The cell of claim 8, wherein the protein of the sugar catabolic pathway is selected from the group consisting of galactokinase (GALK), galactose-1-phosphate uridyl transferase (GALT) and UDP-galactose-4-epimerase (GALE).

10. The cell of claim 8, wherein the protein of the hexosamine pathway is selected from the group consisting of glutamine: fructose-6-phosphate amidotransferase (GFAT), the sodium-calcium exchanger (NCX) and the sodium-hydrogen exchanger (NHE).

11. The cell of claim 8, wherein the protein of the sugar chain synthesis pathway is selected from the group consisting of β-1,3-galactosyltransferase (β-1,3-GT), β-1,4-galactosyltransferase (β-1,4-GT), α-1,4-galactosyltransferase (α-1,4-GT), N-acetylgalactosaminyltransferases (GalNAcT), and N-acetylglucosaminyltransferases (GlcNAc-T).

12. The cell of claim 1, wherein the toxic metabolite comprises UDP-galactose.

13. The cell of claim 1, wherein the toxic metabolite comprises UDP-N-acetyl-D-galactosamine.

14. A transgenic animal comprising the cell of claim 1.

15. An organ derived from the transgenic animal of claim 14.

16. A tissue derived from the transgenic animal of claim 14.

17. An organ or tissue derived from the transgenic animal of claim 14, wherein the organ or tissue is used for xenotransplantation.

18. The organ or tissue of claim 17, wherein the transgenic animal is a pig.

19. The animal, organ or tissue of claims 14, 15 or 16 wherein the galactose deficiency comprises inactivation of at least one allele of a gene, wherein the gene is selected from the group consisting of: alpha-1,3-galactosyltransferase, Forssman synthetase and isoGloboside 3 synthase.

20. The animal, organ or tissue of claims 14, 15 or 16 wherein the galactose metabolic pathway is selected from the group consisting of the following: the sugar catabolic pathway, the hexosamine pathway and the sugar chain synthesis pathway.

21. A method to reduce the toxic accumulation of galactose metabolites in a galactose deficient cell comprising expressing a protein of a galactose metabolic pathway wherein the expression of the protein reduces the accumulation of the toxic metabolite.

22. The method of claim 21, wherein the galactose deficiency comprises inactivation of at least one allele of a gene, wherein the gene is selected from the group consisting of alpha-1,3-galactosyltransferase, Forssman synthetase and isoGloboside 3 synthase gene.

23. The method of claim 21 or 22, wherein the galactose metabolic pathway is selected from the group consisting of the sugar catabolic pathway, the hexosamine pathway and the sugar chain synthesis pathway.

24. The method of claim 23, wherein the protein of the sugar catabolic pathway is selected from the group consisting of galactokinase (GALK), galactose-1-phosphate uridyl transferase (GALT) and UDP-galactose-4-epimerase (GALE).

25. The method of claim 23, wherein the protein of the hexosamine pathway is selected from the group consisting of glutamine: fructose-6-phosphate amidotransferase (GFAT), the sodium-calcium exchanger (NCX) and the sodium-hydrogen exchanger (NHE).

26. The method of claim 23, wherein the protein of the sugar chain synthesis pathway is selected from the group consisting of β-1,3-galactosyltransferase (β-1,3-GT), β-1,4-galactosyltransferase (β-1,4-GT), α-1,4-galactosyltransferase (α-1,4-GT), N-acetylgalactosaminyltransferases (GalNAcT), and N-acetylglucosaminyltransferases (GlcNAc-T).

27. A method to prepare a cell for xenotransplantation comprising:
(a) inactivating at least one allele of a gene, wherein the gene is selected from the group consisting of alpha-1,3-galactosyltransferase, Forssman synthetase and isoGloboside 3 synthase wherein inactivation of the gene results in toxic accumulation of a galactose metabolite; and
(b) expressing a protein of a galactose metabolic pathway in the cell wherein the expression of the protein reduces the accumulation of the toxic metabolite.

28. The method of claim 27, wherein the galactose deficiency comprises inactivation of at least one allele of a gene, wherein the gene is selected from the group consisting of alpha-1,3-galactosyltransferase, Forssman synthetase and isoGloboside 3 synthase.

29. The method of claim 27 or 28, wherein the galactose metabolic pathway is selected from the group consisting of the sugar catabolic pathway, the hexosamine pathway and the sugar chain synthesis pathway.

30. The method of claim 29, wherein the protein of the sugar catabolic pathway is selected from the group consisting of galactokinase (GALK), galactose-1-phosphate uridyl transferase (GALT) and UDP-galactose-4-epimerase (GALE).

31. The method of claim 29, wherein the protein of the hexosamine pathway is selected from the group consisting of glutamine: fructose-6-phosphate amidotransferase (GFAT), the sodium-calcium exchanger (NCX) and the sodium-hydrogen exchanger (NHE).

32. The method of claim 29, wherein the protein of the sugar chain synthesis pathway is selected from the group consisting of β-1,3-galactosyltransferase (β-1,3-GT), β-1,4-galactosyltransferase (β-1,4-GT), α-1,4-galactosyltransferase (α-1,4-GT), N-acetylgalactosaminyltransferases (GalNAcT), and N-acetylglucosaminyltransferases (GlcNAc-T).

33. The method of claim 27, wherein the cell is transplanted into a human.

34. The method of claim 27, wherein the cell is used to produce a transgenic animal.

35. The method of claim 23 or 27, wherein the cell is a porcine cell.


SEQUENCE LISTING (66 sequences)

SEQ ID NO: 1 - DNA, 1471 bp, Homo sapiens
gactctccagtcctcagtcaccttggacaaagaagtgtggatcctcagattccatcttttccaactccaaggtgccatggcagagaaggtgctggtaacaggtggggctggctacattggcagccacacggtgctggagctgctggaggctggctacttgcctgtggtcatcgataacttccataatgccttccgtggagggggctccctgcctgagagcctgcggcgggtccaggagctgacaggccgctctgtggagtttgaggagatggacattttggaccagggagccctacagcgtctcttcaaaaagtacagctttatggcggtcatccactttgcggggctcaaggccgtgggcgagtcggtgcagaagcctctggattattacagagttaacctgaccgggaccatccagcttctggagatcatgaaggcccacggggtgaagaacctggtgttcagcagctcagccactgtgtacgggaacccccagtacctgccccttgatgaggcccaccccacgggtggttgtaccaacccttacggcaagtccaagttcttcatcgaggaaatgatccgggacctgtgccaggcagacaagacttggaacgtagtgctgctgcgctatttcaaccccacaggtgcccatgcctctggctgcattggtgaggatccccagggcatacccaacaacctcatgccttatgtctcccaggtggcgatcgggcgacgggaggccctgaatgtctttggcaatgactatgacacagaggatggcacaggtgtccgggattacatccatgtcgtggatctggccaagggccacattgcagccttaaggaagctgaaagaacagtgtggctgccggatctacaacctgggcacgggcacaggctattcagtgctgcagatggtccaggctatggagaaggcctctgggaagaagatcccgtacaaggtggtggcacggcgggaaggtgatgtggcagcctgttacgccaaccccagcctggcccaagaggagctggggtggacagcagccttagggctggacaggatgtgtgaggatctctggcgctggcagaagcagaatccttcaggctttggcacgcaagcctgaggaccctcccctaccaaggaccaggaaaagcagcagctgcctgctctccagcctctggaggaactcagggccctggagctgctggggccaagccaagggcctcccctacctcaaaccccagctgggcccgcttagcccaccaggcatgaggccaaggctccactgaccaggaggccgaggtctctaactcttatcttccacagggtccaagagttcatcaggacccccaagagtgagtgagggggcaaggctctggcacaaaacctcctcctcccaggcactcatttatattgctctgaaagagctttccaaagtatttaaaaataaaaacaagttttcttacactgg

SEQ ID NO: 2 - DNA, 2168 bp, Homo sapiens
ggctacgcagcttgctcctggcacgggcaccttgaatctcctcctcacacagatggagaccatgcttgatttcctgaacttgtagtaagaagaaggaaaacacagcacgctggagccaacagagttaagaggaagatttatgagtcatggaaccctccatcagatttggaagaaagtagaatgagcgcagaggtgacagacagccactgaggcccatggacaatctccacctcacgcttctctatcaaacttgaagatttattagtaatatgctgcctttggaagatgaaaacaaactagtgccaaggaggcgtattcttcaatatttggaatagacgtgttctcaagacaatggcttcaaaggtctcctgtttgtatgttttgacagttgtgtgctgggccagcgctctctggtacttgagtataactcgccctacttcttcttacactggctccaaaccattcagccacctaacagttgccaggaaaaacttcacctttggcaacataagaactcgacctatcaacccacattcttttgaatttcttatcaacgagcccaataaatgtgagaaaaacattccttttcttgttatcctcatcagcaccactcacaaggaatttgatgcccgtcaggcaatcagagagacgtggggggatgagaacaactttaaggggatcaagatagccaccctgttcctcctgggcaagaatgctgatcctgttctcaatcagatggtggagcaagagagccaaatcttccatgatatcatcgtggaggactttattgactcctaccataaccttaccctcaaaacattaatggggatgagatgggtggccactttttgttcaaaagccaagtatgtcatgaaaacagacagcgacatttttgtaaacatggacaatcttatttataaattactgaaaccctccaccaagccacgaagaaggtattttactggctatgtcattaatggaggaccgattcgggatgtccgcagtaaatggtatatgcccagggatttgtacccagacagtaactacccacctttctgttcggggactggctacatcttttcagccgatgtagctgaactcatttacaagacctcactccacacaaggctgcttcaccttgaagacgtatatgtgggactgtgtcttcgaaagctgggcatacatcctttccagaacagtggcttcaatcactggaaaatggcctacagtttgtgtaggtatcgccgagttatcactgtgcatcagatctctccagaagaaatgcacagaatctggaatgacatgtcaagcaagaaacatctcagatgttaggatttttaccaatgtaaatatgtttcttttctttttttaagaaatgggacctaaggtgttggtattttccaggtgtcgggggaaatgaactggtgaaggggttttgtaaagtttttgcttcctgctataagttcttttcttggattaccaatttatgaatgttagactctggtcatagaaacaataaatgagttagaagggccagatttcattctcagtcccagagcattgctatttatctcaaaaagtgacttccaaacaactcttaggattgacgtaccgtgcatctgagataaaaatttggttctgggaaactgaaactcacagtaatgtgtcatatcatccctgcaaaaattaatacacaaatagaaaccattttcaaaagcaattcagaaaggatgcacagtcaggaagacacactggatgtgattattaatatcgtgtgtgttgttacattatatttttacatatattcccatgtaatgtgtacagtctttgcagttccaccaagaaatgaacttggtacctgcagagtggctgcagttaaatagatgggagtttaaatttgagaatcaaacattctatgtgtttggaagacaactctgcttgctcatccaaggattaaatctggtcagcaggtggaatgtgtataaaatgctacttaacaaagtaaacaaaagattttttttttctttttttttctttcttttttgttttgctctttcagaacaaacattaaatggtgcctccaaggaaactttgccaaatataatctcacctgcttccttccagacagtgtcgctaagtgcatttcacagtttttggatctggcaggc

SEQ ID NO: 3 - DNA, 4080 bp, Homo sapiens
gcgcctgcggcgccgcgggcgggtcgcctcccctcctgtagcccacacccttcttaaagcggcggcgggaagatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagcgttttggtacacggataagagacctgaaattagccagggacctctgctgtgtgtctctgccaatctgctgggctggtccctctcatttttaccagtctgagtgacagctccccttggctcatcattcagatggctttccagatgaccaggacaggtgggatattttgcccccaacttggctcggcatgtgaattcttagctctgcaaggtgtttatgcctttgcgggtttcttgatgtgttcgcagtgtcacccaagagtcagaactgtagacatcccaaaatttggtggccgtggaacacattcccggtgatagaattgctaaattgtcgtgaaataggttagaatttttctttaaattatggttttcttattcgcgaaaattcggagagtgctgctaaaattggattggtgtcatctttttggtagttgtaatttaacagaaaaacacaaaatttcaaccattcttaatgttacgtcctccccccacccccttctttcagtggtatgcaaccactgcaatcaatgtgtcatatgtcttttcttagcaaaaggatttaaaacttgagccctggaccttttgcctatgtgtgtggattccagggcaactctagcatcagagcaaaagccttgggtttctcgcattcagtggcctatctccagattgtctgatttctgaatgtaaagttgttgtgtttttttttaaatagtaggtttgtagtattttaaagaaagaacagatcgagttctaattatgatctagcttgattttgtgttgatccaaatttgcatagctgtttaatgttaagtcatgacaatttatttttcttggcatgctatgtaaacttgaatttcctaagtatttttattctggtgttttaaatatggggaggggtattgagcattttttagggagaaaaataaatatatgctgtagtggccacaaataggcctatgatttagctggcaggccaggttttctcaagagcaaaatcaccctctggccccttggcaggtaaggcctcccggtcagcattatcctgccagacctcggggaggatacctgggagacagaagcctctgcacctactgtgcagaactctccacttccccaaccctccccaggtgggcagggcggagggagcctcagcctccttagactgacccctcaggcccctaggctggggggttgtaaataacagcagtcaggttgtttaccagccctttgcacctccccaggcagagggagcctctgttctggtgggggccacctccctcagaggctctgctagccacactccgtggcccaccctttgttaccagttcttcctccttcctcttttcccctgcctttctcattccttccttcgtctccctttttgttcctttgcctcttgcctgtcccctaaaacttgactgtggcactcagggtcaaacagactatccattccccagcatgaatgtgccttttaattagtgatctagaaagaagttcagccgcacccacaccccaactccctcccaagaacttcggtcctaaagcctcctgttccacctcaggttttcacaggtgctcacaccacagttgaggctcacacacaggtctgtctgtcacaaacccacctctgttgggagctattgagccacctgggatgagatgacacaagacactcctaccactgagcgcctttgtccaggtgccagcctgggctcaggttccaagactcagctgcctaatcccagggttgagccttgtgctcgtgtcggaccccaaaccactgccctcctggtaccagccctcagtgtggaggctgagctggtgcctggccccagtcttatctgtgcctttactgctttgcgcatctcagatgctaacttggttctttttccagaaggctttgtattggttaaaaattattttctattgcagagagcagctgtgactcatgcaaaaagtattttctctgtcagatccccactctataccaaggatattattaaaactagaaatgactgcattgagagggagttgtgggaaataagaagaatgaaagcctctctttctgtccgcagatcctgacttttccaaagtgccttaaaagaaatcagacaaatgccctgagtggtaacttctgtgttattttactcttaaaaccaaactctaccttttcttgtttttttttttttttttttttttttttttggttaccttctcattcatgtcaagtatgtggttcattcttagaaccaagggaaatactgctccccccatttgctgacgtagtgctctcatgggctcacctgggcccaaggcacagccagggcacagttaggcctggatgtttgcctggtccgtgagatgccgcgggtcctgtttccttactggggatttcagggctgggggttcagggagcatttccttttcctgggagttatgtaccgcgaagtgtgtcatgtgccgtgcccttttctgtttctgtgtatcctattgctggtgactctgtgtgaactggcctttgggaaagatcagagaggcagaggtggcacaggacagtaaaggagatgctgtgctgcctacagcctggacagggtctctgctgtactgccaggggcgggggctctgcatagccaggatgacgcctttcatgtcccagagacctgttgtgctgtgtattttgatttcctgtgtatgcaaatgtgtgtatttaccattgtgtagggggctgtgtctgatcttggtgttcaaaacagaactgtatttttgcctttaaaattaaataatataacgtgaataaatgaccctaactttgt

SEQ ID NO: 4 - DNA, 2065 bp, Homo sapiens
cgcgccgcccgcccgccgccgctggagctagagatggatttgcagccgctgcaagtgtgtggaagggccgtgttcgtgttggcaaagaaggtcggctgctgagccagggcgtgtctcccggaggcctgtgggctgccaggatccccacctctctgcaatgggctgcccaggctgaccagccggttcctgctggaagctcctggtctgatctggggataccatgtccaagccccccgacctcctgctgcggctgctccggggcgccccaaggcagcgggtctgcaccctgttcatcatcggcttcaagttcacgtttttcgtctccatcatgatctactggcacgttgtgggagagcccaaggagaaagggcagctctataacctgccagcagagatcccctgccccaccttgacaccccccaccccaccctcccacggccccactccaggcaacatcttcttcctggagacttcagaccggaccaaccccaacttcctgttcatgtgctcggtggagtcggccgccagaactcaccccgaatcccacgtgctggtcctgatgaaagggcttccgggtggcaacgcctctctgccccggcacctgggcatctcacttctgagctgcttcccgaatgtccagatgctcccgctggacctgcgggagctgttccgggacacacccctggccgactggtacgcggccgtgcaggggcgctgggagccctacctgctgcccgtgctctccgacgcctccaggatcgcactcatgtggaagttcggcggcatctacctggacacggacttcattgttctcaagaacctgcggaacctgaccaacgtgctgggcacccagtcccgctacgtcctcaacggcgcgttcctggccttcgagcgccggcacgagttcatggcgctgtgcatgcgggacttcgtggaccactacaacggctggatctggggtcaccagggcccgcagctgctcacgcgggtcttcaagaagtggtgttccatccgcagcctggccgagagccgcgcctgccgcggcgtcaccaccctgccccctgaggccttctaccccatcccctggcaggactggaagaagtactttgaggacatcaaccccgaggagctgccgcggctgctcagtgccacctatgctgtccacgtgtggaacaagaagagccagggcacgcggttcgaggccacgtccagggcactgctggcccagctgcatgcccgctactgccccacgacgcacgaggccatgaaaatgtacttgtgaggggcccgccaggtcacctccccaacctgctcctgatggggcactgggccgcccttcccggggaggcaagattgagggcccgggagagggaggcccgagctgccaccgggcttaggcaggctgttgaggagctgtgggagcaggcccagtgggaggctgtggacaccccgaggacagtgtcctgtctcgaggcagggctgacacatggtgccatagccagcggagggcgctcagtgagtgccccgggccttctagacaacaggcaggaaggatgaacctcagggcacccccaggtggtgcggaaagccaggcagttgggacagaggtgcccacgagggcagaggccggtgctaaggggatggggaagaagggacaagattcccagagaggagaggaggctgttggtaggaaagtggcagggctgggggagacccagccccaagggtccggggcggaggatgctttgttcttttctggttttggttcctctttcgcggggggtgggggaggtcaacagggactgagtggggcagaggcccagaagtgccagcctggggagccgtttgggggcagccccttctgcccaccccatccttcttcctctccagagatgccaggggggcgtgtatgctctaccccttccctcagacaggggctgggtggggaggctctttaggctcaggagaagcattttaaagaaacccccaccctgccgcccgcattataaacacaggagaataatcaatagaataaaagtgaccgactgtcaaaaaaaaaaaaaaa

SEQ ID NO: 5 - DNA, 2166 bp, Rattus norvegicus
tggatcacagtctccatcgactgactcaggatgcggctggaccgccgggccctctatgcgctagttctgctgcttgcctgcgcctcgctgggtctcctgtacgccagcacccgagacgcgccaggtctcccgaaccctctggcattgtggtcacccccacaaggtcccccgaggctcgatctgctagaccttgccactgagcctcgctacgcacacatcccagtcaggatcaaggagcaagtggtggggctgctggctcagaacaattgcagttgtgagtccagcggaggacgctttgccttgccgttcctgaggcaggtccgggcgattgacttcactaaagcctttgacgccgaggagctgagggctgtttctatctccagagagcaggaataccaggccttccttgcaaggagccggtccctggctgaccagctgctgatagcccctgccaactcccccttacagtatcccctgcagggtgtggaggttcagcccctcaggagcatcctggtgccagggctaagtctgcaggaagcttctgttcaggaaatatatcaggtgaacctgattgcttcccttggcacctgggatgtggcaggggaagtaacaggggtgactctcactggagaggggcagtcggacctcacccttgccagcccaattctggataaactcaaccgacagctgcaactggtgacttacagcagccggagctaccaagccaacacagcagacacagtccggttctccaccaagggacatgaagtggccttcaccatcctcataagacatcctcccaacccccggctgtacccaccatcatccctaccccaaggagcccagtacaacatcagtgctctggttaccgttgccaccaagacctttcttcgttatgatcggctacgggcactcattgccagcatcagacgcttttaccctacggtcaccatagtaatcgctgacgacagcgacaaaccggagcgaattagcgacccccatgtggagcactatttcatgcccttcggcaagggttggtttgcaggtcggaacctggcggtgtcccaagtaaccaccaaatacgtgctgtgggtggacgacgactttgtcttcacggcgcgcacgcggctggagaagcttgtggatgtcctggagaggacgcccctggacttggttgggggcgcggtgcgggagatctcgggctacgctaccacctaccgacagctgctaagtgtggagccgggcgccccaggctttgggaactgcctccggcaaaagcagggcttccaccacgagctcgctggctttccaaactgcgtggtcaccgacggcgtagtcaacttcttcctggcgcgcacagataaagtgcgccaggtgggctttgacccacgcctcaaccgggtggctcatctggaattcttcctggatggtcttggttcccttcgagttggctcctgctctgatgttgttgtggatcatgcgtcaaaggtgaagctgccttggacatcaaaggatccaggggctgaactttatgcccgttaccgttacccgggatcactggaccaaagtcaggtggccaaacatcgactgctcttcttcaaacaccggctacagtgcatgaccgccgagtaacgtctgatttgggccttcacactgtcaggctgggcctgcctcctccctgccaggaatttccagcaaccacccccccccaatccctgagcaccccactgatgaacaccctggcttcccgaccctctccaccaatctgattcctaacaggggcttgtcctggtgacacccttcctttctgtgagtgaccagaggccagatggagccatatcctcccccacagccagtgccaagtcctccccaaccccactcctatggggcaggaaatggggaggttcactttccaagtgccaaagagcccagacggactctaagaccctcaagtggaaacactctcacctcctgaggtgggcagggaaactcccaatttgcaaccccagggacatgcaccccaccccagctctggatccagcaccatgtgtcccggctccaacatacccctacagaaagcactgtgactgtagttctgtggggctggtgaacacacggtggaagccaaaaaaaaaaaaaaaaaaaaaaaaggggggggggggatcc

SEQ ID NO: 6 - DNA, 3778 bp, Homo sapiens
tttttaaattttgcatttgacttaaagtgccatgagaaaatttgcatactgcaaggtggtcctagccacctccttgatttgggtactcttggatatgttcctgctgctttacttcagtgaatgcaacaaatgtgatgaaaaaaaggagagaggacttcctgctggagatgttctagagccagtacaaaagcctcatgaaggtcctggagaaatggggaaaccagtcgtcattcctaaagaggatcaagaaaagatgaaagagatgtttaaaatcaatcagttcaatttaatggcaagtgagatgattgcactcaacagatctttaccagatgttaggttagaagggtgtaaaacaaaggtgtatccagataatcttcctacaacaagtgtggtgattgttttccacaatgaggcttggagcacacttctgcgaactgtccatagtgtcattaatcgctcaccaagacacatgatagaagaaattgttctagtagatgatgccagtgaaagagactttttgaaaaggcctttagagagttatgtgaaaaaactaaaagtaccagttcatgtaattcgaatggaacaacgttctggattgatcagagctagattaaaaggagctgctgtgtctaaaggccaagtgatcaccttcctggatgcccattgtgagtgtacagtgggatggctggagcctctcttggccaggatcaaacatgacaggagaacagtggtgtgtcccatcatcgatgtgatcagtgatgatacttttgagtacatggcaggctctgatatgacctatggtgggttcaactggaagctcaattttcgctggtatcctgttccccaaagagaaatggacagaaggaaaggtgatcggactcttcctgtcaggacacctaccatggcaggaggccttttttcaatagacagagattactttcaggaaattggaacatatgatgctggaatggatatttggggaggagaaaacctagaaatttcctttaggatttggcagtgtggaggaactttggaaattgttacatgctcacatgttggacatgtgtttcggaaagctacaccttacacgtttccaggaggcacagggcagattatcaataaaaataacagacgacttgcagaagtgtggatggatgaattcaagaatttcttctatataatttctccaggtgttacaaaggtagattatggagatatatcgtcaagagttggtctaagacacaaactacaatgcaaacctttttcctggtacctagagaatatatatcctgattctcaaattccacgtcactatttctcattgggagagatacgaaatgtggaaacgaatcagtgtctagataacatggctagaaaagagaatgaaaaagttggaatttttaattgccatggtatggggggtaatcaggttttctcttatactgccaacaaagaaattagaacagatgacctttgcttggatgtttccaaacttaatggcccagttacaatgctcaaatgccaccacctaaaaggcaaccaactctgggagtatgacccagtgaaattaaccctgcagcatgtgaacagtaatcagtgcctggataaagccacagaagaggatagccaggtgcccagcattagagactgcaatggaagtcggtcccagcagtggcttcttcgaaacgtcaccctgccagaaatattctgagaccaaatttacaaaaaaacgaaaaaaataaggattgactgggctacctcagcatacatttctgccacattcttaagtagcaaaaaaggaaaagtgctttcctcctctgcaggatgtaaggtttatcagccattaaaacttagacttctctagcttttcactagctgtgaaccagccttcctgtccatggacgtgaaactgcatagtaatgagactgtgcacactgatgtttacaagattgaaagagtctttctccgaaaatcatggtaaagaatactgagacaatgaaaaaaaatcaacaaaatatgctttctggagaactgtaccttctatggtttgcttgcacatcagtagtttctgctgaacgtgctgtcataatgaagagatttccaagattttttttcctgattagaacgggtagccagtatattaaatattgatagaaaaataaaagaactggaaccagattcagaatcttgaaaacaacattttttacaacaaacaaaaaaactatattaaacagggtttaaaggaaaattaaaacagaactatgaagaagtacaatttgttatagtatagtatcaaatttctatatagattttatacctcagtggggaaaaataactgattccaatgacattcattttgttttcatctgtgatagtcatggatgcttttattttccttggggtgctgaaattgagctgaaaaaaaaaggctctttgaatatagttttaatttctctctacagttttttttgtttggtttgtgggctgttggaattgtaatttttaattgccttctaaaaaatggaaatttaacaatgtctgatctcagctgaacaaattagatgtttcagttgctcttgggtcaactggcttacagatttacatgtgcacacacacacaaatttcttatcacattttcgacttcttcacttgacctaactgattatgcgaaatacccaagattcatgctactgttccacatttgttttcacagcaataaatcttcagttctgttgtttatgattccacttaacaaggggcctgcaaatgtgatttattatttgggtatttggagataatacatttgagggttttttggaaaacctttttcactccatactcaaatatgcttcattgtcaaatgcatatttaaattaaattattgaattgtaatgtttatctgctgctttttttaaataaaatttgactgaaaatgtttaattggcattttttaatgacttacccaagaaaagtgcagctattattccatattaataggcttgcatttcttttcctaaatcttatttaggctaaatcagttttattgtcctctgattttttttaataccacagaaatcacctgagtgtcaattgaaaagttgtcaattaaaaggtaaccttttaactctcgtaggaggaatctcattaagacatttttcctgatatgtagagcagtctgttggcaaaaatgcatatattttctttcatatttgtaaaattatatttaatggaattcttttctttgattatcaaggactttcactgcaggcagtgctatttcttgtgcctaagaatgtttccaaaagtcgcatcgctaatgatatttgccaagttgagtgtacacaaagtttctcatatcctgttcaagttaatcaacatcaaacacatggggatgctttagggtgagtctataatacaaaatgcataaaccatgtccccaggaaatttgaaaggaagcaagtgctgaatggaatttttttccttttccatgagctgtgttaattctatctccagtaggcctaatgcttgaaataagcaagatgtctaatcaataaattattttcatgctcagaatttcaggtttttgtactccagcatagcttggtcttatttcttactgtatgaaagcttaacagcaatgtgatttaaggttttgttttaaatgggagatgtaagtgatttaattcatgggtacttttagaacctgatagataatcccattgcctttatttttctaattaaagaatcctaaatactttgaaaatacaaaatattcctg

SEQ ID NO: 7 - DNA, 2318 bp, Homo sapiens
attaactgggttttcctatttatctatcctctcgcattacttctctgagtcagagcctcttctctctaagtcacgggaactgcccttgctacttgtgacctgccctttactcagcagtttttgttctgggaagccctgggattctgctaatacctatcactgtaggtgctgaagggaaacagatgaagaacatgacctcaaggagcttcctgtcaatgagaagaccaagctgacgcctggcaaagatattaaagaggagcctgaaactgttccttggacatcttatgaatgtcagaaaataccttttggagggttagaagatcaggggacatggttgttcacatttgctgccacggaacaccgccagtcttcacttggaaacagaatcacgccttgtgaagagatcatccctaagcaggagagaagctactaaaggattgtgtcctcctccaccttccctgtgctcggtctccacctgtctcccattctgtgacgatggttcaatggaagagactctgccagctgcattacttgtgggctctgggctgctatatgctgctgccactgtggctctgaaactttctttcaggttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttgagacacactatgagagcgttgctacctgtggggcaagagcatgtacaaacatgctcagaacttgctgggacagtgtgggtgggagaccagggctttgcaattcgtggcatcctttaggataagagggctgctattagattgtgggtaagtagatcttttgccttgcaaattgctgcctgggtgaatgctgcttgttctctcacccctaaccctagtagttcctccactaactttctcactaagtgagaatgagaactgctgtgatagggagagtgaaggagggatatgtggtagagcacttgatttcagttgaatgcctgctggtagcttttccattctgtggagctgccgttcctaataattccaggtttggtagcgtggaggagaactttgatggaaagagaaccttcccttctgtactgttaacttaaaaataaatagctcctgattcaaagtattacctctactttttgcctagtatgccagaaataatataaatctaaacaga

SEQ ID NO: 8 - DNA, 1361 bp, Homo sapiens
aacagggcaggagtgagtggagtatgttgcaaaataagaactcagagaaacgagtgagtttggaaaaaagacttacagattttgacggtctcttgacatttcacccttctttgaggcatgcctttatcaatgcgttacctcttcataatttctgtctctagtgtaattatttttatcgtcttctctgtgttcaattttgggggagatccaagcttccaaaggctaaatatctcagaccctttgaggctgactcaagtttgcacatcttttatcaatggaaaaacacgtttcctgtggaaaaacaaactaatgatccatgagaagtcttcttgcaaggaatacttgacccagagccactacatcacagcccctttatctaaggaagaagctgactttcccttggcatatataatggtcatccatcatcactttgacacctttgcaaggctcttcagggctatttacatgccccaaaatatctactgtgttcatgtggatgaaaaagcaacaactgaatttaaagatgcggtagagcaactattaagctgcttcccaaacgcttttctggcttccaagatggaacccgttgtctatggagggatctccaggctccaggctgacctgaactgcatcagagatctttctgccttcgaggtctcatggaagtacgttatcaacacctgtgggcaagacttccccctgaaaaccaacaaggaaatagttcagtatctgaaaggatttaaaggtaaaaatatcaccccaggggtgctgcccccagctcatgcaattggacggactaaatatgtccaccaagagcacctgggcaaagagctttcctatgtgataagaacaacagcgttgaaaccgcctcccccccataatctcacaatttactttggctctgcctatgtggctctatcaagagagtttgccaactttgttctgcatgacccacgggctgttgatttgctccagtggtccaaggacactttcagtcctgatgagcatttctgggtgacactcaataggattccaggtgttcctggctctatgccaaatgcatcctggactggaaacctcagagctataaagtggagtgacatggaagacagacacggaggctgccacggccactatgtacatggtatttgtatctatgaaaacggagacttaaagtggctggttaattcaccaagcctgtttgctaacaagtttgagcttaatacctacccccttactgtggaatgcctagaactgaggcatcgcgaaagaaccctcaatcagagtgaaactgcgatacaacccagctggtatttttgagctattcatgagctactcatgactgaagggaaactgcagct

SEQ ID NO: 9 - DNA, 2010 bp, Homo sapiens
gcggtaaatccgggcttgcggccgctggcgtagtctgtggccgggtggtcgttgctgcgcgccccgagccccgagagccatgcagatgtcctacgccatccggtgcgccttctaccagctgctgctggccgcgctcatgctggtggcgatgctgcagctgctctacctgtcgctgctgtccggactgcacgggcaggaggagcaagaccaatattttgagttctttcccccgtccccacggtccgtggaccaggtcaaggcgcagctccgcaccgcgctggcctctggaggcgtcctggacgctagcggcgattaccgcgtctacaggggcctgctgaagaccaccatggaccccaacgatgtgatcctggccacgcacgccagcgtggacaacctgctgcacctgtcgggtctgctggagcgctgggagggcccgctgtccgtgtcggtgttcgcggccaccaaggaggaggcgcagctggccacggtgctggcctacgcgctgagcagccactgccccgacatgcgcgccagggtcgccatgcacctcgtgtgcccctcgcgttacgaggcagccgtgcccgacccccgggagccgggggagtttgccctgctgcggtcctgccaggaggtctttgacaagctagccagggtggcccagcccgggattaattatgcgctgggcaccaatgtctcctaccccaataacctgctgaggaatctggctcgtgagggggccaactatgccctggtgatcgatgtggacatggtgcccagcgaggggctgtggagaggcctgcgggaaatgctggatcagagcaaccagtggggaggcaccgcgctggtggtgcctgccttcgaaatccgaagagcccgccgcatgcccatgaacaaaaacgagctggtgcagctctaccaggttggcgaggtgcggcccttctattatgggttgtgcaccccctgccaggcacccaccaactattcccgctgggtcaacctgccggaagagagcttgctgcggcccgcctacgtggtaccttggcaggacccctgggagccattctacgtggcaggaggcaaggtgcccaccttcgacgagcgctttcggcagtacggcttcaaccgaatcagccaggcctgcgagctgcatgtggcggggtttgattttgaggtcctgaacgaaggtttcttggttcataagggcttcaaagaagcgttgaagttccatccccaaaaggaggctgaaaatcagcacaataagatcctatatcgccagttcaaacaggagttgaaggccaagtaccccaactctccccgacgctgctgagcccttccctcccctaatctgagaagtcagcctcttggctcctcaggccaccatttaggcctgactggggtaagaaatgtcgctccactttacagaggtagctgtggtgttgaaacactggacttggatatggggtgctgggatcgattcctagctttaccactaactagctgtgtggccttgagtaaatcccgttacctctctgagcctcggttaccctgtctgtaaaaagggaggtgagaatacctacctcacggaactgttgggaggctcagatgagatgctatatgtgaaaacattctgtaagcttcgtacaaatgtgaagtattaatattatcgcagtattattgttgttattattattgttattattaacaatcttgggtgggtagtaggagagcaaaaagtatgaatgggatggagctaagaagtctgaatacttaatgaaatggactttttggaaagaaatcagatgaaggcataaaatttagttcttagctcttgaacagaagcctaaaattcctggttctctcgggcttcgccttcaagggttctggaggagggaagggtctgcaggttccatgggtgacagcctgagatctgtcccttcaacgggctgggctgggtatgtgcctaccgatgacaatgtgtaaataaatgcgtgttcacacccacaaaaaaaa

SEQ ID NO: 10 - DNA, 2880 bp, Homo sapiens
atgaggctcctccgcagacgccacatgcccctgcgcctggccatggtgggctgcgcctttgtgctcttcctcttcctcctgcatagggatgtgagcagcagagaggaggccacagagaagccgtggctgaagtccctggtgagccggaaggatcacgtcctggacctcatgctggaggccatgaacaaccttagagattcaatgcccaagctccaaatcagggctccagaagcccagcagactctgttctccataaaccagtcctgcctccctgggttctataccccagctgaactgaagcccttctgggaacggccaccacaggaccccaatgcccctggggcagatggaaaagcatttcagaagagcaagtggacccccctggagacccaggaaaaggaagaaggctataagaagcactgtttcaatgcctttgccagcgaccggatctccctgcagaggtccctggggccagacacccgaccacctgagtgtgtggaccagaagttccggcgctgccccccactggccaccaccagcgtgatcattgtgttccacaacgaagcctggtccacactgctgcgaacagtgtacagcgtcctacacaccacccctgccatcttgctcaaggagatcatactggtggatgatgccagcacagaggagcacctaaaggagaagctggagcagtacgtgaagcagctgcaggtggtgagggtggtgcggcaggaggagcggaaggggttgatcaccgcccggctgctgggggccagcgtggcacaggcggaggtgctcacgttcctggatgcccactgtgagtgcttccacggctggctggagcccctcctggctcgaatcgctgaggacaagacagtggtggtgagcccagacatcgtcaccatcgaccttaatacttttgagttcgccaagcccgtccagaggggcagagtccatagccgaggcaactttgactggagcctgaccttcggctgggaaacacttcctccacatgagaagcagaggcgcaaggatgaaacataccccatcaaatccccgacgtttgctggtggcctcttctccatccccaagtcctactttgagcacatcggtacctatgataatcagatggagatctggggaggggagaacgtggaaatgtccttccgggtgtggcagtgtgggggccagctggagatcatcccctgctctgtcgtaggccatgtgttccggaccaagagcccccacaccttccccaagggcactagtgtcattgctcgcaatcaagtgcgcctggcagaggtctggatggacagctacaagaagattttctataggagaaatctgcaggcagcaaagatggcccaagagaaatccttcggtgacatttcggaacgactgcagctgagggaacaactgcactgtcacaacttttcctggtacctgcacaatgtctacccagagatgtttgttcctgacctgacgcccaccttctatggtgccatcaagaacctcggcaccaaccaatgcctggatgtgggtgagaacaaccgcggggggaagcccctcatcatgtactcctgccacggccttggcggcaaccagtactttgagtacacaactcagagggaccttcgccacaacatcgcaaagcagctgtgtctacatgtcagcaagggtgctctgggccttgggagctgtcattcactggcaagaatagccaggtccccaaggacgaggaatgggaattggcccaggatcagctcatcaggaactcaggatctggtacctgcctgacatcccaggacaaaaagccagccatggccccctgcaatcccagtgacccccatcagttgtggctctttgtctaggacccagatcatccccagagagagcccccacaagctcctcaggaaacaggattgctgatgtctgggaacctgatcaccagcttctctggaggccgtaaagatggatttctaaacccactgggtggcaaggcaggaccttcctaatccttgcaacaacattgggcccattttctttccttcacaccgatggaagagaccattaggacatatatttagcctagcgttttcctgttctagaaatagaggctcccaaagtagggaaggcagctgggggagggttcagggcagcaatgctgagttcaagaaaagtacttcaggctgggcacagtggctcatgcctgaaatcctagcactttgggaagacaatgtgggagaatggcttgagcccaggagttcaagaccggcctgagcaacatagtgaggatcccatctctacgcccaccctccccccggcaaaaaaaaaagctgggtatggtggcttatgcctgtagtcgcagctactcagaaggctgaggtgggaggattgcttgttccccggaggttgaagctacagtgagccttgattgtgtcactgcactccagcctgggcaacaggtaagactctgtctcaaaaaaaaaacaaaaaagaagaagaaaagtacttctacagccatgtcctattccttgatcatccaaagcacctgcagagtccagtgaaatgatatattctggctgggcacagtggctcacacctgtaatcctagcactttgggaggccaaggcaggtggatcacctgaggtcagaagtttgaaaccagcctggactacatggtgaaactccatctctactaaaagtacaaaaattagctgggcatgatggcacgcacctgcagtcccagctacttgggaggctgaggcaggagaatcactcgaacccaggaggcagaggttgcagtgagccaagacagcaccattgcaccccagcctgagcaacaagagcgaaactccatctcaggaaaaaaaaaaaaaaaaa

SEQ ID NO: 11 - DNA, 1553 bp, Homo sapiens
attcccacctcctccagaagccccgcccactcccgagccccgagagctccgcgcacctgggcgccatccgccctggctccgctgcacgagctccacgcccgtaccccggcgtcacgctcagcccgcggtgctcgcacacctgagactcatctcgcttcgaccccgccgccgccgccgcccggcatcctgagcacggagacagtctccagctgccgttcatgcttcctccccagccttccgcagcccaccagggaaggggcggtaggagtggccttttaccaaagggaccggcgatgctctgcaggctgtgctggctggtctcgtacagcttggctgtgctgttgctcggctgcctgctcttcctgaggaaggcggccaagccgcaggagaccccacggcccaccagcctttctgggctcccccaacaccccgtcacagccggtgtccacccaaccacacagtgtctagcgcctctctgtccctgcctagccgtcaccgtctcttcttgacctatcgtcactgccgaaatttctctatcttgctggagccttcaggctgttccaaggataccttcttgctcctggccatcaagtcacagcctggtcacgtggagcgacgtgcggctatccgcagcacgtggggcagggtggggggatgggctaggggccggcagctgaagctggtgttcctcctaggggtggcaggatccgctcccccagcccagctgctggcctatgagagtagggagtttgatgacatcctccagtgggacttcactgaggacttcttcaacctgacgctcaaggagctgcacctgcagcgctgggtggtggctgcctgcccccaggcccatttcatgctaaagggagatgacgatgtctttgtccacgtccccaacgtgttagagttcctggatggctgggacccagcccaggacctcctggtgggagatgtcatccgccaagccctgcccaacaggaacactaaggtcaaatacttcatcccaccctcaatgtacagggccacccactacccaccctatgctggtgggggaggatatgtcatgtccagagccacagtgcggcgcctccaggctatcatggaagatgctgaactcttccccattgatgatgtctttgtgggtatgtgcctgaggaggctggggctgagccctatgcaccatgctggcttcaagacatttggaatccggcggcccctggaccccttagacccctgcctgtatagggggctcctgctggttcaccgcctcagccccctcgagatgtggaccatgtgggcactggtgacagatgaggggctcaagtgtgcagctggccccataccccagcgctgaagggtgggttgggcaacagcctgagagtggactcagtgttgattctctatcgtgatgcgaaattgatgcctgctgctctacagaaaatgccaacttggttttttaactcctctcaccctgttagctctgattaaaaacactgcaacccaaaaaaaaaaaaaaaaaaaaaaaaaaaaaaaaaaaaaaaaaaaaaaaaaaa

SEQ ID NO: 12 - DNA, 2462 bp, Porcine
  misc_feature (580)..(580): n is a, c, g, or t
aggcctaaacctagaactcctgaccctgaagctaaggaatataatcttgaaggtgttttccagtcagtagaataacacagagtttccacacatgcgtgggtctctttctaggttgcttattctgttccattggtccaataaaccatcctggcgctaatgctatactgagttcactgcgtttcatggtctgtcttggtatctggtggaacaagagcccaactctcccctccctgctttgtcaagactgccttggttatatctggccccttcccgctgctgtccaaattttaagaatagctggccaagctcccccaaaactctgttggcatttgtcttgagtttataggttgatgcatggagaattgttgccttcgtgatgctgatgctttccagtgctcactcgggggtctctttccttccacctaaagacttctgcacatggttctgcttgggtcactcttccccaagccttcacctagtgaactcctcctcctcctggtctcagggtctcctgcacccttatttcttccttagagccctgatcacaatggtcctgaaatcactcattgcgtgggtcttngtgacagatagtaggtcccagtaaatatctgttaaaagaatgaaggaagtttaggtaggaaggtcttcgggacctggagcaccttggccatagttagagggatggtgaccagaggtacttaacttgcctgtgccttggctttcttcctacaaaaccgggatgtgatcagaatgtgtataagatgaagtgagctcagctaggccgtgaggcaagtggagcaaagcctggcaagggatcagagctacttgtttacctgccctgcccttctgctcagtgaatcttcagtcctgcactcctgtgatgctcctggaggctccaacactctttccccagcagtgatcccgtcttgactccacctctcctatgaactagtcaccttatttctactcagcatatgacacaaatgagtctcaggaagaatgactcataaggccttaaacctagaactcctgaccctgaagctaaggaatataatcttgaaggtgttttccagtcagtagaattgctagttagatttggggagctacatagttctcaaaagaaaacaaaacttccggacccgccgtgttaatttgaattatttttatcttattgttactgaaataggtataaacctagaactaagaatgaagtcctcatgctcctagctctgcacacctaccatgataccaaagcaaatcttttaagtaggtgcaattacagccacaaaaccaataaaatccaaattagcaacgttaaatttatgcaactgatgacatggtgctgaaatcaaacctcttgcattgagtctaatggtagcagagtgatgtttttacatgtttcattccctgtgtcatcatcttttgattttgatcctgatgagctatcacttcagccatggtcagaattaccgtcataattttcactaaaaaaaaaacccaaaaaacacatttattatccaatttgatgggctgagcaatttaaacactggatcctcaagtgcaataatgacaactgggaaatactttgctaacatcactccttgtgtatttatttactgcatcattaaagacctagtgcaagtgagttcaccgatgacaataatggcgcagtttatgcttttgcaaaggatccattgttcggattgtcatggagctcctcattcctgagctaccctgtggggctgatgattcaactctcccaccctttagtccactgaacccatcaggaaagttcattatcccaagctccaagatgtcacttggctccctgcagcctctctgcaaccgtcaagtattcaatcagatctctgttcttttcaaatcaggatgaaacagttaaaattatacatcacactcaggttctgtgccattttcatgtcacaattccaatgccttaaaatatttaagaaactaatttcttagtctctgaagtcccgtggtgaatgatcctggcaaaagcaagttctgaattttgcagcagtaaaatagatggtccgggaccccaaggagtcttgtaaaggctgagtgagggcagccggatgtgcctacaccagctcatcagaagtgaactgttgtcacactgggcactaaagcaccaactctgaaatataatttttgattatgttccctcctaaaataactaaagcacaaactctgaaatataattttcgtttacgttctctccctctactaatattccagcagagaacagagcccgcgccaggtgtccagtacccagcccctcatatccgaagctcaggacttgggggtttcgggagagagcggctccagcgcgtcgggttgtagctactgcatctgtgctcttccttccccaggaaacaaatggtggatcggacctcccaggctcttcgcgccccgccacccctccccgtgttagcagg

SEQ ID NO: 13 - DNA, 554 bp, Porcine
gcgcagggctccggggcccctccctgcagtactgggtgatagaccccactccaccctccgggtccctccacccccaccacgtgcaggccagagaaggcaaagaggcccagccaccctcaccagggaatttcttttctttttttgctggtttcaggcttttttctgcctgagtgaaaatgaaacaaacaccccctgcgcctcccggccaccagacacacacgcgcaccggcactcgcgcactcgcgccctcggcctcctagcggccgtgtctggggcgggacccgctctgcacaaacagccgcgggccgggtggagcggggagctcgccgcccgccgcccagtgcccgccggcttcctcgcgcccctgcccgccaccccggaggagcacacagcggccggcgggccggagcgcaggcggcacaccccgccccggcacgccctgccgagctcaggagcacgccgcgcgccactgttccctcagccgaggacgccgccggggggccgggagccgaggtgtgggccatccccgagcgcacccagcttctgccgatcag

SEQ ID NO: 14 - DNA, 5680 bp, Porcine
gtgggtcccgctgggcgctgcccgagcccctggaggccgcgagtcccgcccggcccggggctgcgggcgccgtggaggcagcgcggggagaggacaggccaccgcgccggccctgccctgttgctgccctgccgtgtccccgcttttgttctcgtcgttacctctgtgctcaactctgaccccgtctctgtccccatcttgtcgggcctgaggggctgcgggcttccacggggtccgccggatggaggcgggagaggggaggctcggggcgcgcagaggaggaggactgcccgggaagtctcgaaaggagggaggggtctgtctcccaatgtggggcaggggaggcggaggcctccctcgcccgggactaggtgggaagaggatgcctccgcaagagggaacctgagagtgaagtggggggcacagaaaccctgaacgcacagagagggagaagtcggggaactcagagagcggaggaccgaacccgaaacccggccgggggaaactttggaacgccgaaactttggcggcgaaaaaggccgctgtatcgggtgacaggaagcaaagggtccttcagactttaagccacacgttccaggagggagggaggcgcggagaccgtctgcgggcgccgctcctccccccaggaaagacaagagacccggacggttgcttttgtggttttgcttgtcgtcgtttgccctcctcttggcccctgagcgggccttgtcgccttgttcttgtgcttggaaatgggtgggtctcggagcgctggacgtgcggggaccgggggggtgggggcgaggaggagtcggggccgggacgcctcctagctggcaaacccttttccagggagaatccgtttccacaaacctgaaatagagagactgctggaagtaaggaaatgccaagtgcgaagaggttgtgtgtgtgtgtggtggggggggatgtggatgctttaaaatctgattttgatctgatttggctagtttatcacagtccatccttacctggtcaaattcacatacttctgctgcctgcctggctcctgtaggctttcactcagcattaattcagcaaatatttactgaacatctgatagatgtcaaatactgttccaggtaccaggaaagcccagaagtgaccaagacagaagacaagtgctccctcccaccccccaaagagcttgggttctagtggaatctggttcatgaccctcttcttgttctgcctccgttagcatccccagcttggtctgacttcaccaccaccaggggtgtacaaggctgaggtgggacagactcacagaaagacctcaaacttgtcttccattccagggctgctgactcataccatacgactctgtaagtttcttccctgatcttcagttccctttcttataacttggggcttgtaatatttcacctacttagcctctatgttatgtggcttttgtggatggcagtgggctctaaacggggcgtgggtgtgaccttgacggaagatgagcttatcacgtgttcaaaaagcagtcctgctttgaggcagggagctgacttacctgactttgaggttctctctgctgaggaaagagtgagaacttctgtggggggtcgggggcaagggtaccccctggcacctactgcccaattgtgaataaggagcaggtgcctctttctcacctccatctggggtacttggcctgaggaaggggtgagaaggaccaagagagggtaggaatagagcggtttccttgggtggggaaatcctccagtcacctgtgctggtgctcaagcccaggctgtcatcagtacccgggcctcgcccttccgtgggagcgcctcacatctccccagctgtcaacaaagccagcttctttcttctctaggaagagtctgacctatagagcttgaaggactgacatgagccccagagagggacttcctggtgtgcaggaggagggctgaggctcaggatggatgcttgcagaggcaggagtgcttcagcatggctttggtggagtctgtcctggagttacctggggcagaggcagatctcaagatgattagcaatgtactggcctggaaagagtcatcatgatttcatttttccagctcttctcaaggaaatagacttatagatgcaacctctcttgactgccgttatttattatgtgggcttttgccaagatcgtttcagctctgatactcacaggcgtgtgtggggggcagtacttaacagtaacggaaacgtcgtgccaggaacccttccctccgtacctttccccacctgcagggttacatggtcaaaatgactatttgatacacaaatgtaaactccaaggagctgcagcctcggattaatagaacagcagagacggacaatgattgagcacctcaagcacttttccgggcgtgtctccttacttcttgcaatattgggtaatacgtatctctagacacttaccatgtgccagctaccatccagctgctgttgttcccattgtgcagccgtagaaacagagacacagagaggttaagcacattgcccaggatcgcatatgggcaggcctgggactcgaactccggcagcctgggcccagagtccacattcataaccacggtgctctaggcccctcacccaccccgagcggtggggattataattatcctcaccacacggaagaggaaaccaactaaactgctccatcactcacaagtgacagcaagaatgtcttatacctgccttaaacgtatttaggattaaaagtgacagctgcaacctttgtatctgtagcactttttgccaagaacacttaatcctccctctcccacagggtgggaatccggacctttgtgtttctcagctggaaggggtctggggcatgaagccgggacccttcacacctgggctgcagctgctgagccgcagctccaaggccctgcactcctctgcaggggacatggcagatggacaggctctgaatgctggctgtcatctgacaggcctatggactgttagggctggaaggggccttggggaacattgagtgatgagattagtcggcctggctgggctgggaaacgtgccaaactcctacctggatggccactggcctcctttgatcagcagacctgaggctcacttgctacagttccctgcctctccatgaaggaatggccggaagtacatgcttccttgttttgagagtctgggcatcagggtatgtcggagaaggaggaaggtcatgtcggatcctctggaagttgaattttctgccttccaagtttgcatactctgtcgtgctctgattcatgaacctggagcctctaattccacgaacctgtagggtgttccccagaggcagctcaggaggaagggcagcatcagacccaccagccggcaactttgagcaagtcacagaggctcccagtgcctccctcccttccctgacccggggcgggtgagcctgaggatttgctgagttaaaggagagaggctgctttgtaaactggaaggtggcaaccatgatgggtgcttgcttttttttgttgttgttgttttgtttttttgtctttttgccttttctagggccgctcctgcagcatatggaggttcccagcaggctaggggtcaagttggagctgtagctgccagcctacgccagagccacagcaacgtgggatctgagccgcgtctgcaacctacaccgcagttcacggcaacactggatccttaacccactgagcgaggccagggattggacccgcaacctcatggttcctagtcagatttgttaaccactgagcctcgatgggaactcctgggtgcttgcttcttgaaaggaccagtttatcttagcccagttcctgagcctccaaatgctgtgaactttccctcccagttgaccacagtccagctgcctgcatcatttaatgtgaaagatcttccctgagtccgtacttaggtgctctgtggtgcttggtattggggcgttgaacccaagagaaggaaaaaacggggtctatccacgaccctgtggccctgagaccctgtagactcaggggaagtcagaattcccaagagaaggcagcttccagcaggaagatttctgtgcatctttgtttttaacacacacactgaaagggaatgtttgtgaggcattttcccaaggtggacacacctgcataaccactacctggctcgagaaacaacatgacaagcccccccccctcccccagcagctctctgagcctccccttcccagtctctaccactcccactctgacttctggcaccacagattggttttgtcttttttttttttttgtctttttagggctacacttggggcatatggaagttcccaggctaggggtccaattggagctgtggctgttggcctacaccacagccacagcaacatgggatccgagccgcatctgcaacctacaccacagctggtggcaatactggatccttaacccactgagtgaggccagggatcgaacttgcattctcgtacatactggtcagatttgtttctgctgagccaccatgggaactccctggttttgtctatttttttttttttttttgtcttttttgccatttcttgggccgctcttgcggcatatggaggttcccaggctaagggtccaatcggagccgtagccccagcctacgccagagccacagcaacgtgggatccgagccgagtctgcaacctacaccacagctcgcggcaacgccagatcccttaacccactgagcaaggccagggaccgaacccgcaacctcatggttcttagtcggattcgttaaccactgcgccacgacgggaactcccggttttgtctatttttgaacgttaaataaatgcaagcatccagggctgctttgactcagtaccatgtgtgagatttaccctgttgatgtcagcagctgtggctggttccttctcacggatgtgtgtgaccctcacctggaccacacctgatctggctgatgatgggccttggggtttttccagcttttggtcccaggtcacgtctctgtttgaacttaaatgcacttgctttcaggtattaatctggggcggaatgactggaacatgaggtgtggttggttcagctttagtacatgccagcagggaggatttcagtagtttattaagcagatcttgaagactgtggtcaactagctcatgccccacaggagggggcggtgaatttcttccccagaacaggagtgacaagctaaattaggcatccatccgctggaagctgagggggcagttcttggctcctttctgtcaggtttcggccccttctccttagtctggggtttctaggctctactcccaggaagtgtctggggccacttgggaacaatgggtgggggggctctgagcccctacttacttcatttccctccttcagccaaagccccctgtgtcctctgttttacatagtggggttctgagaatgacttcattttttttttttttttttttaaagctttagctgttgcgacatttacaaatccactgctgtgaggtctcttccaggtaggaaattgtattttgggagcaggaggtgggtgtggggagggttaagcattattcagccaaagagttgggttgggcctcagtgaccttttgaagttcttatagcttggc

SEQ ID NO: 15 - DNA, 94 bp, Porcine
ttgccatgcaggagatctcagaacattctataaaaatagtgttcaaacagaacaacttctgaagcctaaaggatgcgaacaagaggctcggaag

SEQ ID NO: 16 - DNA, 427 bp, Porcine
gtagcatttcaacgggagttttgaggatgctctcctttagccacccctctccattttctgcccccttctttttaaattctccattggctgtccctgctagttgtcatttggggtggtttgggttcagaatggttctcattttcgccgaggagtgggtgatgtgggcggcctgtgtgtctctcccaagggtggtggctgtccctcctccaccaccaggcctagtttggacctgtagtttcgcttagtgaaggaggccgggccgatcctgggccggagagagacgtctctgccttggcatgcagctctgagtcaacaggcctgataaacagcccacttcccagggcgagcaaggaggaacaaggcccctggctgctgtgggatccgtctgcgctcctcttcgtgaaaccgctgtttattcttttgacag

SEQ ID NO: 17 - DNA, 112 bp, Porcine
gagttggaacgcagcaccttcccttcctcccagccctgcctccttctgcagagcagagctcactagaacttgtttcgccttttactctggggggagagaagcagaggatgag

SEQ ID NO: 18 - DNA, 3666 bp, Porcine
gtacgtgaaacgttgaaatgatttacctccgctttgctggggtcaccgggggggtgggtatcatgagctggctgcagcgtggagagaggagcccccctctccccctgacttcttgctgctccccccagttgttctgaaagaagacaaagtcctccagtccccggcatcggatctaggagtgggagctggcaggatgctggctcagtcactgttggttctgctttcgttggctgcccggcaggacctcacggggtgtggctacagcctggggttctctgtgtgggccacacagtgccattgtggggccaggaggacgagtctcaggcccgggacctgtgctgggggcggacatagtgccctctcagggcagcaccgatccttcatgtacctcgccctatttctcttggaaaaactcttgcaccatgatttctgagccaggcagcaaggagaagctggctggatccaggcttcagatttttgaaggggattcaagaaaggggcctacaagatgtccctccgagaacaggtctgtgatggctggagcgacagctgtgaaaaaaataagtggaaagagccttcggtgcggtactccccccccacccctgccccccaaattataccatgtttcttccaacagggagcatttccctgtaatgcaagccaatttaaattcttgagggtgcacattttggttttatttcaactgattattagtgtagaggagtataagataacatttctttaaaaaccatcaacacaaacccatcactcgtgattcaattgtttaggagaggagggaactccgcctcgtataccaaatacagtctgctctcggtgcagcgtgcagtcccagcaaggccctctcctcgaactcacacagctcttgtctccagcggcttccttcccatgtcttggctaggctgggctttcttagtaaccccaaaggcggagaatcaaattcacagattttttttttctggatatttagatcttgtattttaagccacactatttataaggctcagagatacatttaaactctgactagggcttcttataaaagtgatatctggaaagaaggtctggctttaacagagtaagggtcagacccccccttttcccattaatgactccaggaatgctctggaagactgaagtggaggcaaagaaggacttgaatttgcatgacctgatcttgaatccaggctaaatttttcctggctgtgcgcctttaggtgggtcatttacctcccctaattctcaggtggctcacttcatcatctattcttttactgaggcagagaggtccctctaccaccaggttgaatgagctcagtgacctctgaaaactccaaagtgctgcacagatcaaggtggtatgaggtagaagaggaagggaaaaaggaatgagtaggatcaaagaaagaaggagtgaaaagaagcagagtggagagacagagccaacacaaggatctgggtaccacttctggattagggtcagggcttagaagatgacattgatggttgggtctttttcactacacagagaatagagctgaccattagacttggcccggagccagtcattgtgaaagaaatcaatattcagattatcatgacaactaccatttgtgtaattttaattcacaggatcactttttctggcccacgaggttgaaataagaatggctggtcagattgactggggcggtccgactggcctgtgcttgagagttgaccatgagctccctgccatctagcgtgtatgtcacccagacttttaactcaccatctggactgaccctcgagaacttgatgccatttgagagcacccaaggggtccagaggaccttatcaaatcctctgactcctctgtgcaggctgttggccagcttatactccttcccatccaacgtgatgttcctttggcaatttgctttgccaccctgccaaccactgctccaaagtagggatgcttttggaggtacccttccaattcagcaaagccaagcaccacatctgaggctctgccttgcctgtctttgacctccagggccgtgatggtgcagcccgaggagatgatttccactcccagtgttgttcagcccgaggagatgatttccaattcccagttggtctgcttgcagctggaatttttccatgttccttgcccccaaggggagttctccaaacacagatcttgtaactgaaaccatgaggaaagcttggggtgtgtaggtgctccaggtccttcaaacgccccatcttttggcagtttcttgctcaggtgggtccagccagagtcctggagaattcagctctttgatcctggctggagtggggggtgcaccaccaggtgattgtgaggtctggatcgtgacctgtgagcagggagccaagtagcatcatgttcagctccttctccttgggatcaaagtgagaggctccaaggagctcagcaaggtctacctggatggggcaggttgctcctaggacccaggtaggtgcggggagcagggtcagtacctgggctccacctgcagccccaggacaggcacccaggctggaacgattcccccaggcaggggcagcacctcacctggaggaagcatttgggccttgcccactccacaccccaggcctgcctgggggcctgacccggaggcttctgggtgaagtggcctgagggctcaacacattttgtgggcaatcctatctctttttttatttttatttttttattttttgctttttagggccgtacccgctgcatatagaagtttcctggctaggggtcaaatcggagctacagctgccagcctacaccacagccacagcaacacaggatccaagccgcgtctgtgacctacaccacagctcatggcaatgccggatccttaacccactgagcgaggccagggatcgaacccgcaacctcatggttcctagtcagattcatttccgttgcgtcatgacggaaactctggcaatcctatcttttgatcaccacttctaggaatctgtggccactgcagcaagttgagctccagtgaacctgtcctcataaaaggagccttcagctctgtggctgccttctcatacaggtcttggctcattcaggggaagttaagcccacaggacatgtttcaaaggacgggaaatgcactgggttttagcacagtctgcacgaggcccgggagtgggggtgcaagtggtttcttttggaaaccgctgcaggggctgagttgtgggagtggcccaggagcagagagaaatggcaaacgccttggcaggagggcctgtgggatggtgggagggctcaggtggaactgggcccgctgggttcacctgatcctctgagggctggggcccaggtggtgctgaggtggttacactctcccttataagacaggatgctagtgctctctaggctctaatcctgtgctctccctcttccatgagaaatgtagaagcaacccccacttttcctatttggtgggtaagatagtcaaccaccaatcttgagaattagagagttttgaaaattctgtgacaaacacatccgtgaagggcttttagaccacatgggctgccaaatgcctcattttaatccagagagaaaaataaaattgttttaattttcccttctccttttcttttcccagg

SEQ ID NO: 19 - DNA, 87 bp, Porcine
agaaaataatgaatgtcaaaggaagagtggttctgtcaatgctgcttgtctcaactgtaatggttgtgttttgggaatacatcaaca

SEQ ID NO: 20 - DNA, 2254 bp, Porcine
ggtaattatgaaacatgatgaaatgatgttgatgaaagtctcctctaatctcctagttatcagccaagtcaccagcttgcattaaaagtaggattcactgacaccgtaaagaaagcattccagaagcttttaaggactctaagccttcatttttctttttttttttcctatcttcgacttggttgctaggaagcttagagcaaagtattgtgcttaaatgcttgcattttccttggccttcattttttttaaaacattttttcttattaaagtatagctgatttatagtagccttcatctgatatgatttatcccctggtgttaaatcctggcttttgttagatgccatgggatcttggcaatttgctcaaactcattttgccaatatcttagctatgaagtaaaaataaagttaaagattttgttctcacagagtggctgggatgaccaaagtcatgtgaaaacacccgagtgactaaaatgtttctctgtttcgttttgttttgttttgattcttgtattgttttcctatttatcgtaaccacactttcttcataagccatttcaagcacttcctgaaagtagatggactttaagtttcttggacttccagttgtggcgcagtgcaaacaaatctgactagtatccatgaggatgcatcttcgatccctggccttgctcagtgggttaaggatctggtgctgctgtgacctgtggtgtaggtcacagaggcggctcagattccaagttgctgtggctgtggcgtaggccggcagctacagctccaattagacccctagcctgggaacttccacatgccgcagggtgcaaccccaaaagataaatgaataaataaataaatatgcgaccttcctttcttggggcccttgcatgtttttctctctgttaggcacactcttgctaatccctcttcactgggcctcctatgtatccttcagaactcagctaaaacatcatcccctcccctggggagccttcgaggtcttcctgttaagtgctcctatgctttcttggagttttgaagtcctataatgatgtgtttatcaaaatagggtccaccctccctgccagcttctttacaccacagacacatggtgtctgtttcagtcaacactgtatgtctggcacttgacatgtaacgcatgctcagcaggtatttgttgaatgaatggaggcggtctgctagagtcgtcatatatttactgatcccgtcttgtaggatggtctcactgcttttgttagcttaagaagtaccttttttttttttttttttttaatggccacacccatggcatatagaaattccacgaaggaaggaagaaagaaagaaagaaagaaggaaattcctgggtcagggattgaatccaagccacaggtgcaacctgagctgcagttgcggcaacaccacatcttttaacccactgtgctgggccagggatcatacctgtgcatctacagcgacccaagccacggcagtcagattctttttctgcctttctttctttcttttcttttttttttttttttttttttttgtctttttgccttttctaggtgcggcatatggaggttcccaggctaggtgtcgaatcagagctgtagacgccggcctaaaccacggccacagcaacacaggatccaagccttgtctgtgacctacaccacagctcaacggcaacgttggatccttaacccgttgagcgaggccagggattgaacccgcaacctcatggttcttagttggattcgttaaccactgagccatgatgggaactcctgcagtcagattcttaacccaccatgccacagcaggaactcctagaagtgccctttgaggctactctgtagacagctttgagccagcgaggcaagacctgtttttctggaggaagataaatcctgggtgagggatgggtgggctgtggtcttcctgggacccatctctggagcctctctccctcagcaaagccaccttggacaataagagctgccatctattttttttttctttaaactaagatttgatattttccagagacctccctcccaccgttcgatctgagtaattctgaaatgacgagagccccgtgatatcattttttcgatctcgaaggtggaaacctgggagtagccacaacccaggctctcagctcagcctagggtttcaatgataatgattgcaaaatagcttttctctgcgttccaagtaacatgatatgtttttatttccatt

SEQ ID NO: 21 - DNA, 45 bp, Porcine
tgcttttagcccagaaggttctttgttctggatataccagtcaaa

SEQ ID NO: 22 - DNA, 545 bp, Porcine
gtaagtgctttgaattccaaatatctctaggtcaccttccatgtgaccctggtggccctacagtccattcttaacatggcaggtggtgacgcacttgtggtcctaggtggaggagagggatggggttccaggggtctgagctgtacttctccagcccctagacttgcctttctagagcatgagttgtgtttttcctttgcttctcatcaagtatctatctctttaagtgatgttgtttggagaacattcctgccttgctcataaaaaagaatcagagtagatattatccattatgctacctactacatgtggtataaagacccttgcccagaaattttgccaagacaaaggattaggaagaaaggctgggtgtcctgataaactaagtgtgtgtattattattatttaatattattactaatactgggtgatttaagggactcctaaggccttcaatttttccttttttctttttttttccctaatcttccgacctttggtttgcctaatttctaaaaaatgtttgtcatctttttcatttctta

SEQ ID NO: 23 - DNA, 55 bp, Porcine
gaaacccagaagttggcagcagtgctcagaggggctggtggtttccgagctggtt

SEQ ID NO: 24 - DNA, 190 bp, Porcine
taacaatgggtaagactgggaaacggccatctgtgtatctgctcaaggctgtagagtccaaataaaatggtttcacagccatgaccttcatgaccttctccagtcgcgtcgtccttctggcttattggacattctggcacatgggtcaccctccctgccttcctcagcttgttttccgtttgtacgtagg

SEQ ID NO: 25 - DNA, 104 bp, Porcine
actcacagttaccacgaagaagaagacgctataggcaacgaaaaggaacaaagaaaagaagacaacagaggagagcttccgctagtggactggtttaatcctga

SEQ ID NO: 26 - DNA, 294 bp, Porcine
gtaagaaaagaagcgttgccctatttcagtaaatccaagcagaacagggggacggaagtacatacacgttgtacaggtacgatccccaaagggccaccagggcagcccgcagaggcacttgggccagagcctcctgtccttcccccagaagatgccgcaatgtcacaccaccagctgactggggctaaaatacagtcaggattcaaggccagtcccacaagccatgactgacccatgttcccccagactgtcgtaccttagcaaagccatcctgactctatgttttgtcaccag

SEQ ID NO: 27 - DNA, 128 bp, Porcine
gaaacgcccagaggtcgtgaccataaccagatggaaggctccagtggtatgggaaggcacttacaacagacgtcttagataattattatgccaaacagaaaattaccgtgggcttgacggtttttgct

SEQ ID NO: 28 - DNA, 653 bp, Porcine
gtcggaaggtaggtgttgctaataaaactggccttgagtttttccccttccactatcagaggatgggtgaggggcccctgggtttacagaggctgttcatgtcatgtctgaattagtggagaggagaatggtgtcacagggccattttagactcccttctgctgaggtccccaaaggctaagaataaaactagtcagagggtcaactctttcccacctcagggtgaggggcttgggttgcagggaagaaaatctgctatacccactgcacccaaagtcgacagtacacccacagccacctccaccctgacctccacggccctctgtggaaattcctgcaatgcccagagcagctgaaaacacatgttctctctgcctggttggcttccaagagtgagagaggaaggagcagggctgagcatgcccagccaccctgccagaatcaccagtcaggtaagccactccacctccccaaagctgaatgactgaatggtggagagtagctgggaatgttacagcaacagacgtctctcatccaggatggggaaaaatcattcctttcctaaactgcaaaatacagactagatgataatagcatattgtctcctctagaaatcccagaggttacatttaccccattcttctttatttcag

SEQ ID NO: 29 - DNA, 685 bp, Porcine
atacattgagcattacttggaggagttcttaatatctgcaaatacatacttcatggttggccacaaagtcatcttttacatcatggtggatgatatctccaggatgcctttgatagagctgggtcctctgcgttcctttaaagtgtttgagatcaagtccgagaagaggtggcaagacatcagcatgatgcgcatgaagaccatcggggagcacatcctggcccacatccagcacgaggtggacttcctcttctgcatggacgtggatcaggtcttccaaaacaactttggggtggagaccctgggccagtcggtggctcagctacaggcctggtggtacaaggcacatcctgacgagttcacctacgagaggcggaaggagtccgcagcctacattccgtttggccagggggatttttattaccacgcagccatttttgggggaacacccactcaggttctaaacatcactcaggagtgcttcaagggaatcctccaggacaaggaaaatgacatagaagccgagtggcatgatgaaagccatctaaacaagtatttccttctcaacaaacccactaaaatcttatccccagaatactgctgggattatcatataggcatgtctgtggatattaggattgtcaagatagcttggcagaaaaaagagtataatttggttagaaat

SEQ ID NO: 30 - DNA, 1961 bp, Porcine
aacatctgactttaaattgtgccagcagttttctgaatttgaaagagtattactctggctacttctccagagaagtagcacctaattttaacttttaaaaaaatactaacaaaataccaacacagtaagtacatattattcttccttgcaactttgagccttgtcaaatgggggaatgactctgtggtaatcagatgtaaattcccaatgatttcttatctgttctgggttgagggggtatatactattaactgaaccaaaaaaaaaattgtcataggcaaagaaaaagtcagagacactctacatgtcatactggagaaaagtatgcaaagggaagtgtttggcaacaaaataagattgggaggggtcgtcctcttgattttagcgtcttcctgtctctgctaagtctaaagcaacagagttgctttgcagcaggagatcagagtctaccttagcaatcctcagatgatttcaacagcagaggacttcaggttatttgaagtccatgtccttttcgcatcagggttttgtttggcttctgcgcaggatactgatcaagattcccaatgtgaatgttggagttacagggaatccgaatgaaccaatgggagctcagcacgaaataaaagcacagcttctaagtaagtttgccatgaagtagcgaagacagattggaaagagagggggctgatcactgtggggcaatgccatttctaagagacacagggcatggagttggcatgtacatacagcttggatccaggcactgaatgggaggcaatgagagtggctccagcctcctcaaccatatgacaactagagcagcactgtcttagaagatgcttcttgctttggccaagtcatattcagtctgccagactctggaacttgtgtctacaaatccttgctcagaggaagtggatgatgtcagagtggacagaggcctacattgggttgaagtgacttcctagaccttggcttcatgacaatcaggcatcagcaagccctgctgccacctgctctaactctcagagtccctcagcccatcatgggcaacttgagagccaccgtcaaggagtggactagaggaaaagcctgcttatcagggaacctctcatttcccctgccccagctgcactactgaagtgtaactgccggacatgtttaataaagtggttaattgattttatatcaaagtagagaggatggcaatgggagacccagtcctcatgactaaacagcttttcaatccctttctctaagaaaagctatgagatcttacatgtaatttaaagttaagcagtttggtgtaaaggaagttaggaggcaatatttacatctgcaggtatgtgatatacttttgcttgtgttccagtttaggtcatttgtgtccattttcaaatgatttacttgaagagccattgcactgacttgatgttcagcacgatgggcttctttgataaaatgaaacctacattttctctactgtttccctgggcctcctactcttcaattcttgctaaaaatttttgcaacccagcaaaataactcaacaaaataacccaacaaaataactcaacaaaaatcctggagaagtagtcttgtaaaagaaaaaggaaatcacaagtcaattaggactcttgtttctctataacgcaagtttatggaatccattctggagtgcagagacttcatggtgcaagttccaaactacagaaatgattcgttctcaaagattaaagaaaaggactgatatttccttttgaaggaatcttgatttttaaaaaaaaaatcatttaaatttaaatttcaaatggacaaattcaagatcttattaatagttcaatattaaaaaataaaaattcctgatttaaaattaaataaattattttctcagtatattctggtctggtcatggattgtggcttttttcccaaagatgttcagaactgtcatttaca

SEQ ID NO: 31 - DNA, 1401 bp, Porcine
ccttgttctaaccctttagcagggattaactcaacatccaggacagccctccaaagtaggtgttcttaggacccacctttctagatgaggaaactcaggtgcggaggtccagaaccttgcctgaggtcagacagctaagaagtggtggcctgggattcgaacccagggggtcttgctccagcagtcttgcttctcaccctaggggtccagtctgtctagaaacaccagcacccagcaggggtgaggagagatggaagagatccccccagaggagcttattcaaattcttcatttttgggcccttctggaaaacagccaaccacgctccaatcctaaagtactcctcctctgagccagcaaaggggctggtacctctgctggaggtacctggcttggggactaagagccaccatagacacagagtccctgagcacaggtggccctccgtgcagcccagcaatgcatctctaagccccagagagctctcaactcctagcttccaagccacaaacttccctgcatccctctcagactctcccctgcccaaggtcagtcctacacactgcctggacgaagcgccccaccccctaatggttactgtcacttgagtgtgcctactgggaaaagcaaagaattaaacatctaaatgctcatcaaaagggacctgggtgaggtaaagtgatgccccctcccgtcaatggcatgttaggcagctggaaaaaggggtgaggaagcgcttcaaaaataggaagttccccattgtggctcagggggaaacaaaccccgccttgtaccccatgaggatacgggttcgatccccggcctcgctcagtgggttaaggatccggtgtcgctgtgagctgcagtgtcagttgcaggcatggctcgagtcctgcgttgccgtggctggggcataggccagcagctgcagctctgatttagcccctagcctgggaacctccacatgccataggtgcggccctaaaaagcaaaaaaaaaaaaaaaaaaaaagagagagagagagagagagatggaataaactcaaagacataatggtcagtggaaaatacaaggcaaggaagagcatatcagcaggctaccgtgtgtgggaggaaaagcacaggaagagaaggagagagcgcatttgctaccgtatttacatttgcctgcatatacacgactgtccccatgcagaggaacaggaaagactgcactgtctatactctctaggacctttgaatgtctgccatgtgcacagagtaatatattcatagtcaaagcaaataaaatgaaacattaaattatatactttcccatatatatgtatatatgtggaaattacacacacacacatatatattttgtgttgctaatgtccctccctactccccgcccacccag

SEQ ID NO: 32 - DNA, 84 bp, Porcine
ggcctggaagagaatcctctggtggttgatcctacttgcacttgacctcttagggctgctcctgtttggcctccctgctgtcag

SEQ ID NO: 33 - DNA, 201 bp, Porcine
gtacaacccccttcccctagtgctcaagatgggaccagcaggggagggttaaagtggctctttcccagtgcctccttaagggatagagagtgctggctctctcctgcacaagtgtccttgcgggctctcccccttgtaaggagcaaagccacagggctcctgagcaggctgacacccctcactgctgcccccatcccccag

SEQ ID NO: 34 - DNA, 90 bp, Porcine
gcatctggaagtccttgtccccgtgggtgtctgccctttgaccagaacacccctgctgggagacaactccacgggtcccctgcatccttg

SEQ ID NO: 35 - DNA, 284 bp, Porcine
gtaaggagctgccatctccaggatctctgggcctccagcaccccacccccaagtccctgccctcctcgcatcccccaccctggcagggctaggcgctccaccccagggccccagcaggttacacatctcgaaataccctgctggatctggggtagagagttctagggcagggcctgggtgtgacccacttgcaagtccctggggcccaggcctggggaggtgacagtgaccacgcacgaagcaggtggataatggacgaatccctccatccctgccctggctag

SEQ ID NO: 36 - DNA, 138 bp, Porcine
ggcccggcctgaagtcctgacctgcacctcctgggggggccccattatatgggacggcaccttcgacccagatgtggcccagcaagaggctacccagcagaacctcaccattggcctgacggtctttgctgtgggcag

SEQ ID NO: 37 - DNA, 2553 bp, Porcine
gtaaggcctgggaggcgagcagtgctgtccaagcgaagggttgggaggggcgtgcatgtgaagcagggcgtggggtgccccattctccggggccacagcatcccaagcggaagcagaaggcaaagacagcacctcctgggcaagactccaagggtgaggcaggaccgacccctccttcccttcctccctggacaccagcaccatggagcccagccagcgcaggcagccgggggctcaggaccatgtcctggaaggaacctggctagtggtgagaaaacaatggagtttttcaggcgaaagtgagaagaggtgagaactgggtaagtagaggggatgacccagctgcagtgagcgccccgcccccatggaggtcagtggctcaggcgcaggttagggagggaggaagattcaccaagcaagtctgatggtgggactggggccgggggacggagggctcttgcaagggagtggatctgggctgagtaaagagaaacgtgaagaaatggggatgcaacagtaacgaacctgactaggacccatgaggacccgggttcaatccctggcctcgctcagtgggttaaggatccagcgttgccgtgactgtggagtagtcgcagacatggttcggatcccgagttgctgtggctgtggcgtaggtgggcagttgcagctccagcctgacccctagactgggaacttccatatgccgggggtgcgcccccccaaaaaaagaaagggggatgttgagagtggcagggtcagcaggccagagggctcagtgagggaggactatggggggtggtatcaggaagcgggctggaaggacggggctgctgagggggacgagtgaggccgcagtttgggagggaaggcagactgatgatgagcaagctgagggagaggtcatgggggcaggtggctcaggagagggaaggacagactctctccaggagaggaggccaatcgaggaagtgagaggcccccaggtatggaggaggaacctggaatggtaggtggagaactcacaagggtgctggtctccccatctcccgattagggatggcggggggtccaagctgggtactcactttccagtagtgatgcaaatgggactcctggctgagagtggcacttagatcctatagtcctaaggctcagagaggtagagttcaggacaatttaagggagcgtttaataatggaagaagctgctttcgggaggcagtaaaaagctttgcatcccggaaaagatatccaaaagtatctgatgaattcagctcctccaaatgactcctctctgtccctcacaccctagacgggagaaagccaggaggacccctgggaggccagggtgcaaagaggaccaaggtggacggaactgctggcctctccagggccttgatgtccccacttccgttctggatgctgagtagggtgttcccataccagccctctgggtccagaaattccagagtcttgagatccaaattccaaggttctatgagtccaacactctgggatgctgaggcttccaaggtctctcattccagttttcacagttccaccaggaatagaacaagtgcaggtaaagctatgggctccactgccaagcagggttcaaatcctggcttcatacctaccagctgtgtgcgagggtgcatgagttcctaaagctcttggagactgtttcctcaccaggaaacggaactaataatggtgaggattaaatgagataatacacattactttgaacactctcacatgataaatgttcaaaaagatcaggcattattattattattttagaaccttaggatcccaaagtctgttcatacagtttccagtattctggatgtctcgattatctgtgtaaggaatcactacaaacgcagtagctgaaggcagttcactattatcatagctcatgactttgtggctcaagaattccgactgctcagcagcaaaggttcatcacttctctcaaacagctgggtctcctgtgagacagccgcctgaggaagactggcagggtgcctctccatggctagcttgggttctctcactctgtggcagtatcggagttccaggacttcttatgcgaagggtcagagctctaaagggacagaggctaacgcgcgggtcttcccaaggcccagcatggcatcccttccttgtgcctctattgatcaaaggggtccgggagagccgagttcaagggaagggacacaggggctctaggggcagggctggcaaacaatggacaattgttatgattattatttaccacaccttccgcatgaggaagttcttgggccaggattccaacccaggccagggatcaaacccgtgacccaagccacagtagtaacaacgccagatccttaacttgctgagccaccaaggaactccaattggcaattaattttaatttgcctccaacggggactgccctttccggagttcctgggcctggggtcgcagggtcaccagaacggacatgggggcggctgggaagggcgcagtgaccagctgactcggacggcccgctccgcag

SEQ ID NO: 38 - DNA, 1128 bp, Porcine
gtacctggagaagtacctggcacacttcctggagacagcagagcagcacttcatggtgggccagtgcgtcgcgtactacgtgttcaccgagcgccctgcagccatgccccgcctgctgctgggccccgaccgtgggctacggatggagcacttggcgcgtgagcggcgctggcaggacgtgtccatggcgcgcatgcgcgcgctgcacccggcgctcggggggcgcctgggccacggggcgtgcttcgtgttctgcatggacgtggatcagcacttcagtggcgccttcgggcccgaggcgctggccgagtcggtggcgcagctgcacgcctggcactaccgctggccgcggtggctgctgccctttgagcgtgacacgcgctcggccgccgtgctgggcccgggcgagggcgacctctactaccatgcggccgtgttcgggggcagcgtggccgcgctgcggcgtctgacggcgcactgcgcccggggcctgcggcgggaccgctcgcgcggcctagaggcgcgctggcacgacaagagccacctcaataagttcttctggctgcacaagcccaccaagctgctgtcgcctgagttttgctggagccccgatcttggccgctgggctgagatccactgcccgcgcctgctctgggcgcccaaggagtatgccctgctgcaaagctagcaatgccggtgagggcccttctggaagcagcggggcactgggggtggggggagactgcgtgaacgcctcccccgctgcggcatggctgcaggaagctgggcctttgggacgtggctcccggaggaggatgagccatccctttccatcgagacccgggcacctccagctgcctggagaccattcacctctgaccttactgagttcagcggaggccctctgaagagatgttttagccccttccccatatcccctacgctttatatggtactgaggcgccaaaagggaacatgatggcccgaggacccagaggatctatgagtcagcctgtgaggtcagcagctggagagcaagactgaccctcaggccaaatacatctgcttctaggcacaagccccagatgaagaaactcagtggcatccggttccctgactttgctggtt

SEQ ID NO: 39 - DNA, 43 bp, Porcine
tgaattctagctccgtctgcctacgctggtccgaccgcaaggg

SEQ ID NO: 40 - DNA, 1115 bp, Porcine
gtgagtctgcagccggtaaggacaatcgcgctccctccgctgcgccttgtccctgccccgcgcccagccggaggaagagcgccgcgagtccccagcccgcagtggtagtcgagatgtgtgtcttcggccccaggctcctgggtgcagatccccggctggggcggaccgagctcggccctggctgtgagtcggcagagcgtccccggcggcctgggccccgcgggagggagaatctcgcggagccaactgtcgaggggggccttggaggacgcttcgccccaaaccgggatgggaaaactgaggtctgtagagggagggagagggattgggaacggccttgcagaggccaccgaatgagcagggccaaagccccagaactctggcccggggatctttgacctcgagcggatccccacagagcggccaggggtccggtgctcactgcttactgtgacacaaccctcccggtacatcagggagtgcgtattgcgtcttgtcccctgcaccaagccccctctagccgaggaggaccccgacgctgtggcggagcggggacgagagtgacttgcccaagattatcgccgagcgggtgcgagctgaagctcgttcctgcggtccccgggagagtccaggctgccgcctcctggagcaacgccctgctgccacccctgcccctgctccccgcccggggggatcgcggccgcccctcgctgcgcagcatcccgcttcccaggcccggcgtgtccccgctgtgccggctcagagcttaatttcggcgtcctcattgtctccctggggaatccctctccaagatcagcccaagcgctgttgccctggtccggaggatggccgcccttcgctcgccgcaggagtttgggagggagacctgagagccaaggcaggggaccggtccttggggcacggctgcaggcttcgggtgagcaatgagcctctgtccccgggtcaacttgccagaactgccccatctgggcctagggtccagcaggatgagaagatgacctggaatccacagtcccctagcggggctgcccgggggagggcggagcagcaaggctggggcaactatcctccagataaggagcattcctttgcag

SEQ ID NO: 41 - DNA, 191 bp, Porcine
gtctcctccggaccccgaagacacaagctcagagcctgacggcccctgagagaggtgggcggatccgccaagtcacacccaggctctgcaggtgctcaggcccagacgctgcacccagagatgcgctgccgcagactagccctgggcctggggttcggcctgctggtgggcgtggccctctgctctctgtg

SEQ ID NO: 42 - DNA, 564 bp, Porcine
gtgagcatgccccgtggagccctccggccccacccgactcctccctctctcagcatctcaacccccaagcctgacccttcactgaactcccagggctctcatccgcctctcctgacacacctgtccttctggcgccgtaagagatgaactagtctggacttacggattttgctttgcactggctctttcctctgcctggactattcttctagccatgttaacgaggaactccagtttatgctccaaaattcaccccaatgtgttctttctgcaaagttcctggcccccccacccccaccccccacccccgccccttgtgtgcagggtctggcatcaggaacattcctgccccaggaatgaagggctgcatggctctataataactgtgttgccacagaccgggggctttgccatccacggttcgccagacccaaggagtgattggtggggtgggggtgggggtcccaggtgcacccctgggggccttcattcccactaacatggaccaagtgggttttcagcctcaggttcaaagtcgagtcagccagtgttcttccctcccag

SEQ ID NO: 43 - DNA, 66 bp, Porcine
gctgtatgtggagaacgtgccgccgccggtctatatcccctattacctcccctgccctgagatctt

SEQ ID NO: 44 - DNA, 2558 bp, Porcine
gtgagtatgagacggggagaatgggcgagatgggaggggtttttaaggccgctttgcaggttcttacattctcagctcaggattctgatcagtgtgattaaacagtgaggcaatttatgaacggctgcaaatgtggagtaaaaactcccctgtttcagtcccgaggggtgccctttggcatgttgtgtggctctgagcctcacttgctgcacgtgtaaaagggggcgatagatggtacctgtgaccgtgctggtgtcacccctggcacataggaggtgcccaggaaagagtgcttttaggacaagacctttttgctcaatttggtgttctgcgtggattcgaggaacaaggtgcccagtctctcccacatggcaaggctgactttttgacagctaagtgtgacacagatcaagtgtgatgtaggttgggacagtcccgagggtgcatctggccccctggtcttttgctgtccatgacagcagaaggaaagtaaagcatgcatcgcaagggaagttcctgtcgtggctcagtggaaatggatctgacgcgtatccatgaggatgcaggttcgatccctggcctcactcagtgggttaaggatccggtgttgccgtgagctgtggtgtagattgcagacacgactcggatctggcatggctgtggctgtggtgtaggccaggggctacagctccccggaacctccatatgctgcgggtgcggccctaaaaagacaaccaaaaaaagcatgcatcacagggagttccctggtagtctagtggttaggattcagtgcttatgttctaaaaaagcagaaaggctgcttgcttttgaaaacagttgtgaccacaatgtttttggatttttatcctgtttccccggatttggccttatttttggcatctggtcaccattattttattctaacctgggtctgggccccctgaacccctttcccaccaacaactttgaagcatttaggtggtttccaggtgcccagcgttctaaattagtttgtaatgagcagctctggacataaagctttttcccgcctaaagatcctttcatctggtatgttcctgagccaaaggatatggctgggttctcatccgcttgctctccagagggaccagaccgtcccacactcacgctcatccccgcacccctacgcacccccgccccagcagctgcgccgccgctgggctaggactggacataccagctgtcatgagaaacaaaacccaaaccacctcgctgattggagagatgggaaatgcagtctggtgtaaattacgcttctttgatttgttcggggccctcatttcccccaggcctttccatgaattgaattctgcctccatgaacttgccctctcacctccttccctcccgggcctctttgctgtcctctgtccccacccttgtatttgctacctctttttttttttttttttttttttttttccttttgccatttcttggccgctcccccgacatatggaggttcccaggctaggggtcgaatcggactgtagccaccagcctacgccagagccacagcaacatgggatccaagccccgtctgcgacctacaccacagttcacggcaacgccagatccttaacccacgagtgaggacggggatcgaacccgccacctcatggttcctagtcggattcatcaatcactgagccacaacgggaactccagtatttgctacatcttgctacttttttttttctttctagtttgtctacctcttggttcttctgagggtttgtgtgtgtgtgttgtgatagattgaggctggagatttgtgactttatttaatgtttagttatgtatgtatttattggccacacccacggcatatggaagttcccaggcgaggggttgaatcggagccccagctgccagcctacaccacagccacagcaacacaggatccgagctgcgtctgtgacctataccccagctcacggcagcgctggatccttaactcactgagtgagaccagggatcgaacctgcgtcctcatggatactagtcgggtttgttaccactgagccacgacgggaactcccgaggatagtctttatataaggtcagctggtgtcggcgttactcacatgtgcaaaatacagaccttcacagccgtgcctggattgatggccgtgtaactgggtcccacaaccacccatcaccgtgggctcaggttaagcaactcgcccaggctagaaagtggcagaaccgggcttactgggcctttgcagcttctcagtccttctacccaatgcccaggcccttccagagcaacatgtttgcaagagagacagaaaaagactttggagacaagtggtaccgggtttgaatcacagcaaccccggacagaccgcctctgtagaagcccagcccctgcagtgggggaggtctaagagagtctgcgtggagcctggtggggagggggtacctgtcccgtgggggggttcatcttggcttccctgccgagcatccctgcccccggccccggcactaatggctgtgtctcgcctctcccaccag

SEQ ID NO: 45 - DNA, 51 bp, Porcine
caacatgaagctccagtacaagggggtgaagccattccagcccgtggcaca

SEQ ID NO: 46 - DNA, 82 bp, Porcine
gtaagcagactgtcacttcccccttggtggcccccgggggtgggggcggcctccccttaccaccggcccttcttggttgcag

SEQ ID NO: 47 - DNA, 36 bp, Porcine
gtcccagtaccctcagcccaagctgcttgagccaaa

SEQ ID NO: 48 - DNA, 849 bp, Porcine
gtaggtgtcaattaggggcggggcacagaagggagactcctggggcggaggtgggggggacagagcgctgattgacaagttggggtggtggaggggtcaggtggccttgggagccgggtggtctggcacctgggctccagtccagccctgtcactagctgtgtggcctacccaactgctctgagcttttcctgcgtgggtggatagtaatacccccacctggagcgttcccgctgtggctcagcaggtgaaggacccagtgaggtctccgtgaggatgcgggctccatccctggcctcgctcagtgggttaaggacctggcgtggctgcaagctgtgccacaggtcgcatatgcggctcagggctggtgtggctgtggctgtggcgtaggccgaagctgcagctccagttctccacccctggcccgggaacttccatgcgccacaggtacggccatactgataataataacaataatagtaataatgataatacccacctcataggaggttacagggcccgacgagatggtgtttgcaaaacgcagggcactgtgcctgcgccctacggggtgcccgacccaccgttaataatggtatcaatgactcccgtttctgaggcacttggcagacaccagaaatgccaggcctttccagaccctggacgcctggtcctcccgaccatgctgagaagtagctgttactacccacactttccacgtgaggctcctggagcccagagacaggagtgaagctgcccagggccacacagcacaggaggcaggaccaggatgagactgaggctttcacaaggggagcgtctcagcccccacggcctcctgtgctgccag

SEQ ID NO: 49 - DNA, 135 bp, Porcine
gccctcagagctcctgacgctcacgtcctggttggcacccatcgtctccgagggcaccttcgaccctgagcttcttcatcacatctaccagccactgaacctgaccatcgggctcacggtgtttgccgtggggaa

SEQ ID NO: 50 - DNA, 1434 bp, Porcine
gtgagtcgtgggctgggcgtggggagggtgggtatagattctgaaccccaggaatgtatggtctggggacagacaggaccccgcccaggcaccagggaggccctgagccaggtgctgagcaggtgggaagcacagggtcgagcgtgatggttgcaggggggcttcctggaggaagggggtctggctctggcagcgaagcaggggagcggcccaggtgagagatcgatggcacctttgtcaggagacaccttgtccccttaccccttctgcttcccctgagccgcccaggcaggtggggagggatagaaagccccccaaccacctcccataaatgggggtccctggtcgggccacacgcaggtcaagagacctgggcagagcagcccggcccccaggagcctctctccaacacgccctcccccggcgggcccgctgccctctgttcagcctgttctcccctctcctccctcagcctgcctggcatttcctaaattaaccgccacctggcagcttccctcggggaccctttctgggagtcctgagagaggggccctaatggggtcctaatgcccaaagcgctgtccagatgctggatggctcagcgggggtcaagaccccccctcccccgccaccccagcccagtcagcacccagcatcacaccttccctcgatgcagccactcaccgcctgtgtctataagatgggtgtgtggtccctgcctcctagggagttgacgaggcctgaaggagtcccttaaaacaggagtcccttagaacactgcctggcacttagtaagtgctcaataaaagttagctcaggagttccctggtagcctagcggttaaggtcctggtgttgtcactgctgtggcgcggattggctccctggactgagaacttccacatgttgtgggtgcggggaaaaagaaagttagctctggagttcccatcgtgactcagtggttaatgaatctgactagcatccatgaggacgcaggttcgatcccaggcctcgctcagtgagttaaggatccgacattgccatgagctgtggtgtaggtcgcagacacggctcggatctggcatgactgtggctgtggcgtaggccgtcggctacagctctgattggacccctagcctggaaacctccatatgccgtgggtgcagccctcaaaagacaaacaaaaaaggttagctcagtctgtgaatgtaagactcctcgagggtcagcctaggacggtcttaagaggctggtgctgtgagtgtgggaatttgacaagtaaggactcggaggagcctcttgagccgggaagctgggaggtggaccccagcctggccgaccctgggctctgtgccccgtgtggtgccagcccgtggtggggactcaggcagtggccctgctgaggcggtggtggccactgggctctcgtccacag

SEQ ID NO: 51 - DNA, 3160 bp, Homo sapiens
ggtggcggagcccgggaggcggagaaggctgtcgttgccttggccgtcgcatccccgagggagtcgtgtcggcgccaccccggcccccgagcccgcagattgcccaccgaagctcgtgtgtgcacccccgatcccgccagccactcgcccctggcctcgcgggccgtgtctccggcatcatgtgtggtatatttgcttacttaaactaccatgttcctcgaacgagacgagaaatcctggagaccctaatcaaaggccttcagagactggagtacagaggatatgattctgctggtgtgggatttgatggaggcaatgataaagattgggaagccaatgcctgcaaaatccagcttattaagaagaaaggaaaagttaaggcactggatgaagaagttcacaagcaacaagatatggatttggatatagaatttgatgtacaccttggaatagctcatacccgttgggcaacacatggagaacccagtcctgtcaatagccacccccagcgctctgataaaaataatgaatttatcgttattcacaatggaatcatcaccaactacaaagacttgaaaaagtttttggaaagcaaaggctatgacttcgaatctgaaacagacacagagacaattgccaagctcgttaagtatatgtatgacaatcgggaaagtcaagataccagctttactaccttggtggagagagttatccaacaattggaaggtgcttttgcacttgtgtttaaaagtgttcattttcccgggcaagcagttggcacaaggcgaggtagccctctgttgattggtgtacggagtgaacataaactttctactgatcacattcctatactctacagaacaggcaaagacaagaaaggaagctgcaatctctctcgtgtggacagcacaacctgccttttcccggtggaagaaaaagcagtggagtattactttgcttctgatgcaagtgctgtcatagaacacaccaatcgcgtcatctttctggaagatgatgatgttgcagcagtagtggatggacgtctttctatccatcgaattaaacgaactgcaggagatcaccccggacgagctgtgcaaacactccagatggaactccagcagatcatgaagggcaacttcagttcatttatgcagaaggaaatatttgagcagccagagtctgtcgtgaacacaatgagaggaagagtcaactttgatgactatactgtgaatttgggtggtttgaaggatcacataaaggagatccagagatgccggcgtttgattcttattgcttgtggaacaagttaccatgctggtgtagcaacacgtcaagttcttgaggagctgactgagttgcctgtgatggtggaactagcaagtgacttcctggacagaaacacaccagtctttcgagatgatgtttgctttttccttagtcaatcaggtgagacagcagatactttgatgggtcttcgttactgtaaggagagaggagctttaactgtggggatcacaaacacagttggcagttccatatcacgggagacagattgtggagttcatattaatgctggtcctgagattggtgtggccagtacaaaggcttataccagccagtttgtatcccttgtgatgtttgcccttatgatgtgtgatgatcggatctccatgcaagaaagacgcaaagagatcatgcttggattgaaacggctgcctgatttgattaaggaagtactgagcatggatgacgaaattcagaaactagcaacagaactttatcatcagaagtcagttctgataatgggacgaggctatcattatgctacttgtcttgaaggggcactgaaaatcaaagaaattacttatatgcactctgaaggcatccttgctggtgaattgaaacatggccctctggctttggtggataaattgatgcctgtgatcatgatcatcatgagagatcacacttatgccaagtgtcagaatgctcttcagcaagtggttgctcggcaggggcggcctgtggtaatttgtgataaggaggatactgagaccattaagaacacaaaaagaacgatcaaggtgccccactcggtggactgcttgcagggcattctcagcgtgatccctttacagttgctggctttccaccttgctgtgctgagaggctatgatgttgatttcccacggaatcttgccaaatctgtgactgtagagtgaggaatatctatacaaaatgtacgaaactgtatgattaagcaacacaagacaccttttgtatttaaaaccttgatttaaaatatcaccacttgaagcctttttttagtaaatccttatttatatatcagttataattattccactcaatatgtgatttttgtgaagttacctcttacattttcccagtaatttgtggaggactttgaataatggaatctatattggaatctgtatcagaaagattctagctattattttctttaaagaatgctgggtgttgcatttctggaccctccacttcaatctgagaagacaatatgtttctaaaaattggtacttgtttcaccatacttcattcagaccagtgaaagagtagtgcatttaattggagtatctaaagccagtggcagtgtatgctcatacttggacagttagggaagggtttgccaagttttaagagaagatgtgatttattttgaaatttgtttctgttttgtttttaaatcaaactgtaaaacttaaaactgaaaaattttattggtaggatttatatctaagtttggttagccttagtttctcagacttgttgtctattatctgtaggtggaagaaatttaggaagcgaaatattacagtagtgcattggtgggtctcaatccttaacatatttgcacaattttatagcacaaactttaaattcaagctgctttggacaactgacaatatgattttaaatttgaagatgggatgtgtacatgttgggtatcctactactttgtgttttcatctcctaaaagtggtttttatttccttgtatctgtagtcttttattttttaaatgactgctgaatgacatattttatcttgttctttaaaatcacaacacagagctgctattaaattaatattgatatattcaaaaaaaaaaaaaaaaa

SEQ ID NO: 52 - DNA, 2663 bp, Homo sapiens
atgggcctggggcctgcctgggtcacacagccttgcctggtcactgactcccagcctgatgcggaattactctcctcaagagcaccctgcctaggtcggcggtgctgctggtccccgggcagaggaggcgtgggcggctccgggaccacggagcctggtgacgcggcgctcccctgcccgggtcgggttgcccaggcgccgccgcggcggctgctgctgctgctgccgctgctgctgggtaggggacttcgagtaacggccgaggcctcggcctcctcctctggggcggcggtcgagaacagcagcgccatggaggagctcgtcactgagaaggaggcggaagagagccaccggccagacagtgtgagcctgctcaccttcatcctgctgctcacgctggccatcctcaccatatggctcttcaagtactgccgggtgcactttctgcatgagaccgggctggccatgatctgtgggctcatcgttggggtgatcctgaggtatggtacccctggcaccaggggccgtgacaaattactcaattgcactcaagaagatcaggccttcagcactttagtagtggatgtcagcggtaaattcttcgaatacaccctgaaaagagaaatcagccctggcaagatcaacagcgtaaagcagaatgacatgctagggaaggtaacattcgacccataggtatttttcaacattcttctgcctccagttattttccatgctggatacagcttaaagagacacttttttagaaatcttgggtcactccttcttggggactgctgtttcgtgcttccgtattggaaatctcaggtatggtatggtgaagctcatgaggattatgagacagctctcagataaattttactacacacattgtctcttttttagagcaatcatctctgccactgacccagtgactgtgctggtgatatcaatgaattgcatgcagacatggatctttatgtacttctgtttggagagagcatcctaaatgacgttgttatgttgtactttcctcatctattgttggctaccagccagcaggactgaacttcaactcacgcctttgatgctgctgcctttttaaagtcagttggcatttttctaggtatatttagtggctgttttaccatgggagctgtgactggtgttgtgactgctttagtgaccaagtttaccaaactggactgctttcccctgctggagacggcgctcttcttcctcatgtcctggagcacgtttctcttggcagaagcttgcggatttacaggcgttgtagctgtccttttctgtggaatcacacaagctcattacaccttcaacaatctgtcggtggaatcaagaagtcgaagcaagcagctctttgaggcagagaacttcatcttctcctgcatgatcctggcgctatttaccttccagaagcacgttttcagccctgttttcatcattggagcttttgttgctgtcttcctgggcagagccgcccatatctacccgctctctttcttcctcagcttgggcagaaggcataagattggctggaattttcaacacacgatgatgttttcaggcctcaggggagcaatggcatttgcgttggccatctgtgacacggcatcctatgctcgccagatgacgttccccaccacgcctttcatcgtgttcttcaccatctggatcattggaggaggcacgacacccatgttgtcatggcttaatatcagagttagcatcaaggagccctccaaagaggaccacaacgaacaccaccgacagtacttcagagttggtgttgaccctgatcaagatccaccacccaacaatgacagctttcaagtcttacaaggggacagcccagattctgccagaggaaactggacaaaacaggagagcacatggatattcaggcggtggtacagctttgatcacaattacctgaagcccatcctcacacacagcggctccccgctaaccaccactctcccgcctggtggagacacagcggctccccgctaaccaccactctcctgcctggtgtagacaaagcggctccccgccaaccaccactctcccgcctggtgtagcttgctagcttgatgtctgaccagtccccaggtgtacgataaccaagagccactgagagagggaaactctgattttattctgactgaaggcgacctcacattgacctatggggacagcacagtgactgcaaatggcttctcaggttcccacactgcctccacgagtctggagggcagctggagaatgaagagcagctcagaggaagtgctggagcaggacgtgggaatgggaaaccagaaggtttcgagccagggtacccgcctagtgtttcctctggaagataatgtttgactttccctgcaaaccctggcacgatggggtaggctcccaatggggtgaggatggcttcaagccctaatgttgcttgaggtggggcagtgactagattgaattaactcttctattttattggggtctgaagttattgtaacacttaaaatttaactcatgatgcagatggtgaggcaaaagtgtctctaaattcagacaaatgtagacctatttctactttttttcacacagtagtgcgctgtttcagagttaaacaaacaaaaaaatagcat

SEQ ID NO: 53 - DNA, 1309 bp, Porcine
gtacacccagttcgtccagcgcttcctggagtcggccgagcgcttcttcatgcagggctaccgggtgcactactacatctttaccagcgaccccggggccgttcctggggtcccgctgggcccgggccgcctcctcagcgtcatcgccatccggagaccctcccgctgggaggaggtctccacacgccggatggaggccatcagccagcacattgccgccagggcgcaccgggaggtcgactacctcttctgcctcagcgtggacatggtgttccggaacccatggggccccgagaccttgggggacctggtggctgccattcacccgggctacttcgccgcgccccgccagcagttcccctacgagcgccggcatgtttctaccgccttcgtggcggacagcgagggggacttctattatggtggggcggtcttcggggggcgggtggccagggtgtacgagttcacccagggctgccacatgggcatcctggcggacaaggccaatggcatcatggcggcctggcaggaggagagccacctgaaccgccgcttcatctcccacaagccctccaaggtgctgtcccccgagtacctctgggatgaccgcaggccccagccccccagcctgaagctgatccgcttttccacactggacaaagacaccaactggctgaggagctgacagcacagccggggctgctgtgcatgcggggggaccccaagccctgcccccagctcgccccagcagcgcctcctcacccggacgcctcacttcccaagccttctgtgaaaccagccctgcgctgcctacctctcaggctgccagcagactccgaggcctgtgtaaactgtgaagggctgtgcccttgtgagaacacacagcctgtgagccagaaacggtcagacgggaggagacggaccagaggtagaagaagacgggacccgcagtcctcacccagcccacgtgcctttggggtgggcgctggagggtcagccctgcccagtgcctgacgtcccgcccaccccccttttgtggccgtttgtacctctgacacatgagagaggtatcctggacccctgtcctctggctgcaggggccccggggactgttctgtccccctgccacaaggagccagtacctcactcaggaccccgaccgagccttcgaaatggaccccgcctgggctctctcgttccacgtccagcccacctctgcagtggaccacgctccctggtgcccaccgcctcctttgcaagggggtttgggcagctttttaatacaggtggcatgtgctcagccctaacc

SEQ ID NO: 54 - DNA, 28 bp, Artificial
  Primer
gatggccactagtctcctttgatcagca

SEQ ID NO: 55 - DNA, 27 bp, Artificial
  Primer
gttctagtgagctctgctctgcagaag

SEQ ID NO: 56 - DNA, 27 bp, Artificial
  Primer
cttctgcagagcagagctcactagaac

SEQ ID NO: 57 - DNA, 25 bp, Artificial
  Primer
tctcaagcacaggccagtcgggccg

SEQ ID NO: 58 - DNA, 22 bp, Artificial
  Primer
gcggactagttcggccctggct

SEQ ID NO: 59 - DNA, 25 bp, Artificial
  Primer
tgcagcgtctgagctcgagcacctg

SEQ ID NO: 60 - DNA, 25 bp, Artificial
  Primer
cctcagagctcctgacgctcacgtc

SEQ ID NO: 61 - DNA, 26 bp, Artificial
  Primer
cgtctggccgttctggcccacaggct

SEQ ID NO: 62 - DNA, 28 bp, Artificial
  Primer
agctctcaactactagtttccaagccac

SEQ ID NO: 63 - DNA, 27 bp, Artificial
  Primer
acggggacaagtacttccagatgcctg

SEQ ID NO: 64 - DNA, 25 bp, Artificial
  Primer
atgcgaagggtcagagctctaaagg

SEQ ID NO: 65 - DNA, 25 bp, Artificial
  Primer
ttggcctagagggccagtcttgctc

SEQ ID NO: 66 - DNA, 900 bp, Sus scrofa
ttgtcacactgggcactaaagcaccaactctgaaatataatttttgattatgttccctcctaaaataactaaagcacaaactctgaaatataattttcgtttacgttctctccctctactaatattccagcagagaacagagcccgcgccaggtgtccagtacccagcccctcatatccgaagctcaggacttgggggtttcgggagagagcggctccagcgcgtcgggttgtagctactgcatctgtgctcttccttccccaggaaacaaatggtggatcggacctcccaggctcttcgcgccccgccacccctccccgtgttagcagggcgcagggctccggggcccctccctgcagtactgggtgatagaccccactccaccctccgggtccctccacccccaccacgtgcaggccagagaaggcaaagaggcccagccaccctcaccagggaatttcttttctttttttgctggtttcaggcttttttctgcctgagtgaaaatgaaacaaacaccccctgcgcctcccggccaccagacacacacgcgcaccggcactcgcgcactcgcgccctcggcctcctagcggccgtgtctggggcgggacccgctctgcacaaacagccgcgggccgggtggagcggggagctcgccgcccgccgcccagtgcccgccggcttcctcgcgcccctgcccgccaccccggaggagcacacagcggccggcgggccggagcgcaggcggcacaccccgccccggcacgccctgccgagctcaggagcacgccgcgcgccactgttccctcagccgaggacgccgccggggggccgggagccgaggtgtgggccatccccgagcgcacccagcttctgccgatcaggtgggtcccgctgggc